(19) United States
(12) Patent Application Publication
(10) Pub. No.: US 2005/0186568 A1
(43) Pub. Date: Aug. 25, 2005

(54) Kinases and phosphatases

(76) Inventors: Olga Bandman, Mountain View, CA (US); Mariah R Baughn, Los Angeles, CA (US); Shanya D Becha, San Francisco, CA (US); Mark L Borowsky, Needham, MA (US); Brendan M Duggan, Sunnyvale, CA (US); Brooke M Emerling, Chicago, IL (US); Ian J Forsythe, Edmonton, CA (US); Ameena R Ganhi, San Francisco, CA (US); Jennifer A Griffin, Fremont, CA (US); Rajagopal Gururajan, San Jose, CA (US); April J.A. Hafalia, Daly City, CA (US); Farrah A Khan, Canton, MI (US); Preeti G Lal, Santa Clara, CA (US); Ernestine A Lee, Kensington, CA (US); Soo Yeun Lee, Mountain View, CA (US); Erika A Lindquist, Alameda, CA (US); Dyung Aina M Lu, San Jose, CA (US); Yan Lu, Mountain View, CA (US); Joseph P Marquis, San Jose, CA (US); Danniel B Nguyen, San Jose, CA (US); Chandra S Arvizu, San Diego, CA (US); Jayalaxmi Ramkumar, Fremont, CA (US); Shirley A Recipon, San Francisco, CA (US); Thomas W Richardson, Redwood City, CA (US); Anita Swarnakar, San Francisco, CA (US); Y.Tom Tang, San Jose, CA (US); Michael B Thornton, Oakland, CA (US); Uyen K Tran, San Jose, CA (US); Narinder K Chawla, Union City, CA (US); Bridget A Warren, San Marcos, CA (US); Junming Yang, San Jose, CA (US); Monique G Yao, Mountain View, CA (US); Henry Yue, Sunnyvale, CA (US); Yeganeh Zebarjadian, San Francisco, CA (US)

Correspondence Address: INCYTE CORPORATION;EXPERIMENTAL STATION, ROUTE 141 & HENRY CLAY ROAD, BLDG. E336, WILMINGTON, DE, 19880, US

(21) Appl. No.: 10/491,467

(22) PCT Filed: Oct. 17, 2002
(86) PCT No.: PCT/US02/33723

Related U.S. Application Data
(60) Provisional application No. 60/345,474, filed on Oct. 19, 2001.
(60) Provisional application No. 60/343,910, filed on Nov. 2, 2001.
(60) Provisional application No. 60/333,098, filed on Nov. 13, 2001.
(60) Provisional application No. 60/332,424, filed on Nov. 16, 2001.
(60) Provisional application No. 60/334,288, filed on Nov. 30, 2001.

(51) Int. Cl. C12Q 1/68; C07H 21/04; C12N 9/12; C12N 9/16

(52) U.S. Cl. 435/6; 435/69.1; 435/194; 435/196; 435/320.1; 435/325; 536/23.2

(57) ABSTRACT

Various embodiments of the invention provide human kinases and phosphatases (KPP) and polynucleotides which identify and encode KPP. Embodiments of the invention also provide expression vectors, host cells, antibodies, agonists, and antagonists. Other embodiments provide methods for diagnosing, treating, or preventing disorders associated with aberrant expression of KPP.

### TECHNICAL FIELD

The invention relates to novel nucleic acids, kinases and phosphatases encoded by these nucleic acids, and to the use of these nucleic acids and proteins in the diagnosis, treatment, and prevention of cardiovascular diseases, immune system disorders, neurological disorders, disorders affecting growth and development, lipid disorders, cell proliferative disorders, and cancers. The invention also relates to the assessment of the effects of exogenous compounds on the expression of nucleic acids and kinases and phosphatases.

### BACKGROUND OF THE INVENTION

Reversible protein phosphorylation is the ubiquitous strategy used to control many of the intracellular events in eukaryotic cells. It is estimated that more than ten percent of proteins active in a typical mammalian cell are phosphorylated. Kinases catalyze the transfer of high-energy phosphate groups from adenosine triphosphate (ATP) to target proteins on the hydroxyamino acid residues serine, threonine, or tyrosine. Phosphatases, in contrast, remove these phosphate groups. Extracellular signals including hormones, neurotransmitters, and growth and differentiation factors can activate kinases, which can occur as cell surface receptors or as the activator of the final effector protein, as well as other locations along the signal transduction pathway. Cascades of kinases occur, as well as kinases sensitive to second messenger molecules. This system allows for the amplification of weak signals (low abundance growth factor molecules, for example), as well as the synthesis of many weak signals into an all-or-nothing response. Phosphatases, then, are essential in determining the extent of phosphorylation in the cell and, together with kinases, regulate key cellular processes such as metabolic enzyme activity, proliferation, cell growth and differentiation, cell adhesion, and cell cycle progression.

### Kinases

Kinases comprise the largest known enzyme superfamily and vary widely in their target molecules. Kinases catalyze the transfer of high energy phosphate groups from a phosphate donor to a phosphate acceptor. Nucleotides usually serve as the phosphate donor in these reactions, with most kinases utilizing adenosine triphosphate (ATP). The phosphate acceptor can be any of a variety of molecules, including nucleosides, nucleotides, lipids, carbohydrates, and proteins. Proteins are phosphorylated on hydroxyamino acids. Addition of a phosphate group alters the local charge on the acceptor molecule, causing internal conformational changes and potentially influencing intermolecular contacts. Reversible protein phosphorylation is the primary method for regulating protein activity in eukaryotic cells. In general, proteins are activated by phosphorylation in response to extracellular signals such as hormones, neurotransmitters, and growth and differentiation factors. The activated proteins initiate the cell's intracellular response by way of intracellular signaling pathways and second messenger molecules such as cyclic nucleotides, calcium-calmodulin, inositol, and various mitogens, that regulate protein phosphorylation.

Kinases are involved in all aspects of a cell's function, from basic metabolic processes, such as glycolysis, to cell-cycle regulation, differentiation, and communication with the extracellular environment through signal transduction cascades. Inappropriate phosphorylation of proteins in cells has been linked to changes in cell cycle progression and cell differentiation. Changes in the cell cycle have been linked to induction of apoptosis or cancer. Changes in cell differentiation have been linked to diseases and disorders of the reproductive system, immune system, and skeletal muscle.

There are two classes of protein kinases. One class, protein tyrosine kinases (PIKs), phosphorylates tyrosine residues, and the other class, protein serine/threonine kinases (STKs), phosphorylates. serine and threonine residues. Some PTKs and STKs possess structural characteristics of both families and have dual specificity for both tyrosine and serine/threonine residues. Almost all kinases contain a conserved 250-300 amino acid catalytic domain containing specific residues and sequence motifs characteristic of the kinase family. The protein kinase catalytic domain can be further divided into 11 subdomains. N-terminal subdomains I-IV fold into a two-lobed structure which binds and orients the ATP donor molecule, and subdomain V spans the two lobes. C-terminal subdomains VI-XI bind the protein substrate and transfer the gamma phosphate from ATP to the hydroxyl group of a tyrosine, serine, or threonine residue. Each of the 11 subdomains contains specific catalytic residues or amino acid motifs characteristic of that subdomain. For example, subdomain I contains an 8-amino acid glycine-rich ATP binding consensus motif, subdomain II contains a critical lysine residue required for maximal catalytic activity, and subdomains VI through IX comprise the highly conserved catalytic core. PTKs and STKs also contain distinct sequence motifs in subdomains VI and VIII which may confer hydroxyamino acid specificity.

In addition, kinases may also be classified by additional amino acid sequences, generally between 5 and 100 residues, which either flank or occur within the kinase domain. These additional amino acid sequences regulate kinase activity and determine substrate specificity. (Reviewed in Hardie, G. and S. Hanks (1995) The Protein Kinase Facts Book, Vol I, pp. 17-20 Academic Press, San Diego Calif.). In particular, two protein kinase signature sequences have been identified in the kinase domain, the first containing an active site lysine residue involved in ATP binding, and the second containing an aspartate residue important for catalytic activity. If a protein analyzed includes the two protein kinase signatures, the probability of that protein being a protein kinase is close to 100% (PROSITE: PDOC00100, November 1995).

Protein Tyrosine Kinases

Protein tyrosine kinases (PTKs) may be classified as either transmembrane, receptor PTKs or nontransmembrane, nonreceptor PTK proteins. Transmembrane tyrosine kinases function as receptors for most growth factors. Growth factors bind to the receptor tyrosine kinase (RTK), which causes the receptor to phosphorylate itself (autophosphorylation) and specific intracellular second messenger proteins. Growth factors (GF) that associate with receptor PTKs include epidermal GF, platelet-derived GF, fibroblast GF, hepatocyte GF, insulin and insulin-like GFs, nerve GF, vascular endothelial GF, and macrophage colony stimulating factor.

Nontransmembrane, nonreceptor PTKS lack transmembrane regions and, instead, form signaling complexes with the cytosolic domains of plasma membrane receptors. Receptors that function through non-receptor PTKs include those for cytokines and hormones (growth hormone and prolactin), and antigen-specific receptors on T and B lymphocytes.

Many PTKs were first identified as oncogene products in cancer cells in which PTK activation was no longer subject to normal cellular controls. In fact, about one third of the known oncogenes encode PTKs. Furthermore, cellular transformation (oncogenesis) is often accompanied by increased tyrosine phosphorylation activity (Charbonneau, H. and N. K. Tonks (1992) Annu. Rev. Cell Biol. 8:463-493). Regulation of PTK activity may therefore be an important strategy in controlling some types of cancer.

Protein Serine/Threonine Kinases

Protein serine/threonine kinases (STKs) are nontransmembrane proteins. A subclass of STKs are known as ERKs (extracellular signal regulated kinases) or MAPs (mitogen-activated protein kinases) and are activated after cell stimulation by a variety of hormones and growth factors. Cell stimulation induces a signaling cascade leading to phosphorylation of MEK (MAP/ERK kinase) which, in turn, activates ERK via serine and threonine phosphorylation. A varied number of proteins represent the downstream effectors for the active ERK and implicate it in the control of cell proliferation and differentiation, as well as regulation of the cytoskeleton. Activation of ERK is normally transient, and cells possess dual specificity phosphatases that are responsible for its down-regulation. Also, numerous studies have shown that elevated ERK activity is associated with some cancers. Other STKs include the second messenger dependent protein kinases such as the cyclic-AMP dependent protein kinases (PKA), calcium-calmodulin (CaM) dependent protein kinases, and the mitogen-activated protein kinases (MAP); the cyclin-dependent protein kinases; checkpoint and cell cycle kinases; Numb-associated kinase (Nak); human Fused (hFu); proliferation-related kinases; 5′-AMP-activated protein kinases; and kinases involved in apoptosis.

One member of the ERK family of MAP kinases, ERK 7, is a novel 61-kDa protein that has motif similarities to ERK1 and ERK2, but is not activated by extracellular stimuli as are ERK1 and ERK2 nor by the common activators, c-Jun N-terminal kinase (JNK) and p38 kinase. ERK7 regulates its nuclear localization and inhibition of growth through its C-terminal tail, not through the kinase domain as is typical with other MAP kinases (Abe, M. K. (1999) Mol. Cell. Biol. 19:1301-1312).

The second messenger dependent protein kinases primarily mediate the effects of second messengers such as cyclic AMP (cAMP), cyclic GMP, inositol triphosphate, phosphatidylinositol, 3,4,5-triphosphate, cyclic ADP ribose, arachidonic acid, diacylglycerol and calcium-calmodulin. The PKAs are involved in mediating hormone-induced cellular responses and are activated by cAMP produced within the cell in response to hormone stimulation. cAMP is an intracellular mediator of hormone action in all animal cells that have been studied. Hormone-induced cellular responses include thyroid hormone secretion, cortisol secretion, progesterone secretion, glycogen breakdown, bone resorption, and regulation of heart rate and force of heart muscle contraction. PKA is found in all animal cells and is thought to account for the effects of cAMP in most of these cells. Altered PKA expression is implicated in a variety of disorders and diseases including cancer, thyroid disorders, diabetes, atherosclerosis, and cardiovascular disease (Isselbacher, K. J. et al. (1994) Harrison's Principles of Internal Medicine, McGraw-Hill, New York N.Y., pp. 416-431, 1887).

The casein kinase I (CKI) gene family is another subfamily of serine/threonine protein kinases. This continuously expanding group of kinases have been implicated in the regulation of numerous cytoplasmic and nuclear processes, including cell metabolism and DNA replication and repair. CKI enzymes are present in the membranes, nucleus, cytoplasm and cytoskeleton of eukaryotic cells, and on the mitotic spindles of mammalian cells (Fish, K. J. et al. (1995) J. Biol. Chem. 270:14875-14883).

The CKI family members all have a short amino-terminal domain of 9-76 amino acids, a highly conserved kinase domain of 284 amino acids, and a variable carboxyl-terminal domain that ranges from 24 to over 200 amino acids in length (Cegielska, A. et al. (1998) J. Biol. Chem. 273:1357-1364). The CKI family is comprised of highly related proteins, as seen by the identification of isoforms of casein kinase I from a variety of sources. There are at least five mammalian isoforms, α, β, γ, δ, and ε. Fish et al. identified CKI-epsilon from a human placenta cDNA library. It is a basic protein of 416 amino acids and is closest to CKI-delta. Through recombinant expression, it was determined to phosphorylate known CKI substrates and was inhibited by the CKI-specific inhibitor CKI-7. The human gene for CKI-epsilon was able to rescue yeast with a slow-growth phenotype caused by deletion of the yeast CKI locus, HRR250 (Fish et al., supra).

The mammalian circadian mutation tau was found to be a semidominant autosomal allele of CKI-epsilon that markedly shortens period length of circadian rhythms in Syrian hamsters. The tau locus is encoded by casein kinase I-epsilon, which is also a homolog of the Drosophila circadian gene double-time. Studies of both the wildtype and tau mutant CKI-epsilon enzyme indicated that the mutant enzyme has a noticeable reduction in the maximum velocity and autophosphorylation state. Further, in vitro, CKI-epsilon is able to interact with mammalian PERIOD proteins, while the mutant enzyme is deficient in its ability to phosphorylate PERIOD. Lowrey et al. have proposed that CKI-epsilon plays a major role in delaying the negative feedback signal within the transcription-translation-based autoregulatory loop that composes the core of the circadian mechanism. Therefore the CKI-epsilon enzyme is an ideal target for pharmaceutical compounds influencing circadian rhythms, jet-lag and sleep, in addition to other physiologic and metabolic processes under circadian regulation (Lowrey, P. L. et al. (2000) Science 288:483-491).

Homeodomain-interacting protein kinases (HIPKs) are serine/threonine kinases and novel members of the DYRK kinase subfamily (Hofmann, T. G. et al. (2000) Biochimie 82:1123-1127). HIPKs contain a conserved protein kinase domain separated from a domain that interacts with homeoproteins. HIPKs are nuclear kinases, and HIPK2 is highly expressed in neuronal tissue (Kim, Y. H. et al. (1998) J. Biol. Chem. 273:25875-25879; Wang, Y. et al. (2001) Biochim. Biophys. Acta 1518:168-172). HIPKs act as corepressors for homeodomian transcription factors. This corepressor activity is seen in posttranslational modifications such as ubiquitination and phosphorylation, each of which are important in the regulation of cellular protein function (Kim, Y. H. et al. (1999) Proc. Natl. Acad. Sci. USA 96:12350-12355).

The human h-warts protein, a homolog of Drosophila warts tumor suppressor gene, maps to chromosome 6q24-25.1. It has a serine/threonine kinase domain and is localized to centrosomes in interphase cells. It is involved in mitosis and functions as a component of the mitotic apparatus (Nishiyama, Y. et al. (1999) FEBS Lett. 459:159-165).

The Cdc42/Rac-binding p21-activated kinase (PAK) and Rho-binding kinase (ROK) act as morphological effectors for Rho GTPases which function in actin reorganization. The 190-kDa myotonic dystrophy kinase-related Cdc42-binding kinase (MRCK) is a brain Cdc42-binding serine/threonine kinase whose p21-binding domain resembles that of PAK whereas the kinase domain resembles that of myotonic dystrophy kinase-related ROK. MRCK phosphorylates nonmuscle myosin light chain at serine 19, crucial for activating actin-myosin contractility. It is involved in peripheral actin formation and neurite outgrowth in HeLa and PC12 cells, respectively (Tan, I. et al. (2001) Mol. Cell. Biol. 21:2767-2778; Tan, I. et al. (2001) J. Biol. Chem. 276:21209-21216; Leung, T. (1998) Mol. Cell. Biol. 18:130-140).

The EMK (ELKL Motif Kinase) is a small family of serine/threonine protein kinases involved in the control of cell polarity, microtubule stability and cancer. EMK1 (ELKL motif kinase 1, MARK2) has two isoforms, one of which contains a 162-bp alternative exon and one which does not. Both forms are coexpressed in cell lines and tissue samples examined. Human EMK1 is ubiquitously expressed. EMK1 contains a minimum of 16 small exons (Espinosa, L. and Navarro, E. (1998) Cytogenet. Cell Genet. 81:278-282).

Calcium-Calmodulin Dependent Protein Kinases

Calcium-calmodulin dependent (CaM) kinases are involved in regulation of smooth muscle contraction, glycogen breakdown (phosphorylase kinase), and neurotransmission (CaM kinase I and CaM kinase II). CaM dependent protein kinases are activated by calmodulin, an intracellular calcium receptor, in response to the concentration of free calcium in the cell. Many CaM kinases are also activated by phosphorylation. Some CaM kinases are also activated by autophosphorylation or by other regulatory kinases. CaM kinase I phosphorylates a variety of substrates including the neurotransmitter-related proteins synapsin I and II, the gene transcription regulator, CREB, and the cystic fibrosis conductance regulator protein, CFTR (Haribabu, B. et al. (1995) EMBO J. 14:3679-3686). CaM kinase II also phosphorylates synapsin at different sites and controls the synthesis of catecholamines in the brain through phosphorylation and activation of tyrosine hydroxylase. CaM kinase II controls the synthesis of catecholamines and seratonin, through phosphorylation/activation of tyrosine hydroxylase and tryptophan hydroxylase, respectively (Fujisawa, H. (1990) BioEssays 12:27-29). The mRNA encoding a calmodulin-binding protein kinase-like protein was found to be enriched in mammalian forebrain. This protein is associated with vesicles in both axons and dendrites and accumulates largely postnatally. The amino acid sequence of this protein is similar to CaM-dependent STKs, and the protein binds calmodulin in the presence of calcium (Godbout, M. et al. (1994) J. Neurosci. 14:1-13).

Mitogen-Activated Protein Kinases

The mitogen-activated protein kinases (MAP), which mediate signal transduction from the cell surface to the nucleus via phosphorylation cascades, are another STK family that regulates intracellular signaling pathways. Several subgroups have been identified, and each manifests different substrate specificities and responds to distinct extracellular stimuli (Egan, S. E. and R. A. Weinberg (1993) Nature 365:781-783). There are three kinase modules comprising the MAP kinase cascade: MAPK (MAP), MAPK kinase (MAP2K, MAPKK, or MKK), and MKK kinase (MAP3K, MAPKKK, OR MEKK) (Wang, X. S. et al (1998) Biochem. Biophys. Res. Commun. 253:33-37). The extracellular-regulated kinase (ERK) pathway is activated by growth factors and mitogens, for example, epidermal growth factor (EGF), ultraviolet light, hyperosmolar medium, heat shock, or endotoxic lipopolysaccharide (LPS). The closely related though distinct parallel pathways, the c-Jun N-terminal kinase (JNK), or stress-activated kinase (SAPK) pathway, and the p38 kinase pathway are activated by stress stimuli and proinflammatory cytokines such as tumor necrosis factor (TNF) and interleukin-1 (IL-1). Altered MAP kinase expression is implicated in a variety of disease conditions including cancer, inflammation, immune disorders, and disorders affecting growth and development. MAP kinase signaling pathways are present in mammalian cells as well as in yeast.

Cyclin-Dependent Protein Kinases

The cyclin-dependent protein kinases (CDKs) are STKs that control the progression of cells through the cell cycle. The entry and exit of a cell from mitosis are regulated by the synthesis and destruction of a family of activating proteins called cyclins. Cyclins are small regulatory proteins that bind to and activate CDKs, which then phosphorylate and activate selected proteins involved in the mitotic process. CDKs are unique in that they require multiple inputs to become activated. In addition to cyclin binding, CDK activation requires the phosphorylation of a specific threonine residue and the dephosphorylation of a specific tyrosine residue on the CDK.

Another family of STKs associated with the cell cycle are the NIMA (never in mitosis)-related kinases (Neks). Both CDKs and Neks are involved in duplication, maturation, and separation of the microtubule organizing center, the centrosome, in animal cells (Fry, A. M. et al. (1998) EMBO J. 17:470-481).

Checkpoint and Cell Cycle Kinases

In the process of cell division, the order and timing of cell cycle transitions are under control of cell cycle checkpoints, which ensure that critical events such as DNA replication and chromosome segregation are carried out with precision. If DNA is damaged, e.g. by radiation, a checkpoint pathway is activated that arrests the cell cycle to provide time for repair. If the damage is extensive, apoptosis is induced. In the absence of such checkpoints, the damaged DNA is inherited by aberrant cells which may cause proliferative disorders such as cancer. Protein kinases play an important role in this process. For example, a specific kinase, checkpoint kinase 1 (Chk1), has been identified in yeast and mammals, and is activated by DNA damage in yeast. Activation of Chk1 leads to the arrest of the cell at the G2/M transition (Sanchez, Y. et al. (1997) Science 277:1497-1501). Specifically, Chk1 phosphorylates the cell division cycle phosphatase CDC25, inhibiting its normal function which is to dephosphorylate and activate the cyclin-dependent kinase Cdc2. Cdc2 activation controls the entry of cells into mitosis (Peng, C.-Y. et al. (1997) Science 277:1501-1505). Thus, activation of Chk1 prevents the damaged cell from entering mitosis. A deficiency in a checkpoint kinase, such as Chk1, may also contribute to cancer by failure to arrest cells with damaged DNA at other checkpoints such as G2/M.

Proliferation-Related Kinases

Proliferation-related kinase is a serum/cytokine inducible STK that is involved in regulation of the cell cycle and cell proliferation in human megakarocytic cells (Li, B. et al. (1996) J. Biol. Chem. 271:19402-19408). Proliferation-related kinase is related to the polo (derived from Drosophila polo gene) family of STKs implicated in cell division. Proliferation-related kinase is downregulated in lung tumor tissue and may be a proto-oncogene whose deregulated expression in normal tissue leads to oncogenic transformation.

5′-AMP-activated Protein Kinase

A ligand-activated STK protein kinase is 5′-AMP-activated protein kinase (AMPK) (Gao, G. et al. (1996) J. Biol Chem. 271:8675-8681). Mammalian AMPK is a regulator of fatty acid and sterol synthesis through phosphorylation of the enzymes acetyl-CoA carboxylase and hydroxymethylglutaryl-CoA reductase and mediates responses of these pathways to cellular stresses such as heat shock and depletion of glucose and ATP. AMPK is a heterotrimeric complex comprised of a catalytic alpha subunit and two non-catalytic beta and gamma subunits that are believed to regulate the activity of the alpha subunit. Subunits of AMPK have a much wider distribution in non-lipogenic tissues such as brain, heart, spleen, and lung than expected. This distribution suggests that its role may extend beyond regulation of lipid metabolism alone.

Kinases in Apoptosis

Apoptosis is a highly regulated signaling pathway leading to cell death that plays a crucial role in tissue development and homeostasis. Deregulation of this process is associated with the pathogenesis of a number of diseases including autoimmune diseases, neurodegenerative disorders, and cancer. Various STKs play key roles in this process. ZIP kinase is an STK containing a C-terminal leucine zipper domain in addition to its N-terminal protein kinase domain. This C-terminal domain appears to mediate homodimerization and activation of the kinase as well as interactions with transcription factors such as activating transcription factor, ATF4, a member of the cyclic-AMP responsive element binding protein (ATP/CREB) family of transcriptional factors (Sanjo, H. et al. (1998) J. Biol. Chem. 273:29066-29071). DRAK1 and DRAK2 are STKs that share homology with the death-associated protein kinases (DAP kinases), known to function in interferon-γ induced apoptosis (Sanjo et al., supra). Like ZIP kinase, DAP kinases contain a C-terminal protein-protein interaction domain, in the form of ankyrin repeats, in addition to the N-terminal kinase domain. ZIP, DAP, and DRAK kinases induce morphological changes associated with apoptosis when transfected into NIH3T3 cells (Sanjo et al., supra). However, deletion of either the N-terminal kinase catalytic domain or the C-terminal domain of these proteins abolishes apoptosis activity, indicating that in addition to the kinase activity, activity in the C-terminal domain is also necessary for apoptosis, possibly as an interacting domain with a regulator or a specific substrate.

RICK is another STK recently identified as mediating a specific apoptotic pathway involving the death receptor, CD95 (Inohara, N. et al. (1998) J. Biol. Chem. 273:12296-12300). CD95 is a member of the tumor necrosis factor receptor superfamily and plays a critical role in the regulation and homeostasis of the immune system (Nagata, S. (1997) Cell 88:355-365). The CD95 receptor signaling pathway involves recruitment of various intracellular molecules to a receptor complex following ligand binding. This process includes recruitment of the cysteine protease caspase-8 which, in turn, activates a caspase cascade leading to cell death. RICK is composed of an N-terminal kinase catalytic domain and a C-terminal “caspase-recruitment” domain that interacts with caspase-like domains, indicating that RICK plays a role in the recruitment of caspase-8. This interpretation is supported by the fact that the expression of RICK in human 293T cells promotes activation of caspase-8 and potentiates the induction of apoptosis by various proteins involved in the CD95 apoptosis pathway (Inohara et al., supra).

Mitochondrial Protein Kinases

A novel class of eukaryotic kinases, related by sequence to prokaryotic histidine protein kinases, are the mitochondrial protein kinases (MPKs) which seem to have no sequence similarity with other eukaryotic protein kinases. These protein kinases are located exclusively in the mitochondrial matrix space and may have evolved from genes originally present in respiration-dependent bacteria which were endocytosed by primitive eukaryotic cells. MPKs are responsible for phosphorylation and inactivation of the branched-chain alpha-ketoacid dehydrogenase and pyruvate dehydrogenase complexes (Harris, R. A. et al. (1995) Adv. Enzyme Regul. 34:147-162). Five MPKs have been identified. Four members correspond to pyruvate dehydrogenase kinase isozymes, regulating the activity of the pyruvate dehydrogenase complex, which is an important regulatory enzyme at the interface between glycolysis and the citric acid cycle. The fifth member corresponds to a branched-chain alpha-ketoacid dehydrogenase kinase, important in the regulation of the pathway for the disposal of branched-chain amino acids. (Harris, R. A. et al. (1997) Adv. Enzyme Regul. 37:271-293). Both starvation and the diabetic state are known to result in a great increase in the activity of the pyruvate dehydrogenase kinase in the liver, heart and muscle of the rat. This increase contributes in both disease states to the phosphorylation and inactivation of the pyruvate dehydrogenase complex and conservation of pyruvate and lactate for gluconeogenesis (Harris (1995) supra).

### Kinases with Non-protein Substrates

GK2, a human galactokinase, has a predicted length of 458 amino acids with 29% identity to galactokinase of Saccharomyces carlsbergensis. It has been mapped to chromosome 15, whereas GK1 was mapped to chromosome 17q23-25 (Lee, R. T. et al. (1992) Proc Natl Acad Sci U S A 89:10887-10891).

Lipid and Inositol Kinases

Lipid kinases phosphorylate hydroxyl residues on lipid head groups. A family of kinases involved in phosphorylation of phosphatidylinositol (PI) has been described, each member phosphorylating a specific carbon on the inositol ring (Leevers, S. J. et al. (1999) Curr. Opin. Cell. Biol. 11:219-225). The phosphorylation of phosphatidylinositol is involved in activation of the protein kinase C signaling pathway. The inositol phospholipids (phosphoinositides) intracellular signaling pathway begins with binding of a signaling molecule to a G-protein linked receptor in the plasma membrane. This leads to the phosphorylation of phosphatidylinositol (PI) residues on the inner side of the plasma membrane by inositol kinases, thus converting PI residues to the biphosphate state (PIP₂). PIP₂ is then cleaved into inositol triphosphate (IP₃) and diacylglycerol. These two products act as mediators for separate signaling pathways. Cellular responses that are mediated by these pathways are glycogen breakdown in the liver in response to vasopressin, smooth muscle contraction in response to acetylcholine, and thrombin-induced platelet aggregation.

PI3-kinase (PI3K), which phosphorylates the D3 position of PI and its derivatives, has a central role in growth factor signal cascades involved in cell growth, differentiation, and metabolism. PI3K is a heterodimer consisting of an adapter subunit and a catalytic subunit. The adapter subunit acts as a scaffolding protein, interacting with specific tyrosine-phosphorylated proteins, lipid moieties, and other cytosolic factors. When the adapter subunit binds tyrosine phosphorylated targets, such as the insulin responsive substrate (IRS)-1, the catalytic subunit is activated and converts PI (4,5) bisphosphate (PIP₂) to PI (3,4,5) P₃ (PIP₃). PIP₃ then activates a number of other proteins, including PKA, protein kinase B (PKB), protein kinase C (PKC), glycogen synthase kinase (GSK)-3, and p70 ribosomal s6 kinase. PI3K also interacts directly with the cytoskeletal organizing proteins, Rac, rho, and cdc42 (Shepherd, P. R. et al. (1998) Biochem. J. 333:471-490). Animal models for diabetes, such as obese and fat mice, have altered PI3K adapter subunit levels. Specific mutations in the adapter subunit have also been found in an insulin-resistant Danish population, suggesting a role for PI3K in type-2 diabetes (Shepard, supra).

An example of lipid kinase phosphorylation activity is the phosphorylation of D-erythro-sphingosine to the sphingolipid metabolite, sphingosine-1-phosphate (SPP). SPP has emerged as a novel lipid second-messenger with both extracellular and intracellular actions (Kohama, T. et al. (1998) J. Biol. Chem. 273:23722-23728). Extracellularly, SPP is a ligand for the G-protein coupled receptor EDG-1 (endothelial-derived, G-protein coupled receptor). Intracellularly, SPP regulates cell growth, survival, motility, and cytoskeletal changes. SPP levels are regulated by sphingosine kinases that specifically phosphorylate D-erythro-sphingosine to SPP. The importance of sphingosine kinase in cell signaling is indicated by the fact that various stimuli, including platelet-derived growth factor (PDGF), nerve growth factor, and activation of protein kinase C, increase cellular levels of SPP by activation of sphingosine kinase, and the fact that competitive inhibitors of the enzyme selectively inhibit cell proliferation induced by PDGF (Kohama et al., supra).

Purine Nucleotide Kinases

The purine nucleotide kinases, adenylate kinase (ATP:AMP phosphotransferase, or AdK) and guanylate kinase (ATP:GMP phosphotransferase, or GuK) play a key role in nucleotide metabolism and are crucial to the synthesis and regulation of cellular levels of ATP and GTP, respectively. These two molecules are precursors in DNA and RNA synthesis in growing cells and provide the primary source of biochemical energy in cells (ATP), and signal transduction pathways (GTP). Inhibition of various steps in the synthesis of these two molecules has been the basis of many antiproliferative drugs for cancer and antiviral therapy (Pillwein, K. et al. (1990) Cancer Res. 50:1576-1579).

AdK is found in almost all cell types and is especially abundant in cells having high rates of ATP synthesis and utilization such as skeletal muscle. In these cells AdK is physically associated with mitochondria and myofibrils, the subcellular structures that are involved in energy production and utilization, respectively. Recent studies have demonstrated a major function for AdK in transferring high energy phosphoryls from metabolic processes generating ATP to cellular components consuming ATP (Zeleznikar, R. J. et al. (1995) J. Biol. Chem. 270:7311-7319). Thus AdK may have a pivotal role in maintaining energy production in cells, particularly those having a high rate of growth or metabolism such as cancer cells, and may provide a target for suppression of its activity in order to treat certain cancers. Alternatively, reduced AdK activity may be a source of various metabolic, muscle-energy disorders that can result in cardiac or respiratory failure and may be treatable by increasing AdK activity.

GuK, in addition to providing a key step in the synthesis of GTP for RNA and DNA synthesis, also fulfills an essential function in signal transduction pathways of cells through the regulation of GDP and GTP. Specifically, GTP binding to membrane associated G proteins mediates the activation of cell receptors, subsequent intracellular activation of adenyl cyclase, and production of the second messenger, cyclic AMP. GDP binding to G proteins inhibits these processes. GDP and GTP levels also control the activity of certain oncogenic proteins such as p21^{ras} known to be involved in control of cell proliferation and oncogenesis (Bos, J. L. (1989) Cancer Res. 49:4682-4689). High ratios of GTP:GDP caused by suppression of GuK cause activation of p21^{ras} and promote oncogenesis. Increasing GuK activity to increase levels of GDP and reduce the GTP:GDP ratio may provide a therapeutic strategy to reverse oncogenesis.

GuK is an important enzyme in the phosphorylation and activation of certain antiviral drugs useful in the treatment of herpes virus infections. These drugs include the guanine homologs acyclovir and buciclovir (Miller, W. H. and R. L. Miller (1980) J. Biol. Chem. 255:7204-7207; Stenberg, K. et al. (1986) J. Biol. Chem. 261:2134-2139). Increasing GuK activity in infected cells may provide a therapeutic strategy for augmenting the effectiveness of these drugs and possibly for reducing the necessary dosages of the drugs.

Pyrimidine Kinases

The pyrimidine kinases are deoxycytidine kinase and thymidine kinase 1 and 2. Deoxycytidine kinase is located in the nucleus, and thymidine kinase 1 and 2 are found in the cytosol (Johansson, M. et al. (1997) Proc. Natl. Acad. Sci. USA 94:11941-11945). Phosphorylation of deoxyribonucleosides by pyrimidine kinases provides an alternative pathway for de novo synthesis of DNA precursors. The role of pyrimidine kinases, like purine kinases, in phosphorylation is critical to the activation of several chemotherapeutically important nucleoside analogues (Arner E. S. and S. Eriksson (1995) Pharmacol. Ther. 67:155-186).

### Phosphatases

Protein phosphatases are generally characterized as either serine/threonine- or tyrosine-specific based on their preferred phospho-amino acid substrate. However, some phosphatases (DSPs, for dual specificity phosphatases) can act on phosphorylated tyrosine, serine, or threonine residues. The protein serine/threonine phosphatases (PSPs) are important regulators of many cAMP-mediated hormone responses in cells. Protein tyrosine phosphatases (PTPs) play a significant role in cell cycle and cell signaling processes. Another family of phosphatases is the acid phosphatase or histidine acid phosphatase (HAP) family whose members hydrolyze phosphate esters at acidic pH conditions.

PSPs are found in the cytosol, nucleus, and mitochondria and in association with cytoskeletal and membranous structures in most tissues, especially the brain. Some PSPs require divalent cations, such as Ca²⁺ or Mn²⁺, for activity. PSPs play important roles in glycogen metabolism, muscle contraction, protein synthesis, T cell function, neuronal activity, oocyte maturation, and hepatic metabolism (reviewed in Cohen, P. (1989) Annu. Rev. Biochem. 58:453-508). PSPs can be separated into two classes. The PPP class includes PP1, PP2A, PP2B/calcineurin, PP4, PP5, PP6, and PP7. Members of this class are composed of a homologous catalytic subunit bearing a very highly conserved signature sequence, coupled with one or more regulatory subunits (PROSITE PDOC00115). Further interactions with scaffold and anchoring molecules determine the intracellular localization of PSPs and substrate specificity. The PPM class consists of several closely related isoforms of PP2C and is evolutionarily unrelated to the PPP class.

PP1 dephosphorylates many of the proteins phosphorylated by cyclic AMP-dependent protein kinase (PKA) and is an important regulator of many cAMP-mediated hormone responses in cells. A number of isoforms have been identified, with the alpha and beta forms being produced by alternative splicing of the same gene. Both ubiquitous and tissue-specific targeting proteins for PP1 have been identified. In the brain, inhibition of PP1 activity by the dopamine and adenosine 3′,5′-monophosphate-regulated phosphoprotein of 32 kDa (DARPP-32) is necessary for normal dopamine response in neostriatal neurons (reviewed in Price, N. E. and M. C. Mumby (1999) Curr. Opin. Neurobiol. 9:336-342). PP1, along with PP2A, has been shown to limit motility in microvascular endothelial cells, suggesting a role for PSPs in the inhibition of angiogenesis (Gabel, S. et al. (1999) Otolaryngol. Head Neck Surg. 121:463-468).

PP2A is the main serine/threonine phosphatase. The core PP2A enzyme consists of a single 36 kDa catalytic subunit (C) associated with a 65 kDa scaffold subunit (A), whose role is to recruit additional regulatory subunits (B). Three gene families encoding B subunits are known (PR55, PR61, and PR72), each of which contain multiple isoforms, and additional families may exist (Millward, T. A et al. (1999) Trends Biosci. 24:186-191). These “B-type” subunits are cell type- and tissue-specific and determine the substrate specificity, enzymatic activity, and subcellular localization of the holoenzyme. The PR55 family is highly conserved and bears a conserved motif (PROSITE PDOC00785). PR55 increases PP2A activity toward mitogen-activated protein kinase (MAPK) and MAPK kinase (MEK). PP2A dephosphorylates the MAPK active site, inhibiting the cell's entry into mitosis. Several proteins can compete with PR55 for PP2A core enzyme binding, including the CKII kinase catalytic subunit, polyomavirus middle and small T antigens, and SV40 small t antigen. Viruses may use this mechanism to commandeer PP2A and stimulate progression of the cell through the cell cycle (Pallas, D. C. et al. (1992) J. Virol. 66:886-893). Altered MAP kinase expression is also implicated in a variety of disease conditions including cancer, inflammation, immune disorders, and disorders affecting growth and development. PP2A, in fact, can dephosphorylate and modulate the activities of more than 30 protein kinases in vitro, and other evidence suggests that the same is true in vivo for such kinases as PKB, PKC, the calmodulin-dependent kinases, ERK family MAP kinases, cyclin-dependent kinases, and the IκB kinases (reviewed in Millward et al., supra). PP2A is itself a substrate for CKI and CKII kinases, and can be stimulated by polycationic macromolecules. A PP2A-like phosphatase is necessary to maintain the G1 phase destruction of mammalian cyclins A and B (Bastians, H. et al. (1999) Mol. Biol. Cell 10:3927-3941). PP2A is a major activity in the brain and is implicated in regulating neurofilament stability and normal neural function, particularly the phosphorylation of the microtubule-associated protein tau. Hyperphosphorylation of tau has been proposed to lead to the neuronal degeneration seen in Alzheimer's disease (reviewed in Price and Mumby, supra).

PP2B, or calcineurin, is a Ca²⁺-activated dimeric phosphatase and is particularly abundant in the brain. It consists of catalytic and regulatory subunits, and is activated by the binding of the calcium/calmodulin complex. Calcineurin is the target of the immunosuppressant drugs cyclosporine and FK506. Along with other cellular factors, these drugs interact with calcineurin and inhibit phosphatase activity. In T cells, this blocks the calcium dependent activation of the NF-AT family of transcription factors, leading to immunosuppression. This family is widely distributed, and it is likely that calcineurin regulates gene expression in other tissues as well. In neurons, calcineurin modulates functions which range from the inhibition of neurotransmitter release to desensitization of postsynaptic NMDA-receptor coupled calcium channels to long term memory (reviewed in Price and Mumby, supra).

Other members of the PPP class have recently been identified (Cohen, P. T. (1997) Trends Biochem. Sci. 22:245-251). One of them, PP5, contains regulatory domains with tetratricopeptide repeats. It can be activated by polyunsaturated fatty acids and anionic phospholipids in vitro and appears to be involved in a number of signaling pathways, including those controlled by atrial natriuretic peptide or steroid hormones (reviewed in Andreeva, A. V. and M. A. Kutuzov (1999) Cell Signal. 11:555-562).

PP2C is a ˜42 kDa monomer with broad substrate specificity and is dependent on divalent cations (mainly Mn²⁺ or Mg²⁺) for its activity. PP2C proteins share a conserved N-terminal region with an invariant DGH motif, which contains an aspartate residue involved in cation binding (PROSITE PDOC00792). Targeting proteins and mechanisms regulating PP2C activity have not been identified. PP2C has been shown to inhibit the stress-responsive p38 and Jun kinase (JNK) pathways (Takekawa, M. et al. (1998) EMBO J. 17:4744-4752).

The human skeletal muscle PP2C gamma more closely resembles PP2Cs from Paramecium tetraurelia and Schizosaccharomyces pombe than mammalian PP2Cs. PP2Cgamma is widely expressed, especially in testis, skeletal muscle, and heart. It requires Mg2+ or Mn2+ for activity and has a highly acidic domain with 75% of the 54 residues being glutamate or aspartate (Travis, S. M. and Welsh, M. J. (1997) FEBS lett. 412:415-419). PP2Cgamma localizes to the nucleus in vivo and is associated with the spliceosome in vitro throughout the splicing reaction. It is also required for efficient formation of the A complex during the early stages of spliceosome assembly. Research indicated that at least one specific dephosphorylation event catalyzed by PP2Cgamma is required for formation of the spliceosome (Murry, M. V. et al. (1999) Genes Dev. 13:87-97).

In contrast to PSPs, tyrosine-specific phosphatases (PTPs) are generally monomeric proteins of very diverse size (from 20 kDa to greater than 100 kDa) and structure that function primarily in the transduction of signals across the plasma membrane. PTPs are categorized as either soluble phosphatases or transmembrane receptor proteins that contain a phosphatase domain. All PTPs share a conserved catalytic domain of about 300 amino acids which contains the active site. The active site consensus sequence includes a cysteine residue which executes a nucleophilic attack on the phosphate moiety during catalysis (Neel, B. G. and N. K. Tonks (1997) Curr. Opin. Cell Biol. 9:193-204). Receptor PTPs are made up of an N-terminal extracellular domain of variable length, a transmembrane region, and a cytoplasmic region that generally contains two copies of the catalytic domain. Although only the first copy seems to have enzymatic activity, the second copy apparently affects the substrate specificity of the first. The extracellular domains of some receptor PTPs contain fibronectin-like repeats, immunoglobulin-like domains, MAM domains (an extracellular motif likely to have an adhesive function), or carbonic anhydrase-like domains (PROSITE PDOC 00323). This wide variety of structural motifs accounts for the diversity in size and specificity of PTPs.

PTPs play important roles in biological processes such as cell adhesion, lymphocyte activation, and cell proliferation. PTPs μ and κ are involved in cell-cell contacts, perhaps regulating cadherin/catenin function. A number of PTPs affect cell spreading, focal adhesions, and cell motility, most of them via the integrin/tyrosine kinase signaling pathway (reviewed in Neel and Tonks, supra). CD45 phosphatases regulate signal transduction and lymphocyte activation (Ledbetter, J. A. et al. (1988) Proc. Natl. Acad. Sci. USA 85:8628-8632). Soluble PTPs containing Src-homology-2 domains have been identified (SHPs), suggesting that these molecules might interact with receptor tyrosine kinases. SHP-1 regulates cytokine receptor signaling by controlling the Janus family PTKs in hematopoietic cells, as well as signaling by the T-cell receptor and c-Kit (reviewed in Neel and Tonks, supra). M-phase inducer phosphatase plays a key role in the induction of mitosis by dephosphorylating and activating the PTK CDC2, leading to cell division (Sadhu, K. et al. (1990) Proc. Natl. Acad. Sci. USA 87:5139-5143). In addition, the genes encoding at least eight PTPs have been mapped to chromosomal regions that are translocated or rearranged in various neoplastic conditions, including lymphoma, small cell lung carcinoma, leukemia, adenocarcinoma, and neuroblastoma (reviewed in Charbonneau, H. and N. K. Tonks (1992) Annu. Rev. Cell Biol. 8:463-493). The PTP enzyme active site comprises the consensus sequence of the MTM1 gene family. The MTM1 gene is responsible for X-linked recessive myotubular myopathy, a congenital muscle disorder that has been linked to Xq28 (Kioschis, P. et al., (1998) Genomics 54:256-266). Many PTKs are encoded by oncogenes, and it is well known that oncogenesis is often accompanied by increased tyrosine phosphorylation activity. It is therefore possible that PTPs may serve to prevent or reverse cell transformation and the growth of various cancers by controlling the levels of tyrosine phosphorylation in cells. This is supported by studies showing that overexpression of PTKs can suppress transformation in cells and that specific inhibition of PTP can enhance cell transformation (Charbonneau and Tonks, supra).

TPTE (transmembrane phosphatase with tensin homology) is a novel protein with a predicted polypeptide of 551 amino acids and at least two transmembrane domains and a tyrosine phosphatase motif. It is homologous to tumor suppressor PTEN/MMAC1 protein. The TPTE gene is located close to the human centromeric sequences. It has up to seven copies in the male haploid human genome and up to six in the female. TPTE has highly homologous copies on chromosomes HC13, 15,22, and Y, in addition to its HC21 copy or copies. The cDNA has sequence homology to chicken tensin, bovine auxilin and rat cyclin-G associated kinase (GAK). Research suggests that the biological function of TPTE is involved in signal transduction pathways of the endocrine system or in spermatogenetic function of the testis (Chen, H. et al. (1999) Hum. Genet. 105:399-409).

Dual specificity phosphatases (DSPs) are structurally more similar to the PTPs than the PSPs. DSPs bear an extended PTP active site motif with an additional 7 amino acid residues. DSPs are primarily associated with cell proliferation and include the cell cycle regulators cdc25A, B, and C. The phosphatases DUSP1 and DUSP2 inactivate the MAPK family members ERK (extracellular signal-regulated kinase), JNK (c-Jun N-terminal kinase), and p38 on both tyrosine and threonine residues (PROSITE PDOC 00323, supra). In the activated state, these kinases have been implicated in neuronal differentiation, proliferation, oncogenic transformation, platelet aggregation, and apoptosis. Thus, DSPs are necessary for proper regulation of these processes (Muda, M. et al. (1996) J. Biol. Chem. 271:27205-27208). The tumor suppressor PTEN is a DSP that also shows lipid phosphatase activity. It seems to negatively regulate interactions with the extracellular matrix and maintains sensitivity to apoptosis. PTEN has been implicated in the prevention of angiogenesis (Giri, D. and M. Itttmann (1999) Hum. Pathol. 30:419-424) and abnormalities in its expression are associated with numerous cancers (reviewed in Tamura, M. et al. (1999) J. Natl. Cancer Inst. 91:1820-1828).

Histidine acid phosphatase (HAP; EXPASY EC 3.1.3.2), also known as acid phosphatase, hydrolyzes a wide spectrum of substrates including alkyl, aryl, and acyl orthophosphate monoesters and phosphorylated proteins at low pH. HAPs share two regions of conserved sequences, each centered around a histidine residue which is involved in catalytic activity. Members of the HAP family include lysosomal acid phosphatase (LAP) and prostatic acid phosphatase (PAP), both sensitive to inhibition by L-tartrate (PROSITE PDOC00538).

Synaptojanin, a polyphosphoinositide phosphatase, dephosphorylates phosphoinositides at positions 3, 4 and 5 of the inositol ring. Synaptojanin is a major presynaptic protein found at clathrin-coated endocytic intermediates in nerve terminals, and binds the clathrin coat-associated protein, EPS15. This binding is mediated by the C-terminal region of synaptojanin-170, which has 3 Asp-Pro-Phe amino acid repeats. Further, this 3 residue repeat had been found to be the binding site for the EH domains of EPS15 (Haffner, C. et al. (1997) FEBS Lett. 419:175-180). Additionally, synaptojanin may potentially regulate interactions of endocytic proteins with the plasma membrane, and be involved in synaptic vesicle recycling (Brodin, L. et al. (2000) Curr. Opin. Neurobiol. 10:312-320). Studies in mice with a targeted disruption in the synaptojanin 1 gene (Synj1) were shown to support coat formation of endocytic vesicles more effectively than was seen in wild-type mice, suggesting that Synj1 can act as a negative regulator of membrane-coat protein interactions. These findings provide genetic evidence for a crucial role of phosphoinositide metabolism in synaptic vesicle recycling (Cremona, O. et al. (1999) Cell 99:179-188).

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

The potential application of gene expression profiling is particularly relevant to improving diagnosis, prognosis, and treatment of disease. For example, both the levels and sequences expressed in tissues from subjects with Alzheimer's disease may be compared with the levels and sequences expressed in normal brain tissue. Alzheimer's disease is a progressive neurodegenerative disorder that is characterized by the formation of senile plaques and neurofibrillary tangles containing amyloid beta peptide. These plaques are found in limbic and association cortices of the brain, including hippocampus, temporal cortices, cingulate cortex, amygdala, nucleus basalis and locus caeruleus. Early in Alzheimer's pathology, physiological changes are visible in the cingulate cortex (Minoshima, S. et al. (1997) Annals of Neurology 42:85-94). The hippocampus is part of the limbic system and plays an important role in learning and memory. In subjects with Alzheimer's disease, accumulating plaques damage the neuronal architecture in limbic areas and eventually cripple the memory process.

The potential application of gene expression profiling is also relevant to measuring the toxic response to potential therapeutic compounds and of the metabolic response to therapeutic agents. For instance, diseases treated with steroids and disorders caused by the metabolic response to treatment with steroids include adenomatosis, cholestasis, cirrhosis, hemangioma, Henoch-Schonlein purpura, hepatitis, hepatocellular and metastatic carcinomas, idiopathic thrombocytopenic purpura, porphyria, sarcoidosis, and Wilson disease. It is desirable to measure the toxic response to potential therapeutic compounds and of the metabolic response to therapeutic agents.

Steroids are a class of lipid-soluble molecules, including cholesterol, bile acids, vitamnin D, and hormones, that share a common four-ring structure based on cyclopentanoperhydrophenanthrene and that carrry out a wide variety of functions. Steroid hormones, produced by the adrenal cortex, ovaries, and testes, include glucocorticoids, mineralocorticoids, androgens, and estrogens. Steroid hormones are widely used for fertility control and in anti-inflammatory treatments for physical injuries and diseases such as arthritis, asthma, and auto-immune disorders. Progesterone, a naturally occurring progestin, is primarily used to treat amenorrhea, abnormal uterine bleeding, or as a contraceptive. Medroxyprogesterone (MAH), also known as 6α-methyl-17-hydroxyprogesterone, is a synthetic progestin with a pharmacological activity about 15 times greater than progesterone. MAH is used for the treatment of renal and endometrial carcinomas, amenorrhea, abnormal uterine bleeding, and endometriosis associated with hormonal imbalance. MAH has a stimulatory effect on respiratory centers and has been used in cases of low blood oxygenation caused by sleep apnea, chronic obstructive pulmonary disease, or hypercapnia. Beclomethasone is a synthetic glucocorticoid that is used to treat steroid-dependent asthma, to relieve symptoms associated with allergic or nonallergic (vasomotor) rhinitis, or to prevent recurrent nasal polyps following surgical removal. Budesonide is a corticosteroid used to control symptoms associated with allergic rhinitis or asthma. Dexamethasone is a synthetic glucocorticoid used in anti-inflammatory or immunosuppressive compositions. Prednisone is metabolized in the liver to its active form, prednisolone, a glucocorticoid with anti-inflammatory properties. Betamethasone is a synthetic glucocorticoid with anti-inflammatory and immunosuppressive activity and is used to treat psoriasis and fungal infections, such as athlete's foot and ringworm. By comparing both the levels and sequences expressed in tissues from subjects exposed to or treated with steroid compounds with the levels and sequences expressed in normal untreated tissue it is possible to determine tissue responses to steroids.

Osteosarcoma is a malignant primary neoplasm of bone composed of a malignant connective tissue stroma with evidence of malignant, osteoid, bone, or cartilage formation. Classical osteosarcoma is a poorly differentiated tumor affecting mainly young adults, most often involving the long bones, and is classified as osteoblastic, chondroblastic, or fibroblastic according to which histologic component predominates.

Lung Cancer

Lung cancer is the leading cause of cancer death in the United States, affecting more than 100,000 men and 50,000 women each year. The vast majority of lung cancer cases are attributed to smoking tobacco, and increased use of tobacco products in third world countries is projected to lead to an epidemic of lung cancer in these countries. Nearly 90% of the patients diagnosed with lung cancer are cigarette smokers. Tobacco smoke contains thousands of noxious substances that induce carcinogen metabolizing enzymes and covalent DNA adduct formation in the exposed bronchial epithelium. Exposure of the bronchial epithelium to tobacco smoke appears to result in changes in tissue morphology, which are thought to be precursors of cancer. In nearly 80% of patients diagnosed with lung cancer, metastasis has already occurred. Most commonly lung cancers metastasize to pleura, brain, bone, pericardium, and liver. The decision to treat with surgery, radiation therapy, or chemotherapy is made on the basis of tumor histology, response to growth factors or hormones, and sensitivity to inhibitors or drugs. With current treatments, most patients die within one year of diagnosis. Earlier diagnosis and a systematic approach to identification, staging, and treatment of lung cancer could positively affect patient outcome.

Lung cancers progress through a series of morphologically distinct stages from hyperplasia to invasive carcinoma. Malignant lung cancers are divided into two groups comprising four histopathological classes. The Non Small Cell Lung Carcinoma (NSCLC) group includes squamous cell carcinomas, adenocarcinomas, and large cell carcinomas and accounts for about 70% of all lung cancer cases. Adenocarcinomas typically arise in the peripheral airways and often form mucin secreting glands. Squamous cell carcinomas typically arise in proximal airways. The histogenesis of squamous cell carcinomas may be related to chronic inflammation and injury to the bronchial epithelium, leading to squamous metaplasia. The Small Cell Lung Carcinoma (SCLC) group accounts for about 20% of lung cancer cases. SCLCs typically arise in proximal airways and exhibit a number of paraneoplastic syndromes including inappropriate production of adrenocorticotropin and anti-diuretic hormone.

Lung cancer cells accumulate numerous genetic lesions, many of which are associated with cytologically visible chromosomal aberrations. The high frequency of chromosomal deletions associated with lung cancer may reflect the role of multiple tumor suppressor loci in the etiology of this disease. Deletion of the short arm of chromosome 3 is found in over 90% of cases and represents one of the earliest genetic lesions leading to lung cancer. Deletions at chromosome arms 9p and 17p are also common. Other frequently observed genetic lesions include overexpression of telomerase, activation of oncogenes such as K-ras and c-myc, and inactivation of tumor suppressor genes such as RB, p53 and CDKN2.

Genes differentially regulated in lung cancer have been identified by a variety of methods. Using mRNA differential display technology, Manda et al. (1999; Genomics 51:5-14) identified five genes differentially expressed in lung cancer cell lines compared to normal bronchial epithelial cells. Among the known genes, pulmonary surfactant apoprotein A and alpha 2 macroglobulin were down regulated whereas nm23H1 was upregulated. Petersen et al. (2000; Int J. Cancer, 86:512-517) used suppression subtractive hybridization to identify 552 clones differentially expressed in lung tumor derived cell lines, 205 of which represented known genes. Among the known genes, thrombospondin-1, fibronectin, intercellular adhesion molecule 1, and cytokeratins 6 and 18 were previously observed to be differentially expressed in lung cancers. Wang et al. (2000; Oncogene 19:1519-1528) used a combination of microarray analysis and subtractive hybridization to identify 17 genes differentially overexpresssed in squamous cell carcinoma compared with normal lung epithelium. Among the known genes they identified were keratin isoform 6, KOC, SPRC, IGFb2, connexin 26, plakofillin 1 and cytokeratin 13.

Breast Cancer

There are more than 180,000 new cases of breast cancer diagnosed each year, and the mortality rate for breast cancer approaches 10% of all deaths in females between the ages of 45-54 (K. Gish (1999) AWIS Magazine 28:7-10). However the survival rate based on early diagnosis of localized breast cancer is extremely high (97%), compared with the advanced stage of the disease in which the tumor has spread beyond the breast (22%). Current procedures for clinical breast examination are lacking in sensitivity and specificity, and efforts are underway to develop comprehensive gene expression profiles for breast cancer that may be used in conjunction with conventional screening methods to improve diagnosis and prognosis of this disease (Perou C. M. et al. (2000) Nature 406:747-752).

Breast cancer is a genetic disease commonly caused by mutations in breast epithelial cells. Mutations in two genes, BRCA1 and BRCA2, are known to greatly predispose a woman to breast cancer and may be passed on from parents to children (Gish, supra). However, this type of hereditary breast cancer accounts for only about 5% to 9% of breast cancers, while the vast majority of breast cancer is due to noninherited mutations that occur in breast epithelial cells.

A good deal is already known about the expression of specific genes associated with breast cancer. For example, the relationship between expression of epidermal growth factor (EGF) and its receptor, EGFR, to human mammary carcinoma has been particularly well studied. (See Khazaie, K. et al. (1993) Cancer and Metastasis Rev. 12:255-274), and references cited therein for a review of this area.) Overexpression of EGFR, particularly coupled with down-regulation of the estrogen receptor, is a marker of poor prognosis in breast cancer patients. In addition, EGFR expression in breast tumor metastases is frequently elevated relative to the primary tumor, suggesting that EGFR is involved in tumor progression and metastasis. This is supported by accumulating evidence that EGF has effects on cell functions related to metastatic potential, such as cell motility, chemotaxis, secretion and differentiation. Changes in expression of other members of the erbB receptor family, of which EGFR is one, have also been implicated in breast cancer. The abundance of erbB receptors, such as HER-2/neu, HER-3, and HER4, and their ligands in breast cancer points to their functional importance in the pathogenesis of the disease, and may therefore provide targets for therapy of the disease (Bacus, S. S. et al. (1994) Am. J. Clin. Pathol. 102:S13-S24). Other known markers of breast cancer include a human secreted frizzled protein mRNA that is downregulated in breast tumors; the matrix G1a protein which is overexpressed is human breast carcinoma cells; Drg1 or RTP, a gene whose expression is diminished in colon, breast, and prostate tumors; maspin, a tumor suppressor gene downregulated in invasive breast carcinomas; and CaN19, a member of the S100 protein family, all of which are down regulated in mammary carcinoma cells relative to normal mammary epithelial cells (Zhou Z. et al. (1998) Int. J. Cancer 78:95-99; Chen, L. et al. (1990) Oncogene 5:1391-1395; Ulrix W. et al (1999) FEBS Lett. 455:23-26; Sager, R. et al. (1996) Curr. Top. Microbiol. Immunol. 213:51-64; and Lee, S. W. et al. (1992) Proc. Natl. Acad. Sci. USA 89:2504-2508).

Cell lines derived from human mammary epithelial cells at various stages of breast cancer provide a useful model to study the process of malignant transformation and tumor progression as it has been shown that these cell lines retain many of the properties of their parental tumors for lengthy culture periods (Wistuba, I. I. et al. (1998) Clin. Cancer Res. 4:2931-2938). Such a model is particularly useful for comparing phenotypic and molecular characteristics of human mammary epithelial cells at various stages of malignant transformation.

Ovarian Cancer

Ovarian cancer is the leading cause of death from a gynecologic cancer. The majority of ovarian cancers are derived from epithelial cells, and 70% of patients with epithelial ovarian cancers present with late-stage disease. As a result, the long-term survival rates for this disease is very low. Identification of early-stage markers for ovarian cancer would significantly increase the survival rate. The molecular events that lead to ovarian cancer are poorly understood. Some of the known aberrations include mutation of p53 and microsatellite instability. Since gene expression patterns are likely to vary when normal ovary is compared to ovarian tumors, examination of gene expression in these tissues to identify possible markers for ovarian cancer is particularly relevant to improving diagnosis, prognosis, and treatment of this disease.

Colon Cancer

Colorectal cancer is the second leading cause of cancer deaths in the United States. Colon cancer is associated with aging, since 90% of the total cases occur in individuals over the age of 55. A widely accepted hypothesis is that several contributing genetic mutations must accumulate over time in an individual who develops the disease. To understand the nature of genetic alterations in colorectal cancer, a number of studies have focused on the inherited syndromes. The first known inherited syndrome, Familial Adenomatous Polyposis (FAP), is caused by mutations in the Adenomatous Polyposis Coli gene (APC), resulting in truncated or inactive forms of the protein. This tumor suppressor gene has been mapped to chromosome 5q. The second known inherited syndrome is hereditary nonpolyposis colorectal cancer (HNPCC), which is caused by mutations in mismatch repair genes.

Although hereditary colon cancer syndromes occur in a small percentage of the population and most colorectal cancers are considered sporadic, knowledge from studies of the hereditary syndromes can be generally applied. For instance, somatic mutations in APC occur in at least 80% of indiscriminate colon tumors. APC mutations are thought to be the initiating event in the disease. Other mutations occur subsequently. Approximately 50% of colorectal cancers contain activating mutations in ras, while 85% contain inactivating mutations in p53. Changes in these genes lead to gene expression changes in colon cancer. Less is understood about downstream targets of these mutations and the role they may play in cancer development and progression.

Preadipocyte Cells

The most important function of adipose tissue is its ability to store and release fat during periods of feeding and fasting. White adipose tissue is the major energy reserve in periods of excess energy use. Its primary purpose is mobilization during energy deprivation. Understanding how various molecules regulate adiposity and energy balance in physiological and pathophysiological situations may lead to the development of novel therapeutics for human obesity. Adipose tissue is also one of the important target tissues for insulin. Adipogenesis and insulin resistance in type II diabetes are linked and present intriguing relations. Most patients with type II diabetes are obese and obesity in turn causes insulin resistance.

The majority of research in adipocyte biology to date has been done using transformed mouse preadipocyte cell lines. The culture condition which stimulates mouse preadipocyte differentiation is different from that for inducing human primary preadipocyte differentiation. In addition, primary cells are diploid and may therefore reflect the in vivo context better than aneuploid cell lines. Understanding the gene expression profile during adipogenesis in humans will lead to understanding the fundamental mechanism of adiposity regulation. Furthermore, through comparing the gene expression profiles of adipogenesis between donor with normal weight and donor with obesity, identification of crucial genes, potential drug targets for obesity and type II diabetes, will be possible.

Peroxisome Proliferator-activated Receptor Gamma Aponist

Thiazolidinediones (TZDs) act as agonists for the peroxisome-proliferator-activated receptor gamma (PPARγ), a member of the nuclear hormone receptor superfamily. TZDs reduce hyperglycemia, hyperinsulinemia, and hypertension, in part by promoting glucose metabolism and inhibiting gluconeogenesis. Roles for PPARγ and its agonists have been demonstrated in a wide range of pathological conditions including diabetes, obesity, hypertension, atherosclerosis, polycystic ovarian syndrome, and cancers such as breast, prostate, liposarcoma, and colon cancer.

The mechanism by which TZDs and other PPARγ agonists enhance insulin sensitivity is not fully understood, but may involve the ability of PPARγ to promote adipogenesis. When ectopically expressed in cultured preadipocytes, PPARγ is a potent inducer of adipocyte differentiation. TZDs, in combination with insulin and other factors, can also enhance differentiation of human preadipocytes in culture (Adams et al. (1997) J. Clin. Invest. 100:3149-3153). The relative potency of different TZDs in promoting adipogenesis in vitro is proportional to both their insulin sensitizing effects in vivo, and their ability to bind and activate PPARγ in vitro. Interestingly, adipocytes derived from omental adipose depots are refractory to the effects of TZDs. It has therefore been suggested that the insulin sensitizing effects of TZDs may result from their ability to promote adipogenesis in subcutaneous adipose depots (Adams et al., supra). Further, dominant negative mutations in the PPARγ gene have been identified in two non-obese subjects with severe insulin resistance, hypertension, and overt non-insulin dependent diabetes mellitus (NIDDM) (Barroso et al. (1998) Nature 402:880-883).

NIDDM is the most common form of diabetes mellitus, a chronic metabolic disease that affects 143 million people worldwide. NIDDM is characterized by abnormal glucose and lipid metabolism that result from a combination of peripheral insulin resistance and defective insulin secretion. NIDDM has a complex, progressive etiology and a high degree of heritability. Numerous complications of diabetes including heart disease, stroke, renal failure, retinopathy, and peripheral neuropathy contribute to the high rate of morbidity and mortality.

At the molecular level, PPARγ functions as a ligand activated transcription factor. In the presence of ligand, PPARγ forms a heterodimer with the retinoid X receptor (RXR) which then activates transcription of target genes containing one or more copies of a PPARγ response element (PPRE). Many genes important in lipid storage and metabolism contain PPREs and have been identified as PPAR targets, including PEPCK, aP2, LPL, ACS, and FAT-P (Auwerx, J. (1999) Diabetologia 42:1033-1049). Multiple ligands for PPARγ have been identified. These include a variety of fatty acid metabolites; synthetic drugs belonging to the TZD class, such as Pioglitazone and Rosiglitazone (BRIA9653); and certain non-glitazone tyrosine analogs such as GI262570 and GW1929. The prostaglandin derivative 15-dPGJ2 is a potent endogenous ligand for PPARγ.

Expression of PPARγ is very high in adipose but barely detectable in skeletal muscle, the primary site for insulin stimulated glucose disposal in the body. PPARγ is also moderately expressed in large intestine, kidney, liver, vascular smooth muscle, hematopoietic cells, and macrophages. The high expression of PPARγ in adipose suggests that the insulin sensitizing effects of TZDs may result from alterations in the expression of one or more PPARγ regulated genes in adipose tissue. Identification of PPARγ target genes will contribute to better drug design and the development of novel therapeutic strategies for diabetes, obesity, and other conditions.

Systematic attempts to identify PPARγ target genes have been made in several rodent models of obesity and diabetes (Suzuki et al. (2000) Jpn. J. Pharmacol. 84:113-123; Way et al. (2001) Endocrinology 142:1269-1277). However, a serious drawback of the rodent gene expression studies is that significant differences exist between human and rodent models of adipogenesis, diabetes, and obesity (Taylor (1999) Cell 97:9-12; Gregoire et al. (1998) Physiol. Reviews 78:783-809). Therefore, an unbiased approach to identifying TZD regulated genes in primary cultures of human tissues is necessary to fully elucidate the molecular basis for diseases associated with PPARγ activity.

Tangier Disease

Tangier disease (TD) is a rare genetic disorder characterized by near absence of circulating high density lipoprotein (HDL) and the accumulation of cholesterol esters in many tissues, including tonsils, lymph nodes, liver, spleen, thymus, and intestine. Low levels of HDL represent a clear predictor of premature coronary artery disease and homozygous TD correlates with a four- to six-fold increase in cardiovascular disease compared to controls. The major cardio-protective activity of HDL is ascribed to its role in reverse cholesterol transport, the flux of cholesterol from peripheral cells such as tissue macrophages, through plasma lipoproteins to the liver. The HDL protein, apolipoprotein A-I, plays a major role in this process, interacting with the cell surface to remove excess cholesterol and phospholipids. Recent studies have shown that this pathway is severely impaired in TD and the defect lies in a specific gene, the ABC1 transporter. This gene is a member of the family of ATP-binding cassette transporters, which utilize ATP hydrolysis to transport a variety of substrates across membranes.

There is a need in the art for new compositions, including nucleic acids and proteins, for the diagnosis, prevention, and treatment of cardiovascular diseases, immune system disorders, neurological disorders, disorders affecting growth and development, lipid disorders, cell proliferative disorders, and cancers.

### SUMMARY OF THE INVENTION

Various embodiments of the invention provide purified polypeptides, kinases and phosphatases, referred to collectively as ‘KPP’ and individually as ‘KPP-1,’ ‘KPP-2,’ ‘KPP-3,’ ‘KPP-4,’ ‘KPP-5,’ ‘KPP-6,’ ‘KPP-7,’ ‘KPP-8,’ ‘KPP-9,’ ‘KPP-10,’ ‘KPP-11,’ ‘KPP-12,’ ‘KPP-13,’ ‘KPP-14,’ ‘KPP-15,’ ‘KPP-16,’ ‘KPP-17,’ ‘KPP-18,’ ‘KPP-19,’ ‘KPP-20,’ ‘KPP-21,’ ‘KPP-22,’ ‘KPP-23,’ ‘KPP-24,’ ‘KPP-25,’ ‘KPP-26,’ ‘KPP-27,’ ‘KPP-28,’ ‘KPP-29,’ ‘KPP-30,’ ‘KPP-31,’ ‘KPP-32,’ ‘KPP-33,’ ‘KPP-34,’ ‘KPP-35,’ ‘KPP-36,’ ‘KPP-37,’ ‘KPP-38,’ ‘KPP-39,’ ‘KPP-40,’ ‘KPP-41,’ ‘KPP-42,’ ‘KPP-43,’ ‘KPP-44,’ ‘KPP-45,’ ‘KPP46,’ ‘KPP-47,’ ‘KPP-48,’ ‘KPP-49,’ ‘KPP-50,’ ‘KPP-51,’ and ‘KPP-52’ and methods for using these proteins and their encoding polynucleotides for the detection, diagnosis, and treatment of diseases and medical conditions. Embodiments also provide methods for utilizing the purified kinases and pbosphatases and/or their encoding polynucleotides for facilitating the drug discovery process, including determination of efficacy, dosage, toxicity, and pharmacology. Related embodiments provide methods for utilizing the purified kinases and phosphatases and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-52, b) a polypeptide comprising a naturally occurring amino acid sequence at least 90% identical or at least about 90% identical to an amino acid sequence selected from the group consisting of SEQ ID NO:1-52, c) a biologically active fragment of a polypeptide having an amino acid sequence selected from the group consisting of SEQ ID NO:1-52, and d) an immunogenic fragment of a polypeptide having an amino acid sequence selected from the group consisting of SEQ ID NO:1-52. Another embodiment provides an isolated polypeptide comprising an amino acid sequence of SEQ ID NO:1-52.

Still another embodiment provides an isolated polynucleotide encoding a polypeptide selected from the group consisting of a) a polypeptide comprising an amino acid sequence selected from the group consisting of SEQ ID NO:1-52, b) a polypeptide comprising a naturally occurring amino acid sequence at least 90% identical or at least about 90% identical to an amino acid sequence selected from the group consisting of SEQ ID NO:1-52, c) a biologically active fragment of a polypeptide having an amino acid sequence selected from the group consisting of SEQ ID NO:1-52, and d) an immunogenic fragment of a polypeptide having an amino acid sequence selected from the group consisting of SEQ ID NO:1-52. In another embodiment, the polynucleotide encodes a polypeptide selected from the group consisting of SEQ ID NO:1-52. In an alternative embodiment, the polynucleotide is selected from the group consisting of SEQ ID NO:53-104.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-52, b) a polypeptide comprising a naturally occurring amino acid sequence at least 90% identical or at least about 90% identical to an amino acid sequence selected from the group consisting of SEQ ID NO:1-52, c) a biologically active fragment of a polypeptide having an amino acid sequence selected from the group consisting of SEQ ID NO:1-52, and d) an immunogenic fragment of a polypeptide having an amino acid sequence selected from the group consisting of SEQ ID NO:1-52. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-52, b) a polypeptide comprising a naturally occurring amino acid sequence at least 90% identical or at least about 90% identical to an amino acid sequence selected from the group consisting of SEQ ID NO:1-52, c) a biologically active fragment of a polypeptide having an amino acid sequence selected from the group consisting of SEQ ID NO:1-52, and d) an immunogenic fragment of a polypeptide having an amino acid sequence selected from the group consisting of SEQ ID NO:1-52. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-52, b) a polypeptide comprising a naturally occurring amino acid sequence at least 90% identical or at least about 90% identical to an amino acid sequence selected from the group consisting of SEQ ID NO:1-52, c) a biologically active fragment of a polypeptide having an amino acid sequence selected from the group consisting of SEQ ID NO:1-52, and d) an immunogenic fragment of a polypeptide having an amino acid sequence selected from the group consisting of SEQ ID NO:1-52.

Still yet another embodiment provides an isolated polynucleotide selected from the group consisting of a) a polynucleotide comprising a polynucleotide sequence selected from the group consisting of SEQ ID NO:53-104, b) a polynucleotide comprising a naturally occurring polynucleotide sequence at least 90% identical or at least about 90% identical to a polynucleotide sequence selected from the group consisting of SEQ ID NO:53-104, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:53-104, b) a polynucleotide comprising a naturally occurring polynucleotide sequence at least 90% identical or at least about 90% identical to a polynucleotide sequence selected from the group consisting of SEQ ID NO:53-104, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:53-104, b) a polynucleotide comprising a naturally occurring polynucleotide sequence at least 90% identical or at least about 90% identical to a polynucleotide sequence selected from the group consisting of SEQ ID NO:53-104, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-52, b) a polypeptide comprising a naturally occurring amino acid sequence at least 90% identical or at least about 90% identical to an amino acid sequence selected from the group consisting of SEQ ID NO:1-52, c) a biologically active fragment of a polypeptide having an amino acid sequence selected from the group consisting of SEQ ID NO:1-52, and d) an immunogenic fragment of a polypeptide having an amino acid sequence selected from the group consisting of SEQ ID NO:1-52, and a pharmaceutically acceptable excipient. In one embodiment, the composition can comprise an amino acid sequence selected from the group consisting of SEQ ID NO:1-52. Other embodiments provide a method of treating a disease or condition associated with decreased or abnormal expression of functional KPP, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-52, b) a polypeptide comprising a naturally occurring amino acid sequence at least 90% identical or at least about 90% identical to an amino acid sequence selected from the group consisting of SEQ ID NO:1-52, c) a biologically active fragment of a polypeptide having an amino acid sequence selected from the group consisting of SEQ ID NO:1-52, and d) an immunogenic fragment of a polypeptide having an amino acid sequence selected from the group consisting of SEQ ID NO:1-52. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional KPP, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-52, b) a polypeptide comprising a naturally occurring amino acid sequence at least 90% identical or at least about 90% identical to an amino acid sequence selected from the group consisting of SEQ ID NO:1-52, c) a biologically active fragment of a polypeptide having an amino acid sequence selected from the group consisting of SEQ ID NO:1-52, and d) an immunogenic fragment of a polypeptide having an amino acid sequence selected from the group consisting of SEQ ID NO:1-52. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional KPP, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-52, b) a polypeptide comprising a naturally occurring amino acid sequence at least 90% identical or at least about 90% identical to an amino acid sequence selected from the group consisting of SEQ ID NO:1-52, c) a biologically active fragment of a polypeptide having an amino acid sequence selected from the group consisting of SEQ ID NO:1-52, and d) an immunogenic fragment of a polypeptide having an amino acid sequence selected from the group consisting of SEQ ID NO:1-52. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-52, b) a polypeptide comprising a naturally occurring amino acid sequence at least 90% identical or at least about 90% identical to an amino acid sequence selected from the group consisting of SEQ ID NO:1-52, c) a biologically active fragment of a polypeptide having an amino acid sequence selected from the group consisting of SEQ ID NO:1-52, and d) an immunogenic fragment of a polypeptide having an amino acid sequence selected from the group consisting of SEQ ID NO:1-52. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:53-104, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ D NO:53-104, ii) a polynucleotide comprising a naturally occurring polynucleotide sequence at least 90% identical or at least about 90% identical to a polynucleotide sequence selected from the group consisting of SEQ ID NO:53-104, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:53-104, ii) a polynucleotide comprising a naturally occurring polynucleotide sequence at least 90% identical or at least about 90% identical to a polynucleotide sequence selected from the group consisting of SEQ ID NO:53-104, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

### Definitions

“KPP” refers to the amino acid sequences of substantially purified KPP obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of KPP. Agonists may include proteins, nucleic acids, carbohydrates, small molecules, or any other compound or composition which modulates the activity of KPP either by directly interacting with KPP or by acting on components of the biological pathway in which KPP participates.

An “allelic variant” is an alternative form of the gene encoding KPP. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding KPP include those sequences with deletions, insertions, or substitutions of different nucleotides, resulting in a polypeptide the same as KPP or a polypeptide with at least one functional characteristic of KPP. Included within this definition are polymorphisms which may or may not be readily detectable using a particular oligonucleotide probe of the polynucleotide encoding KPP, and improper or unexpected hybridization to allelic variants, with a locus other than the normal chromosomal locus for the polynucleotide encoding KPP. The encoded protein may also be “altered,” and may contain deletions, insertions, or substitutions of amino acid residues which produce a silent change and result in a functionally equivalent KPP. Deliberate amino acid substitutions may be made on the basis of one or more similarities in polarity, charge, solubility, hydrophobicity, hydrophilicity, and/or the amphipathic nature of the residues, as long as the biological or immunological activity of KPP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of KPP. Antagonists may include proteins such as antibodies, anticalins, nucleic acids, carbohydrates, small molecules, or any other compound or composition which modulates the activity of KPP either by directly interacting with KPP or by acting on components of the biological pathway in which KPP participates.

The term “antibody” refers to intact immunoglobulin molecules as well as to fragments thereof, such as Fab, F(ab′)₂, and Fv fragments, which are capable of binding an epitopic determinant. Antibodies that bind KPP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH2), which may improve a desired property, e.g., resistance to nucleases or longer lifetime in blood. Aptamners may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic KPP, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding KPP or fragments of KPP may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (Accelrys, Burlington Mass.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

!Original Residue? Conservative Substitution?

| | |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable laber” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of KPP or a polynucleotide encoding KPP which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:53-104 can comprise a region of unique polynucleotide sequence that specifically identifies SEQ ID NO:53-104, for example, as distinct from any other sequence in the genome from which the fragment was obtained. A fragment of SEQ ID NO:53-104 can be employed in one or more embodiments of methods of the invention, for example, in hybridization and amplification technologies and in analogous methods that distinguish SEQ ID NO:53-104 from related polynucleotides. The precise length of a fragment of SEQ ID NO:53-104 and the region of SEQ ID NO:53-104 to which the fragment corresponds are routinely determinable by one of ordinary skill in the art based on the intended purpose for the fragment.

A fragment of SEQ ID NO:1-52 is encoded by a fragment of SEQ ID NO:53-104. A fragment of SEQ ID NO:1-52 can comprise a region of unique amino acid sequence that specifically identifies SEQ ID NO:1-52. For example, a fragment of SEQ ID NO:1-52 can be used as an immunogenic peptide for the development of antibodies that specifically recognize SEQ ID NO:1-52. The precise length of a fragment of SEQ ID NO:1-52 and the region of SEQ ID NO:1-52 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, alternatively, sequence identity, between two or more polynucleotide sequences or two or more polypeptide sequences.

The terms “percent identity” and “% identity,” as applied to polynucleotide sequences, refer to the percentage of identical residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nlh.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:
Matrix: BLOSUM62
Reward for match: 1
Penalty for mismatch: −2
Open Gap: 5 and Extension Gap: 2 penalties
Gap×drop-off: 50
Expect: 10
Word Size: 11
Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of identical residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. The phrases “percent similarity” and “% similarity,” as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:
Matrix: BLOSUM62
Open Gap: 11 and Extension Gap: 1 penalties
Gap×drop-off: 50
Expect: 10
Word Size: 3
Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ng sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. and D. W. Russell (2001; Molecular Cloning: A Laboratory Manual, 3rd ed., vol. 1-3, Cold Spring Harbor Press, Cold Spring Harbor N.Y., ch. 9).

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of KPP which is capable of eliciting an immune response when introduced into a living organism, for example, a mammal. The term “immunogenic fragment” also includes any polypeptide or oligopeptide fragment of KPP which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of KPP. For example, modulation may cause an increase or a decrease in protein activity, binding characteristics, or any other biological, functional, or immunological properties of KPP.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an KPP may involve lipidation, glycosylation, phosphorylation, acetylation, racemization, proteolytic cleavage, and other modifications known in the art These processes may occur synthetically or biochemically. Biochemical modifications will vary by cell type depending on the enzymatic milieu of KPP.

“Probe” refers to nucleic acids encoding KPP, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Trimers” are short nucleic acids, usually DNA oligonucleotides, which maybe annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported. by the specification, including the tables, figures, and Sequence Listing, may be used.

Methods for preparing and using probes and primers are described in, for example, Sambrook, J. and D. W. Russell (2001; Molecular Cloning: A Laboratory Manual, 3rd ed., vol. 1-3, Cold Spring Harbor Press, Cold Spring Harbor N.Y.), Ausubel, F. M. et al. (1999; Short Protocols in Molecular Biology, 4^{th} ed., John Wiley & Sons, New York N.Y.), and Innis, M. et al. (1990; PCR Protocols, A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook and Russell (supra). The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing KPP, nucleic acids encoding KPP, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook and Russell (supra).

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity or sequence similarity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity or sequence similarity over a certain defined length of one of the polypeptides.

### The Invention

Various embodiments of the invention include new human kinases and phosphatases (KPP), the polynucleotides encoding KPP, and the use of these compositions for the diagnosis, treatment, or prevention of cardiovascular diseases, immune system disorders, neurological disorders, disorders affecting growth and development, lipid disorders, cell proliferative disorders, and cancers.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to polypeptide embodiments of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Acceirys, Burlington Mass.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are kinases and phosphatases. For example, SEQ ID NO:1 is 96% identical, from residue MI to residue G215, and 100% identical, from residue Y212 to residue P458, to human lymphocyte-specific protein tyrosine kinase (GenBank ID g187034) as determined by the Basic Local Alignment Search Tool (BLAST). (See Table 2.) The BLAST probability score is 2.4e-248, which indicates the probability of obtaining the observed polypeptide sequence alignment by chance. SEQ ID NO:1 is localized to the plasma membrane, has kinase and transferase activity, and is a tyrosine kinase, as determined by BLAST analysis using the PROTEOME database. SEQ ID NO:1 also contains SH2, SH3 and protein kinase domains as determined by searching for statistically significant matches in the hidden Markov model (HMM)-based PFAM database of conserved protein family domains. (See Table 3.) Data from BLIMPS, MOTIFS, BLAST and PROFILESCAN analyses provide further corroborative evidence that SEQ ID NO:1 is a protein tyrosine kinase. In another example, SEQ ID NO:4 is 82% identical, from residue Ml to residue W38, and 98% identical, from residue K32 to residue V353, to human protein tyrosine phosphatase (GenBank D) g1871531) as determined by the Basic Local Alignment Search Tool (BLAST). (See Table 2.) The BLAST probability score is 1.8e-186, which indicates the probability of obtaining the observed polypeptide sequence alignment by chance. SEQ ID NO:4 has phosphatase and hydrolase activity, and is a tyrosine phosphatase, as determined by BLAST analysis using the PROTEOME database. SEQ ID NO:4 also contains a protein tyrosine phosphatase domain as determined by searching for statistically significant matches in the hidden Markov model (HMM)-based PFAM database of conserved protein family domains. (See Table 3.) Data from BLIMPS, MOTIFS, BLAST and PROFILESCAN analyses provide further corroborative evidence that SEQ ID NO:4 is a protein tyrosine kinase. In another example, SEQ ID NO:14 is 100% identical, from residue G19 to residue K286, to human protein phosphatase 1 (GenBank ID g14124968) as determined by the Basic Local Alignment Search Tool (BLAST). (See Table 2.) The BLAST probability score is 3.4e-157, which indicates the probability of obtaining the observed polypeptide sequence alignment by chance. SEQ ID NO:14 has phosphatase and hydrolase activity, and is a protein phosphatase, as determined by BLAST analysis using the PROTEOME database. SEQ ID NO:14 also contains a serine/threonine phosphatase domain as determined by searching for statistically significant matches in the hidden Markov model (HMM)-based PFAM database of conserved protein family domains. (See Table 3.) Data from BLIMPS, PROFILESCAN, MOTIFS, and further BLAST analyses provide further corroborative evidence that SEQ ID NO:14 is a serine/threonine protein phosphatase. In another example, SEQ ID NO:16 is 82% identical, from residue E592 to residue T1634 and 94% identical, from residue C83 to E592, to mouse protein kinase (GenBank ID g406058) as determined by the Basic Local Alignment Search Tool (BLAST). (See Table 2.) The BLAST probability score is 0.0, which indicates the probability of obtaining the observed polypeptide sequence alignment by chance. SEQ ID NO:16 is localized to the cytoskeleton, has protein kinase function, and is a protein kinase which interacts with microtubules as determined by BLAST analysis using the PROTEOME database. SEQ ID NO:16 also contains a PDZ (also known as DHR or GLGF) domain and a protein kinase domain as determined by searching for statistically significant matches in the hidden Markov model (HMM)-based PFAM database of conserved protein family domains. (See Table 3.) Data from BLIMPS, MOTIFS, other BLAST, and PROFILESCAN analyses provide further corroborative evidence that SEQ ID NO:16 is a protein kinase. In another example, SEQ ID NO:27 is 97% identical, from residue M1 to residue L731, to human serine/threonine protein kinase, EMK1 (GenBank ID g1749794) as determined by the Basic Local Alignment Search Tool (BLAST). (See Table 2.) The BLAST probability score is 0.0, which indicates the probability of obtaining the observed polypeptide sequence alignment by chance. SEQ ID NO:27 is homologous to proteins which are localized to the cytoplasm, function as protein kinases involved in microtubule stability, and are serine/threonine kinases with strong similarity to human EMK1, as determined by BLAST analysis using the PROTEOME database. SEQ ID NO:27 also contains a kinase-associated domain, a UBA/TS-N domain, and a protein kinase domain as determined by searching for statistically significant matches in the hidden Markov model (HMM)-based PFAM database of conserved protein family domains. (See Table 3.) Data from BLIMPS, MOTIFS, PROFILESCAN, and other BLAST analyses provide further corroborative evidence that SEQ ID NO:27 is a serine/threonine protein kinase. In another example, SEQ ID NO:43 is 44% identical, from residue Y29 to residue W216, and 26% identical, from residue R460 to residue L526, to human protein serine/threonine kinase (GenBank ID g348245) as determined by the Basic Local Alignment Search Tool (BLAST). (See Table 2.) The BLAST probability score is 1.2e-42, which indicates the probability of obtaining the observed polypeptide sequence alignment by chance. SEQ ID NO:43 also has homology to proteins that are localized to the cytoplasm, have serine/threoinine kinase activity, and that are involved in regulation of the cell cycle, as determined by BLAST analysis using the PROTEOME database. SEQ ID NO:43 also contains a protein kinase domain as determined by searching for statistically significant matches in the hidden Markov model (HMM)-based PFAM database of conserved protein family domains. (See Table 3.) Data from BLIMPS, MOTIFS, BLAST, and PROFILESCAN analyses provide further corroborative evidence that SEQ ID NO:43 is a protein kinase. SEQ ID NO:2-3, SEQ ID NO:5-13, SEQ ID NO:15, SEQ ID NO:17-26, SEQ ID NO:28-42, and SEQ ID NO:44-52 were analyzed and annotated in a similar manner. The algorithms and parameters for the analysis of SEQ ID NO:1-52 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:53-104 or that distinguish between SEQ ID NO:53-104 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N₁_{_{—}}N₂_{_{—}}YYYYY_N₃_{_{—}}N₄_{_{—}} represents a “stitched” sequence in which XXXXXX is the identification number of the cluster of sequences to which the algorithm was applied, and YYYYY is the number of the prediction generated by the algorithm, and N_{1,2,3 . . .} , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXX being the Incyte project identification number, gAAAAA being the GenBank identification number of the human genomic sequence to which the “exon-stretching” algorithm was applied, GBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were band-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, GFG, | Exon prediction from genomic sequences using, |
| ENST | for example, GENSCAN (Stanford University, |
| | CA, USA) or FGENES (Computer Genomics |
| | Group, The Sanger Centre, Cambridge, UK). |
| GBI | Hand-edited analysis of genomic sequences. |
| FL | Stitched or stretched genomic sequences (see Example V). |
| INCY | Full length transcript and exon prediction from |
| | mapping of EST sequences to the genome. |
| | Genomic location and EST composition |
| | data are combined to predict the exons |
| | and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were-used to construct the cDNA libraries shown in Table 5 are described in Table 6.

The invention also encompasses KPP variants. Various embodiments of KPP variants can have at least about 80%, at least about 90%, or at least about 95% amino acid sequence identity to the KPP amino acid sequence, and can contain at least one functional or structural characteristic of KPP.

Various embodiments also encompass polynucleotides which encode KPP. In a particular embodiment, the invention encompasses a polynucleotide sequence comprising a sequence selected from the group consisting of SEQ ID NO:53-104, which encodes KPP. The polynucleotide sequences of SEQ ID NO:53-104, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding KPP. In particular, such a variant polynucleotide will have at least about 70%, or alternatively at least about 85%, or even at least about 95% polynucleotide sequence identity to a polynucleotide encoding KPP. A particular aspect of the invention encompasses a variant of a polynucleotide comprising a sequence selected from the group consisting of SEQ ID NO:53-104 which has at least about 70%, or alternatively at least about 85%, or even at least about 95% polynucleotide sequence identity to a nucleic acid sequence selected from the group consisting of SEQ ID NO:53-104. Any one of the polynucleotide variants described above can encode a polypeptide which contains at least one functional or structural characteristic of KPP.

In addition, or in the alternative, a polynucleotide variant of the invention is a splice variant of a polynucleotide encoding KPP. A splice variant may have portions which have significant sequence identity to a polynucleotide encoding KPP, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding KPP over its entire length; however, portions of the splice variant will have at least about 70%, or alternatively at least about 85%, or alternatively at least about 95%, or alternatively 100% polynucleotide sequence identity to portions of the polynucleotide encoding KPP. For example, a polynucleotide comprising a sequence of SEQ ID NO:95 and a polynucleotide comprising a sequence of SEQ ID NO:96 are splice variants of each other. Any one of the splice variants described above can encode a polypeptide which contains at least one functional or structural characteristic of KPP.

It will be appreciated by those skilled in the art that as a result of the degeneracy of the genetic code, a multitude of polynucleotide sequences encoding KPP, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring KPP, and all such variations are to be considered as being specifically disclosed.

Although polynucleotides which encode KPP and its variants are generally capable of hybridizing to polynucleotides encoding naturally occurring KPP under appropriately selected conditions of stringency, it may be advantageous to produce polynucleotides encoding KPP or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding KPP and its derivatives without altering the encoded amino acid sequences include the production of RNA transcripts having more desirable properties, such as a greater half-life, than transcripts produced from the naturally occurring sequence.

The invention also encompasses production of polynucleotides which encode KPP and KPP derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding KPP or any fragment thereof.

Embodiments of the invention can also include polynucleotides that are capable of hybridizing to the claimed polynucleotides, and, in particular, to those having the sequences shown in SEQ ID NO:53-104 and fragments thereof, under various conditions of stringency (Wahl, G. M. and S. L. Berger (1987) Methods Enzymol. 152:399-407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno N.V.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding KPP may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(I) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode KPP may be cloned in recombinant DNA molecules that direct expression of KPP, or fragments or functional equivalents thereof, in appropriate host cells. Due to the inherent degeneracy of the genetic code, other polynucleotides which encode substantially the same or a functionally equivalent polypeptides may be produced and used to express KPP.

The polynucleotides of the invention can be engineered using methods generally known in the art in order to alter KPP-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of KPP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding KPP may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, KPP itself or a fragment thereof may be synthesized using chemical methods known in the art. For example, peptide synthesis can be performed using various solution-phase or solid-phase techniques (Creighton, T. (1984) Proteins, Structures and Molecular Properties, WH Freeman, New York N.Y., pp. 55-60; Roberge, J. Y. et al. (1995) Science 269:202-204). Automated synthesis may be achieved using the ABI 431A peptide synthesizer (Applied Biosystems). Additionally, the amino acid sequence of KPP, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Creighton, supra, pp. 28-53).

In order to express a biologically active KPP, the polynucleotides encoding KPP or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding KPP. Such elements may vary in their strength and specificity. Specific initiation signals may also be used to achieve more efficient translation of polynucleotides encoding KPP. Such signals include the ATG initiation codon and adjacent sequences, e.g. the Kozak sequence. In cases where a polynucleotide sequence encoding KPP and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding KPP and appropriate transcriptional and translational control elements. These methods include in vitro recombinant DNA techniques, synthetic techniques, and in vivo genetic recombination (Sambrook and Russell, supra, ch. 1-4, and 8; Ausubel et al., supra, ch. 1, 3, and 15).

A variety of expression vector/host systems may be utilized to contain and express polynucleotides encoding KPP. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook and Russell, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding KPP. For example, routine cloning, sucloning, and propagation of polynucleotides encoding KPP can be achieved using a multifunctional E. coli vector such as PBLUESCRIPT (Stratagene, La Jolla Calif.) or PSPORT1 plasmid (Invitrogen). Ligation of polynucleotides encoding KPP into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of KPP are needed, e.g. for the production of antibodies, vectors which direct high level expression of KPP may be used. For example, vectors containing the strong, inducible SP6 or T7 bacteriophage promoter may be used.

Yeast expression systems may be used for production of KPP. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Biotechnology 12:181-184).

Plant systems may also be used for expression of KPP. Transcription of polynucleotides encoding KPP maybe driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters maybe used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding KPP may be ligated into an adenovirus transcription/translation complex consisting of the late promoter and tripartite leader sequence. Insertion in a non-essential E1 or E3 region of the viral genome may be used to obtain infective virus which expresses KPP in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

For long term production of recombinant proteins in mammalian systems, stable expression of KPP in cell lines is preferred. For example, polynucleotides encoding KPP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk and apr cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding KPP is inserted within a marker gene sequence, transformed cells containing polynucleotides encoding KPP can be identified by the absence of marker gene function. Alternatively, a marker gene can be placed in tandem with a sequence encoding KPP under the control of a single promoter. Expression of the marker gene in response to induction or selection usually indicates expression of the tandem gene as well.

In general, host cells that contain the polynucleotide encoding KPP and that express KPP may be identified by a variety of procedures known to those of skill in the art These procedures include, but are not limited to, DNA-DNA. or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of KPP using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on KPP is preferred, but a competitive binding assay may be employed. These and other assays are well known in the art (Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding KPP include oligolabeling, nick translation, end-labeling, or PCR amplification using a labeled nucleotide. Alternatively, polynucleotides encoding KPP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures maybe conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding KPP may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode KPP may be designed to contain signal sequences which direct secretion of KPP through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding KPP may be ligated to a heterologous sequence resulting in translation of a fusion protein in any of the aforementioned host systems. For example, a chimeric KPP protein containing a heterologous moiety that can be recognized by a commercially available antibody may facilitate the screening of peptide libraries for inhibitors of KPP activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the KPP encoding sequence and the heterologous protein sequence, so that KPP may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled KPP may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

KPP, fragments of KPP, or variants of KPP may be used to screen for compounds that specifically bind to KPP. One or more test compounds may be screened for specific binding to KPP. In various embodiments, 1, 2, 3, 4, 5, 10, 20, 50, 100, or 200 test compounds can be screened for specific binding to KPP. Examples of test compounds can include antibodies, anticalins, oligonucleotides, proteins (e.g., ligands or receptors), or small molecules.

In related embodiments, variants of KPP can be used to screen for binding of test compounds, such as antibodies, to KPP, a variant of KPP, or a combination of KPP and/or one or more variants KPP. In an embodiment, a variant of KPP can be used to screen for compounds that bind to a variant of KPP, but not to KPP having the exact sequence of a sequence of SEQ ID NO:1-52. KPP variants used to perform such screening can have a range of about 50% to about 99% sequence identity to KPP, with various embodiments having 60%, 70%, 75%, 80%, 85%, 90%, and 95% sequence identity.

In an embodiment, a compound identified in a screen for specific binding to KPP can be closely related to the natural ligand of KPP, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor KPP (Howard, A. D. et al. (2001) Trends Pharmacol. Sci.22:132-140; Wise, A. et al. (2002) Drug Discovery Today 7:235-246).

In other embodiments, a compound identified in a screen for specific binding to KPP can be closely related to the natural receptor to which KPP binds, at least a fragment of the receptor, or a fragment of the receptor including all or a portion of the ligand binding site or binding pocket. For example, the compound may be a receptor for KPP which is capable of propagating a signal, or a decoy receptor for KPP which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (TNF) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to KPP, fragments of KPP, or variants of KPP. The binding specificity of the antibodies thus screened can thereby be selected to identify particular fragments or variants of KPP. In one embodiment, an antibody can be selected such that its binding specificity allows for preferential identification of specific fragments or variants of KPP. In another embodiment, an antibody can be selected such that its binding specificity allows for preferential diagnosis of a specific disease or condition having increased, decreased, or otherwise abnormal production of KPP.

In an embodiment, anticalins can be screened for specific binding to KPP, fragments of KPP, or variants of KPP. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit KPP involves producing appropriate cells which express KPP, either as a secreted protein or on the cell membrane. Preferred cells can include cells from mammals, yeast, Drosophila, or E. coli. Cells expressing KPP or cell membrane fractions which contain KPP are then contacted with a test compound and binding, stimulation, or inhibition of activity of either KPP or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with KPP, either in solution or affixed to a solid support, and detecting the binding of KPP to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

KPP, fragments of KPP, or variants of KPP may be used to screen for compounds that modulate the activity of KPP. Such compounds may include agonists, antagonists, or partial or inverse agonists. In one embodiment, an assay is performed under conditions permissive for KPP activity, wherein KPP is combined with at least one test compound, and the activity of KPP in the presence of a test compound is compared with the activity of KPP in the absence of the test compound. A change in the activity of KPP in the presence of the test compound is indicative of a compound that modulates the activity of KPP. Alternatively, a test compound is combined with an in vitro or cell-free system comprising KPP under conditions suitable for KPP activity, and the assay is performed. In either of these assays, a test compound which modulates the activity of KPP may do so indirectly and need not come in direct contact with the test compound. At least one and up to a plurality of test compounds may be screened.

In another embodiment, polynucleotides encoding KPP or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding KPP may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding KPP can also be used to create “knockin” humanized animals (pigs) or transgenic animals (mice or rats) to model human disease. With knockin technology, a region of a polynucleotide encoding KPP is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress KPP, e.g., by secreting KPP in its milk, may also serve as a convenient source of that protein (Janne, J. et al. (1998) Biotechnol. Annu. Rev. 4:55-74).

### Therapeutics

Chemical and structural similarity, e.g., in the context of sequences and motifs, exists between regions of KPP and kinases and phosphatases. In addition, examples of tissues expressing KPP can be found in Table 6 and can also be found in Example XI. Therefore, KPP appears to play a role in cardiovascular diseases, immune system disorders, neurological disorders, disorders affecting growth and development, lipid disorders, cell proliferative disorders, and cancers. In the treatment of disorders associated with increased KPP expression or activity, it is desirable to decrease the expression or activity of KPP. In the treatment of disorders associated with decreased KPP expression or activity, it is desirable to increase the expression or activity of KPP.

Therefore, in one embodiment, KPP or a fragment or derivative thereof may be administered to a subject to treat or prevent a disorder associated with decreased expression or activity of KPP. Examples of such disorders include, but are not limited to, a cardiovascular disease such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation, congenital lung anomalies, atelectasis, pulmonary congestion and edema, pulmonary embolism, pulmonary hemorrhage, pulmonary infarction, pulmonary hypertension, vascular sclerosis, obstructive pulmonary disease, restrictive pulmonary disease, chronic obstructive pulmonary disease, emphysema, chronic bronchitis, bronchial asthma, bronchiectasis, bacterial pneumonia, viral and mycoplasmal pneumonia, lung abscess, pulmonary tuberculosis, diffuse interstitial diseases, pneumoconioses, sarcoidosis, idiopathic pulmonary fibrosis, desquamative interstitial pneumonitis, hypersensitivity pneumonitis, pulmonary eosinophilia bronchiolitis obliterans-organizing pneumonia, diffuse pulmonary hemorrhage syndromes, Goodpasture's syndromes, idiopathic pulmonary hemosiderosis, pulmonary involvement in collagen-vascular disorders, pulmonary alveolar proteinosis, lung tumors, inflammatory and noninflammatory pleural effusions, pneumothorax, pleural tumors, drug-induced lung disease, radiation-induced lung disease, and complications of lung transplantation; an immune system disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a disorder affecting growth and development such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a lipid disorder such as fatty liver, cholestasis, primary biliary cirrhosis, carnitine deficiency, carnitine palintoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, diabetes mellitus, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoffs disease, hyperlipidemia, hyperlipemia, lipid myopathies, and obesity; and a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, uterus, leukemias such as multiple myeloma, and lymphomas such as Hodgkin's disease.

In another embodiment, a vector capable of expressing KPP or a fragment or derivative thereof may be administered to a subject to treat or prevent a disorder associated with decreased expression or activity of KPP including, but not limited to, those described above.

In a further embodiment, a composition comprising a substantially purified KPP in conjunction with a suitable pharmaceutical carrier may be administered to a subject to treat or prevent a disorder associated with decreased expression or activity of KPP including, but not limited to, those provided above.

In still another embodiment, an agonist which modulates the activity of KPP may be administered to a subject to treat or prevent a disorder associated with decreased expression or activity of KPP including, but not limited to, those listed above.

In a further embodiment, an antagonist of KPP may be administered to a subject to treat or prevent a disorder associated with increased expression or activity of KPP. Examples of such disorders include, but are not limited to, those cardiovascular diseases, immune system disorders, neurological disorders, disorders affecting growth and development, lipid disorders, cell proliferative disorders, and cancers described above. In one aspect, an antibody which specifically binds KPP may be used directly as an antagonist or indirectly as a targeting or delivery mechanism for bringing a pharmaceutical agent to cells or tissues which express KPP.

In an additional embodiment, a vector expressing the complement of the polynucleotide encoding KPP may be administered to a subject to treat or prevent a disorder associated with increased expression or activity of KPP including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of KPP may be produced using methods which are generally known in the art. In particular, purified KPP may be used to produce antibodies or to screen libraries of pharmaceutical agents to identify those which specifically bind KPP. Antibodies to KPP may also be generated using methods that are well known in the art. Such antibodies may include, but are not limited to, polyclonal, monoclonal, chimeric, and single chain antibodies, Fab fragments, and fragments produced by a Fab expression library. In an embodiment, neutralizing antibodies (i.e., those which inhibit dimer formation) can be used therapeutically. Single chain antibodies (e.g., from camels or llamas) may be potent enzyme inhibitors and may have application in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with KPP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to KPP have an amino acid sequence consisting of at least about 5 amino acids, and generally will consist of at least about 10 amino acids. It is also preferable that these oligopeptides, peptides, or fragments are substantially identical to a portion of the amino acid sequence of the natural protein. Short stretches of KPP amino acids may be fused with those of another protein, such as KLH, and antibodies to the chimeric molecule may be produced.

Monoclonal antibodies to KPP may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31742; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce KPP-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for KPP may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between KPP and its specific antibody. A two-site, monoclonal-based immunoassay utilizing monoclonal antibodies reactive to two non-interfering KPP epitopes is generally used, but a competitive binding assay may also be employed (Pound, supra).

Various methods such as Scatchard analysis in conjunction with radioimmunoassay techniques may be used to assess the affinity of antibodies for KPP. Affinity is expressed as an association constant, Kₐ, which is defined as the molar concentration of KPP-antibody complex divided by the molar concentrations of free antigen and free antibody under equilibrium conditions. The Kₐ determined for a preparation of polyclonal antibodies, which are heterogeneous in their affinities for multiple KPP epitopes, represents the average affinity, or avidity, of the antibodies for KPP. The Kₐ determined for a preparation of monoclonal antibodies, which are monospecific for a particular KPP epitope, represents a true measure of affinity. High-affinity antibody preparations with Kₐ ranging from about 10⁹ to 10¹² L/mole are preferred for use in immunoassays in which the KPP-antibody complex must withstand rigorous manipulations. Low-affinity antibody preparations with Kₐ ranging from about 10⁶ to 10⁷ L/mole are preferred for use in immunopurification and similar procedures which ultimately require dissociation of KPP, preferably in active form, from the antibody (Catty, D. (1988) Antibodies, Volume I: A Practical Approach, IRL Press, Washington DC; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of KPP-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding KPP, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding KPP. Such technology is well known in the art, and antisense oligonucleotides or larger fragments can be designed from various locations along the coding or control regions of sequences encoding KPP (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469-475; Scanlon, K. J. et al. (1995) 9:1288-1296). Antisense sequences can also be introduced intracellularly through the use of viral vectors, such as retrovirus and adeno-associated virus vectors (Miller, A. D. (1990) Blood 76:271; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding KPP may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor DC deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in KPP expression or regulation causes disease, the expression of KPP from an appropriate population of transduced cells may alleviate the clinical manifestations caused by the genetic deficiency.

In a further embodiment of the invention, diseases or disorders caused by deficiencies in KPP are treated by constructing mammalian expression vectors encoding KPP and introducing these vectors by mechanical means into KPP-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Recipon (1998) Curr. Opin. Biotechnol. 9:445-450).

Expression vectors that may be effective for the expression of KPP include, but are not limited to, the PCDNA 3.1, EPITAG, PRCCMV2, PREP, PVAX, PCR2-TOPOTA vectors (Invitrogen, Carlsbad Calif.), PCMV-SCRIWF, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). KPP may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding KPP from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to KPP expression are treated by constructing a retrovirus vector consisting of (i) the polynucleotide encoding KPP under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD⁴⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding KPP to cells which have one or more genetic abnormalities with respect to the expression of KPP. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding KPP to target cells which have one or more genetic abnormalities with respect to the expression of KPP. The use of herpes simplex virus (HSV)-based vectors may be especially valuable for introducing KPP to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding KPP to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for KPP into the alphavirus genome in place of the capsid-coding region results in the production of a large number of KPP-coding RNAs and the synthesis of high levels of KPP in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of KPP into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding KPP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding KPP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

In other embodiments of the invention, the expression of one or more selected polynucleotides of the present invention can be altered, inhibited, decreased, or silenced using RNA interference (RNAi) or post-transcriptional gene silencing (PTGS) methods known in the art. RNAi is a post-transcriptional mode of gene silencing in which double-stranded RNA (dsRNA) introduced into a targeted cell specifically suppresses the expression of the homologous gene (i.e., the gene bearing the sequence complementary to the dsRNA). This effectively knocks out or substantially reduces the expression of the targeted gene. PTGS can also be accomplished by use of DNA or DNA fragments as well. RNAi methods are described by Fire, A. et al. (1998; Nature 391:806-811) and Gura, T. (2000; Nature 404:804-808). PTGS can also be initiated by introduction of a complementary segment of DNA into the selected tissue using gene delivery and/or viral vector delivery methods described herein or known in the art.

RNAi can be induced in mammalian cells by the use of small interfering RNA also known as siRNA. SiRNA are shorter segments of dsRNA (typically about 21 to 23 nucleotides in length) that result in vivo from cleavage of introduced dsRNA by the action of an endogenous ribonuclease. SiRNA appear to be the mediators of the RNAi effect in mammals. The most effective siRNAs appear to be 21 nucleotide dsRNAs with 2 nucleotide 3′ overhangs. The use of siRNA for inducing RNAi in mammalian cells is described by Elbashir, S. M. et al. (2001; Nature 411:494-498).

SiRNA can either be generated indirectly by introduction of dsRNA into the targeted cell, or directly by mammalian transfection methods and agents described herein or known in the art (such as liposome-mediated transfection, viral vector methods, or other polynucleotide delivery/introductory methods). Suitable SiRNAs can be selected by examining a transcript of the target polynucleotide (e.g., mRNA) for nucleotide sequences downstream from the AUG start codon and recording the occurrence of each nucleotide and the 3′ adjacent 19 to 23 nucleotides as potential siRNA target sites, with sequences having a 21 nucleotide length being preferred. Regions to be avoided for target siRNA sites include the 5′ and 3′ untranslated regions (UTRs) and regions near the start codon (within 75 bases), as these may be richer in regulatory protein binding sites. UTR-binding proteins and/or translation initiation complexes may interfere with binding of the siRNP endonuclease complex. The selected target sites for siRNA can then be compared to the appropriate genome database (e.g., human, etc.) using BLAST or other sequence comparison algorithms known in the art. Target sequences with significant homology to other coding sequences can be eliminated from consideration. The selected SiRNAs can be produced by chemical synthesis methods known in the art or by in vitro transcription using commercially available methods and kits such as the SILENCER siRNA construction kit (Ambion, Austin Tex.).

In alternative embodiments, long-term gene silencing and/or RNAi effects can be induced in selected tissue using expression vectors that continuously express siRNA. This can be accomplished using expression vectors that are engineered to express hairpin RNAs (shRNAs) using methods known in the art (see, e.g., Brummelkamp, T. R. et al. (2002) Science 296:550-553; and Paddison, P. J. et al. (2002) Genes Dev. 16:948-958). In these and related embodiments, shRNAs can be delivered to target cells using expression vectors known in the art. An example of a suitable expression vector for delivery of siRNA is the PSILENCER1.0-U6 (circular) plasmid (Ambion). Once delivered to the target tissue, shRNAs are processed in vivo into siRNA-like molecules capable of carrying out gene-specific silencing.

In various embodiments, the expression levels of genes targeted by RNAi or PTGS methods can be determined by assays for mRNA and/or protein analysis. Expression levels of the mRNA of a targeted gene, can be determined by northern analysis methods using, for example, the NORTHERNMAX-GLY kit (Ambion); by microarray methods; by PCR methods; by real time PCR methods; and by other RNA/polynucleotide assays known in the art or described herein. Expression levels of the protein encoded by the targeted gene can be determined by Western analysis using standard techniques known in the art.

An additional embodiment of the invention encompasses a method for screening for a compound which is effective in altering expression of a polynucleotide encoding KPP. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased KPP expression or activity, a compound which specifically inhibits expression of the polynucleotide encoding KPP may be therapeutically useful, and in the treatment of disorders associated with decreased KPP expression or activity, a compound which specifically promotes expression of the polynucleotide encoding KPP may be therapeutically useful.

In various embodiments, one or more test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding KPP is exposed to at least one test compound thus obtained. The sample may comprise, for example, an intact or permeabilized cell, or an in vitro cell-free or reconstituted biochemical system. Alterations in the expression of a polynucleotide encoding KPP are assayed by any method commonly known in the art. Typically, the expression of a specific nucleotide is detected, by hybridization with a probe having a nucleotide sequence complementary to the sequence of the polynucleotide encoding KPP. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of KPP, antibodies to KPP, and mimetics, agonists, antagonists, or inhibitors of KPP.

In various embodiments, the compositions described herein, such as pharmaceutical compositions, may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery allows administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising KPP or fragments thereof. For example, liposome preparations containing a cell-impermeable macromolecule may promote cell fusion and intracellular delivery of the macromolecule. Alternatively, KPP or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example KPP or fragments thereof, antibodies of KPP, and agonists, antagonists or inhibitors of KPP, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

### Diagnostics

In another embodiment, antibodies which specifically bind KPP may be used for the diagnosis of disorders characterized by expression of KPP, or in assays to monitor patients being treated with KPP or agonists, antagonists, or inhibitors of KPP. Antibodies useful for diagnostic purposes may be prepared in the same manner as described above for therapeutics. Diagnostic assays for KPP include methods which utilize the antibody and a label to detect KPP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring KPP, including ELISAs, RIAs, and FACS, are known in the art and provide a basis for diagnosing altered or abnormal levels of KPP expression. Normal or standard values for KPP expression are established by combining body fluids or cell extracts taken from normal mammalian subjects, for example, human subjects, with antibodies to KPP under conditions suitable for complex formation. The amount of standard complex formation may be quantitated by various methods, such as photometric means. Quantities of KPP expressed in subject, control, and disease samples from biopsied tissues are compared with the standard values. Deviation between standard and subject values establishes the parameters for diagnosing disease.

In another embodiment of the invention, polynucleotides encoding KPP may be used for diagnostic purposes. The polynucleotides which may be used include oligonucleotides, complementary RNA and DNA molecules, and PNAs. The polynucleotides may be used to detect and quantify gene expression in biopsied tissues in which expression of KPP may be correlated with disease. The diagnostic assay may be used to determine absence, presence, and excess expression of KPP, and to monitor regulation of KPP levels during therapeutic intervention.

In one aspect, hybridization with PCR probes which are capable of detecting polynucleotides, including genonic sequences, encoding KPP or closely related molecules may be used to identify nucleic acid sequences which encode KPP. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding KPP, allelic variants, or related sequences.

Probes may also be used for the detection of related sequences, and may have at least 50% sequence identity to any of the KPP encoding sequences. The hybridization probes of the subject invention may be DNA or RNA and may be derived from the sequence of SEQ ID NO:53-104 or from genomic sequences including promoters, enhancers, and introns of the KPP gene.

Means for producing specific hybridization probes for polynucleotides encoding KPP include the cloning of polynucleotides encoding KPP or KPP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotides encoding KPP may be used for the diagnosis of disorders associated with expression of KPP. Examples of such disorders include, but are not limited to, a cardiovascular disease such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation, congenital lung anomalies, atelectasis, pulmonary congestion and edema, pulmonary embolism, pulmonary hemorrhage, pulmonary infarction, pulmonary hypertension, vascular sclerosis, obstructive pulmonary disease, restrictive pulmonary disease, chronic obstructive pulmonary disease, emphysema, chronic bronchitis, bronchial asthma, bronchiectasis, bacterial pneumonia, viral and mycoplasmal pneumonia, lung abscess, pulmonary tuberculosis diffuse interstitial diseases, pneumoconioses, sarcoidosis, idiopathic pulmonary fibrosis, desquamative interstitial pneumonitis, hypersensitivity pneumonitis, pulmonary eosinophilia bronchiolitis obliterans-organizing pneumonia, diffuse pulmonary hemorrhage syndromes, Goodpasture's syndromes, idiopathic pulmonary hemosiderosis, pulmonary involvement in collagen-vascular disorders, pulmonary alveolar proteinosis, lung tumors, inflammatory and noninflammatory pleural effusions, pneumothorax, pleural tumors, drug-induced lung disease, radiation-induced lung disease, and complications of lung transplantation; an immune system disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erytdroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a disorder affecting growth and development such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a lipid disorder such as fatty liver, cholestasis, primary biliary cirrhosis, carnitine deficiency, carnitine palmtoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, diabetes mellitus, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoffs disease, hyperlipidemia, hyperlipemia, lipid myopathies, and obesity; and a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, uterus, leukemias such as multiple myeloma, and lymphomas such as Hodgkin's disease. Polynucleotides encoding KPP may be used in Southern or northern analysis, dot blot, or other membrane-based technologies; in PCR technologies; in dipstick, pin, and multiformat ELISA-like assays; and in microarrays utilizing fluids or tissues from patients to detect altered KPP expression. Such qualitative or quantitative methods are well known in the art.

In a particular embodiment, polynucleotides encoding KPP may be used in assays that detect the presence of associated disorders, particularly those mentioned above. Polynucleotides complementary to sequences encoding KPP may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding KPP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of KPP, a normal or standard profile for expression is established. This may be accomplished by combining body fluids or cell extracts taken from normal subjects, either animal or human, with a sequence, or a fragment thereof, encoding KPP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding KPP may involve the use of PCR. These oligomers may be chemically synthesized, generated enzymatically, or produced in vitro. Oligomers will preferably contain a fragment of a polynucleotide encoding KPP, or a fragment of a polynucleotide complementary to the polynucleotide encoding KPP, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding KPP maybe used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding KPP are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of KPP include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, KPP, fragments of KPP, or antibodies specific for KPP may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at http:f/www.niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for KPP to quantify the levels of KPP expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding KPP may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding KPP on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, KPP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between KPP and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with KPP, or fragments thereof, and washed. Bound KPP is then detected by methods well known in the art Purified KPP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding KPP specifically compete with a test compound for binding KPP. In this manner, antibodies can be used to detect the presence of any peptide which shares one or more antigenic determinants with KPP.

In additional embodiments, the nucleotide sequences which encode KPP may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent The following embodiments are, therefore, to be construed as merely illustrative, and not limitative of the remainder of the disclosure in any way whatsoever.

The disclosures of all patents, applications, and publications mentioned above and below, including U.S. Ser. No. 60/345,474 U.S. Ser. No. 60/343,910, U.S. Ser. No. 60/333,098, U.S. Ser. No. 60/332,424, and U.S. Ser. No. 60/334,288, are hereby expressly incorporated by reference.

### EXAMPLES

### I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art (Ausubel et al., supra, ch. 5). Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen, Carlsbad Calif.), PCDNA2.1 plasmid (Invitrogen), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Invitrogen.

### II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

### III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific), or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (Ausubel et al., supra, ch. 7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families; see, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (MiraiBio, Alameda Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:53-104. Fragments from about 20 to about 4000 nucleotides which are useful in hybridization and amplification technologies are described in Table 4, column 2.

### IV. Identification and Editing of Coding Sequences from Genomic DNA

Putative kinases and phosphatases were initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode kinases and phosphatases, the encoded polypeptides were analyzed by querying against PFAM models for kinases and phosphatases. Potential kinases and phosphatases were also identified by homology to Incyte cDNA sequences that had been annotated as kinases and phosphatases. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

### V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example m were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

### VI. Chromosomal Mapping of KPP Encoding Polynucleotides

The sequences which were used to assemble SEQ ID NO:53-104 were compared with sequences from the Incyte LIFESEQ database and public domain databases using BLAST and other implementations of the Smith-Waterman algorithm. Sequences from these databases that matched SEQ ID NO:53-104 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap '99” World Wide Web site (http://www.ncbi.nlm.nih.gov/genemap/), can be employed to determine if previously identified disease genes map within or in proximity to the intervals indicated above.

### VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook and Russell, supra, ch. 7; Ausubel et al., supra, ch. 4).

Analogous computer techniques applying BLAST were used to search for identical or related molecules in databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding KPP are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding KPP. cDNA sequences and cDNA library/tissue information are found in the LIFESEQ GOLD database (Incyte Genomics, Palo Alto Calif.).

### VIII. Extension of KPP Encoding Polynucleotides

Full length polynucleotides are produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

### IX. Identification of Single Nucleotide Polymorphisms in KPP Encoding Polynucleotides

Common DNA sequence variants known as single nucleotide polymorphisms (SNPs) were identified in SEQ ID NO:53-104 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper triming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

### X. Labeling and Use of Individual Hybridization Probes

Hybridization probes derived from SEQ ID NO:53-104 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 107 counts per minute of the labeled probe is used in a typical membrane-based hybridization analysis of human genomic DNA digested with one of the following endonucleases: AseI, BglII, EcoRI, PstI, XbaI, or PvuII (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

### XI. Microarrays

The linkage or synthesis of array elements upon a microarray can be achieved utilizing photolithography, piezoelectric printing (ink-jet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21 mer), 1× first strand buffer, 0.03 units/μl RNase inhibitor, 500 μM dATP, 500 μM dGTP, 500 μM dTTP, 40 μM dCTWP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte Genomics). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (Clontech, Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a comer of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte Genomics). Array elements that exhibit at least about a two-fold change in expression, a signal-to-background ratio of at least about 2.5, and an element spot size of at least about 40%, are considered to be differentially expressed.

Expression

SEQ ID NO:57 showed differential expression in liver tumor derived cells treated with the hormones progesterone and beclamethasone, as determined by microarray analysis. The C3A line is a clonal derivative of the Hep G2 hepatoma cell line isolated from a 15-year-old male with a liver tumor. The C3A cells express insulin receptor and insulin-like growth factor 11 receptor. Progesterone is a naturally occurring progestin, which is metabolized hepatically. Beclamethasone is a synthetic glucocorticoid used for treating steroid-dependent asthma. Glucocorticoids are naturally occurring hormones that prevent or suppress inflammation and immune responses when administered at pharmacological doses. Early confluent C3A cells were treated with progesterone at 100 μM or beclamethasone at 10 μM, for 1, 3 and 6 hours and compared to untreated C3A cells. The expression of SEQ ID NO:57 was increased by at least two-fold at all time points in both treatments. These experiments indicate that SEQ ID NO57 is useful in diagnostic assays for diseases involving kinases and phosphatases, as a potential biological marker and therapeutic agent in the treatment of diseases involving kinases and phosphatases, and in monitoring the effects of glucocorticoids on the liver.

SEQ ID NO:65 showed differential expression, as determined by microarray analysis, in Alzheimer Disease (AD). In a comparison of anterior hippocampal tissue from a 79-year-old female with severe AD to anterior hippocampal tissue from a normal 61-year-old female, the expression of SEQ ID NO:65 was decreased at least two-fold. Therefore, SEQ ID NO:65 is useful in diagnostic assays for AD and as a potential biological marker and therapeutic agent in the treatment of AD.

SEQ ID NO:67 showed differential expression, as determined by microarray analysis, in liver C3A cells treated with one of the following steroids: beclomethasone, dexamethasone, progesterone, medroxyprogesterone, budesonide, prednisone, betamethasone. The human C3A cell line is a clonal derivative of HepG2/C3 and has been established as an in vitro model of the mature human liver (Mickelson et al. (1995) Hepatology 22:866-875; Nagendra et al. (1997) Am J Physiol 272:G408-G416). SEQ ID NO:67 showed at least a two-fold decrease in expression in early confluent C3A cells treated with progesterone, beclomethasone, medroxyprogesterone, budesonide, prednisone, dexamethasone, or betamethasone, for 1, 3, or 6 hours. These experiments indicate that SEQ ID NO:67 is useful in diagnostic assays for liver diseases and as a potential biological marker and therapeutic agent in the treatment of liver diseases and disorders.

SEQ ID NO:67 also showed differential expression in prostate carcinoma cell lines versus normal prostate epithelial cells as determined by microarray analysis. The prostate carcinoma cell line DU 145 was isolated from a metastatic site in the brain of a 69 year old male with widespread metastatic prostate carcinoma. DU 145 has no detectable sensitivity to hormones; forms colonies in semi-solid medium; is only weakly positive for acid phosphatase; and cells are negative for prostate specific antigen (PSA). The normal epithelial cell line, PrEC, is a primary prostate epithelial cell line isolated from a normal donor. The microarray experiments showed that the expression of SEQ ID NO:67 was increased by at least two fold in the prostate carcinoma line DU 145 relative to cells from the normal prostate epithelial cell line, PrEC. Therefore, SEQ ID NO:67 is useful as a diagnostic marker or as a potential therapeutic target for certain prostate cancers.

In another example, SEQ ID NO:68, SEQ ID NO:70, and SEQ ID NO:72 showed differential expression in tumorous tissue versus non-tumorous tissues, as determined by microarray analysis. The expression of cDNAs from lung, ovarian, and colon tumor tissue from several donors was compared with that of normal lung, ovarian, and colon tissue from the same donor, respectively.

The expression of SEQ ID NO:68 was increased at least 2.8-fold in a lung squamous cell carcinoma when matched with normal tissue from the same donor. The tumorous lung tissue was obtained from the lung of a 68-year-old female with lung squamous cell carcinoma. Normal lung tissue was obtained from grossly uninvolved tissue from the lung of the same donor. Therefore, SEQ ID NO:68 is useful in diagnostic assays for lung adenocarcinoma.

Further, the expression of SEQ ID NO:70 was decreased at least 2.3-fold in an ovarian adenocarcinoma when matched with normal tissue from the same donor. The tumorous ovary tissue was obtained from ovarian adenocarcinoma from a 79-year-old female. Normal ovary tissue was obtained from ovary from the same donor. Therefore, SEQ ID NO:70 is useful in diagnostic assays for ovarian adenocarcinoma.

The expression of SEQ ID NO:72 was decreased at least two-fold in human colon adenocarcinoma tissue from two donors when matched with normal tissue from the same donor, respectively. The colon adenocarcinoma tissue was obtained from an 85-year old female with colon adenocarcinoma or from an 85-year old male with colon adenocarcinoma. Normal colon tissue was obtained from grossly uninvolved pooled normal colon tissue or from grossly uninvolved colon tissue from the same donor, respectively. The expression of SEQ ID NO:72 also was decreased at least 3.5-fold in human rectal tumor tissue when matched with normal rectal tissue from the same donor. The rectal tumor tissue was obtained from a male (age unknown) with rectal cancer. Normal rectal tissue was obtained from grossly uninvolved rectal tissue from the same donor. Further, SEQ ID NO:72 was decreased at least 8-fold in human sigmoid colon tumor tissue matched with normal tissue form the same donor. The sigmoid colon tissue was obtained from a 48-year old female with sigmoid color tumor originating from a metastatic gastric sarcoma (stromal tumor). Normal sigmoid colon tissue was obtained from grossly uninvolved sigmoid colon tissue from the same donor. Therefore, SEQ ID NO:72 is useful in diagnostic assays for colon cancer, rectal cancer, and sigmoid colon cancer.

Matched normal and tumorigenic colon and ovary tissue samples are provided by the Huntsman Cancer Institute, (Salt Lake City, Utah). Matched normal and tumorigenic lung tissue samples are provided by the Roy Castle International Centre for Lung Cancer Research (Liverpool, UK).

In another example, the expression of SEQ ID NO:79 was decreased at least two-fold in human cancerous colon tissue matched with normal tissue from the same donors. Colon adenocarcinoma tissue was obtained from an 59-year-old male with a tubulovillous adenoma hyperplastic polyp of the colon and was matched with normal colon tissue obtained from grossly uninvolved pooled colon tissue from the same donor. Therefore, SEQ ID NO:79 is useful in diagnostic assays for colon cancer. Matched normal and tumorigenic colon tissue samples are provided by the Huntsman Cancer Institute, (Salt Lake City, Utah).

In another example, the expression of SEQ ID NO:82 in several tumor cell lines representing various stages of breast tumor progression was compared with that in the non-malignant mammary epithelial cell line, MCF-10A. For example, the expression of SEQ ID NO:82 from five tumor cell lines (BT20, MCF7, MDA-mb-231, Sk-BR-3, and T-47D) was compared with that in MCF-10A cells grown in the supplier's recommended medium or grown in defined serum-free H14 medium to 70-80% confluence prior to comparison. MCF-10A is a breast mammary gland (luminal ductal characteristics) cell line that was isolated from a 36-year-old woman with fibrocystic breast disease. MCF-10A expresses cytoplasmic keratins, epithelial sialomucins, and milkfat globule antigens. This cell lines exhibits three-dimensional growth in collagen and forms domes in confluent culture. MCF7 is a nonmalignant breast adenocarcinoma cell line isolated from the pleural effusion of a 69-year-old female. MCF7 has retained characteristics of the mammary epithelium such as the ability to process estradiol via cytoplasmic estrogen receptors and the capacity to form domes in culture. T-47D is a breast carcinoma cell line isolated from a pleural effusion obtained from a 54-year-old female with an infiltrating ductal carcinoma of the breast. Sk-BR-3 is a breast adenocarcinoma cell line isolated from a malignant pleural effusion of a 43-year-old female. It forms poorly differentiated adenocarcinoma when injected into nude mice. BT-20 is a breast carcinoma cell line derived in vitro from cells emigrating out of thin slices of the tumor mass isolated from a 74-year-old female. MDA-mb-231 is a breast tumor cell line isolated from the pleural effusion of a 51-year old female. It forms poorly differentiated adenocarcinoma in nude mice and ALS treated BALB/c mice. It also expresses the Wnt3 oncogene, EGF, and TGF-α. MDA-mb435S is a spindle shaped strain that evolved from the parent line (435) as isolated in 1976 by R. Cailleau from the pleural effusion of a 31-year-old female with metastatic, ductal adenocarcinoma of the breast. SEQ ID NO:82 showed at least two-fold increased expression when comparing MCF-10A cells versus BT-20, MCF7, and Sk-BR-3 cells. These experiments indicate that SEQ ID NO:82 was significantly under-expressed in the breast tumor cell lines tested, further establishing the utility of SEQ ID NO:82 as a diagnostic marker or as a potential therapeutic target for breast cancer.

Further, the expression of SEQ ID NO:82 was increased at least two-fold in treated human adipocytes from an obese donor when compared to non-treated adipocytes from the same donor. The obese human primary subcutaneous preadipocytes were isolated from adipose tissue of a 40-year-old healthy female with a body mass index (BMI) of 32.47. The preadipocytes were cultured and induced to differentiate into adipocytes by culturing them in the differentiation medium containing the active components, PPAR-γ agonist and human insulin. Human preadipocytes were treated with human insulin and PPAR-γ agonist for three days and subsequently were switched to medium containing insulin alone for a total duration of 24 hours, 48 hours, four days, 8 days or 15 days before the cells were collected for analysis. Differentiated adipocytes were compared to untreated preadipocytes maintained in culture in the absence of inducing agents. Between 80% and 90% of the preadipocytes finally differentiated to adipocytes as observed under phase contrast microscope. Thus, SEQ ID NO:82 is useful for the diagnosis, prognosis, or treatment of diabetes mellitus and other disorders, such as obesity, hypertension, atherosclerosis, polycystic ovarian syndrome, and cancers including breast, prostate, and colon.

The expression of SEQ ID NO:83 was decreased at least two-fold in cancerous lung tissue compared to normal tissue from the same donor. Moderately differentiated adenocarcinoma tissue from the right lung was obtained from a 60-year-old donor and matched with normal right lung tissue obtained from grossly uninvolved tissue from the same donor. Therefore, SEQ ID NO:83 is useful in diagnostic assays for lung cancer. Further, SEQ ID NO:83 was decreased at least 2.4-fold in cancerous ovarian tissue compared to normal tissue from the same donor. Ovarian adenocarcinoma was obtained from a 79-year-old female and matched with normal ovary tissue from the same donor. Therefore, SEQ ID NO:83 is useful in diagnostic assays for ovarian cancer. Matched normal and tumorigenic lung and ovarian tissue samples are provided by the Huntsman Cancer Institute, (Salt Lake City, Utah).

The expression of SEQ ID NO:84 was increased at least two-fold in Tangier disease-derived fibroblasts compared to normal fibroblasts. In addition, both types of cells were cultured in the presence of cholesterol and compared with the same cell type cultured in the absence of cholesterol. Human fibroblasts were obtained from skin explants from both normal subjects and two patients with homozygous Tangier disease. Cell lines were immortalized by transfection with human papillomavirus 16 genes E6 and E7 and a neomycin resistance selectable marker. TD derived cells are deficient in an assay of apoA-I mediated tritiated cholesterol efflux. Therefore, SEQ ID NO:84 is useful in diagnostic assays for Tangier disease.

The expression of SEQ ID NO:86 in several tumor cell lines representing various stages of breast tumor progression was compared with that in the non-malignant mammary epithelial cell lines, HMEC and MCF-10A. For example, the expression of SEQ ID NO:86 from six cell lines (BT20, MCF7, MDA-mb-231, Sk-BR-3, MDA-mb-435S, and T-47D) was compared with that in HMEC cells or MCF-10A cells grown in the supplier's recommended medium to 70-80% confluence prior to comparison. SEQ ID NO:86 was decreased at least two-fold in five of six cell lines (MCF7, MDA-mb-231, Sk-BR-3, MDA-mb-435S, and T-47D) when compared with HMEC cells and in two of six cell lines (MDA-mb-231 and T-47D) when compared with MCF-10A cells. These experiments indicate that SEQ ID NO:86 was significantly under-expressed in the breast tumor cell lines tested, establishing the utility of SEQ ID NO:86 as a diagnostic marker or as a potential therapeutic target for breast cancer.

In another example, SEQ ID NO:98 showed differential expression associated with breast cancer as determined by microarray analysis. The gene expression profile of a nonmalignant mammary epithelial cell line was compared to the gene expression profiles of breast carcinoma cell lines representing different stages of tumor progression. The cell lines compared included: a) BT-20, a breast carcinoma cell line derived in vitro from the cells emigrating out of thin slices of tumor mass isolated from a 74-year-old female, b) BT-474, a breast ductal carcinoma cell line that was isolated from a solid, invasive ductal carcinoma of the breast obtained from a 60-year-old woman, c) BT-483, a breast ductal carcinoma cell line that was isolated from a papillary invasive ductal tumor obtained from a 23-year-old normal, menstruating, parous female with a family history of breast cancer, d) Hs 578T, a breast ductal carcinoma cell line isolated from a 74-year-old female with breast carcinoma, e) MCF7, a nonmalignant breast adenocarcinoma cell line isolated from the pleural effusion of a 69-year-old female, f) MCF-10A, a breast mammary gland (luminal ductal characteristics) cell line isolated from a 36-year-old woman with fibrocystic breast disease, and g) HMEC, a primary breast epithelial cell line isolated from a normal donor. The expression of SEQ ID NO:98 was at least two-fold lower in all of the breast carcinoma cell lines compared to the HMEC cell line. Therefore SEQ ID NO:98 is useful in diagnostic assays and disease staging assays for cell proliferative disorders, including breast cancer.

In another example, SEQ ID NO:100 showed differential expression associated with osteosarcoma as determined by microarray analysis. Messenger RNA from normal human osteoblasts (primary culture, NHOst 5488) was compared with mRNA from biopsy specimens and osteosarcoma tissues. The expression of SEQ ID NO:100 was increased by at least two-fold in femur bone tumor tissue from patients with osteosarcoma compared to normal osteoblasts. Therefore, SEQ ID NO:100 is useful in monitoring treatment of and diagnostic assays for osteosarcoma.

In another example, SEQ ID NO:101 showed differential expression associated with lung cancer. The expression of SEQ ID NO:101 was compared in normal and cancerous tissue samples from ten patients with lung tumors, including three patients with adenocarcinoma and five patients with squamous cell carcinoma. SEQ ID NO:101 showed at least a two-fold increase in expression in lung tissue from three out of five patients with lung squamous cell carcinoma compared to matched microscopically normal tissue from the same donors as determined by microarray analysis. In addition, SEQ ID NO:101 showed differential expression associated with Alzheimer's disease. SEQ ID NO:101 showed at least a two fold decrease in expression in cells or tissues of brains from subjects with Alzheimer's disease compared to normal brain tissue. Therefore, SEQ ID NO:101 is useful in disease staging and diagnostic assays for lung cancer, particularly squamous cell carcinoma, and for neurological disorders such as Alzheimer's disease.

### XII. Complementary Polynucleotides

Sequences complementary to the KPP-encoding sequences, or any parts thereof, are used to detect, decrease, or inhibit expression of naturally occurring KPP. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of KPP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the KPP-encoding transcript.

XIII. Expression of KPP

Expression and purification of KPP is achieved using bacterial or virus-based expression systems. For expression of KPP in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express KPP upon induction with isopropyl beta-D-thiogalactopyranoside (U7G). Expression of KPP in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding KPP by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum. Gene Ther. 7:1937-1945).

In most expression systems, KPP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from KPP at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified KPP obtained by these methods can be used directly in the assays shown in Examples XVII, XVIII, XIX, XX, and XXI, where applicable.

### XIV. Functional Assays

KPP function is assessed by expressing the sequences encoding KPP at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endotheilal or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of KPP on gene expression can be assessed using highly purified populations of cells transfected with sequences encoding KPP and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding KPP and other genes of interest can be analyzed by northern analysis or microarray techniques.

### XV. Production of KPP Specific Antibodies

KPP substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the KPP amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity (Ausubel et al., supra). Rabbits are immunized with the oligopeptide-KLH complex in complete Freund's adjuvant Resulting antisera are tested for antipeptide and anti-KPP activity by, for example, binding the peptide or KPP to a substrate, blocking with 1% BSA, reacting with rabbit antisera, washing, and reacting with radio-iodinated goat anti-rabbit IgG.

### XVI. Purification of Naturally Occurring KPP Using Specific Antibodies

Naturally occurring or recombinant KPP is substantially purified by immunoaffinity chromatography using antibodies specific for KPP. An immunoaffinity column is constructed by covalently coupling anti-KPP antibody to an activated chromatographic resin, such as CNBr-activated SEPHAROSE (Amersham Biosciences). After the coupling, the resin is blocked and washed according to the manufacturer's instructions.

Media containing KPP are passed over the immunoaffinity column, and the column is washed under conditions that allow the preferential absorbance of KPP (e.g., high ionic strength buffers in the presence of detergent). The column is eluted under conditions that disrupt antibody/KPP binding (e.g., a buffer of pH 2 to pH 3, or a high concentration of a chaotrope, such as urea or thiocyanate ion), and KPP is collected.

### XVII. Identification of Molecules Which Interact with KPP

KPP, or biologically active fragments thereof, are labeled with ¹²⁵I Bolton-Hunter reagent (Bolton, A. E. and W. M. Hunter (1973) Biochem. J. 133:529-539). Candidate molecules previously arrayed in the wells of a multi-well plate are incubated with the labeled KPP, washed, and any wells with labeled KPP complex are assayed. Data obtained using different concentrations of KPP are used to calculate values for the number, affinity, and association of KPP with the candidate molecules.

Alternatively, molecules interacting with KPP are analyzed using the yeast two-hybrid system as described in Fields, S. and O. Song (1989; Nature 340:245-246), or using commercially available kits based on the two-hybrid system, such as the MATCHMAKER system (Clontech).

KPP may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

### XVIII. Demonstration of KPP Activity

Generally, protein kinase activity is measured by quantifying the phosphorylation of a protein substrate by KPP in the presence of [γ-³²P]ATP. KPP is incubated with the protein substrate, ³²P-ATP, and an appropriate kinase buffer. The ³²P incorporated into the substrate is separated from free ³²P-ATP by electrophoresis and the incorporated ³²P is counted using a radioisotope counter. The amount of incorporated ³²P is proportional to the activity of KPP. A determination of the specific amino acid residue phosphorylated is made by phosphoamino acid analysis of the hydrolyzed protein.

In one alternative, protein kinase activity is measured by quantifying the transfer of gamma phosphate from adenosine triphosphate (ATP) to a serine, threonine or tyrosine residue in a protein substrate. The reaction occurs between a protein kinase sample with a biotinylated peptide substrate and gamma ³²P-ATP. Following the reaction, free avidin in solution is added for binding to the biotinylated ³²P-peptide product. The binding sample then undergoes a centrifugal ultrafiltration process with a membrane which will retain the product-avidin complex and allow passage of free gamma ³²P-ATP. The reservoir of the centrifuged unit containing the ³²P-peptide product as retentate is then counted in a scintillation counter. This procedure allows the assay of any type of protein kinase sample, depending on the peptide substrate and kinase reaction buffer selected. This assay is provided in kit form (ASUA, Affinity Ultrafiltration Separation Assay, Transbio Corporation, Baltimore Md., U.S. Pat. No. 5,869,275). Suggested substrates and their respective enzymes include but are not limited to: Histone H1 (Sigma) and p34^{cdc2}kinase, Annexin I, Angiotensin (Sigma) and EGF receptor kinase, Annexin II and src kinase, ERK1 & ERK2 substrates and MEK, and myelin basic protein and ERK (Pearson, J. D. et al. (1991) Methods Enzymol. 200:62-81).

In another alternative, protein kinase activity of KPP is demonstrated in an assay containing KPP, 50 μI of kinase buffer, 1 μg substrate, such as myelin basic protein (MBP) or synthetic peptide substrates, 1 mM DTr, 10 μg ATP, and 0.5 μCi [γ-³²P]ATP. The reaction is incubated at 30° C. for 30 minutes and stopped by pipetting onto P81 paper. The unincorporated [γ-³²P]ATP is removed by washing and the incorporated radioactivity is measured using a scintillation counter. Alternatively, the reaction is stopped by heating to 100° C. in the presence of SDS loading buffer and resolved on a 12% SDS polyacrylamide gel followed by autoradiography. The amount of incorporated ³²P is proportional to the activity of KPP.

In yet another alternative, adenylate kinase or guanylate kinase activity of KPP may be measured by the incorporation of ³²P from [γ-³²P]ATP into ADP or GDP using a gamma radioisotope counter. KPP, in a kinase buffer, is incubated together with the appropriate nucleotide mono-phosphate substrate (AMP or GMP) and ³²P-labeled ATP as the phosphate donor. The reaction is incubated at 37° C. and terminated by addition of trichioroacetic acid. The acid extract is neutralized and subjected to gel electrophoresis to separate the mono-, di-, and triphosphonucleotide fractions. The diphosphonucleotide fraction is excised and counted. The radioactivity recovered is proportional to the activity of KPP.

In yet another alternative, other assays for KPP include scintillation proximity assays (SPA), scintillation plate technology and filter binding assays. Useful substrates include recombinant proteins tagged with glutathione transferase, or synthetic peptide substrates tagged with biotin. Inhibitors of KPP activity, such as small organic molecules, proteins or peptides, may be identified by such assays.

In another alternative, phosphatase activity of KPP is measured by the hydrolysis of paranitrophenyl phosphate (PNPP). KPP is incubated together with PNPP in HEPES buffer pH 7.5, in the presence of 0.1% β-mercaptoethanol at 37° C. for 60 min. The reaction is stopped by the addition of 6 ml of 10 N NaOH (Diamond, R. H. et al. (1994) Mol. Cell. Biol. 14:3752-62). Alternatively, acid phosphatase activity of KPP is demonstrated by incubating KPP-containing extract with 100 μl of 10 mM PNPP in 0.1 M sodium citrate, pH 4.5, and 50 μl of 40 mM NaCl at 37° C. for 20 min. The reaction is stopped by the addition of 0.5 ml of 0.4 M glycine/NaOH, pH 10.4 (Saftig, P. et al. (1997) J. Biol. Chem. 272:18628-18635). The increase in light absorbance at 410 nm resulting from the hydrolysis of PNPP is measured using a spectrophotometer. The increase in light absorbance is proportional to the activity of KPP in the assay.

In the alternative, KPP activity is determined by measuring the amount of phosphate removed from a phosphorylated protein substrate. Reactions are performed with 2 or 4 nM KPP in a final volume of 30 μl containing 60 mM Tris, pH 7.6, 1 mM EDTA, 1 mM EGTA, 0.1% β-mercaptoethanol and 10 μM substrate, ³²P-labeled on serine/threonine or tyrosine, as appropriate. Reactions are initiated with substrate and incubated at 30° C. for 10-15 min. Reactions are quenched with 450 μl of 4% (w/v) activated charcoal in 0.6 M HCl, 90 mM Na₄P₂O₇, and 2 mM NaH₂PO₄, then centrifuged at 12,000×g for 5 min. Acid-soluble ³²Pi is quantified by liquid scintillation counting (Sinclair, C. et al. (1999) J. Biol. Chem. 274:23666-23672).

### XIX. Kinase Binding Assay

Binding of KPP to a FLAG-CD44 cyt fusion protein can be determined by incubating KPP with anti-KPP-conjugated immunoaffinity beads followed by incubating portions of the beads (having 10-20 ng of protein) with 0.5 ml of a binding buffer (20 mM Tris-HCL (pH 7.4), 150 MM NaCl, 0.1% bovine serum albumin, and 0.05% Triton X-100) in the presence of ¹²⁵I-labeled FLAG-CD44cyt fusion protein (5,000 cpm/ng protein ) at 4° C. for 5 hours. Following binding, beads were washed thoroughly in the binding buffer and the bead-bound radioactivity measured in a scintillation counter (Bourguignon, L. Y. W. et al. (2001) J. Biol. Chem. 276:7327-7336). The amount of incorporated ³²P is proportional to the amount of bound KPP.

### XX. Identification of KPP Inhibitors

Compounds to be tested are arrayed in the wells of a 384-well plate in varying concentrations along with an appropriate buffer and substrate, as described in the assays in Example XVII. KPP activity is measured for each well and the ability of each compound to inhibit KPP activity can be determined, as well as the dose-response kinetics. This assay could also be used to identify molecules which enhance KPP activity.

### XXI. Identification of KPP Substrates

A KPP “substrate-trapping” assay takes advantage of the increased substrate affinity that may be conferred by certain mutations in the PTP signature sequence of protein tyrosine phosphatases. KPP bearing these mutations form a stable complex with their substrate; this complex may be isolated biochemically. Site-directed mutagenesis of invariant residues in the PTP signature sequence in a clone encoding the catalytic domain of KPP is performed using a method standard in the art or a commercial kit, such as the MUTA-GENE kit from BIO-RAD. For expression of KPP mutants in Escherichia coli, DNA fragments containing the mutation are exchanged with the corresponding wild-type sequence in an expression vector bearing the sequence encoding KPP or a glutathione S-transferase (GST)-KPP fusion protein. KPP mutants are expressed in E. coli and purified by chromatography.

The expression vector is transfected into COS1 or 293 cells via calcium phosphate-mediated transfection with 20 μg of CsCl-purified DNA per 10-cm dish of cells or 8 μg per 6-cm dish. Forty-eight hours after transfection, cells are stimulated with 100 ng/ml epidermal growth factor to increase tyrosine phosphorylation in cells, as the tyrosine kinase EGFR is abundant in COS cells. Cells are lysed in 50 mM Tris.HCl, pH 7.5/5 mM EDTA/150 mM NaCl/1% Triton X-100/5 mM iodoacetic acid/10 mM sodium phosphate/10 mM NaF/5 μg/ml leupeptin/5 μg/ml aprotinin/1 mM benzamidine (1 ml per 10-cm dish, 0.5 ml per 6-cm dish). KPP is immunoprecipitated from lysates with an appropriate antibody. GST-KPP fusion proteins are precipitated with glutathione-Sepharose, 4 μg of mAb or 10 μl of beads respectively per mg of cell lysate. Complexes can be visualized by PAGE or further purified to identify substrate molecules (Flint, A. J. et al. (1997) Proc. Natl. Acad. Sci. USA 94:1680-1685).

### XXII. KPP Secretion Assay

A high throughput assay may be used to identify polypeptides that are secreted in eukaryotic cells. In an example of such an assay, polypeptide expression libraries are constructed by fusing 5′-biased cDNAs to the 5′-end of a leaderless β-lactamase gene. β-lactamase is a convenient genetic reporter as it provides a high signal-to-noise ratio against low endogenous background activity and retains activity upon fusion to other proteins. A dual promoter system allows the expression of β-lactamase fusion polypeptides in bacteria or eukaryotic cells, using the lac or CMV promoter, respectively.

Libraries are first transformed into bacteria, e.g., E. coli, to identify library members that encode fusion polypeptides capable of being secreted in a prokaryotic system. Mammalian signal sequences direct the translocation of β-lactamase fusion polypeptides into the periplasm of bacteria where it confers antibiotic resistance to carbenicillin. Carbenicillin-selected bacteria are isolated on solid media, individual clones are grown in liquid media, and the resulting cultures are used to isolate library member plasmid DNA.

Mammalian cells, e.g., 293 cells, are seeded into 96-well tissue culture plates at a density of about 40,000 cells/well in 100 μl phenol red-free DME supplemented with 10% fetal bovine serum (FBS) (Life Technologies, Rockville, Md.). The following day, purified plasmid DNAs isolated from carbenicillin-resistant bacteria are diluted with 15 μl OPTI-MEM I medium (Life Technologies) to a volume of 25 μl for each well of cells to be transfected. In separate plates, 1 μl LF2000 Reagent (Life Technologies) is diluted into 25 μl/well OPTI-MEM I. The 25 μl diluted LF2000 Reagent is then combined with the 25 μl diluted DNA, mixed briefly, and incubated for 20 minutes at room temperature. The resulting DNA-LF2000 reagent complexes are then added directly to each well of 293 cells. Cells are also transfected with appropriate control plasmids expressing either wild-type β-lactamase, leaderless β-lactamase, or, for example, CD4-fused leaderless β-lactamase. 24 hrs following transfection, about 90 μl of cell culture media are assayed at 37° C. with 100 μM Nitrocefin (Calbiochem, San Diego, Calif.) and 0.5 mM oleic acid (Sigma Corp. St. Louis, Mo.) in 10 mM phosphate buffer (pH 7.0). Nitrocefin is a substrate for β-lactamase that undergoes a noticeable color change from yellow to red upon hydrolysis. β-lactamase activity is monitored over 20 min in a microtiter plate reader at 486 nm. Increased color absorption at 486 nm corresponds to secretion of a β-lactamase fusion polypeptide in the transfected cell media, resulting from the presence of a eukaryotic signal sequence in the fusion polypeptide. Polynucleotide sequence analysis of the corresponding library member plasmid DNA is then used to identify the signal sequence-encoding cDNA. (Described in U.S. patent application Ser. No. 09/803,317, filed Mar. 9, 2001.)

For example, SEQ ID NO:12 was shown to be a secreted protein using this assay.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | | | Incyte | |
|---|---|---|---|---|---|
| | Polypeptide | Incyte | Polynucleotide | Polynucleotide | Incyte Full Length |
| Incyte Project ID | SEQ ID NO: | Polypeptide ID | SEQ ID NO: | ID | Clones |
| 7499969 | 1 | 7499969CD1 | 53 | 7499969CB1 | 90040723CA2, |
| | | | | | 90040822CA2 |
| 7499974 | 2 | 7499974CD1 | 54 | 7499974CB1 | |
| 7499976 | 3 | 7499976CD1 | 55 | 7499976CB1 | |
| 7499954 | 4 | 7499954CD1 | 56 | 7499954CB1 | 90046507CA2, |
| | | | | | 90046615CA2, |
| | | | | | 90046639CA2, |
| | | | | | 90046647CA2 |
| 7500827 | 5 | 7500827CD1 | 57 | 7500827CB1 | |
| 7948585 | 6 | 7948585CD1 | 58 | 7948585CB1 | |
| 7500002 | 7 | 7500002CD1 | 59 | 7500002CB1 | 4210225CA2 |
| 7500012 | 8 | 7500012CD1 | 60 | 7500012CB1 | |
| 1664071 | 9 | 1664071CD1 | 61 | 1664071CB1 | 90176867CA2, |
| | | | | | 90176883CA2 |
| 6214577 | 10 | 6214577CD1 | 62 | 6214577CB1 | 90217051CA2 |
| 7502149 | 11 | 7502149CD1 | 63 | 7502149CB1 | |
| 7503480 | 12 | 7503480CD1 | 64 | 7503480CB1 | |
| 7500017 | 13 | 7500017CD1 | 65 | 7500017CB1 | 90063987CA2, |
| | | | | | 90064063CA2 |
| 7499955 | 14 | 7499955CD1 | 66 | 7499955CB1 | 95034696CA2 |
| 7504025 | 15 | 7504025CD1 | 67 | 7504025CB1 | |
| 7503203 | 16 | 7503203CD1 | 68 | 7503203CB1 | |
| 7503260 | 17 | 7503260CD1 | 69 | 7503260CB1 | |
| 2969494 | 18 | 2969494CD1 | 70 | 2969494CB1 | |
| 7503201 | 19 | 7503201CD1 | 71 | 7503201CB1 | |
| 7503262 | 20 | 7503262CD1 | 72 | 7503262CB1 | 90136351CA2, |
| | | | | | 90178943CA2, |
| | | | | | 90179047CA2, |
| | | | | | 90186060CA2 |
| 7503409 | 21 | 7503409CD1 | 73 | 7503409CB1 | |
| 7503499 | 22 | 7503499CD1 | 74 | 7503499CB1 | 1591316CA2 |
| 90031281 | 23 | 90031281CD1 | 75 | 90031281CB1 | 90031281CA2, |
| | | | | | 90031289CA2, |
| | | | | | 90031389CA2 |
| 90061570 | 24 | 90061570CD1 | 76 | 90061570CB1 | 90061570CA2 |
| 7500027 | 25 | 7500027CD1 | 77 | 7500027CB1 | |
| 7504546 | 26 | 7504546CD1 | 78 | 7504546CB1 | 90079443CA2, |
| | | | | | 90079527CA2, |
| | | | | | 95039151CA2, |
| | | | | | 95039167CA2, |
| | | | | | 95039203CA2 |
| 7503246 | 27 | 7503246CD1 | 79 | 7503246CB1 | |
| 7505729 | 28 | 7505729CD1 | 80 | 7505729CB1 | |
| 7487334 | 29 | 7487334CD1 | 81 | 7487334CB1 | |
| 7503109 | 30 | 7503109CD1 | 82 | 7503109CB1 | 90187767CA2 |
| 7503128 | 31 | 7503128CD1 | 83 | 7503128CB1 | |
| 7503191 | 32 | 7503191CD1 | 84 | 7503191CB1 | |
| 7503196 | 33 | 7503196CD1 | 85 | 7503196CB1 | |
| 7503254 | 34 | 7503254CD1 | 86 | 7503254CB1 | 3322204CA2 |
| 7503531 | 35 | 7503531CD1 | 87 | 7503531CB1 | |
| 7490021 | 36 | 7490021CD1 | 88 | 7490021CB1 | |
| 7503180 | 37 | 7503180CD1 | 89 | 7503180CB1 | |
| 7503206 | 38 | 7503206CD1 | 90 | 7503206CB1 | |
| 7503227 | 39 | 7503227CD1 | 91 | 7503227CB1 | |
| 7504473 | 40 | 7504473CD1 | 92 | 7504473CB1 | |
| 7503200 | 41 | 7503200CD1 | 93 | 7503200CB1 | |
| 7500465 | 42 | 7500465CD1 | 94 | 7500465CB1 | 90014556CA2, |
| | | | | | 90014564CA2, |
| | | | | | 90014572CA2, |
| | | | | | 90014580CA2, |
| | | | | | 90014586CA2, |
| | | | | | 90014588CA2 |
| 7503256 | 43 | 7503256CD1 | 95 | 7503256CB1 | 90153409CA2 |
| 7503257 | 44 | 7503257CD1 | 96 | 7503257CB1 | 1406660CA2 |
| 7504472 | 45 | 7504472CD1 | 97 | 7504472CB1 | |
| 7504475 | 46 | 7504475CD1 | 98 | 7504475CB1 | 2641061CA2 |
| 7503104 | 47 | 7503104CD1 | 99 | 7503104CB1 | 90176833CA2 |
| 7503106 | 48 | 7503106CD1 | 100 | 7503106CB1 | 4972070CA2 |
| 7503176 | 49 | 7503176CD1 | 101 | 7503176CB1 | |
| 7503202 | 50 | 7503202CD1 | 102 | 7503202CB1 | |
| 7503249 | 51 | 7503249CD1 | 103 | 7503249CB1 | |
| 7505890 | 52 | 7505890CD1 | 104 | 7505890CB1 | 95115904CA2 |

TABLE 2

| | | GenBank ID NO: | | |
|---|---|---|---|---|
| Polypeptide | Incyte | or PROTEOME | Probability | |
| SEQ ID NO: | Polypeptide ID | ID NO: | Score | Annotation |
| 1 | 7499969CD1 | g187034 | 2.4E−248 | [Homo sapiens] lymphocyte-specific protein tyrosine kinase |
| | | | | Perlmutter, R. M., et al. (1988) J. Cell. Biochem. 38: 117-126 |
| | | | | Structure and expression of lck transcripts in human lymphoid cells |
| | | 342146\|LCK | 2.1E−249 | [Homo sapiens][Protein kinase; Transferase] Lymphocyte-specific protein tyrosine |
| | | | | kinase that is required for antigen-activation of T-cells; corresponding gene is a |
| | | | | proto-oncogene associated with leukemias |
| | | 336312\|LYN | 1.8E−156 | [Homo sapiens][Protein kinase; Transferase; Receptor(signalling); Small molecule |
| | | | | binding protein][Plasma membrane] Tyrosine kinase with similarity to murine T- |
| | | | | lymphocyte-specific tyrosine kinase p56 lck, the v-yes protein, and the gene |
| | | | | products of v-fgr and v-src |
| 2 | 7499974CD1 | g8272557 | 0 | [Rattus norvegicus] protein kinase WNK1 |
| | | | | Xu, B., et al. (2000) J. Biol. Chem. 275: 16795-16801 |
| | | | | WNK1, a novel mammalian serine/threonine protein kinase lacking the catalytic |
| | | | | lysine in subdomain II |
| | | 241101\| | 8.9E−150 | [Caenorhabditis elegans][Protein kinase; Transferase] Serine/threonine protein |
| | | C46C2.1 | | kinase with similarity to human p21-activated kinases |
| | | 594177\| | 9.2E−135 | [Homo sapiens] Putative mitogen-activated MAPKK serine/threonine protein |
| | | LOC54745 | | kinase |
| 3 | 7499976CD1 | g3133291 | 1.9E−105 | [Homo sapiens] mitogen activated protein kinase activated protein kinase |
| | | 344568\| | 1.7E−106 | [Homo sapiens][Protein kinase; Transferase] MAPkinase-activated protein kinase, |
| | | MAPKAPK5 | | phosphorylated by the p38 (CSBP1) MAP kinase and in turn phosphorylates |
| | | | | HSP27, probably has a role in stress response |
| | | 346970\| | 6.8e−25 | [Homo sapiens][Protein kinase; Transferase][Nuclear] Protein kinase that is |
| | | MAPKAPK2 | | activated by MAP kinase, has a proline-rich N-terminal region, two SH3 binding |
| | | | | sites, and a nuclear localization signal (NLS) |
| 4 | 7499954CD1 | g1871531 | 1.8E−186 | [Homo sapiens] protein-tyrosine-phosphatase |
| | | | | Kim, Y. W., et al. (1996. Oncogene 13: 2275-2279 |
| | | | | Characterization of the PEST family protein tyrosine phosphatase BDP1 |
| | | 424446\| | 1.5E−187 | [Homo sapiens][Protein phosphatase; Hydrolase] Tyrosine phosphatase that |
| | | PTPN18 | | contains a PEST motif |
| 5 | 7500827CD1 | g7302790 | 9.2e−85 | [Drosophila melanogaster] EDTP gene product |
| | | | | Adams, M. D., et al. (2000) Science 287: 2185-2195 |
| | | | | The genome sequence of Drosophila melanogaster |
| | | 619805\| | 0.00072 | [Homo sapiens][Protein phosphatase; Hydrolase][Cytoplasmic] Dual-specificity |
| | | MTMR3 | | protein phosphatase, dephosphorylates substrate proteins at Ser/Thr and Tyr |
| | | | | residues, widely distributed in tissues |
| | | 339652\| | 0.0023 | [Homo sapiens][Protein phosphatase; Other phosphatase; |
| | | MTM1 | | Hydrolase][Cytoplasmic] Myotubularin, protein phosphatase which catalyzes the |
| | | | | dephosphorylation of phosphatidylinositol 3-phosphate to phosphatidylinositol, |
| | | | | plays a critical role in myogenesis; mutation of the corresponding gene is |
| | | | | associated with X-linked myotubular myopathy |
| 6 | 7948585CD1 | g3719236 | 1.1e−203 | [Rattus norvegicus] brain-enriched guanylate kinase-associated protein 1; BEGA1 |
| | | | | Deguchi, M., et al. (1998) J. Biol. Chem. 273: 26269-26272 |
| | | | | BEGAIN (brain-enriched guanylate kinase-associated protein), a novel neuronal |
| | | | | PSD-95/SAP90-binding protein |
| | | 685227\| | 2.0e−245 | [Homo sapiens] KIAA1446 protein |
| | | KIAA1446 | | |
| 7 | 7500002CD1 | g14424799 | 5.5e−85 | [Homo sapiens] adenylate kinase 2 |
| | | 334112\|AK2 | 4.9e−86 | [Homo sapiens][Transferase; Other kinase] Adenylate kinase, anisoenzyme |
| | | | | expressed in heart but not skeletal muscle |
| | | 724822\|1ak2_{—} | 1.6e−78 | [Protein Data Bank] Adenylate Kinase Isoenzyme-2 |
| 8 | 7500012CD1 | g2506080 | 0.0 | [Homo sapiens] HsGAK |
| | | | | Kimura, S. H., et al. (1997) Genomics 44: 179-187 |
| | | | | Structure, expression, and chromosomal localization of human GAK |
| | | 342050\|GAK | 0.0 | [Homo sapiens][Protein kinase; Transferase] Serine/threonine protein kinase, |
| | | | | predicted to bind CDK/cyclin G complexes |
| | | 346332\| | 1.9e−123 | [Homo sapiens] Protein with moderate similarity to GAK, which is a |
| | | DNAJC6 | | serine/threonine protein kinase that binds cyclin G and may be involved in |
| | | | | regulation of cell cycle |
| 9 | 1664071CD1 | g12656142 | 2.80e−74 | [Mus musculus] magnesium-dependent phosphatase-1 |
| 10 | 6214577CD1 | g5732662 | 0 | [Homo sapiens] dual-specificity phosphatase |
| | | | | Wong, A. K. C., et al. (1999) Genomics 59: 248-251 |
| | | | | Genomic structure, chromosomal location, and mutation analysis of the human |
| | | | | CDC14A gene |
| | | 334558\| | 0 | [Homo sapiens][Protein phosphatase; Hydrolase][Nuclear] Dual specificity |
| | | CDC14A | | protein phosphatase, has similarity to S. cerevisiae Cdc14p, which has an essential |
| | | | | function late in the cell cycle |
| 11 | 7502149CD1 | g7108919 | 0 | [Homo sapiens] GR AF-1 specific protein phosphatase |
| | | 345082\| | 3.90E−32 | [Homo sapiens][Guanine nucleotide exchange factor] Homolog of murine |
| | | HERC2 | | Mm.20929, which is a guanine nucleotide exchange factor involved in |
| | | | | intracellular protein transport; duplicated and truncated copies of the |
| | | | | corresponding gene are associated with deletion breakpoints in Prader-Willi and |
| | | | | Angelman syndromes |
| | | 341506\| | 1.50E−19 | [Homo sapiens][Guanine nucleotide exchange factor][Golgi; Cytoplasmic] HECT |
| | | H7ERC1 | | (homologous to E6-AP (UBE3A) carboxy terminus) domain and RCC1 (CHCl)- |
| | | | | like domain (RLD) 1, functions as a guanine-nucleotide exchange factor for Rab |
| | | | | related proteins and ARF1, may be involved in membrane transport processes |
| 12 | 7503480CD1 | g802105 | 0 | [Rattus sp.] PP1M M110 protein phosphatase 1M 110 kda regulatory subunit |
| | | | | Chen, Y. H., et al. (1994) FEBS Lett. 356: 51-55 |
| | | | | Molecular cloning of cDNA encoding the 110 kDa and 21 kDa regulatory |
| | | | | subunits of smooth muscle protein phosphatase 1M |
| | | 336536\| | 0 | [Homo sapiens][Regulatory subunit][Cytoplasmic; Cytoskeletal] Myosin |
| | | PPP1R12A | | phosphatase target subunit 1, involved in Rho-mediated myosin light chain |
| | | | | regulation |
| | | 336538\| | 2.50E−215 | [Homo sapiens][Regulatory subunit; Activator] Myosin phosphatase target subunit |
| | | PPP1R12B | | 2, regulatory subunit of myosin phosphatase that stimulates the activity of the |
| | | | | myosin phosphatase catalytic subunit towards the myosin light chain, may have a |
| | | | | role in the regulation of cardiac muscle function |
| 13 | 7500017CD1 | g2641994 | 6.40E−235 | [Homo sapiens] glycogen synthase kinase 3alpha |
| | | 306377\| | 1.20E−235 | [Homo sapiens][Protein kinase; Transferase] Protein with very strong similarity to |
| | | GSK3A | | rat Rn.36807 (glycogen synthase kinase 3-alpha), which phosphorylates and |
| | | | | regulates proteins in glycogen metabolism |
| | | 335646\| | 1.20E−169 | [Homo sapiens][Protein kinase; Transferase][Nuclear] Glycogen synthase kinase, |
| | | GSK3B | | protein-serine kinase that phosphorylates regulatory proteins, involved indirectly |
| | | | | in cell-fate determination and differentiation |
| 14 | 7499955CD1 | g14124968 | 3.40E−157 | [Homo sapiens] protein phosphatase 1, catalytic subunit, alpha isoform |
| | | 337134\| | 3.00E−158 | [Homo sapiens][Protein phosphatase; Hydrolase] Catalytic subunit of protein |
| | | PPP1CA | | phosphatase 1, regulates mitosis and is a putative tumor suppressor |
| 15 | 7504025CD1 | g7960216 | 0 | [Homo sapiens] RACK-like protein PRKCBP1 |
| | | | | Fossey, S. C., et al. (2000) Mamm. Genome 11: 919-925 |
| | | | | Identification and characterization of PRKCBP1, a candidate RACK-like protein |
| | | 618294\| | 0 | [Homo sapiens][Anchor Protein; Receptor (signalling)] protein kinase C binding |
| | | PRKCBP1 | | protein 1, member of the RACK (receptors for activated C-kinase) family and |
| | | | | interacts specifically with protein kinase C betaI (PRKCB1) |
| | | 365767\|BS69 | 5.00E−18 | [Homo sapiens][Activator; Inhibitor or repressor; DNA-binding protein; |
| | | | | Transcription factor][Nuclear] Adenovirus 5 E1A binding protein, binds |
| | | | | adenovirus E1A and represses E1A-activated transcription, also binds to and |
| | | | | represses transcription by MYB; alternate splice form BRAM1 binds the BMP |
| | | | | receptor (Bmpr1a), and may regulate BMP signaling |
| 16 | 7503203CD1 | g406058 | 0.0 | [Mus musculus] protein kinase |
| | | | | Walden, P. D. and Cowan, N. J. (1993) A Novel 205-kDa Testis-specific |
| | | | | Serine/Threonine Protein Kinase Associated with Microtubules of the Spermatid |
| | | | | Manchette. Mol. Cell. Biol. 13: 7625-7635 |
| | | 742582\| | 0.0 | [Homo sapiens][Protein kinase; Transferase][Cytoskeletal] Protein with strong |
| | | MAST205 | | similarity to murine Mtssk, which is a protein kinase that interacts with |
| | | | | microtubules and facilitates their organization in spermatids, contains a eukaryotic |
| | | | | protein kinase domain and a PDZ domain |
| | | 582149\|Mtssk | 0.0 | [Mus musculus] [Protein kinase; Transferase] [Cytoplasmic; Cytoskeletal] |
| | | | | Microtubule associated testis specific serine/threonine protein kinase, may be |
| | | | | involved in the organization of manchette microtubules in spermatids, may have a |
| | | | | role in spermatid maturation |
| | | 424092\| | 0.0 | [Homo sapiens][Protein kinase; Transferase] Protein with strong similarity to |
| | | KIAA0973 | | murine Mm.9287, which is a Ser/Thr kinase that interacts with microtubules to |
| | | | | facilitate their organization in spermatids, contains a kinase domain and a PDZ |
| | | | | domain, which target signaling proteins to membranes |
| | | 609148\|Sast | 0.0 | [Mus musculus] [Protein kinase; Transferase] [Cytoplasmic; Cytoskeletal] |
| | | | | Syntrophin-associated serine/threonine kinase, interacts with syntrophins via PDZ |
| | | | | domains, associated with microtubules and microtubule-associated proteins and |
| | | | | may link the dystrophin (Dmd)/utrophin (Utrn) network with microtubule |
| | | | | filaments |
| | | 423529\| | 0.0 | [Homo sapiens][Protein kinase; Transferase] Protein with high similarity to |
| | | KIAA0561 | | murine Mtssk, which is a protein kinase that interacts with microtubules and |
| | | | | facilitates their organization in spermatids, contains a eukaryotic protein kinase |
| | | | | domain and a PDZ domain |
| 17 | 7503260CD1 | g2736151 | 3.5E−190 | [Rattus norvegicus] mytonic dystrophy kinase-related Cdc42-binding kinase |
| | | | | Leung, T. et al. (1998) Myotonic dystrophy kinase-related Cdc42-binding kinase |
| | | | | acts as a Cdc42 effector in promoting cytoskeletal reorganization. Mol. Cell. |
| | | | | Biol. 18: 130-140 |
| | | 331270\| | 3.1E−191 | [Rattus norvegicus][Protein kinase; Transferase] Protein kinase of the myotonic |
| | | Rn.10871 | | dystrophy kinase family, binds GTP-bound Cdc42, phosphorylates nonmuscle |
| | | | | myosin light chain, acts as a putative downstream effector of Cdc42 in |
| | | | | cytoskeletal reorganization |
| | | 247765\| | 2.2E−177 | [Caenorhabditis elegans][Protein kinase; Transferase] Serine/threonine protein |
| | | K08B12.5 | | kinase; putative ortholog of human protein kinase PK428, which is related to |
| | | | | myotonic dystrophy protein kinase |
| | | 594363\| | 1.9E−162 | [Homo sapiens] Myotonic dystrophy protein kinase-like protein |
| | | HSMDPKIN | | |
| | | 342960\| | 8.3E−162 | [Homo sapiens][Proteinkinase; Transferase; Hydrolase; GTP-binding |
| | | CDC42BPB | | protein/GTPase] [Cytoplasmic; Cytoskeletal] Protein kinase that has similarity to |
| | | | | myotonic dystrophy kinase, binds to and is a downstream effector of GTP-bound |
| | | | | CDC42, phosphorylates non-muscle myosin light chain and affects actin and |
| | | | | cytoskeleton organization |
| | | 331272\| | 1.4E−161 | [Rattus norvegicus][Protein kinase; Transferase] Protein kinase of the myotonic |
| | | Rn.10872 | | dystrophy kinase family, probably bindsGTP-bound Cdc42 and may act as a |
| | | | | downstream effector of Cdc42 incytoskeletal reorganization |
| 18 | 2969494CD1 | g3168602 | 0.0 | [Homo sapiens] (U88153) p160 |
| | | 426824\|P160 | 0.0 | [Homo sapiens] Has a region of low similarity to a region of murine Nsbp1 |
| | | | | (nucleosome binding protein), which binds to nucleosome core particles and |
| | | | | functions as a transcriptional activator, and may have a role in early embryonic |
| | | | | development |
| | | 587709\|Gabre | 1.5E−29 | [Mus musculus] [Channel (passive transporter); Receptor (signalling); |
| | | | | Transporter] [Plasma membrane] Epsilon subunit of the GABA-A receptor, a |
| | | | | chloride channel that is the major inhibitory neurotransmitter receptor in the brain, |
| | | | | homologous rat Gabre protein is expressed in the heart and brain, particularly in |
| | | | | the locus ceruleus, contains an N-terminal Pro/Glx motif |
| | | 711812\| | 7.9E−24 | [Rattus norvegicus][Regulatory subunit; Channel (passive transporter); |
| | | Cngb1 | | Transporter] Cyclic nucleotide-gated channel beta 1, cyclic nucleotide-gated |
| | | | | cation channel which may play a role in visual photo transduction and olfactory |
| | | | | signal transduction; mutations in human CNGB1 gene are associated with |
| | | | | autosomal recessive retinitis pigmentosa |
| | | 626566\|Prp | 1.1E−23 | [Mus musculus] [Extracellular (excluding cell wall)] Proline rich protein with |
| | | | | tandem repeats, expression is induced in salivary glands by isoproterenol and |
| | | | | feeding tannins |
| | | 328994\|Lot1 | 3.4E−23 | [Rattus norvegicus][DNA-binding protein] Zinc-finger protein, expression in |
| | | | | tumorigenic ovarian surface epithelial cell lines isreduced relative to normal |
| | | | | ovarian surface epithelial cell lines |
| 19 | 7503201CD1 | g1657458 | 2.0E−282 | [Sus scrofa] calcium/calmodulin-dependent protein kinase II isoform gamma-B |
| | | | | Singer, H. A. et al. (1997) Novel Ca2+/calmodulin-dependent protein kinase II |
| | | | | gamma-subunit variants expressed in vascular smooth muscle, brain, and |
| | | | | cardiomyocytes. J. Biol. Chem. 272: 9393-9400 |
| | | 331400\| | 7.8E−261 | [Rattus norvegicus][Protein kinase; Transferase] Calcium/calmodulin-dependent |
| | | Rn.10961 | | protein kinase II gamma, activated by calmodulin binding and regulates Ca(2+)- |
| | | | | mediated signaling pathways, may play a role in the developing and mature brain |
| | | 604070\| | 1.1E−248 | [Homo sapiens][Protein kinase; Transferase] Calcium calmodulin-dependent |
| | | CAMK2B | | protein kinase II beta subunit, putative roles in signal transduction and cell |
| | | | | growth, increased expression may play a role in schizophrenia; variant forms of |
| | | | | the corresponding gene are expressed in tumor cells |
| | | 327660\| | 3.8E−239 | [Rattus norvegicus][Protein kinase; Transferase] Calcium/calmodulin-dependent |
| | | Camk2b | | protein kinase II delta, member of the multifunctional CAM kinase II family |
| | | | | involved in Ca2+ regulated processes; human CAMK2D isoform delta 3 is |
| | | | | specifically upregulated in the myocardium of patients with heart failure |
| | | 322426\| | 1.0E−227 | [Mus musculus][Protein kinase; Transferase] Calcium calmodulin-dependent |
| | | Camk2b | | protein kinase II beta subunit, may function in signal transduction, may contribute |
| | | | | to learning; overexpression of human CAMK2B may contribute to schizophrenia |
| | | | | and variant forms of the human gene are expressed in tumor cells |
| | | 624454\| | 1.2E−226 | [Rattus norvegicus][Protein kinase; Transferase] Calcium/calmodulin-dependent |
| | | Rn.9743 | | protein kinase II beta, modulates opioid receptor signaling, enhances |
| | | | | amphetamine-induced dopamine release; human CAMK2B is upregulated in the |
| | | | | frontal cortex of patients with schizophrenia |
| 20 | 7503262CD1 | g13529320 | 4.7E−168 | [Mus musculus] Similar to NIMA (never in mitosis gene a)-related expressed |
| | | | | kinase 3 |
| | | 430066\|Nek3 | 5.9E−168 | [Mus musculus][Protein kinase; Transferase] [Cytoplasmic] Serine/threonine |
| | | | | kinase that has similarity to members of the Aspergillus nidulans NimA kinase |
| | | | | family, but is distinct from other members of this family in that expression is |
| | | | | elevated in quiescent cells |
| | | 347286\| | 1.2E−135 | [Homo sapiens][Protein kinase; Transferase] Serine/threonine kinase that has |
| | | NEK3 | | similarity to Aspergillus nidulans NimA kinase, which is required along with the |
| | | | | p34cdc2 kinase for mitosis |
| | | 430068\|Nek4 | 5.4E−66 | [Mus musculus][Protein kinase; Transferase] NIMA-related expressed kinase, a |
| | | | | protein kinase that may be involved with progression of the cell cycle to mitosis, |
| | | | | abundantly expressed in testis |
| | | 338322\|STK2 | 8.7E−64 | [Homo sapiens][Protein kinase; Transferase] Serine/threonine kinase that is most |
| | | | | highly expressed in the heart |
| | | 371743\|fin1 | 9.8E−51 | [Schizosaccharomyces pombe] Protein that promotes chromatin condensation, |
| | | | | homologous to A. nidulans NIMA |
| 21 | 7503409CD1 | g1006659 | 1.6E−220 | [Homo sapiens] FAST kinase |
| | | | | Tian, Q. et al. (1995) Fas-activated serine/threonine kinase (FAST) phosphorylates |
| | | | | TIA-1 during Fas-mediated apoptosis. J. Exp. Med. 182: 865-874 |
| | | 743544\| | 3.7E−173 | [Homo sapiens][Protein kinase; Transferase] Fas-activated serine threonine |
| | | FASTK | | kinase, a serine-threonine kinase that phosphorylates RNA binding protein TIA1 |
| | | | | during Fas mediated apoptosis, upregulated in peripheral blood mononuclear cells |
| | | | | of atopic asthmatics and atopic non asthmatic patients |
| | | 685389\| | 5.7E−22 | [Homo sapiens] Has a region of low similarity to a region of human FASTK, |
| | | MGC5297 | | which is a serine-threonine kinase that phosphorylates TIA-1 as part of a cell |
| | | | | death program mediated by Fas |
| | | 743762\|CPR2 | 6.3E−12 | [Homo sapiens] Protein that suppresses S. cerevisiae pheromone-induced G1 |
| | | | | arrest when ectopically expressed |
| | | 703653\| | 6.3E−12 | [Homo sapiens] Protein with weak similarity to FASTK, which is a |
| | | KIAA0948 | | serine/threonine kinase that phosphorylates TIA-1(RNA-binding protein) as part |
| | | | | of a cell death program mediated by Fas |
| 22 | 7503499CD1 | g183266 | 4.7E−223 | [Homo sapiens] galactokinase |
| | | | | Lee, R. T. et al. (1992) Cloning of a human galactokinase gene (GK2) on |
| | | | | chromosome 15 by complementation in yeast. Proc. Natl. Acad. Sci. U.S.A. |
| | | | | 89; 10887-10891 |
| | | 343304\| | 4.1E−224 | [Homo sapiens][Transferase; Other kinase] N-acetylgalactosamine kinase |
| | | GALK2 | | (galactokinase), phosphorylates the preferred substrate N-acetylgalactosamine, |
| | | | | may also phosphorylate galactose present in high concentrations |
| | | 728650\| | 1.8E−67 | [Caenorhabditis elegans][Transferase] Putative galactokinase, has strong |
| | | M01D7.4 | | similarity to human GALK2 (galactokinase2) |
| | | 5993\|GAL1 | 7.1E−55 | [Saccharomyces cerevisiae][Transferase; Other kinase] [Cytoplasmic] |
| | | | | Galactokinase, catalyzes the first step in galactose metabolism |
| | | 466837\|GAL1 | 6.3E−53 | [Candida parapsilosis][Transferase; Other kinase] Putative galactokinase |
| | | 629750\| | 1.6E−50 | [Schizosaccharomyces pombe] Putative galactokinase |
| | | SPBPB2B2.13 | | |
| 23 | 90031281CD1 | g14041815 | 1.9E−191 | [Homo sapiens] kinase-like protein |
| | | 613343\| | 1.1E−93 | [Homo sapiens][Protein kinase; Transferase] [Endoplasmic |
| | | NTKL | | reticulum; Cytoplasmic] Protein that interacts with protein kinaseB, contains a |
| | | | | potential protein kinase domain |
| | | 251950\| | 1.7E−65 | [Caenorhabditis elegans][Protein kinase] Protein containing an N-terminal |
| | | W07G4.3 | | serine/threonine protein kinase domain, has similarity to S. cerevisiae Yor112p |
| | | 640706\| | 2.9E−27 | [Candida albicans] Has low similarity to uncharacterized S. cerevisiae Yor112P |
| | | orf6.5474 | | |
| 24 | 90061570CD1 | g7768754 | 1.1E−17 | [Homo sapiens] gene similar to rat protein kinase (KID2) |
| | | | | Hattori, M. et al. (2000) The DNA sequence of human chromosome 21. The |
| | | | | chromosome 21 mapping and sequencing consortium. Nature 405: 311-319 |
| | | 741633\| | 1.1E−29 | [Homo sapiens] Protein with low similarity to ratRn.42905, which is a salt- |
| | | KIAA0781 | | inducible serine/threonine kinase highly expressed in adrenocortical tissues |
| | | | | exposed to either corticosteroid treatment or a high-salt diet |
| | | 624428\| | 1.2E−18 | [Rattus norvegicus][Protein kinase, Transferase] Salt-inducible serine, threonine |
| | | LOC59329 | | kinase that is highly expressed in adrenocortical tissues exposed to either |
| | | | | corticosteroid treatmentor a high-salt diet; has very strong similarity to murine |
| | | | | Msk, which is developmentally expressed in cardiac tissue |
| | | 430038\| | 1.2E−18 | [Mus musculus][Protein kinase; Transferase] SNF1-like kinase, a serine-threonine |
| | | Snf1lk | | protein kinase; expression is restricted to developing myocardium |
| | | 606300\| | 8.7E−11 | [Homo sapiens][Protein kinase; Transferase] [Cytoplasmic; Cytoskeletal] |
| | | EMK1 | | Serine/threonine protein kinase, member of the EMK family of proteins that are |
| | | | | involved in the control of cell polarity and microtubule stability and are associated |
| | | | | with cancer |
| 25 | 7500027CD1 | g3406430 | 4.8E−71 | [Homo sapiens] hPRL-3 |
| | | 743118\| | 4.2E−72 | [Homo sapiens][Protein phosphatase; Hydrolase] Protein tyrosine phosphatase |
| | | PTP4A3 | | (type IVA, member 3), a potentially prenylated tyrosine phosphatase which is |
| | | | | preferentially expressed in skeletal muscle and heart and may interfere with |
| | | | | angiotensin II (AGT)signaling |
| | | 582657\| | 6.0E−69 | [Mus musculus] [Protein phosphatase; Hydrolase] [Endosome/Endosomal |
| | | Ptp4a3 | | vesicles; Nuclear; Cytoplasmic; Plasma membrane; Centrosome/spindle pole |
| | | | | body; Apical plasma membrane] Protein tyrosine phosphatase 4a3, preferentially |
| | | | | expressed in skeletal muscle and heart, has C-terminal prenylation site |
| | | 711450\| | 2.4E−52 | [Rattus norvegicus] Protein tyrosine phosphatase, nuclear protein that is highly |
| | | Ptp4a1 | | expressed in regenerating liver, may be involved in regulation of cell growth, |
| | | | | including tumorigenic cell growth |
| | | 344764\| | 2.4E−52 | [Homo sapiens] [Protein phosphatase; Hydrolase] [Nuclear] Type IVA protein |
| | | PTP4A1 | | tyrosine phosphatase that is prenylated and induces tumorigenesis when |
| | | | | overexpressed |
| | | 585633\| | 2.4E−52 | [Mus musculus] [Protein phosphatase; Hydrolase][Endosome/Endosomal |
| | | Ptp4a1 | | vesicles; Nuclear; Cytoplasmic; Plasma membrane; Apical plasma membrane] |
| | | | | Mitogen-induced protein tyrosine phosphatase, highly expressed in regenerating |
| | | | | liver, induces morphological changes and transformation when overexpressed, has |
| | | | | very strong similarity to human PTP4A1, which is prenylated |
| 26 | 7504546CD1 | g3406430 | 2.9E−77 | [Homo sapiens] hPRL-3 |
| | | 743118\| | 7.5E−79 | [Homo sapiens][Protein phosphatase; Hydrolase] Protein tyrosine phosphatase |
| | | PTP4A3 | | (type IVA, member 3), a potentially prenylated tyrosine phosphatase which is |
| | | | | preferentially expressed in skeletal muscle and heart and may interfere with |
| | | | | angiotensin II (AGT) signaling |
| | | 582657\| | 1.1E−75 | [Mus musculus] [Protein phosphatase; Hydrolase] [Endosome/Endosomal |
| | | Ptp4a3 | | vesicles; Nuclear; Cytoplasmic; Plasma membrane; Centrosome/spindle pole |
| | | | | body; Apical plasma membrane] Protein tyrosine phosphatase 4a3, preferentially |
| | | | | expressed in skeletal muscle and heart, has C-terminal prenylation site |
| | | 711450\| | 3.6E−58 | [Rattus norvegicus] Protein tyrosine phosphatase, nuclear protein that is highly |
| | | Ptp4a1 | | expressed in regenerating liver, may be involved in regulation of cell growth, |
| | | | | including tumorigenic cell growth |
| | | 344764\| | 3.6E−58 | [Homo sapiens][Protein phosphatase; Hydrolase] [Nuclear] Type IVA protein |
| | | PTP4A1 | | tyrosine phosphatase that is prenylated and induces tumorigenesis when |
| | | | | overexpressed |
| | | 585633\| | 3.6E−58 | [Mus musculus] [Protein phosphatase; Hydrolase] [Endosome/Endosomal |
| | | Ptp4a1 | | vesicles; Nuclear; Cytoplasmic; Plasma membrane; Apical plasma membrane] |
| | | | | Mitogen-induced protein tyrosine phosphatase, highly expressed in regenerating |
| | | | | liver, induces morphological changes and transformation when overexpressed, has |
| | | | | very strong similarity to human PTP4A1, which is prenylated |
| 27 | 7503246CD1 | g1749794 | 0.0 | [Homo sapiens] serine/threonine protein kinase |
| | | | | Espinosa, L. and Navarro, E. (1998) Human serine/threonine protein kinase |
| | | | | EMK1: genomic structure and cDNA cloning of isoforms produced by alternative |
| | | | | splicing. Cytogenet. Cell Genet. 8: 278-282 |
| | | 606300\| | 0.0 | [Homo sapiens][Protein kinase; Transferase] [Cytoplasmic; Cytoskeletal] |
| | | EMK1 | | Serine/threonine protein kinase, member of the EMK family of proteins that are |
| | | | | involved in the control of cell polarity and microtubule stability and are associated |
| | | | | with cancer |
| | | 321516\|Emk | 0.0 | [Mus musculus][Protein kinase; Transferase] Protein with very strong similarity to |
| | | | | human EMK1, which is a serine/threonine protein kinase that is a member of the |
| | | | | EMK family of proteins involved in the control of cell polarity and microtubule |
| | | | | stabilityand associated with cancer |
| | | 624438\| | 9.6E−248 | [Rattus norvegicus] [Protein kinase; Transferase] Microtubule/MAP-affinity |
| | | LOC60328 | | regulating kinase, a serine/threonine kinase that phosphorylates specific |
| | | | | microtubule-associated proteins, and thereby destabilizes microtubules |
| | | 599876\| | 1.0E−220 | [Homo sapiens][Protein kinase; Transferase] [Cytoplasmic; Cytoskeletal] |
| | | MARK | | Microtubule affinity regulating kinase, a serine/threonine kinase that |
| | | | | phosphorylates microtubule-associated protein tau, leading to disruption of |
| | | | | microtubules |
| | | 332412\| | 5.7E−218 | [Rattus norvegicus][Protein kinase; Transferase] [Cytoplasmic; Cytoskeletal] |
| | | Rn.21430 | | Microtubule affinity regulating kinase, a serine/threonine kinase that |
| | | | | phosphorylates microtubule-associated proteins tau, MAP2, and MAP4, leading to |
| | | | | disruption of microtubules |
| 28 | 7505729CD1 | g11385416 | 0.0 | [Mus musculus] striated muscle-specific serine/threonine protein kinase |
| | | | | Hsieh, C. M. et al. (2000) Striated Muscle Preferentially Expressed Genes alpha |
| | | | | and beta Are Two Serine/Threonine Protein Kinases Derived from the Same Gene |
| | | | | as the Aortic Preferentially Expressed Gene-1. J. Biol. Chem. 275: 36966-36973 |
| | | 619298\| | 1.2E−108 | [Homo sapiens] Protein of unknown function, has a region of low similarity to a |
| | | KIAA1639 | | region of TRAD (duet), which is a serine/threonine kinase with Dbl and pleckstrin |
| | | | | homology domains, E18and which localizes to the actin cytoskeleton |
| | | 302623\| | 4.8E−108 | [Homo sapiens][Protein kinase; Transferase; Small molecule-binding protein] |
| | | MYLK | | Myosin light chain kinase, member of a family of calcium/calmodulin-dependent |
| | | | | kinases that phosphorylate myosin regulatory light chains and thereby increase |
| | | | | myosin ATPase activity, expressed in brain and smooth muscle |
| | | 338724\|TTN | 5.1E−88 | [Homo sapiens][Structural protein] [Cytoplasmic; Cytoskeletal] Titin, a large |
| | | | | myofilament protein that extends from the I band to the Z disk of sarcomeres, |
| | | | | maintains resting tension in muscle |
| | | 253514\| | 8.1E−87 | [Caenorhabditis elegans] [Protein kinase; Transferase] Serine-threonine protein |
| | | unc-22 | | kinase that may regulate contraction, putative member of immunoglobulin |
| | | | | superfamily |
| | | 253515\| | 1.3E−86 | [Caenorhabditis elegans][Protein kinase; Transferase] Serine/threonine protein |
| | | ZK617.1B | | kinase, has strong similarity to human and D. melanogaster myosin light chain |
| | | | | kinase (MLCK) |
| 29 | 7487334CD1 | g18655333 | 0.0 | [f1][Homo sapiens] epidermal growth factor receptor pathway substrate 8 related |
| | | | | protein 2 |
| | | 659020\| | 2.7E−204 | [Homo sapiens] Protein containing an Src homology 3(SH3) domain, which |
| | | FLJ21935 | | binds + E22 proline-rich peptides, has moderate similarity to human EPS8, which is |
| | | | | tyrosine phosphorylated by epidermal growth factor receptor (EGFR) and |
| | | | | enhances EGF-dependent mitogenic signals |
| | | 340492\|EPS8 | 4.5E−83 | [Homo sapiens][Receptor (signalling)] [Nuclear] Epidermal growth factor |
| | | | | receptor pathway substrate 8, SH3 containing protein that is tyrosine |
| | | | | phosphorylated by epidermal growth factor receptor (EGFR) and enhances EGF- |
| | | | | dependent mitogenic signals, has a role in normal and neoplastic cell proliferation |
| | | 319962\|Eps8 | 4.1E−82 | [Mus musculus][Receptor (signalling)] [Nuclear] Epidermal growth factor |
| | | | | receptor pathway substrate 8, SH3 containing protein that is tyrosine |
| | | | | phosphorylated by epidermal growth factor receptor (EGFR) and enhances EGF- |
| | | | | dependent mitogenic signals, has a role in normal and neoplastic cell proliferation |
| | | 690882\| | 1.2E−35 | [Homo sapiens] Protein with low similarity to human EPS8, which is an epidermal |
| | | FLJ21522 | | growth factor receptor pathway substrate that is tyrosine phosphorylated by |
| | | | | epidermal growth factor receptor (EGFR) and enhances EGF-dependent |
| | | | | mitogenic signals |
| | | 252698\| | 2.7E−30 | [Caenorhabditis elegans] Putative epidermal growth factor receptor kinase |
| | | Y57G11C.24A | | substrate with similarity to human EPS8, putative paralog of C. elegans |
| | | | | Y57G11C.24C |
| 30 | 7503109CD1 | g988305 | 0.0 | [Homo sapiens] PYK2 |
| | | | | Lev, S. et al. (1995) Protein tyrosine kinase PYK2 involved in Ca(2+)-induced |
| | | | | regulation of ion channel and MAP kinase functions. Nature 376: 737-745 |
| | | 341114\| | 0.0 | [Homo sapiens][Protein kinase; Transferase; Receptor (signalling)][Cytoplasmic; |
| | | PTK2B | | Plasma membrane; Centrosome/spindle pole body] Protein tyrosine kinase 2 beta, |
| | | | | a focal adhesion kinase that activates the MAP kinase pathway and may play roles |
| | | | | in glucose transport, T cell receptor signaling, cell motility, and apoptosis |
| | | | | inhibition; involved in development of some human malignancies |
| | | 590919\| | 0.0 | [Rattus norvegicus][Protein kinase; Transferase] [Cytoplasmic; Growth cone] |
| | | CAKbeta | | Calcium-dependent protein tyrosine kinase that is a member of the focal adhesion |
| | | | | kinase family, activates c-Jun N-terminal kinase through both stress- and calcium- |
| | | | | dependent pathways |
| | | 328240\|Ptk2 | 2.5E−231 | [Rattus norvegicus][Protein kinase; Transferase; Receptor (signalling)] |
| | | | | [Cytoplasmic; Plasma membrane; Growth cone] Focal adhesion kinase, non- |
| | | | | receptor tyrosine kinase involved in integrin-mediated signaling and cellular |
| | | | | adhesion, migration, chemotaxis, and proliferation, inhibitor of apoptosis; |
| | | | | upregulation of human PTK2 correlates with increased tumorigenicity |
| | | 326674\|Ptk2 | 2.2E−230 | [Mus musculus][Protein kinase; Transferase; Receptor (signalling)] [Plasma |
| | | | | membrane; Cell junction] Focal adhesion kinase, non-receptor tyrosine kinase |
| | | | | involved in integrin-mediated signaling and cellular adhesion, migration, |
| | | | | chemotaxis, and proliferation, inhibitor of apoptosis; upregulation of human PTK2 |
| | | | | correlates with increased tumorigenicity |
| | | 342712\|PTK2 | 1.6E−181 | [Homo sapiens][Protein kinase; Transferase; Receptor (signalling)] [Cytoplasmic; |
| | | | | Cytoskeletal; Plasma membrane; Cell junction] Focal adhesion kinase, non- |
| | | | | receptor tyrosine kinase involved in integrin-mediated signaling and cellular |
| | | | | adhesion, migration, chemotaxis, and proliferation, acts as an inhibitor of |
| | | | | apoptosis; upregulation correlates with increased tumorigenicity |
| 31 | 7503128CD1 | g35479 | 2.1E−168 | [Homo sapiens] protein kinase catalytic subunit type alpha (AA 1-351) |
| | | | | Maldonado, F. and Hanks, S. K. (1988) A cDNA clone encoding human cAMP- |
| | | | | dependent protein kinase catalytic subunit C alpha. Nucleic Acids Res. 16: 8189-8190 |
| | | 337172\| | 1.9E−169 | [Homo sapiens][Protein kinase; Transferase][Nuclear; Cytoplasmic; Extracellular |
| | | PRKACA | | (excluding cell wall)] Catalytic subunit C alpha of cAMP-dependent protein |
| | | | | kinase, plays a role in transcriptional regulation and may mediate suppression of |
| | | | | apoptosis, may also serve as a tumor biomarker; alternative form C alpha 2 may |
| | | | | play a role in sperm development |
| | | 722895\|1cdk_A | 7.1E−168 | [Protein Data Bank] Camp-Dependent Protein Kinase |
| | | 725563\|1ctp_E | 7.1E−168 | [Protein Data Bank] Camp-Dependent Protein Kinase (E.C.2.7.1.37 |
| | | 729216\|1cmk_E | 7.1E−168 | [Protein Data Bank] Camp-Dependent Protein Kinase Catalytic |
| | | 729757\|1stc_E | 1.3E−166 | [Protein Data Bank] Camp-Dependent Protein Kinase |
| 32 | 7503191CD1 | g3290172 | 2.4E−171 | [Homo sapiens] CARD-containing ICE associated kinase |
| | | 337632\| | 2.1E−172 | [Homo sapiens][Protein kinase; Transferase] Receptor-interacting serine- |
| | | RIPK2 | | threonine kinase 2, contains an N-terminal kinase domain and a C-terminal |
| | | | | caspase recruitment domain, part of both the CD40 and the tumor necrosis factor |
| | | | | receptor signaling complex; induces apoptosis and activates NF-kappaB |
| | | 609256\|Rip3 | 4.6E−35 | [Mus musculus][Protein kinase; Transferase] Receptor-interacting protein, a |
| | | | | serine-threonine kinase that activates NF-kappaB; C terminus does not contain a |
| | | | | death domain, but does induce apoptosis upon overexpression |
| | | 428556\| | 1.5E−33 | [Homo sapiens][Protein kinase; Transferase] Receptor-interacting serine- |
| | | RIPK3 | | threonine kinase, C-terminus mediates recruitment to the TNFR-1 signaling |
| | | | | complex, activates NF-kappa Band potently induces apoptosis |
| | | 320794\|Ripk1 | 5.6E−33 | [Mus musculus][Protein kinase; Transferase] Receptor interacting serine threonine |
| | | | | kinase 1, a serine-threonine kinase that contains a C-terminal death domain, |
| | | | | interacts with Fas (Tnfrsf6), interacts with tumor necrosis factor receptor 1 |
| | | | | (Tnfrsf1a), induces apoptosis, and activates NF-kappaB |
| | | 337634\| | 3.4E−29 | [Homo sapiens][Protein kinase; Transferase] Receptor interacting serine threonine |
| | | RIPK1 | | kinase 1, a serine-threonine kinase that contains a C-terminal death domain, |
| | | | | interacts with Fas (TNFRSF6), interacts with tumor necrosis factor receptor 1 |
| | | | | (TNFRSF1A), induces apoptosis, and activates NF-kappaB |
| 33 | 7503196CD1 | g2661106 | 0.0 | [Homo sapiens] CASK |
| | | 626878\|Cask | 0.0 | [Rattus norvegicus][Protein kinase; Anchor Protein; Transferase; Other kinase] |
| | | | | [Cytoplasmic; Plasma membrane] Protein with calcium/calmodulin-dependent |
| | | | | serine protein kinase and guanylate kinase domains, binds to neurexins, has very |
| | | | | strong similarity to human CASK, which probably links the extracellular matrix to |
| | | | | the actin cytoskeleton |
| | | 324254\|Cask | 0.0 | [Mus musculus][Adhesin/agglutinin; Protein kinase; Anchor Protein; Transferase; |
| | | | | Other kinase][Plasma membrane]Protein with very strong similarity to human |
| | | | | CASK, which is a membrane-associated guanylate kinase that also has a serine |
| | | | | protein kinase domain and probably links the extracellular matrix to the actin |
| | | | | cytoskeleton |
| | | 334456\|CASK | 0.0 | [Homo sapiens][Adhesin/agglutinin; Protein kinase; Anchor Protein; Transferase; |
| | | | | Other kinase; Small molecule-binding protein] [Basolateral plasma membrane; |
| | | | | Cytoplasmic; Cytoskeletal; Plasma membrane; Cell junction] Membrane- |
| | | | | associated guanylate kinase that also has a serine protein kinase domain, binds to |
| | | | | the actin-binding protein 4.1 and the extracellular matrix binding protein syndecan |
| | | | | 2 (SDC2), probably links the extracellular matrix to the actin cytoskeleton |
| | | 276422\|lin-2 | 1.0E−225 | [Caenorhabditis elegans] [Cell junction] Component of the LIN-2, LIN-7, LIN-10 |
| | | | | cell junction complex, involved in vulval development, probable ortholog of |
| | | | | human and rat CASK proteins (putative scaffold proteins of the cytoskeletal |
| | | | | membrane involved in signal transduction coordination) |
| | | 276424\|lin-2 | 4.8E−158 | [Caenorhabditis elegans] [Cell junction] Component of the LIN-2, LIN-7, LIN-10 |
| | | | | cell junction complex, involved in vulval development, probable ortholog of |
| | | | | human and rat CASK proteins (putative scaffold proteins of the cytoskeletal |
| | | | | membrane involved in signal transduction coordination) |
| 34 | 7503254CD1 | g9927293 | 0.0 | [Homo sapiens] plaucible mixed-lineage kinase protein |
| | | 476453\|ZAK | 0.0 | [Homo sapiens] Mixed lineage kinase-like protein, stimulates the JNK/SAPK |
| | | | | pathway and activates NF-kappaB, contains a catalytic domain, a leucine zipper, |
| | | | | and a sterile-alpha motif |
| | | 662697\|Zak | 0.0 | [Mus musculus] [Protein kinase; Transferase] MLK-like mitogen-activated protein |
| | | | | triple kinase, activated by osmotic shock, activated alpha splice form disrupts |
| | | | | actin stress fibers, activates the p38, JNK/SAPK, ERK, and ERK5 pathways upon |
| | | | | overexpression |
| | | 336422\| | 1.7E−37 | [Homo sapiens][Protein kinase; Transferase] Mixed lineage kinase-3, |
| | | MAP3K11 | | serine/threonine kinase with similarity to the tyrosine kinase superfamily, mediates |
| | | | | activation of the JNK pathway by members of the Rho-family of small GTPases, |
| | | | | plays a role in melanocyte proliferation and neuronal apoptosis |
| | | 440027\| | 1.9E−37 | [Caenorhabditis elegans][Protein kinase; Transferase] Serine/threonine protein |
| | | F33E2.2 | | kinase with similarity to human leucine zipper-bearing protein kinases, has |
| | | | | similarity to D. melanogaster protein kinase RAF |
| 35 | 7503531CD1 | g5834427 | 6.4E−88 | [Homo sapiens] glycerol kinase |
| | | 335526\|GK | 9.7E−73 | [Homo sapiens][Transferase; Other kinase] Glycerol kinase, metabolizes |
| | | | | endogenous and dietary glycerol, deficiency is associated with hyperglycerolemia |
| | | | | and glyceroluria |
| | | 429848\| | 1.4E−71 | [Mus musculus][Transferase; Other kinase] Glycerol kinase-related protein, in |
| | | Gk-rs1 | | vitro translated protein does not have detectable glycerol kinase activity, |
| | | | | expressed only in the testes |
| | | 583147\|Gyk | 4.7E−71 | [Mus musculus][Transferase; Other kinase][Cytoplasmic; Mitochondrial outer |
| | | | | membrane; Mitochondrial] Glycerol kinase, metabolizes endogenous and dietary |
| | | | | glycerol, has an alternative splice form in the brain |
| | | 429850\| | 7.7E−69 | [Mus musculus][Transferase; Other kinase] Glycerol kinase-related sequence 2, |
| | | Gk-rs2 | | member of the X-encoded glycerol kinase gene family, but expressed protein has |
| | | | | no detectable glycerol kinase activity; expressed only in the testes |
| | | 249102\| | 2.6E−33 | [Caenorhabditis elegans][Transferase; Other kinase] Member of the glycerol |
| | | R11F4.1 | | kinase protein family |
| 36 | 7490021CD1 | g17385401 | 0.0 | [fl][Homo sapiens] TPIP alpha lipid phosphatase |
| | | 432828\| | 8.6E−263 | [Homo sapiens][Protein phosphatase; Hydrolase] Transmembrane phosphatase |
| | | TPTE | | with tensin homology, putative transmembrane tyrosine phosphatase, may be |
| | | | | involved in spermatogenetic function of the testis and-or signal transduction |
| | | | | pathways of the endocrine |
| | | 426429\| | 3.2E−42 | [Homo sapiens][Protein phosphatase; Otherphosphatase; Hydrolase] Phosphatase |
| | | PTENP1 | | and tensin homolog, a protein that is transcribed from a processed pseudogene and |
| | | | | may contribute to glioblastoma; mutation of the corresponding pseudogene may |
| | | | | be associated with small cell lung cancer tumorigenesis |
| | | 717538\|1d5r_A | 1.6E−41 | [Protein Data Bank] Phosphoinositide Phosphotase Pten |
| | | 319746\|Pten | 6.8E−41 | [Mus musculus][Protein phosphatase; Hydrolase] Phosphatase tensin homolog, a |
| | | | | phosphatidyl inositol phosphatase that acts as a tumor suppressor, involved in cell |
| | | | | cycle control and embryonic development; mutation of the human PTEN gene is |
| | | | | associated with Cowden disease and Bannayan-Zonana syndrome |
| | | 332422\| | 6.8E−41 | [Rattus norvegicus][Proteinphosphatase; Hydrolase][Cytoplasmic; Plasma |
| | | Rn.22158 | | membrane; Cell junction] Phosphatase tensin homolog, a phosphatidyl inositol |
| | | | | phosphatase that acts as a tumor suppressor; mutation of the human PTEN gene is |
| | | | | associated with Cowden disease and Bannayan-Zonana syndrome |
| 37 | 7503180CD1 | g14020949 | 4.0E−34 | [Arabidopsis thaliana] phosphatidic acid phosphatase |
| | | 373503\| | 1.7E−26 | [Schizosaccharomyces pombe] Protein with similarity to phosphatidic acid |
| | | SPBC409.18 | | phosphatase |
| | | 9943\|DPP1 | 1.9E−25 | [Saccharomyces cerevisiae][Other phosphatase; Hydrolase] [Lysosome/vacuole] |
| | | | | Diacylglycerol pyrophosphate phosphatase |
| | | 636018\| | 1.4E−23 | [Candida albicans][Other phosphatase; Hydrolase] Member of the phosphatidic |
| | | orf6.3130 | | acid (PA) phosphatase-related phosphoesterase family, has moderate similarity to |
| | | | | S. cerevisiae Dpp1p, which is a diacyl glycerol pyrophosphate phosphatase |
| | | 634140\| | 9.8E−23 | [Candida albicans][Other phosphatase; Hydrolase] Member of the phosphatidic |
| | | orf6.2191 | | acid (PA) phosphatase-related phosphoesterase family, has moderate similarity to |
| | | | | S. cerevisiae Dpp1p, which is a diacylglycerol pyrophosphate phosphatase |
| | | 641758\| | 1.6E−22 | [Candida albicans][Other phosphatase; Hydrolase] Protein with high similarity to |
| | | orf6.6000 | | S. cerevisiae Dpp1p, which is a diacyl glycerol pyrophosphate phosphatase, |
| | | | | member of the phosphatidic acid (PA) phosphatase-related phosphoesterase |
| | | | | family |
| 38 | 7503206CD1 | g2315202 | 6.5E−277 | [Homo sapiens] protein phosphatase 2C gamma |
| | | | | Travis, S. M. and Welsh, M. J. (1997) PP2C gamma: a human protein phosphatase |
| | | | | with a unique acidic domain. FEBS Lett. 412: 415-419 |
| | | 337130\|PPM1G | 5.7E−278 | [Homo sapiens][Protein phosphatase; Hydrolase] [Nuclear] Magnesium or |
| | | | | manganese dependent protein phosphatase, has an acidic domain, has strong |
| | | | | similarity to murine Fin13, overexpression of which inhibits cell proliferation |
| | | 324858\| | 2.4E−259 | [Mus musculus][Protein phosphatase; Hydrolase] [Nuclear] Manganese |
| | | Ppm1g | | dependent, okadaic acid insensitive protein phosphatase, has an acidic domain, |
| | | | | highly expressed in proliferating cells and induced by mitogens in fibroblasts, |
| | | | | overexpression inhibits cell proliferation |
| | | 245117\| | 2.6E−80 | [Caenorhabditis elegans] Member of the protein phosphatase 2C protein family |
| | | F42G9.1 | | |
| | | 369470\|ptc2 | 2.3E−55 | [Schizosaccharomyces pombe] Serine/threonine phosphatase, member of the |
| | | | | PP2C family |
| | | 376576\|ptc3 | 1.6E−51 | [Schizosaccharomyces pombe][Protein phosphatase; Hydrolase] Serine/threonine |
| | | | | phosphatase, member of the PP2C family |
| 39 | 7503227CD1 | g4028575 | 2.6E−148 | [Homo sapiens] protein phosphatase X |
| | | | | Hu, M. C. et al. (1998) Protein phosphatase X interacts with c-Rel and stimulates c- |
| | | | | Rel/nuclear factor kappaB activity. J. Biol. Chem. 273: 33561-33565 |
| | | 368080\| | 2.2E−149 | [Mus musculus][Protein phosphatase; Hydrolase] Protein phosphatase X (protein |
| | | Ppp4c | | phosphatase 4), a serine/threonine protein phosphatase that activates nuclear |
| | | | | factor kappa B and stimulates c-Rel binding to DNA |
| | | 337148\| | 2.2E−149 | [Homo sapiens][Protein phosphatase; Hydrolase] Catalytic subunit of |
| | | PPP4C | | serine/threonine protein phosphatase 4 |
| | | 317361\| | 4.0E−122 | [Caenorhabditis elegans] Putative ser/thr protein phosphatase |
| | | Y75B8A.30 | | |
| | | 370509\| | 2.7E−106 | [Schizosaccharomyces pombe] Serine/threonine protein phosphatase |
| | | SPBC26H8.05c | | |
| | | 341092\| | 1.4E−100 | [Homo sapiens][Protein phosphatase; Hydrolase] Beta isoform of the catalytic |
| | | PPP2CB | | subunit of protein phosphatase 2A, which is a major serine-threonine phosphatase |
| | | | | thought to play a regulatory role in many cellular pathways |
| 40 | 7504473CD1 | g306477 | 8.2E−109 | [Homo sapiens] calmodulin-dependent phosphatase catalytic subunit |
| | | | | Kincaid, R. L. et al. (1990) Cloning and characterization of molecular isoforms of |
| | | | | the catalytic subunit of calcineurin using nonisotopic methods. J. Biol. Chem. |
| | | | | 265: 11312-11319; Muramatsu, T. and Kincaid, R. L. (1993) Molecular cloning of |
| | | | | a full-length cDNA encoding the catalytic subunit of human calmodulin- |
| | | | | dependent protein phosphatase (calcineurin A alpha). Biochim. Biophys. Acta |
| | | | | 1178: 117-120 |
| | | 568418\| | 7.2E−110 | [Homo sapiens][Protein phosphatase; Hydrolase; Small molecule-binding protein] |
| | | PPP3CA | | Catalytic subunit of calmodulin regulated protein phosphatase (calcineurin A |
| | | | | alpha), regulates activity of transcription factors involved in signal |
| | | | | transductionand growth control |
| | | 717099\|laui_A | 7.2E−110 | [Protein Data Bank] Serine/Threonine Phosphatase 2B |
| | | 437757\| | 2.4E−109 | [Rattus norvegicus][Protein phosphatase; Hydrolase; Small molecule-binding |
| | | Ppp3ca | | protein] Catalytic subunit of calmodulin regulated protein phosphatase |
| | | | | (calcineurin A alpha), regulates activity of transcription factors involved in signal |
| | | | | transductionand growth control, regulates long term potentiation |
| | | 318682\| | 2.4E−109 | [Mus musculus][Protein phosphatase; Hydrolase; Small molecule-binding |
| | | Ppp3ca | | protein][Nuclear] Catalytic subunit of calmodulin regulated protein phosphatase |
| | | | | (calcineurin A alpha), regulates activity of transcription factors involved in signal |
| | | | | transductionand growth control, regulates long term potentiation and memory |
| | | 618270\| | 1.8E−75 | [Homo sapiens][Protein phosphatase; Hydrolase; Small molecule-binding protein] |
| | | PPP3CB | | Catalytic subunit of calmodulin regulated protein phosphatase (calcineurin A, beta |
| | | | | isoform), regulates activity of transcription factors involved in signal transduction |
| | | | | and growth control |
| 41 | 7503200CD1 | g13528684 | 4.7E−210 | [Homo sapiens] Similar to ribosomal protein S6 kinase, 52 kD, polypeptide 1 |
| | | 428964\| | 3.8E−35 | [Homo sapiens][Protein kinase; Transferase] Putative ribosomal protein S6 kinase |
| | | RPS6KC1 | | Zhang, H. et al. Genomics 61, 314-8 (1999). |
| | | 586421\| | 3.6E−19 | [Mus musculus][Protein kinase; Transferase] Member of the ribosomal protein S6 |
| | | Rps6ka2 | | kinase (RSK) family of protein kinases |
| | | | | Bjorbaek. C. et al. J. Biol. Chem. 270, 18848-52. (1995). |
| | | | | Zhao, Y. et al. J. Biol. Chem. 271, 29773-9. (1996). |
| | | 341184\| | 6.6E−19 | [Homo sapiens][Protein kinase; Transferase] Member of the ribosomal protein S6 |
| | | RPS6KA3 | | kinase (RSK) family of protein kinases, required for epidermal growth factor |
| | | | | (EGF)-stimulated phosphorylation of histone H3; associated with Coffin-Lowry |
| | | | | syndrome and non-specific mental retardation |
| | | 617956\| | 1.7E−18 | [Homo sapiens][Protein kinase; Transferase][Nuclear] Member of the ribosomal |
| | | RPS6KA2 | | protein S6 kinase (RSK) family of protein kinases, an isoform with unique N- |
| | | | | terminal sequence and distinct substrate specificity |
| 42 | 7500465CD1 | g11177008 | 1.5E−29 | [Homo sapiens] casein kinase 1 gamma 1 |
| | | | | Kusuda, J. et al. Cytogenet. Cell Genet. 90, 298-302 (2000) |
| | | 626061\| | 1.3E−30 | [Homo sapiens][Protein kinase; Transferase] Casein kinase 1gamma 1, putative |
| | | CSNK1G1 | | serine/threonine protein kinase, may play roles in cell growth and in |
| | | | | morphogenesis |
| | | 627048\| | 6.6E−27 | [Rattus norvegicus][Protein kinase; Transferase] Casein kinase 1 gamma 1, a |
| | | Csnk1g1 | | serine/threonine protein kinase, may play roles in cell growth and in |
| | | | | morphogenesis |
| | | 661440\| | 3.1E−16 | [Rattus norvegicus][Protein kinase; Transferase] Casein kinase 1 gamma 3, a |
| | | Csnk1g3 | | serine/threonine protein kinase that may play roles in cell growth and in |
| | | | | morphogenesis |
| | | 340288\| | 1.4E−15 | [Homo sapiens][Protein kinase; Transferase] Casein kinase 1 gamma 3, a putative |
| | | CSNK1G3 | | serine/threonine protein kinase that may play a role in signal transduction |
| | | 344104\| | 1.1E−14 | [Homo sapiens][Protein kinase; Transferase] Casein kinase 1 gamma 2, a putative |
| | | CSNK1G2 | | serine/threonine protein kinase, may play a role in signal transduction |
| 43 | 7503256CD1 | g348245 | 1.2E−42 | [Homo sapiens] protein serine/threonine kinase |
| | | | | Levedakou, E. N., et al. Oncogene 9, 1977-1988 (1994) |
| | | 691374\| | 3.4E−172 | [Homo sapiens] Protein has a region of low similarity to a region of human NEK2, |
| | | FLJ23495 | | which is a serine/threonine kinase that may have a role in the centrosome cycle |
| | | 338322\| | 1.0E−43 | [Homo sapiens][Protein kinase; Transferase] Serine/threonine kinase that is most |
| | | STK2 | | highly expressed in the heart |
| | | | | Cance, W. G. et al. Int J Cancer 54, 571-7 (1993). |
| | | 430068\|Nek4 | 1.1E−42 | [Mus musculus][Protein kinase; Transferase] NIMA-related expressed kinase, a |
| | | | | protein kinase that may be involved with progression of the cell cycle to mitosis, |
| | | | | abundantly expressed in testis |
| | | 430066\|Nek3 | 8.0E−38 | [Mus musculus][Protein kinase; Transferase][Cytoplasmic]NIMA-related kinase 3, |
| | | | | a protein kinase that is involved in cell cycle control |
| | | 347286\| | 1.0E−36 | [Homo sapiens][Protein kinase; Transferase] NIMA-related kinase3, a putative |
| | | NEK3 | | serine/threonine kinase that may be involved in cell cycle control during mitosis |
| 44 | 7503257CD1 | g21955952 | 0.0 | [fl][Homo sapiens] NIMA-related kinase 11L |
| | | 691374\| | 2.2E−115 | [Homo sapiens] Protein has a region of low similarity to a region of human NEK2, |
| | | FLJ23495 | | which is a serine/threonine kinase that may have a role in the centrosome cycle |
| | | 338322\|STK2 | 1.0E−66 | [Homo sapiens][Protein kinase; Transferase] Serine/threonine kinase that is most |
| | | | | highly expressed in the heart |
| | | 430068\|Nek4 | 6.1E−61 | [Mus musculus][Protein kinase; Transferase] NIMA-related expressed kinase, a |
| | | | | protein kinase that may be involved with progression of the cell cycle to mitosis, |
| | | | | abundantly expressed in testis |
| | | 430066\|Nek3 | 1.8E−54 | [Mus musculus][Protein kinase; Transferase][Cytoplasmic]NIMA-related kinase 3, |
| | | | | a protein kinase that is involved in cell cycle control |
| | | 347286\| | 1.9E−52 | [Homo sapiens][Protein kinase; Transferase] NIMA-related kinase 3, a putative |
| | | NEK3 | | serine/threonine kinase that may be involved in cell cycle control during mitosis |
| 45 | 7504472CD1 | g1000125 | 0.0 | [Homo sapiens] PRK2 |
| | | | | Palmer et al. FEBS Lett. 356, 5-8 (1994) |
| | | | | Palmer, R. H. et al. Eur. J. Biochem. 227, 344-351 (1995) |
| | | 343716\| | 0.0 | [Homo sapiens][Protein kinase; Transferase] Protein kinase C-like 2, a serine- |
| | | PRKCL2 | | threonine kinase related to protein kinase C, involved in protein phosphorylation |
| | | | | and may be involved in apoptosis |
| | | | | Yu, W. et al. J. Biol. Chem. 272, 10030-4 (1997). |
| | | | | Cryns, V. L. et al. JJ. Biol. Chem. 272, 29449-53. (1997). |
| | | 437881\| | 1.4E−226 | [Rattus norvegicus][Protein kinase; Transferase] Protein kinase N, serine/threonine |
| | | Prkcl1 | | kinase that requires Rho and fatty acids for activation, mediates insulin receptor |
| | | | | signaling and may be involved in cytoskeletal reorganization |
| | | 337190\| | 1.4E−186 | [Homo sapiens][Protein kinase; Transferase] Protein kinase N, serine/threonine |
| | | PRKCL1 | | kinase that requires Rho and fatty acids for activation, may be involved in |
| | | | | cytoskeletal reorganization; associated with senile plaque and neurofibrillary |
| | | | | tangle pathologies in Alzheimer's disease |
| | | 424910\| | 4.8E−186 | [Homo sapiens][Proteinkinase; Transferase][Golgi; Nuclear; Cytoplasmic] Protein |
| | | pknbeta | | kinase with similarity to PKN alpha, has leucine zipper-like motifs and two |
| | | | | proline-rich SH3-binding domains, expressed specifically in cancer cell lines |
| | | 245468\| | 8.8E−146 | [Caenorhabditis elegans][Protein kinase; Transferase]Serine/threonine protein |
| | | F46F6.2 | | kinase with strong similarity to human, D. melanogaster, and S. cerevisiae protein |
| | | | | kinase C isoforms, important for establishment of embryonic polarity |
| 46 | 7504475CD1 | g14522878 | 5.8E−256 | [Homo sapiens] calcium/calmodulin-dependent protein kinase kinase b2 |
| | | | | Hsu, L. S. et al. J. Biol. Chem. 276, 31113-31123 (2001) |
| | | 332746\| | 5.4E−240 | [Rattus norvegicus][Protein kinase; Transferase]Calcium/calmodulin-dependent |
| | | Rn.30038 | | protein kinase kinase beta, activates calmodulin-dependent protein kinase IV |
| | | | | (Rn.11046) and is expressed in the brain |
| | | | | Edelman, A. M. et al. J. Biol. Chem. 271, 10806-10 (1996). |
| | | | | Anderson, K. A. et al. J. Biol. Chem. 273, 31880-9 (1998). |
| | | 432456\| | 1.1E−177 | [Homo sapiens][Protein kinase; Transferase]Calcium/calmodulin-dependent |
| | | CAMKK2 | | protein kinase kinase beta, a threonine-preferring protein kinase that |
| | | | | phosphorylates calcium/calmodulin-dependent protein kinases I and IV in a |
| | | | | Ca(2+)/CaM-dependent manner, expression is ubiquitous but is highest in brain |
| | | 328668\| | 5.9E−136 | [Rattus norvegicus][Protein kinase; Transferase] CaM-kinaseIV kinase, |
| | | Rn.4851 | | phosphorylates Ca(2+)/calmodulin kinase IV and is expressed in the brain in at |
| | | | | least two isoforms |
| | | 596846\| | 1.4E−118 | [Mus musculus][Protein kinase; Transferase]Calcium/calmodulin-dependent |
| | | Camkk1 | | protein kinase kinase, may have a role in retinoic acid-induced differentiation of |
| | | | | neutrophils |
| | | 418144\| | 9.1E−91 | [Caenorhabditis elegans][Protein kinase; Transferase] Calcium and calmodulin- |
| | | CaM-KK | | dependent protein kinase kinase |
| 47 | 7503104CD1 | g1777757 | 2.3E−75 | [Homo sapiens] protein tyrosine phosphatase PTPCAAX2 |
| | | | | Cates, C. A. et al. Cancer Lett. 110, 49-55 (1996) |
| | | 337392\| | 2.0E−76 | [Homo sapiens][Protein phosphatase; Hydrolase] Protein tyrosine phosphatase 4, |
| | | PTP4A2 | | ubiquitously expressed; has very strong similarity to murine Ptp4a2, a protein |
| | | | | tyrosine phosphatase which has a C-terminal prenylation site |
| | | | | Zhao, Z. et al. Genomics 35, 172-81 (1996). |
| | | | | Zeng, Q. et al. Biochem. Biophys. Res. Commun. 244, 421-7 (1998). |
| | | 323130\| | 2.0E−76 | [Mus musculus][Proteinphosphatase; Hydrolase][Endosome/Endosomalvesicles; |
| | | Ptp4a2 | | Nuclear; Cytoplasmic; Plasma membrane; Apical plasma membrane] Putative |
| | | | | protein tyrosine phosphatase, preferentially expressed in skeletal muscle, has a C- |
| | | | | terminal prenylation site |
| | | 328036\| | 4.7E−75 | [Rattus norvegicus][Protein phosphatase; Hydrolase][Nuclear]Protein tyrosine |
| | | Rn.2045 | | phosphatase, may play a role in endocrine function; has very strong similarity to |
| | | | | murine Ptp4a2, which is potentially prenylated |
| | | 344764\| | 1.4E−67 | [Homo sapiens][Protein phosphatase; Hydrolase][Nuclear] TypeIVA protein |
| | | PTP4A1 | | tyrosine phosphatase that is prenylated and induces tumorigenesis when |
| | | | | overexpressed |
| | | 585633\| | 1.4E−67 | [Mus musculus][Protein phosphatase; Hydrolase][Endosome/Endosomal vesicles; |
| | | Ptp4a1 | | Nuclear; Cytoplasmic; Plasma membrane; Apical plasma membrane] Mitogen- |
| | | | | induced protein tyrosine phosphatase, highly expressed in regenerating liver, |
| | | | | induces morphological changes and transformation when overexpressed, has very |
| | | | | strong similarity to human PTP4A1, which is prenylated |
| 48 | 7503106CD1 | g531476 | 5.7E−110 | [Homo sapiens] protein phosphotase 1 catyltic subunit beta isoform |
| | | | | Barker, H. M. et al. Biochim. Biophys. Acta 1220, 212-218 (1994) |
| | | 337136\| | 4.9E−111 | [Homo sapiens][Protein phosphatase; Hydrolase] Catalytic subunit beta of protein |
| | | PPP1CB | | phosphatase 1, , which is a major serine-threonine phosphatase involved in the |
| | | | | regulation of numerous metabolic processes |
| | | | | Andreassen, P. R. et al. J. Biol. Chem. 141, 1207-15. (1998). |
| | | 430626\| | 4.9E−111 | [Rattus norvegicus][Protein phosphatase; Hydrolase] Catalytic subunit of protein |
| | | Ppp1cb | | phosphatase 1, which is a major serine-threonine phosphatase involved in the |
| | | | | regulation of numerous metabolic processes |
| | | 668091\| | 2.1E−110 | [Mus musculus][Protein phosphatase; Hydrolase][Dendrite]Catalytic subunit of |
| | | Ppp1cb | | protein phosphatase 1, which is a major serine-threonine phosphatase involved in |
| | | | | the regulation of numerous metabolic processes |
| | | 313245\| | 6.1E−98 | [Caenorhabditis elegans][Proteinphosphatase; Hydrolase][Cytoplasmic] PP1-beta |
| | | CeGLC-7a | | serine/threonine protein phosphatase |
| | | 328030\| | 2.6E−95 | [Rattus norvegicus][Protein phosphatase; Hydrolase]Catalytic subunit of protein |
| | | Rn.2024 | | phosphatase 1, expression is increased in proliferating liver and hepatocarcinomas |
| 49 | 7503176CD1 | g3025880 | 1.9E−146 | [Homo sapiens] phosphatidic acid phosphatase type 2 |
| | | 337108\| | 1.7E−147 | [Homo sapiens][Protein phosphatase; Hydrolase][Unspecified membrane] |
| | | PPAP2C | | Phosphatidic acid phosphatase 2c, hydrolyzes phospholipids, may play a role in |
| | | | | signal transduction |
| | | | | Hooks, S. B. et al. FEBS Lett 427, 188-92 (1998). |
| | | 477336\| | 2.9E−111 | [Mus musculus][Other phosphatase; Hydrolase] Phosphatidic acid phosphatase |
| | | Ppap2c | | 2c, may hydrolyze phospholipids, may play a role in signal transduction |
| | | 337104\| | 7.0E−78 | [Homo sapiens][Protein phosphatase; Hydrolase][Plasma membrane] |
| | | PPAP2A | | Phosphatidic acid phosphatase type 2a, catalyzes the dephosphorylation of various |
| | | | | lipid phosphates, regulates the level of lipid phosphates which are involved in |
| | | | | signal transduction |
| | | 327360\| | 2.1E−76 | [Mus musculus][Other phosphatase; Hydrolase][Unspecified membrane] |
| | | Ppap2a | | Phosphatidic acid phosphatase type 2a, catalyzes the dephosphorylation of various |
| | | | | lipid phosphates, may regulate the level of lipid phosphates which are involved in |
| | | | | signal transduction |
| | | 328006\| | 1.9E−75 | [Rattus norvegicus] [Hydrolase] Phosphatidic acid phosphatase type 2a, catalyzes |
| | | Ppap2 | | the dephosphorylation of various lipid phosphates, may regulate the level of lipid |
| | | | | phosphates which are involved in signal transduction |
| 50 | 7503202CD1 | g180709 | 3.1E−281 | [Homo sapiens] calcineurin A2 |
| | | | | Guerini, D. and Klee, C. B. Proc. Natl. Acad. Sci. U.S.A. 86, 9183-9187 (1989) |
| | | 618270\| | 2.7E−282 | [Homo sapiens][Protein phosphatase; Hydrolase; Small molecule-binding protein] |
| | | PPP3CB | | Catalytic subunit of calmodulin regulated protein phosphatase (calcineurin A, beta |
| | | | | isoform), regulates activity of transcription factors involved in signal transduction |
| | | | | and growth control |
| | | | | Giri, P. R. et al. Biochem. Biophys. Res. Commun. 181, 252-8 (1991). |
| | | 437759\| | 1.1E−280 | [Rattus norvegicus][Protein phosphatase; Hydrolase; Smallmolecule-binding |
| | | Ppp3cb | | protein] Catalytic subunit of calmodulin-regulated protein phosphatase |
| | | | | (calcineurin A, beta isoform), has very strong similarity to human PPP3CB, which |
| | | | | regulates activity of transcription factors involved in signal transduction and |
| | | | | growth control |
| | | 320796\| | 1.7E−275 | [Mus musculus] Catalytic subunit of calmodulin-regulated protein phosphatase |
| | | Ppp3cb | | (calcineurin A, beta isoform), plays a role in the skeletal muscle response to |
| | | | | functional overload |
| | | 437757\| | 7.3E−234 | [Rattus norvegicus][Protein phosphatase; Hydrolase; Smallmolecule-binding |
| | | Ppp3ca | | protein] Catalytic subunit of calmodulin regulated protein phosphatase |
| | | | | (calcineurin A alpha), regulates activity of transcription factors involved in signal |
| | | | | transduction and growth control, regulates long term potentiation |
| | | 318682\| | 7.3E−234 | [Mus musculus][Protein phosphatase; Hydrolase; Small molecule-binding |
| | | Ppp3ca | | protein][Nuclear] Catalytic subunit of calmodulin regulated protein phosphatase |
| | | | | (calcineurin A alpha), regulates activity of transcription factors involved in signal |
| | | | | transduction and growth control, regulates long term potentiation and memory |
| 51 | 7503249CD1 | g1418936 | 2.1E−142 | [Homo sapiens] protein-tyrosine-phosphatase |
| | | | | Groom, L. A. et al. EMBO J. 15, 3621-3632 (1996) |
| | | 347310\| | 1.8E−143 | [Homo sapiens][Protein phosphatase; Hydrolase] Dual specificity protein |
| | | DUSP7 | | phosphatase-7, member of a sub-family of phosphatases that selectively |
| | | | | dephosphorylates and inactivates mitogen-activated protein kinase, may be deleted |
| | | | | or mutated in specific cancers |
| | | | | Smith, A. et al. Genomics 42, 524-7 (1997). |
| | | 330198\| | 2.9E−119 | [Rattus norvegicus][Protein phosphatase; Hydrolase] Member of the dual |
| | | Rn.10244 | | specificity protein phosphatase family; human MKP-X selectively |
| | | | | dephosphorylates and inactivates mitogen-activated kinase and may be deleted or |
| | | | | mutated in specific cancers |
| | | 328638\| | 1.8E−115 | [Rattus norvegicus][Protein phosphatase; Hydrolase][Cytoplasmic] Dual |
| | | Rn.4313 | | specificity protein phosphatase, a cytosolic protein that selectively |
| | | | | dephosphorylates and inactivates mitogen-activated protein kinase, induced in |
| | | | | neurons by nerve growth factor |
| | | 662410\| | 5.9E−79 | [Homo sapiens][Protein phosphatase; Hydrolase][Cytoplasmic] Dual specificity |
| | | DUSP6 | | phosphatase 6, a cytosolic phosphatase that selectively dephosphorylates and |
| | | | | inactivates mitogen-activated protein kinases, downregulated in some pancreatic |
| | | | | cancer cell lines |
| | | 335090\| | 2.9E−73 | [Homo sapiens][Protein phosphatase; Hydrolase][Nuclear; Cytoplasmic] Dual |
| | | DUSP9 | | specificity phosphatase 9, inactivates mitogen-activated protein kinases through |
| | | | | dephosphorylation of phosphotyrosine and phosphothreonine residues, plays a |
| | | | | role in MAP kinase signal transduction |
| 52 | 7505890CD1 | g12314230 | 1.1E−51 | [Homo sapiens] dJ846F13.1 (phosphatidic acid phosphatase type 2c) |
| | | 658962\| | 4.7E−173 | [Homo sapiens] Member of the phosphatidic acid phosphatase-related (PAP2) |
| | | FLJ13055 | | phosphoesterase family, has low similarity to phosphatidic acid phosphatase 2c |
| | | | | (human PPAP2C), which hydrolyzes phospholipids |
| | | 599270\| | 1.6E−68 | [Homo sapiens][Other phosphatase; Hydrolase] Member of the phosphatidic acid |
| | | FLJ20300 | | (PA) phosphatase-related family |
| | | 691534\| | 7.8E−30 | [Homo sapiens] Protein of unknown function, has a region of weak similarity to |
| | | FLJ11535 | | phosphatidic acid phosphatase 2c (human PPAP2C), which hydrolyzes |
| | | | | phospholipids |
| | | 337108\| | 6.3E−25 | [Homo sapiens][Protein phosphatase; Hydrolase][membrane] Phosphatidic acid |
| | | PPAP2C | | phosphatase 2c, hydrolyzes phospholipids, may play a role in signal transduction |
| | | | | Hooks, S. B. et al. J. Biol. Chem. 276, 4611-21 (2001). |
| | | 327360\| | 2.6E−22 | [Mus musculus][Other phosphatase; Hydrolase][membrane] Phosphatidic acid |
| | | Ppap2a | | phosphatase type 2a, catalyzes the dephosphorylation of various lipid phosphates, |
| | | | | may regulate the level of lipid phosphates which are involved is signal |
| | | | | transduction |

TABLE 3

| | | | | Potential | | Analytical Methods |
|---|---|---|---|---|---|---|
| SEQ ID NO: | Incyte Polypeptide ID | Amino Acid Residues | Potential Phosphorylation Sites | Glycosylation Sites | Signature Sequences, Domains and Motifs | and Databases |
| 1 | 7499969CD1 | 458 | S7 S133 S166 S194 | N40 N131 N270 | SH2 domain: W127-Y209 | HMMER_PFAM |
| | | | S223 S272 S326 | | | |
| | | | S441 T105 T344 | | | |
| | | | T356 T367 T394 | | | |
| | | | T448 Y343 | | | |
| | | | | | SH3 domain: N64-A119 | HMMER_PFAM |
| | | | | | Protein kinase domain: K218-L439 | HMMER_PFAM |
| | | | | | Receptor tyrosine kinase class II proteins | BLIMPS_BLOCKS |
| | | | | | BL00239: A236-I283, L290-R312, R315-D340, | |
| | | | | | N341-Y390, N395-L439 | |
| | | | | | Receptor tyrosine kinase class III proteins | BLIMPS_BLOCKS |
| | | | | | BL00240: K289-S326, D340-R387, R387-L439 | |
| | | | | | Receptor tyrosine kinase class V proteins | BLIMPS_BLOCKS |
| | | | | | BL00790: T210-I263, A294-R315, A316-E342, | |
| | | | | | G348-T380, E381-G405, Y406-Y454 | |
| | | | | | Protein kinases signatures and profile: | PROFILESCAN |
| | | | | | K289-E342 | |
| | | | | | Receptor tyrosine kinase class II signature: | PROFILESCAN |
| | | | | | N318-G364 | |
| | | | | | Tyrosine kinase catalytic domain signature | BLIMPS_PRINTS |
| | | | | | PR00109: T265-K278, F303-V321, F351-I361, | |
| | | | | | S370-G392, C414-F436 | |
| | | | | | SH2 domain signature | BLIMPS_PRINTS |
| | | | | | PR00401: W127-L141, H148-S158, A160-D171, | |
| | | | | | V177-D187, T198-Y212 | |
| | | | | | SH3 domain signature | BLIMPS_PRINTS |
| | | | | | PR00452: N64-P74, G78-Q93, S94-L103, | |
| | | | | | Q107-A119 | |
| | | | | | KINASE PROTO-ONCOGENE TYROSINE | BLAST_PRODOM |
| | | | | | PROTEIN LCK PHOSPHORYLATION | |
| | | | | | TRANSFERASE ATP-BINDING | |
| | | | | | MYRISTYLATION SH2 DOMAIN | |
| | | | | | PD012180: G2-L65 | |
| | | | | | SH2 DOMAIN KINASE SH3 PROTEIN | BLAST_PRODOM |
| | | | | | PHOSPHORYLATION TYROSINE PROTEIN | |
| | | | | | TRANSFERASE ATP-BINDING TYROSINE | |
| | | | | | PD000093: W127-K222 | |
| | | | | | PROTEIN KINASE DOMAIN | BLAST_DOMO |
| | | | | | DM00004 | |
| | | | | | I48845\|244-486: Y212-F436 | |
| | | | | | P42683\|242-484: Y212-F436 | |
| | | | | | P08631\|261-503: Y213-F436 | |
| | | | | | P51451\|239-481: Y212-F436 | |
| | | | | | Tyrosine protein kinases specific active-site signature: | MOTIFS |
| | | | | | Y309-V321 | |
| 2 | 7499974CD1 | 2108 | S29 S34 S174 S189 | N27 N89 N850 | Protein kinase domain: L221-F479 | HMMER_PFAM |
| | | | S231 S260 S363 | N1019 N1051 | | |
| | | | S378 S469 S588 | N1601 N1771 | | |
| | | | S679 S792 S816 | N1781 N1789 | | |
| | | | S831 S836 S852 | N1877 N1989 | | |
| | | | S902 S946 S1162 | N2089 | | |
| | | | S1614 S1624 S1687 | | | |
| | | | S1738 S1763 S1787 | | | |
| | | | S1791 S1847 S1861 | | | |
| | | | S1966 S1967 S1991 | | | |
| | | | S1996 S2012 T48 | | | |
| | | | T60 T73 T91 T160 | | | |
| | | | T243 T258 T290 | | | |
| | | | T308 T373 T436 | | | |
| | | | T625 T736 T823 | | | |
| | | | T824 T841 T872 | | | |
| | | | T1243 T1380 | | | |
| | | | T1655 T1696 | | | |
| | | | T1854 T1971 Y468 | | | |
| | | | Y1828 | | | |
| | | | | | Protein kinases signatures and profile: | PROFILESCAN |
| | | | | | L324-S378 | |
| | | | | | Tyrosine kinase catalytic domain signature | BLIMPS_PRINTS |
| | | | | | PR00109: T301-K314, H339-I357, V403-C425, | |
| | | | | | A448-I470 | |
| | | | | | KIAA0344 ANTIGEN NYCO43 | BLAST_PRODOM |
| | | | | | PD041299: T1694-P1889 | |
| | | | | | PROTEIN KINASE DOMAIN | BLAST_DOMO |
| | | | | | DM00004 | |
| | | | | | S49611\|39-259: I227-V447 | |
| | | | | | P51957\|8-251: I227-I470 | |
| | | | | | Q05609\|553-797: E226-C459 | |
| | | | | | P41892\|11-249: I227-K471 | |
| | | | | | Serine/Threonine protein kinases active-site signature: | MOTIFS |
| | | | | | I345-I357 | |
| 3 | 7499976CD1 | 232 | S4 S14 S42 S92 | N22 N188 | Protein kinase domain: I21-Q130, V134-Y201 | HMMER_PFAM |
| | | | T24 T43 T120 | | | |
| | | | T128 | | | |
| | | | | | PROTEIN KINASE DOMAIN | BLAST_DOMO |
| | | | | | DM00004 | |
| | | | | | P49137\|66-315: T24-F210 | |
| | | | | | P49071\|21-271: L27-F210 | |
| | | | | | Q06850\|151-398: Q25-K129, Y187-Q216 | |
| | | | | | P08414\|44-285: K26-V202 | |
| 4 | 7499954CD1 | 353 | S7 S10 S31 S87 | | Protein-tyrosine phosphatase: R9-Q183 | HMMER_PFAM |
| | | | S95 S113 S152 | | | |
| | | | S275 T68 T286 | | | |
| | | | Y282 | | | |
| | | | | | Tyrosine specific protein phosphatases proteins | BLIMPS_BLOCKS |
| | | | | | BL00383: Q82-P94, V120-G130, R161-F176 | |
| | | | | | Tyrosine specific protein phosphatases signature and | PROFILESCAN |
| | | | | | profiles: | |
| | | | | | M100-F151 | |
| | | | | | Protein tyrosine phosphatase signature | BLIMPS_PRINTS |
| | | | | | PR00700: F151-A166, A167-L177, R78-S95, P117-V135 | |
| | | | | | PHOSPHATASE HYDROLASE PROTEIN PTP | BLAST_PRODOM |
| | | | | | TYROSINE PROTEIN TYROSINE PTPK1 FETAL | |
| | | | | | LIVER FLP1 | |
| | | | | | PD022097: L177-V353 | |
| | | | | | HYDROLASE PHOSPHATASE PROTEIN | BLAST_PRODOM |
| | | | | | PROTEIN TYROSINE TYROSINE PRECURSOR | |
| | | | | | SIGNAL TRANSMEMBRANE GLYCOPROTEIN | |
| | | | | | RECEPTOR | |
| | | | | | PD000155: R78-Q183 | |
| | | | | | HYDROLASE PHOSPHATASE PROTEIN | BLAST_PRODOM |
| | | | | | PROTEIN TYROSINE PRECURSOR SIGNAL | |
| | | | | | TYROSINE TRANSMEMBRANE | |
| | | | | | GLYCOPROTEIN RECEPTOR | |
| | | | | | PD000167: K32-Y178 | |
| | | | | | PROTEIN-TYROSINE-PHOSPHATASE | BLAST_DOMO |
| | | | | | DM00089 | |
| | | | | | P29352\|22-291: K32-F185 | |
| | | | | | S48748\|14-295: K32-F185 | |
| | | | | | JH06091\|14-296: K32-F185 | |
| | | | | | I48666\|14-296: K32-F185 | |
| | | | | | Tyrosine specific protein phosphatases active site: | MOTIFS |
| | | | | | V120-L132 | |
| 5 | 7500827CD1 | 452 | S121 S144 S196 | N140 | signal_cleavage: M1-S18 | SPSCAN |
| | | | S234 S246 S271 | | | |
| | | | S298 S329 S397 | | | |
| | | | T92 T142 T315 | | | |
| | | | T324 T338 Y177 | | | |
| | | | Y279 | | | |
| | | | | | Signal Peptide: M1-S18 | HMMER |
| | | | | | Tyrosine specific protein phosphatases active site: | MOTIFS |
| | | | | | V242-F254 | |
| 6 | 7948585CD1 | 480 | S83 S90 S133 S152 | N148 | signal_cleavage: M1-A62 | SPSCAN |
| | | | S216 S233 S286 | | | |
| | | | S309 S330 S332 | | | |
| | | | S357 S362 S364 | | | |
| | | | S420 S435 S464 | | | |
| | | | S468 T193 T228 | | | |
| | | | T266 T293 | | | |
| | | | | | BRAIN ENRICHED GUANYLATE KINASE- | BLAST_PRODOM |
| | | | | | ASSOCIATED PROTEIN | |
| | | | | | PD156004: E195-N480 | |
| | | | | | BRAIN ENRICHED GUANYLATE KINASE- | BLAST_PRODOM |
| | | | | | ASSOCIATED PROTEIN | |
| | | | | | PD156002: Q32-S152 | |
| 7 | 7500002CD1 | 197 | S82 S87 S106 S109 | | Adenylate kinase: V32-I164, L20-Q31 | HMMER_PFAM |
| | | | S166 T62 T98 | | | |
| | | | T189 | | | |
| | | | | | Adenylate kinase signature: | PROFILESCAN |
| | | | | | V32-P89 | |
| | | | | | Adenylate kinase signature | BLIMPS_PRINTS |
| | | | | | PR00094: R133-Y148, T150-I164, V19-V32, | |
| | | | | | F54-D70 | |
| | | | | | KINASE ADENYLATE TRANSFERASE ATP- | BLAST_PRODOM |
| | | | | | BINDING ATP/AMP TRANSPHOSPHORYLASE | |
| | | | | | ISOENZYME PROTEIN 3D STRUCTURE | |
| | | | | | MITOCHONDRION | |
| | | | | | PD000657: K28-I164 | |
| | | | | | ADENYLATE KINASE ISOENZYME | BLAST_PRODOM |
| | | | | | MITOCHONDRIAL ATP/AMP | |
| | | | | | TRANSPHOSPHORYLASE TRANSFERASE ATP- | |
| | | | | | BINDING MITOCHONDRION ALTERNATIVE | |
| | | | | | PD022013: H165-I197 | |
| | | | | | ADENYLATE KINASE | BLAST_DOMO |
| | | | | | DM00562\|P08166\|144-228: L101-S186 | |
| | | | | | ADENYLATE KINASE | BLAST_DOMO |
| | | | | | DM00290 | |
| | | | | | P24323\|1-177: K28-L155, I16-Q31 | |
| | | | | | I64062\|1-174: K28-T152, I16-Q31 | |
| | | | | | P08166\|14-142: M1-R100, P13-Q31 | |
| | | | | | Adenylate kinase signature: F54-Q65 | MOTIFS |
| 8 | 7500012CD1 | 1300 | S6 S21 S62 S73 | N677 N724 N809 | Protein kinase domain: L40-E315 | HMMER_PFAM |
| | | | S93 S305 S393 | N959 N1141 | | |
| | | | S456 S530 S540 | | | |
| | | | S551 S661 S726 | | | |
| | | | S737 S738 S784 | | | |
| | | | S811 S906 S965 | | | |
| | | | S1018 S1165 S1179 | | | |
| | | | S1180 S1182 S1289 | | | |
| | | | T155 T186 T382 | | | |
| | | | T414 T459 T611 | | | |
| | | | T680 T776 T805 | | | |
| | | | T949 T1101 T1110 | | | |
| | | | T1189 Y412 | | | |
| | | | | | Protein kinases signatures and profile: | PROFILESCAN |
| | | | | | V148-H200 | |
| | | | | | PROTEIN AUXILIN COAT REPEAT | BLAST_PRODOM |
| | | | | | PHOSPHORYLATION KIAA0473 CYCLIN G | |
| | | | | | ASSOCIATED KINASE TRANSFERASE | |
| | | | | | PD151518: L641-S1140, N809-S1180, R320-E366 | |
| | | | | | PROTEIN PHOSPHORYLATION AUXILIN COAT | BLAST_PRODOM |
| | | | | | REPEAT KIAA0473 CYCLIN G ASSOCIATED | |
| | | | | | KINASE TRANSFERASE | |
| | | | | | PD025411: S456-V640 | |
| | | | | | CYCLIN G ASSOCIATED KINASE | BLAST_PRODOM |
| | | | | | TRANSFERASE SERINE/THREONINE PROTEIN | |
| | | | | | ATP-BINDING HSGAK | |
| | | | | | PD039449: A317-N402 | |
| | | | | | PROTEIN AUXILIN COAT REPEAT | BLAST_PRODOM |
| | | | | | PHOSPHORYLATION KIAA0473 CYCLIN G | |
| | | | | | ASSOCIATED KINASE TRANSFERASE | |
| | | | | | PD010124: Q1160-Q1294 | |
| | | | | | PROTEIN KINASE DOMAIN | BLAST_DOMO |
| | | | | | DM00004 | |
| | | | | | P40494\|23-287: R41-I306 | |
| | | | | | P53974\|23-288: R44-I306 | |
| | | | | | P38080\|36-309: L46-I306 | |
| | | | | | Q09170\|169-423: R44-S305 | |
| | | | | | Serine/Threonine protein kinases active-site signature: | MOTIFS |
| | | | | | I169-L181 | |
| 9 | 1664071CD1 | 176 | S72 S167 S168 | | HYPOTHETICAL 20.4 KD PROTEIN IN | BLAST_PRODOM |
| | | | S172 T23 T36 | | GLC7GDI1 INTERGENIC REGION PD101469: M1-F84 | |
| | | | T102 Y48 | | | |
| 10 | 6214577CD1 | 595 | S15 S17 S39 S78 | N137 N141 N221 | Dual specificity phosphatase, catalytic domain: H196-Q329 | HMMER_PFAM |
| | | | S199 S358 S405 | N368 N463 N519 | | |
| | | | S502 S504 S556 | N538 | | |
| | | | S577 T24 T86 | | | |
| | | | T111 T143 T239 | | | |
| | | | T255 T301 T395 | | | |
| | | | T445 T448 T458 | | | |
| | | | T472 T525 Y116 | | | |
| | | | Y205 | | | |
| | | | | | Tyrosine specific protein phosphatases signature and | PROFILESCAN |
| | | | | | profiles: V261-P314 | |
| | | | | | HYDROLASE CDC14 HOMOLOG CDC14A1 | BLAST_PRODOM |
| | | | | | PHOSPHATASE | |
| | | | | | PD037525: D381-P586 | |
| | | | | | HYDROLASE PROTEIN PHOSPHATASE | BLAST_PRODOM |
| | | | | | CHROMOSOME II ALTERNATIVE SPLICING | |
| | | | | | CDC14 PROBABLE PROTEIN TYROSINE | |
| | | | | | PD006252: V105-S193 | |
| | | | | | HYDROLASE PHOSPHATASE CDC14 | BLAST_PRODOM |
| | | | | | HOMOLOG CDC14A1 CDC14A2 | |
| | | | | | PD021466: E326-E380 | |
| | | | | | HYDROLASE PHOSPHATASE PROTEIN | BLAST_PRODOM |
| | | | | | CHROMOSOME II ALTERNATIVE SPLICING | |
| | | | | | CDC14 PROBABLE PROTEIN TYROSINE | |
| | | | | | PD006832: D18-A104 | |
| | | | | | Tyrosine specific protein phosphatases active site: | MOTIFS |
| | | | | | V277-L289 | |
| 11 | 7502149CD1 | 2171 | S75 S177 S232 | N223 N692 N1025 | HECT-domain (ubiquitin-transferase) | BLIMPS_PFAM |
| | | | S240 S346 S397 | N1040 N1393 | PF00632: F2070-P2097, Y2133-Y2164 | |
| | | | S411 S452 S555 | N1699 N1747 | | |
| | | | S694 S725 S771 | | | |
| | | | S783 S881 S891 | | | |
| | | | S1063 S1078 S1083 | | | |
| | | | S1099 S1284 S1304 | | | |
| | | | S1316 S1370 S1382 | | | |
| | | | S1395 S1413 S1521 | | | |
| | | | S1546 S1560 S1631 | | | |
| | | | S1687 S1802 S1825 | | | |
| | | | S1931 S2000 S2032 | | | |
| | | | S2149 | | | |
| | | | T181 T195 T298 | | PROTEIN LIGASE UBIQUITIN CONJUGATION | BLAST_PRODOM |
| | | | T439 T491 T706 | | REPEAT UBIQUITIN PROTEIN DNA BINDING | |
| | | | T739 T843 T1121 | | PROBABLE ONCOGENIC | |
| | | | T1159 T1194 | | PD002225: F1877-H2163 | |
| | | | T1256 T1327 | | | |
| | | | T1522 T1551 | | | |
| | | | T1572 T1635 | | | |
| | | | T1700 T1815 | | | |
| | | | T1868 T1936 | | | |
| | | | T1971 T2079 Y477 | | | |
| | | | Y1507 Y1780 | | | |
| | | | | | Leucine zipper pattern: L621-L642, L1681-L1702, | MOTIFS |
| | | | | | L1688-L1709 | |
| 12 | 7503480CD1 | 971 | S20 S49 S137 S292 | N311 N330 N384 | signal_cleavage: M1-T53 | SPSCAN |
| | | | S313 S332 S356 | N540 | | |
| | | | S357 S365 S367 | | | |
| | | | S422 S473 S478 | | | |
| | | | S507 S509 S521 | | | |
| | | | S541 S542 S621 | | | |
| | | | S633 S657 S687 | | | |
| | | | S690 S780 S793 | | | |
| | | | S812 S818 S829 | | | |
| | | | S837 S853 S862 | | | |
| | | | S863 S919 S936 | | | |
| | | | T22 T141 T267 | | Ank repeat: D72-N104, S198-Y230, E105-S137, | HMMER_PFAM |
| | | | T316 T371 T435 | | D231-K263, D39-V71, E138-K171 | |
| | | | T529 T593 T637 | | | |
| | | | T641 T689 T702 | | | |
| | | | T794 T823 T894 | | | |
| | | | T909 Y68 Y707 | | | |
| | | | | | Ank repeat proteins. | BLIMPS_PFAM |
| | | | | | PF00023: L110-L125, G232-H241 | |
| | | | | | MYOSIN SUBUNIT PHOSPHATASE SMOOTH | BLAST_PRODOM |
| | | | | | MUSCLE A MYOSIN BINDING OF TARGET | |
| | | | | | REPEAT | |
| | | | | | PD013740: T371-S585 | |
| | | | | | SUBUNIT MYOSIN PHOSPHATASE SMOOTH | BLAST_PRODOM |
| | | | | | MUSCLE A REPEAT MYOSIN BINDING OF | |
| | | | | | TARGET | |
| | | | | | PD015296: S793-R926 | |
| | | | | | SUBUNIT MYOSIN PHOSPHATASE SMOOTH | BLAST_PRODOM |
| | | | | | MUSCLE A REPEAT MYOSIN BINDING OF | |
| | | | | | TARGET | |
| | | | | | PD012330: D273-K350 | |
| | | | | | MYOSIN SUBUNIT PHOSPHATASE PROTEIN | BLAST_PRODOM |
| | | | | | SMOOTH MUSCLE A REPEAT MYOSIN | |
| | | | | | BINDING OF | |
| | | | | | PD010421: M1-V71 | |
| | | | | | LIGHT; M21; MYOSIN; | BLAST_DOMO |
| | | | | | DM05524 | |
| | | | | | A55142\|851-1003: G795-R926 | |
| | | | | | S51022\|1-160: S797-L945 | |
| | | | | | RECOGNITION; TUMOR; PROLYL; NATURAL; | BLAST_DOMO |
| | | | | | DM08077\|P30414\|230-1403: K286-S835, | |
| | | | | | Q808-S863, E173-G200 | |
| | | | | | ANKYRIN REPEAT | BLAST_DOMO |
| | | | | | DM00014\|A55142\|219-252: I219-D253 | |
| | | | | | Leucine zipper pattern: L948-L969 | MOTIFS |
| 13 | 7500017CD1 | 428 | S14 S21 S42 S126 | N72 N221 N295 | Protein kinase domain: Y64-F348 | HMMER_PFAM |
| | | | S211 S244 S269 | N369 N426 | | |
| | | | S327 S334 S338 | | | |
| | | | S346 S371 T317 | | | |
| | | | T411 Y64 | | | |
| | | | | | Protein kinases signatures and profile: Y165-G218 | PROFILESCAN |
| | | | | | Tyrosine kinase catalytic domain signature | BLIMPS_PRINTS |
| | | | | | PR00109: Y179-V197, I246-D268, T317-P339 | |
| | | | | | KINASE TRANSFERASE PROTEIN | BLAST_PRODOM |
| | | | | | SERINE/THREONINE PROTEIN ATP-BINDING II | |
| | | | | | PHOSPHORYLATION CASEIN ALPHA CHAIN | |
| | | | | | PD002608: S227-F348 | |
| | | | | | GLYCOGEN SYNTHASE KINASE 3-ALPHA | BLAST_PRODOM |
| | | | | | GSK3 TRANSFERASE SERINE/THREONINE | |
| | | | | | PROTEIN KINASE ATP-BINDING MULTIGENE | |
| | | | | | PD026219: T29-A63 | |
| | | | | | KINASE PROTEIN TRANSFERASE ATP- | BLAST_PRODOM |
| | | | | | BINDING SERINE/THREONINE PROTEIN | |
| | | | | | PHOSPHORYLATION RECEPTOR TYROSINE | |
| | | | | | PROTEIN PRECURSOR TRANSMEMBRANE | |
| | | | | | PD000001: L120-I246, Y230-F348, K68-R100 | |
| | | | | | PROTEIN KINASE DOMAIN | BLAST_DOMO |
| | | | | | DM00004 | |
| | | | | | P49840\|121-393: D66-P339 | |
| | | | | | P49841\|57-330: T65-P339 | |
| | | | | | P23646\|288-561: T65-P339 | |
| | | | | | P18431\|55-328: T65-P339 | |
| | | | | | Protein kinases ATP-binding region signature: I70-K93 | MOTIFS |
| | | | | | Serine/Threonine protein kinases active-site signature: | MOTIFS |
| | | | | | V185-V197 | |
| 14 | 7499955CD1 | 286 | S2 S4 S163 S240 | | signal_cleavage: M1-G23 | SPSCAN |
| | | | S281 T107 | | | |
| | | | | | Ser/Thr protein phosphatase: G19-K257 | HMMER_PFAM |
| | | | | | Serine/threonine specific protein phosphatases | BLIMPS_BLOCKS |
| | | | | | proteins | |
| | | | | | BL00125: G14-V50, S56-N101, A119-P165, | |
| | | | | | S180-N234 | |
| | | | | | Serine/threonine specific protein phosphatases | PROFILESCAN |
| | | | | | signature: S56-I102 | |
| | | | | | Serine/threonine phosphatase family signature | BLIMPS_PRINTS |
| | | | | | PR00114: G14-S41, Y43-Y70, L76-Y100, | |
| | | | | | D110-L136, M139-D166, D196-K216, | |
| | | | | | Q218-N234 | |
| | | | | | PROTEIN PHOSPHATASE SERINE/THREONINE | BLAST_PRODOM |
| | | | | | HYDROLASE IRON MANGANESE SUBUNIT | |
| | | | | | MULTIGENE FAMILY CATALYTIC | |
| | | | | | PD000252: G19-K257 | |
| | | | | | F58G1.3 PROTEIN | BLAST_PRODOM |
| | | | | | PD000297: V179-P254 | |
| | | | | | SIMILAR TO SERINE/THREONINE PROTEIN | BLAST_PRODOM |
| | | | | | PHOSPHATASE | |
| | | | | | PD112269: G19-P71 | |
| | | | | | PROTEIN PHOSPHATASE PP1 ALPHA | BLAST_PRODOM |
| | | | | | CATALYTIC SUBUNIT HYDROLASE | |
| | | | | | GLYCOGEN METABOLISM ALTERNATIVE | |
| | | | | | SERINE/THREONINE | |
| | | | | | PD004641: N258-K286 | |
| | | | | | PHOSPHOPROTEIN PHOSPHATASE | BLAST_DOMO |
| | | | | | DM00133 | |
| | | | | | P08128\|1-291: G19-G267 | |
| | | | | | P36873\|15-310: G19-K259 | |
| | | | | | P37139\|15-310: G19-K259 | |
| | | | | | C32550\|15-310: G19-K259 | |
| | | | | | Serine/threonine specific protein phosphatases | MOTIFS |
| | | | | | signature: L77-E82 | |
| 15 | 7504025CD1 | 764 | S30 S49 S68 S84 | N61 N444 N529 | MYND finger: C606-C640 | HMMER_PFAM |
| | | | S102 S114 S147 | N620 N661 N725 | | |
| | | | S171 S190 S211 | N744 | | |
| | | | S216 S222 S228 | | | |
| | | | S234 S279 S300 | | | |
| | | | S331 S333 S346 | | | |
| | | | S361 S380 S622 | | | |
| | | | S697 S724 S730 | | | |
| | | | S731 S738 S748 | | | |
| | | | S757 | | | |
| | | | T12 T44 T66 T79 | | ACIDIC SERINE CLUSTER REPEAT | BLAST_DOMO |
| | | | T127 T128 T130 | | DM04746\|S57757\|1-646: T6-K516, P627-P754 | |
| | | | T206 T508 T518 | | | |
| | | | T534 T566 T600 | | | |
| | | | T687 T739 T761 | | | |
| 16 | 7503203CD1 | 1634 | S75 S82 S86 S115 | N1029 N1088 | signal_cleavage: M1-S68 | SPSCAN |
| | | | S119 S140 S152 | N1129 | | |
| | | | S175 S203 S402 | | | |
| | | | S425 S430 S455 | | | |
| | | | S611 S642 S647 | | | |
| | | | S653 S661 S682 | | | |
| | | | S690 S696 S710 | | | |
| | | | S745 S750 S767 | | | |
| | | | S920 S936 | | | |
| | | | S1031 S1041 S1050 | | Signal Peptide M31-A54 | HMMER |
| | | | S1061 S1065 S1066 | | | |
| | | | S1092 S1108 S1168 | | | |
| | | | S1173 S1254 S1261 | | | |
| | | | S1265 S1283 S1295 | | | |
| | | | S1327 S1339 S1340 | | | |
| | | | S1377 S1486 S1493 | | | |
| | | | S1496 S1507 S1534 | | | |
| | | | S1553 S1607 | | | |
| | | | T188 T428 T436 | | PDZ domain (Also known as DHR or GLGF).: P940-L1027 | HMMER_PFAM |
| | | | T487 T503 T595 | | | |
| | | | T622 T651 T707 | | | |
| | | | T752 T761 T785 | | | |
| | | | T850 T872 T876 | | | |
| | | | T953 T1025 T1072 | | | |
| | | | T1080 T1260 | | | |
| | | | T1316 T1511 | | | |
| | | | T1601 | | | |
| | | | | | Protein kinase domain: F434-K580, D593-F621 | HMMER_PFAM |
| | | | | | Protein kinases signatures and profile: F501-M581 | PROFILESCAN |
| | | | | | PROTEIN SH3 DOMAIN REPEAT G990-S1003 | BLIMPS_PRODOM |
| | | | | | MICROTUBULE ASSOCIATED TESTIS SPECIFIC | BLAST_PRODOM |
| | | | | | SERINE/THREONINE PROTEIN KINASE 205 KD | |
| | | | | | TESTIS SPECIFIC SERINE/THREONINE | |
| | | | | | PROTEIN KINASE MAST205 KINASE PD142315: | |
| | | | | | H1149-T1634 | |
| | | | | | MICROTUBULE ASSOCIATED TESTIS SPECIFIC | BLAST_PRODOM |
| | | | | | SERINE/THREONINE PROTEIN KINASE 205 KD | |
| | | | | | TESTIS SPECIFIC SERINE/THREONINE | |
| | | | | | PROTEIN KINASE MAST205 KINASE PD182663: | |
| | | | | | E699-H975 | |
| | | | | | MICROTUBULE ASSOCIATED TESTIS SPECIFIC | BLAST_PRODOM |
| | | | | | SERINE/THREONINE PROTEIN KINASE 205 KD | |
| | | | | | TESTIS SPECIFIC SERINE/THREONINE | |
| | | | | | PROTEIN KINASE MAST205 KINASE PD135564: | |
| | | | | | C83-Y242 | |
| | | | | | PROTEIN KINASE SERINE/THREONINE KIN4 | BLAST_PRODOM |
| | | | | | MICROTUBULE ASSOCIATED TESTIS SPECIFIC | |
| | | | | | TESTIS SPECIFIC MAST205 PD041650: K243-D433 | |
| | | | | | PROTEIN KINASE DOMAIN | BLAST_DOMO |
| | | | | | DM00004\|A54602\|455-712: T436-E592, E592-G608 | |
| | | | | | GLGF DOMAIN DM00224\|A54602\|1032-1126: | BLAST_DOMO |
| | | | | | F930-T1025 | |
| | | | | | SERINE/THREONINE PROTEIN KINASES | BLAST_DOMO |
| | | | | | DM00087\|A54602\|714-794: T609-S690 | |
| | | | | | PROTEIN KINASE DOMAIN DM08046\|P05986\|1-397: | BLAST_DOMO |
| | | | | | S430-K580, E592-E665, D190-P213 | |
| | | | | | Serine/Threonine protein kinases active-site signature: | MOTIFS |
| | | | | | I553-I565 | |
| | | | | | Leucine zipper pattern: L522-L543 | MOTIFS |
| 17 | 7503260CD1 | 1553 | S161 S280 S307 | | signal_cleavage: M1-S37 | SPSCAN |
| | | | S363 S407 S430 | | | |
| | | | S471 S545 S625 | | | |
| | | | S629 S646 S675 | | | |
| | | | S710 S729 S736 | | | |
| | | | S806 S810 S814 | | | |
| | | | S840 S1039 S1143 | | | |
| | | | S1275 S1386 S1395 | | | |
| | | | S1481 S1537 T455 | | | |
| | | | T590 T673 T869 | | | |
| | | | T937 T1069 T1359 | | | |
| | | | | | CNH domain: L1081-K1361 | HMMER_PFAM |
| | | | | | Phorbol esters/diacylglycerol binding dom: H868-C916 | HMMER_PFAM |
| | | | | | PH domain: T937-R1055 | HMMER_PFAM |
| | | | | | Protein kinase domain: F71-F337 | HMMER_PFAM |
| | | | | | Phorbol esters/diacylglycerol binding domain: C881-S944 | PROFILESCAN |
| | | | | | Tyrosine kinase catalytic domain signature PR00109: | BLIMPS_PRINTS |
| | | | | | S185-L203, C257-E279, M148-S161 | |
| | | | | | PHORBOLESTER BINDING KINASE | BLAST_PRODOM |
| | | | | | DYSTROPHY KINASE RELATED CDC42 | |
| | | | | | BINDING SIMILAR SERINE/THREONINE | |
| | | | | | PROTEIN GENGHIS KHAN PD150840: W1336-G1443 | |
| | | | | | PHORBOLESTER BINDING KINASE | BLAST_PRODOM |
| | | | | | DYSTROPHY KINASE RELATED CDC42 | |
| | | | | | BINDING SIMILAR SERINE/THREONINE | |
| | | | | | PROTEIN GENGHIS KHAN PD151400: T1020-R1121 | |
| | | | | | KINASE RHO ASSOCIATED COILED COIL | BLAST_PRODOM |
| | | | | | PROTEIN FORMING PHORBOLESTER BINDING | |
| | | | | | DYSTROPHY KINASE RELATED CDC42 | |
| | | | | | BINDING PD006715: T925-V1019 | |
| | | | | | PROTEIN COILED COIL CHAIN MYOSIN | BLAST_PRODOM |
| | | | | | REPEAT HEAVY ATP BINDING FILAMENT | |
| | | | | | HEPTAD PD000002: Q483-Q680 | |
| | | | | | PROTEIN KINASE DOMAIN DM00004 | BLAST_DOMO |
| | | | | | \|Q09013\|83-336: I73-R325 | |
| | | | | | \|S42867\|75-498: I73-H252 | |
| | | | | | \|I38133\|90-369: E72-L220 | |
| | | | | | \|P53894\|353-658: L74-G215 | |
| | | | | | Leucine zipper pattern: L491-L512 | MOTIFS |
| | | | | | Phorbol esters/diacylglycerol binding domain: H868-C916 | MOTIFS |
| | | | | | Protein kinases ATP-binding region signature: I77-K100 | MOTIFS |
| | | | | | Serine/Threonine protein kinases active-site signature: | MOTIFS |
| | | | | | Y191-L203 | |
| 18 | 2969494CD1 | 1130 | S101 S119 S194 | N60 N84 N355 | PROTEIN COILED COIL CHAIN MYOSIN | BLAST_PRODOM |
| | | | S212 S223 S299 | N884 | REPEAT HEAVY ATP BINDING FILAMENT | |
| | | | S352 S477 S509 | | HEPTAD PD000002: L874-D1071 | |
| | | | S572 S591 S697 | | | |
| | | | S734 S774 S782 | | | |
| | | | S885 S886 S1033 | | | |
| | | | S1073 T104 T426 | | | |
| | | | T488 T544 T1014 | | | |
| | | | T1063 T1082 | | | |
| | | | T1090 T1092 | | | |
| | | | T1126 | | | |
| | | | | | PROTEIN REPEAT TROPOMYOSIN COILED | BLAST_PRODOM |
| | | | | | COIL ALTERNATIVE SPLICING SIGNAL | |
| | | | | | PRECURSOR CHAIN PD000023: L874-E1042 | |
| | | | | | do NEUROFILAMENT; TRIPLET; | BLAST_DOMO |
| | | | | | DM07286\|P16053\|427-608: S886-E1017 | |
| | | | | | VERPROLIN, A PROLINE-RICH PROTEIN | BLAST_DOMO |
| | | | | | INVOLVED IN CYTOSKELETAL | |
| | | | | | ORGANIZATION AND CELLULAR GROWTH IN | |
| | | | | | SACCHAROMYCES CEREVISIAE | |
| | | | | | DM08461\|P37370\|203-451: P643-P858 | |
| | | | | | PROLINE-RICH PROTEIN DM03894\|P05142\|1-134: | BLAST_DOMO |
| | | | | | P792-P871, P797-P872 | |
| | | | | | H-A-P-P REPEAT DM08271\|S25299\|69-249: P663-P848 | BLAST_DOMO |
| 19 | 7503201CD1 | 556 | S36 S51 S79 S109 | N313 N362 N375 | Protein kinase domain: Y14-V272 | HMMER_PFAM |
| | | | S395 S401 S525 | N392 | | |
| | | | T47 T94 T262 | | | |
| | | | T351 T376 T377 | | | |
| | | | T378 T456 | | | |
| | | | | | Protein kinases signatures and profile: F85-Q168 | PROFILESCAN |
| | | | | | Tyrosine kinase catalytic domain signature PR00109: | BLIMPS_PRINTS |
| | | | | | H126-L144, V195-E217, V241-A263 | |
| | | | | | KINASE PROTEIN II CALCIUM/CALMODULIN | BLAST_PRODOM |
| | | | | | DEPENDENT TYPE SUBUNIT CHAIN | |
| | | | | | TRANSFERASE SERINE/THREONINE PROTEIN | |
| | | | | | CALMODULIN BINDING PD004250: E468-Q556 | |
| | | | | | KINASE PROTEIN II CALCIUM/CALMODULIN | BLAST_PRODOM |
| | | | | | DEPENDENT TYPE SUBUNIT CALMODULIN | |
| | | | | | BINDING CHAIN TRANSFERASE | |
| | | | | | SERINE/THREONINE PROTEIN PD001779: V272-L383, | |
| | | | | | R424-V467 | |
| | | | | | CALCIUM/CALMODULIN DEPENDENT | BLAST_PRODOM |
| | | | | | PROTEIN KINASE II ISOFORM GAMMAG | |
| | | | | | PD063143: K318-A352 | |
| | | | | | PROTEIN KINASE DOMAIN DM00004 | BLAST_DOMO |
| | | | | | \|P11798\|15-261: L16-A263 | |
| | | | | | \|JU0270\|16-262: E18-A263 | |
| | | | | | \|A44412\|16-262: E18-A263 | |
| | | | | | \|S57347\|21-266: L20-T262 | |
| | | | | | Binding-protein-dependent transport systems inner | MOTIFS |
| | | | | | membrane comp. sign: V396-R424 | |
| | | | | | Protein kinases ATP-binding region signature: L20-k23 | MOTIFS |
| | | | | | Serine/Threonine protein kinases active-site signature: | MOTIFS |
| | | | | | I132-L144 | |
| 20 | 7503262CD1 | 489 | S47 S148 S206 | N181 N328 N360 | Protein kinase domain: Y4-V257 | HMMER_PFAM |
| | | | S243 S302 S308 | N384 | | |
| | | | S337 T197 T288 | | | |
| | | | T304 T356 T369 | | | |
| | | | T385 T386 T462 | | | |
| | | | | | Protein kinases signatures and profile: M103-M156 | PROFILESCAN |
| | | | | | Tyrosine kinase catalytic domain signature PR00109: | BLIMPS_PRINTS |
| | | | | | M79-K92, H117-L135, S183-N205, Y226-A248 | |
| | | | | | PROTEIN KINASE DOMAIN DM00004 | BLAST_DOMO |
| | | | | | \|P51954\|6-248: L7-S247 | |
| | | | | | \|P51957\|8-251: L7-S247 | |
| | | | | | \|P51955\|10-261: V6-S247 | |
| | | | | | \|Q08942\|22-269: M9-S247 | |
| | | | | | Protein kinases ATP-binding region signature: I10-K33 | MOTIFS |
| | | | | | Serine/Threonine protein kinases active-site signature: | MOTIFS |
| | | | | | V123-L135 | |
| 21 | 7503409CD1 | 408 | S85 S132 S218 | | CELL CYCLE PROGRESSION PROTEIN FAST | BLAST_PRODOM |
| | | | S259 S381 T222 | | KINASE PD041692: Q80-S317 | |
| | | | T251 T328 | | | |
| | | | | | FAST KINASE PD135788: C318-G408 | BLAST_PRODOM |
| | | | | | FAST KINASE PD135789: F29-R79 | BLAST_PRODOM |
| 22 | 7503499CD1 | 431 | S140 S151 S201 | N99 N138 N338 | GHMP kinases putative ATP-binding protei: W80-G156 | HMMER_PFAM |
| | | | S251 S280 S343 | | G156 | |
| | | | S351 T174 | | | |
| | | | | | Galactokinase proteins BL00106: G369-V382, P30-L52, | BLIMPS_BLOCKS |
| | | | | | P78-L88, G102-L123, E152-S181, F213-A224, | |
| | | | | | Q303-F318, L333-C362 | |
| | | | | | GHMP kinases ATP-binding domain proteins | BLIMPS_BLOCKS |
| | | | | | BL00627: I111-S121, R371-C380 | |
| | | | | | GHMP kinases putative ATP-binding domain: N99-E143 | PROFILESCAN |
| | | | | | Galactokinase family signature PR00473: G31-T49, | BLIMPS_PRINTS |
| | | | | | W80-I91, G102-S120, Q303-Q317 | |
| | | | | | Mevalonate kinase signature PR00959: A29-Q53, | BLIMPS_PRINTS |
| | | | | | G109-T131, S151-A170, G369-P386 | |
| | | | | | LmbP protein signature PR00960: N110-T131, R363-V382 | BLIMPS_PRINTS |
| | | | | | KINASE ATP BINDING TRANSFERASE | BLAST_PRODOM |
| | | | | | GALACTOKINASE GALACTOSE METABOLISM | |
| | | | | | MEVALONATE MK BIOSYNTHESIS PROTEIN | |
| | | | | | PD002375: T286-K399 | |
| | | | | | GALACTOKINASE GALACTOSE METABOLISM | BLAST_PRODOM |
| | | | | | ATPBINDING TRANSFERASE KINASE GAL3 | |
| | | | | | PROTEIN MULTIGENE FAMILY PD013932: | |
| | | | | | N138-L252 | |
| | | | | | GALACTOKINASE 2 EC 2.7.1.6 TRANSFERASE | BLAST_PRODOM |
| | | | | | KINASE GALACTOSE METABOLISM ATP | |
| | | | | | BINDING MULTIGENE FAMILY PD124431: L390-A431 | |
| | | | | | GALACTOKINASE 2 EC 2.7.1.6 TRANSFERASE | BLAST_PRODOM |
| | | | | | KINASE GALACTOSE METABOLISM ATP | |
| | | | | | BINDING MULTIGENE FAMILY PD168366: P46-L79 | |
| | | | | | GALACTOKINASE DM01364\|Q01415\|31-454: G20-L428 | BLAST_DOMO |
| | | | | | GALACTOKINASE DM01364\|P09608\|31-501: Q234-V427, | BLAST_DOMO |
| | | | | | D63-229, K24-V40 | |
| | | | | | GALACTOKINASE DM01364\|P04385\|40-524: G249-A425, | BLAST_DOMO |
| | | | | | K48-A228, P23-V40 | |
| | | | | | GALACTOKINASE DM01364\|P13045\|35-517: I36-A228, | BLAST_DOMO |
| | | | | | G249-G424, P23-V40 | |
| | | | | | GHMP kinases putative ATP-binding domain: I111-A122 | MOTIFS |
| 23 | 90031281CD1 | 601 | S193 S269 S315 | N300 | ATP-BINDING TRANSFERASE CHROMOSOME | BLAST_PRODOM |
| | | | S352 S374 S388 | | PROTEIN YOR3240W FROM XV C15A10.13 I | |
| | | | S392 S492 S514 | | W07G4.3 PD025526: N276-E391, F12-G158, L226-E280 | |
| | | | S530 S553 S563 | | | |
| | | | S567 S580 T39 T70 | | | |
| | | | T85 T398 T434 | | | |
| | | | T480 T487 Y184 | | | |
| 24 | 90061570CD1 | 160 | S54 T42 T81 | N97 | Protein kinase domain: Y20-D57 | HMMER_PFAM |
| 25 | 7500027CD1 | 148 | T32 T40 T140 Y29 | | Tyrosine specific protein phosphatases signature and | PROFILESCAN |
| | | | | | profiles: D59-K112 | |
| | | | | | Prenylation: C146-M148 | MOTIFS |
| 26 | 7504546CD1 | 149 | T32 T40 T123 | | Tyrosine specific protein phosphatases signature and | PROFILESCAN |
| | | | T141 Y29 | | profiles: D59-Q111 | |
| | | | | | PROTEIN TYROSINE PHOSPHATASE 4A3 | BLAST_PRODOM |
| | | | | | MPRL3 HPRL3 PD153367: S119-M149 | |
| | | | | | Prenylation: C147-M149 | MOTIFS |
| 27 | 7503246CD1 | 731 | S10 S100 S257 | N57 N352 N448 | Kinase associated domain: T682-L731 | HMMER_PFAM |
| | | | S333 S359 S376 | N541 | | |
| | | | S391 S410 S415 | | | |
| | | | S518 S559 S635 | | | |
| | | | S641 S646 S712 T9 | | | |
| | | | T42 T88 T242 | | | |
| | | | T261 T305 T341 | | | |
| | | | T417 T433 T434 | | | |
| | | | T447 T606 | | | |
| | | | | | UBA/TS-N domain: K291-Y330 | HMMER_PFAM |
| | | | | | Protein kinase domain: Y20-M271 | HMMER_PFAM |
| | | | | | Protein kinases signatures and profile: Y93-T168 | PROFILESCAN |
| | | | | | Tyrosine kinase catalytic domain signature PR00109: | BLIMPS_PRINTS |
| | | | | | M96-V109, Y132-L150, V198-Q220 | |
| | | | | | KINASE SERINE/THREONINE PROTEIN | BLAST_PRODOM |
| | | | | | PROTEIN TRANSFERASE ATP BINDING | |
| | | | | | SERINE/THREONINE PUTATIVE KINI EMK | |
| | | | | | PAR1 PD004300: G617-L731 | |
| | | | | | KINASE SERINE/THREONINE PROTEIN | BLAST_PRODOM |
| | | | | | SERINE/THREONINE PUTATIVE | |
| | | | | | TRANSFERASE ATP BINDING PROTEIN EMK | |
| | | | | | P78 CDC25C PD008571: R524-R612, S365-S543 | |
| | | | | | KINASE SERINE/THREONINE PROTEIN | BLAST_PRODOM |
| | | | | | PUTATIVE SERINE/THREONINE | |
| | | | | | TRANSFERASE ATP BINDING PROTEIN PAR1 | |
| | | | | | KP78 EMK PD005838: M271-R373 | |
| | | | | | KINASE SERINE/THREONINEPROTEIN | BLAST_PRODOM |
| | | | | | PUTATIVE EMK TRANSFERASE ATPBINDING | |
| | | | | | SERINE/THREONINE PROTEIN PD155890: R493-P523 | |
| | | | | | PROTEIN KINASE DOMAIN DM00004 | BLAST_DOMO |
| | | | | | \|I48609\|55-294: L22-L262 | |
| | | | | | \|Q05512\|55-294: L22-L262 | |
| | | | | | \|P27448\|58-297: L22-L262 | |
| | | | | | \|JC1446\|20-261: R21-L262 | |
| | | | | | Protein kinases ATP-binding region signature: I26-K49 | MOTIFS |
| | | | | | Serine/Threonine protein kinases active-site signature: | MOTIFS |
| | | | | | I138-L150 | |
| 28 | 7505729CD1 | 3267 | S73 S211 S313 | N2562 N2734 | Fibronectin type III domain: P1282-S1371, P2678-S2760 | HMMER_PFAM |
| | | | S381 S390 S413 | N2761 N2986 | | |
| | | | S441 S453 S462 | N3186 | | |
| | | | S476 S488 S506 | | | |
| | | | S516 S526 S559 | | | |
| | | | S706 S713 S826 | | | |
| | | | S832 S861 S874 | | | |
| | | | S879 S889 S1030 | | | |
| | | | S1053 S1128 S1164 | | | |
| | | | S1165 S1172 S1186 | | | |
| | | | S1230 S1367 S1424 | | | |
| | | | S1440 S1455 S1489 | | | |
| | | | S1567 S1598 S1623 | | | |
| | | | S1641 S1873 S1883 | | | |
| | | | S1920 S1922 S1924 | | | |
| | | | S1949 | | | |
| | | | S2004 S2014 S2132 | | Immunoglobulin domain: G982-L1043, G883-A944, | HMMER_PFAM |
| | | | S2322 S2355 S2393 | | G736-A796, G2598-A2659, G1499-A1559, G57-A110, | |
| | | | S2410 S2414 S2444 | | A1202-Y1258, G1078-Y1134, L1449-A1466 | |
| | | | S2458 S2465 S2473 | | | |
| | | | S2481 S2496 S2500 | | | |
| | | | S2566 S2578 S2623 | | | |
| | | | S2634 S2763 S2834 | | | |
| | | | S2945 S2949 S3024 | | | |
| | | | S3052 S3058 S3140 | | | |
| | | | S3156 S3208 T7 | | | |
| | | | T124 T133 T144 | | | |
| | | | T168 T180 T193 | | | |
| | | | T205 T348 T349 | | | |
| | | | T505 T541 T544 | | | |
| | | | T700 T810 T825 | | | |
| | | | T849 | | | |
| | | | T870 T941 T995 | | Protein kinase domain: Y2966-L3218, Y1601-F1854 | HMMER_PFAM |
| | | | T1040 T1045 | | | |
| | | | T1057 T1237 | | | |
| | | | T1295 T1363 | | | |
| | | | T1385 T1465 | | | |
| | | | T1501 T1579 | | | |
| | | | T1690 T1749 | | | |
| | | | T1844 T1955 | | | |
| | | | T1969 T2188 | | | |
| | | | T2198 T2380 | | | |
| | | | T2495 T2689 | | | |
| | | | T2743 T2788 | | | |
| | | | T2868 T2956 | | | |
| | | | T2967 T2988 | | | |
| | | | T3031 T3230 | | | |
| | | | T3235 Y792 Y1519 | | | |
| | | | Y1659 Y1709 | | | |
| | | | | | PROTEIN CALPHOTIN CALCIUM BINDING CYT | BLAST_PRODOM |
| | | | | | ADHERENCE HIGH MOLECULAR WEIGHT | |
| | | | | | ACCESSORY STRUCTURAL FILAMENTOUS | |
| | | | | | PD016116: V2777-P2952 | |
| | | | | | PROTEIN SF16 ISOLOG MATRIX SLP76 | BLAST_PRODOM |
| | | | | | TYROSINE PHOSPHOPROTEIN WALL SER/ARG | |
| | | | | | RELATED NUCLEAR PD033173: I2151-A2234 | |
| | | | | | PROTEIN REPEAT MICROTUBULE | BLAST_PRODOM |
| | | | | | ASSOCIATED MICROTUBULES | |
| | | | | | PHOSPHORYLATION BASSOON ALTERNATIVE | |
| | | | | | SPLICING LARGE PROLINE-RICH PD005493: | |
| | | | | | T2771-P2961, P2186-R2315 | |
| | | | | | PROTEIN KINASE DOMAIN DM00004 | BLAST_DOMO |
| | | | | | \|S07571\|5152-5396: D1602-D1839, E2970-L3209 | |
| | | | | | \|P53355\|15-257: Q1605-D1839, E2970-L3209 | |
| | | | | | \|JN0583\|727-969: I1603-D1839, L2969-L3199 | |
| | | | | | \|P07313\|298-541: Q1605-R1840, G2975-S3208 | |
| | | | | | \|P07313\|298-541: Q1605-R1840, G2975-S3208 | |
| | | | | | Cell attachment sequence: R934-D936 | MOTIFS |
| | | | | | Protein kinases ATP-binding region signature: I1607-K1630 | MOTIFS |
| | | | | | Serine/Threonine protein kinases active-site signature: | MOTIFS |
| | | | | | V1715-V1727, V3081-L3093 | |
| 29 | 7487334CD1 | 492 | S56 S68 S98 S110 | N43 | Signal Peptide: M1-S30 | HMMER |
| | | | S128 S136 S174 | | | |
| | | | S268 S338 S367 | | | |
| | | | T14 T97 T212 | | | |
| | | | T331 T471 | | | |
| | | | | | EPIDERMAL GROWTH FACTOR RECEPTOR | BLAST_PRODOM |
| | | | | | KINASE SUBSTRATE EPS8 SH3 DOMAIN | |
| | | | | | PHOSPHORYLATION PD011987: R264-G394, | |
| | | | | | R48-Q222 | |
| 30 | 7503109CD1 | 967 | S77 S143 S199 | N274 | Protein kinase domain: V425-L679 | HMMER_PFAM |
| | | | S240 S307 S332 | | | |
| | | | S337 S375 S520 | | | |
| | | | S667 S678 S746 | | | |
| | | | S747 S881 S892 | | | |
| | | | S893 S925 T15 | | | |
| | | | T110 T264 T290 | | | |
| | | | T409 T458 T603 | | | |
| | | | T604 T701 T874 | | | |
| | | | T895 T900 Y579 | | | |
| | | | Y792 Y839 | | | |
| | | | | | Receptor tyrosine kinase class II proteins BL00239: | BLIMPS_BLOCKS |
| | | | | | G432-E441, E474-L521, L526-R548, A551-D576, | |
| | | | | | E577-F626, N631-L675 | |
| | | | | | Receptor tyrosine kinase class III proteins BL00240: | BLIMPS_BLOCKS |
| | | | | | Q417-L465, T525-C562, D576-K623, K623-L675 | |
| | | | | | Receptor tyrosine kinase class V proteins BL00790: | BLIMPS_BLOCKS |
| | | | | | H447-I500, S530-A551, V552-D578, V584-W616, | |
| | | | | | E617-G641, D642-A690 | |
| | | | | | Protein kinases signatures and profile: L526-E577 | PROFILESCAN |
| | | | | | Receptor tyrosine kinase class II signature: R553-I597 | PROFILESCAN |
| | | | | | Tyrosine kinase catalytic domain signature PR00109: | BLIMPS_PRINTS |
| | | | | | M502-E515, Y539-V557, L587-I597, S606-W628, | |
| | | | | | C650-F672 | |
| | | | | | FOCAL ADHESION KINASE FADK TYROSINE | BLAST_PRODOM |
| | | | | | PROTEIN TRANSFERASE ATP BINDING | |
| | | | | | PHOSPHORYLATION PP125FAK TYROSINE | |
| | | | | | PD007810: R39-V425 | |
| | | | | | KINASE FOCAL ADHESION TYROSINE | BLAST_PRODOM |
| | | | | | PROTEIN TRANSFERASE FADK ATP BINDING | |
| | | | | | PHOSPHORYLATION PP125FAK TYROSINE | |
| | | | | | PD006413: Q736-E967 | |
| | | | | | FOCAL ADHESION KINASE FADK TYROSINE | BLAST_PRODOM |
| | | | | | CELL BETA CAK TYROSINE PROTEIN | |
| | | | | | TRANSFERASE PD155872: Y683-S747 | |
| | | | | | KINASE PROTEIN TRANSFERASE ATP | BLAST_PRODOM |
| | | | | | BINDING SERINE/THREONINE PROTEIN | |
| | | | | | PHOSPHORYLATION RECEPTOR TYROSINE | |
| | | | | | PROTEIN PRECURSOR TRANSMEMBRANE | |
| | | | | | PD000001: Y579-F627, V426-I492, V637-V676, | |
| | | | | | I500-I574 | |
| | | | | | do KINASE; TYROSINE; ADHESION; ATP; | BLAST_DOMO |
| | | | | | DM05081 | |
| | | | | | \|S60248\|29-424: V29-V425 | |
| | | | | | \|A57434\|29-424: V29-V425 | |
| | | | | | PROTEIN KINASE DOMAIN DM00004 | BLAST_DOMO |
| | | | | | \|S60248\|426-671: V426-F672 | |
| | | | | | \|A57434\|426-671: V426-F672 | |
| | | | | | Protein kinases ATP-binding region signature: L431-K457 | MOTIFS |
| | | | | | Tyrosine protein kinases specific active-site signature: | MOTIFS |
| | | | | | C545-V557 | |
| 31 | 7503128CD1 | 316 | S11 S15 S110 S253 | | Protein kinase domain: F44-V276 | HMMER_PFAM |
| | | | S291 S304 T89 | | | |
| | | | | | Protein kinase C terminal domain: Q275-D295 | HMMER_PFAM |
| | | | | | Protein kinases signatures and profile: H143-R195 | PROFILESCAN |
| | | | | | Tyrosine kinase catalytic domain signature PR00109: | BLIMPS_PRINTS |
| | | | | | M121-R134, Y157-I175, G201-I211, V220-D242, | |
| | | | | | F262-F284 | |
| | | | | | KINASE PROTEIN SUBUNIT CAMP- | BLAST_PRODOM |
| | | | | | DEPENDENT TRANSFERASE PKA | |
| | | | | | SERINE/THREONINE PROTEIN ATP BINDING | |
| | | | | | CAMP PHOSPHORYLATION PD004000: G2-Q43 | |
| | | | | | KINASE PROTEIN TRANSFERASE ATP | BLAST_PRODOM |
| | | | | | BINDING SERINE/THREONINE PROTEIN | |
| | | | | | PHOSPHORYLATION RECEPTOR TYROSINE | |
| | | | | | PROTEIN PRECURSOR TRANSMEMBRANE | |
| | | | | | PD000001: T196-F240, Q43-V124, M119-V192 | |
| | | | | | PROTEIN KINASE DOMAIN DM00004 | BLAST_DOMO |
| | | | | | \|P00517\|44-281: E45-E277 | |
| | | | | | \|B35755\|53-290: E45-E277 | |
| | | | | | \|S19028\|46-283: R46-E277 | |
| | | | | | \|S41099\|118-355: K48-E277 | |
| | | | | | Protein kinases ATP-binding region signature: L50-K73 | MOTIFS |
| | | | | | Serine/Threonine protein kinases active-site signature: | MOTIFS |
| | | | | | L163-I175 | |
| 32 | 7503191CD1 | 510 | S34 S58 S102 S180 | N100 N361 N427 | Caspase recruitment domain: A406-L494 | HMMER_PFAM |
| | | | S183 S207 S224 | N507 | | |
| | | | S267 S344 S371 | | | |
| | | | S398 S412 S448 | | | |
| | | | T296 T301 T330 | | | |
| | | | T454 Y23 | | | |
| | | | | | Protein kinase domain: L18-L287 | HMMER_PFAM |
| | | | | | Protein kinases signatures and profile: F122-K169 | PROFILESCAN |
| | | | | | Tyrosine kinase catalytic domain signature PR00109: | BLIMPS_PRINTS |
| | | | | | H261-F283, T95-H108, H136-L154, G188-Y198, | |
| | | | | | H210-V232 | |
| | | | | | SERINE/THREONINE KINASE RICK (a novel | BLAST_PRODOM |
| | | | | | protein kinase containing a caspase recruitment | |
| | | | | | domain, interacts with CLARP and regulates CD95- | |
| | | | | | mediated apoptosis) PD119437: K313-M510 | |
| | | | | | PROTEIN KINASE DOMAIN DM00004 | BLAST_DOMO |
| | | | | | \|Q05609\|553-797: S29-F283 | |
| | | | | | \|I49299\|19-278: L24-F283 | |
| | | | | | \|S29851\|157-404: A28-F283 | |
| | | | | | \|Q07292\|483-735: S29-F283 | |
| | | | | | Serine/Threonine protein kinases active-site signature: | MOTIFS |
| | | | | | L142-L154 | |
| 33 | 7503196CD1 | 909 | S254 S320 S359 | N490 N552 N649 | Guanylate kinase: T758-S862 | HMMER_PFAM |
| | | | S370 S576 S582 | | | |
| | | | S583 S633 S784 | | | |
| | | | S793 T272 T388 | | | |
| | | | T510 T570 T673 | | | |
| | | | T710 T724 T757 | | | |
| | | | T801 T805 T885 | | | |
| | | | Y137 Y290 Y752 | | | |
| | | | | | L27 domain: A411-R464, A352-P407 | HMMER_PFAM |
| | | | | | PDZ domain (Also known as DHR or GLGF).: L496-S576 | HMMER_PFAM |
| | | | | | SH3 domain: I598-W663 | HMMER_PFAM |
| | | | | | Protein kinase domain: Y12-L282 | HMMER_PFAM |
| | | | | | Tyrosine kinase catalytic domain signature PR00109: | BLIMPS_PRINTS |
| | | | | | I251-V273, Y137-L155, V206-T228 | |
| | | | | | PDZ domain proteins (Also known as DHR or GLGF) | BLIMPS_PFAM |
| | | | | | PF00595: L536-N546 | |
| | | | | | PROTEIN SH3 DOMAIN REPEAT PD00289: G539-N552 | BLIMPS_PRODOM |
| | | | | | PROTEIN DOMAIN MEMBRANE SH3 | BLAST_PRODOM |
| | | | | | CALMODULIN BINDING PERIPHERAL PLASMA | |
| | | | | | ERYTHROCYTE P55 CASK PD004835: P658-T757 | |
| | | | | | PROTEIN SH3 DOMAIN PERIPHERAL PLASMA | BLAST_PRODOM |
| | | | | | MEMBRANE CALMODULIN BINDING CASK | |
| | | | | | CAMGUK CALCIUM PD008238: K372-A482 | |
| | | | | | PERIPHERAL PLASMA MEMBRANE PROTEIN | BLAST_PRODOM |
| | | | | | CASK SH3 DOMAIN CALMODULIN BINDING | |
| | | | | | 3D STRUCTURE ALTERNATIVE PD012937: | |
| | | | | | A316-E371 | |
| | | | | | CALCIUM/CALMODULIN DEPENDENT | BLAST_PRODOM |
| | | | | | PROTEIN KINASE PD083070: P469-V538 | |
| | | | | | GUANYLATE KINASE DM00755 | BLAST_DOMO |
| | | | | | \|P54936\|769-955: L717-P904 | |
| | | | | | \|Q00013\|277-460: L717-P904 | |
| | | | | | \|P49697\|278-461: L717-P904 | |
| | | | | | \|A57627\|279-461: P718-P904 | |
| | | | | | ATP/GTP-binding site motif A (P-loop): G53-T60 | MOTIFS |
| | | | | | Guanylate kinase signature: T757-V774 | MOTIFS |
| 34 | 7503254CD1 | 731 | S61 S89 S96 S233 | N97 N159 N303 | SAM domain (Sterile alpha motif): G268-T339 | HMMER_PFAM |
| | | | S288 S333 S355 | N516 N562 N577 | | |
| | | | S456 S494 S498 | | | |
| | | | S534 S599 S605 | | | |
| | | | S622 S628 S664 | | | |
| | | | S688 T271 T305 | | | |
| | | | T453 T486 T507 | | | |
| | | | T597 T700 T716 | | | |
| | | | | | Protein kinase domain: L16-G208 | HMMER_PFAM |
| | | | | | Protein kinases signatures and profile: I107-T162 | PROFILESCAN |
| | | | | | Tyrosine kinase catalytic domain signature PR00109: | BLIMPS_PRINTS |
| | | | | | T82-N95, M123-I141, G168-I178, C187-L209 | |
| | | | | | PROTEIN KINASE DOMAIN DM00004 | BLAST_DOMO |
| | | | | | \|Q05609\|553-797: E20-S233 | |
| | | | | | \|A53800\|119-368: E20-K221 | |
| | | | | | \|JC2363\|126-356: D15-W216 | |
| | | | | | \|A55318\|159-389: D15-W216 | |
| | | | | | Leucine zipper pattern: L225-L246, L232-L253 | MOTIFS |
| | | | | | Cell attachment sequence: R722-D724 | MOTIFS |
| | | | | | Serine/Threonine protein kinases active-site signature: | MOTIFS |
| | | | | | V129-I141 | |
| 35 | 7503531CD1 | 171 | S4 S22 S63 T120 | | FGGY family of carbohydrate kinases, N-terminal | HMMER_PFAM |
| | | | Y109 | | domain: L12-P171 | |
| | | | | | FGGY family of carbohydrate kinases proteins | BLIMPS_BLOCKS |
| | | | | | BL00933: L12-L35, F47-P57 | |
| | | | | | GLYCEROL KINASE ATP: GLYCEROL 3- | BLAST_PRODOM |
| | | | | | PHOSPHOTRANSFERASE GLYCEROKINASE GK | |
| | | | | | METABOLISM TRANSFERASE | |
| | | | | | POLYMORPHISM DISEASE PD014105: E106-P142 | |
| | | | | | XYLULOKINASE DM02388 | BLAST_DOMO |
| | | | | | \|P32189\|9-510: L9-V112, E106-M129 | |
| | | | | | \|P08859\|2-493: A15-R117, P107-M129 | |
| | | | | | \|I64086\|3-494: A15-R117, E115-M129 | |
| | | | | | \|P18157\|1-492: A15-T120 | |
| 36 | 7490021CD1 | 561 | S64 S224 S254 | N286 N456 N501 | Cytosolic domains: M1-I88, R149-D156 | TMHMMER |
| | | | S415 S429 S458 | | Transmembrane domains: V89-A111, Y126-E148, | |
| | | | T231 T243 T320 | | L157-F179 | |
| | | | T552 | | Non-cytosolic domains: D112-E125, D180-D561 | |
| | | | | | PROTEIN HYDROLASE PHOSPHATASE | BLAST_PRODOM |
| | | | | | MULTIPLE ADVANCED CANCERS | |
| | | | | | PHOSPHORYLATION TENSIN PROTEIN | |
| | | | | | TYROSINE PTEN PD007685: L218-C285 | |
| | | | | | PROTEIN PHOSPHORYLATION AUXILIN COAT | BLAST_PRODOM |
| | | | | | REPEAT KIAA0473 CYCLIN G ASSOCIATED | |
| | | | | | KINASE TRANSFERASE PD025411: E296-I427 | |
| 37 | 7503180CD1 | 246 | S164 T218 | | signal_cleavage: M42-T106 | SPSCAN |
| | | | | | PAP2 superfamily: P56-T218 | HMMER_PFAM |
| | | | | | Cytosolic domains: M1-A4, K79-A89, K152-K163, | TMHMMER |
| | | | | | R211-I246 | |
| | | | | | Transmembrane domains: A5-T27, P59-L78, C90-S112, | |
| | | | | | S132-G151, S164-A181, H191-Y210 | |
| | | | | | Non-cytosolic domains: E28-K58, D113-P131, L182-H190 | |
| | | | | | SIMILARITY TO NADH UBIQUINONE | BLAST_PRODOM |
| | | | | | OXIDOREDUCTASE CHAIN 4 UBIQUINONE | |
| | | | | | PD096518: L99-D228 | |
| 38 | 7503206CD1 | 518 | S38 S57 S195 S216 | N395 | Protein phosphatase 2C: S325-Q462, L25-D102 | HMMER_PFAM |
| | | | S218 S243 S245 | | | |
| | | | S260 S278 S285 | | | |
| | | | S448 S461 S474 | | | |
| | | | S489 S509 T10 | | | |
| | | | T106 T122 T152 | | | |
| | | | T177 T207 T428 | | | |
| | | | Y364 Y405 | | | |
| | | | | | Protein phosphatase 2C proteins BL01032: M55-G64, | BLIMPS_BLOCKS |
| | | | | | K89-T106, S327-I336, N345-V384, R389-D402, | |
| | | | | | H433-N445, M1-A20, Q33-H43 | |
| | | | | | PROTEIN PHOSPHATASE 2C GAMMA ISOFORM | BLAST_PRODOM |
| | | | | | PP2C GAMMA HYDROLASE MAGNESIUM | |
| | | | | | MANGANESE MULTIGENE PD035366: I111-F257 | |
| | | | | | PROTEIN PHOSPHATASE 2C MAGNESIUM | BLAST_PRODOM |
| | | | | | HYDROLASE MANGANESE MULTIGENE | |
| | | | | | FAMILY PP2C ISOFORM PD001101: G231-N506, | |
| | | | | | L25-K128 | |
| | | | | | PROTEIN PHOSPHATASE 2C GAMMA ISOFORM | BLAST_PRODOM |
| | | | | | PP2C GAMMA HYDROLASE MAGNESIUM | |
| | | | | | MANGANESE MULTIGENE PD035368: E478-D518 | |
| | | | | | PROTEIN PHOSPHATASE 2C DM00377 | BLAST_DOMO |
| | | | | | \|Q09172\|1-299: M317-R463, M1-E113 | |
| | | | | | \|Q09173\|1-296: S327-I460, M1-E109 | |
| | | | | | \|S62462\|1-297: S327-I460, M1-E109 | |
| | | | | | \|P49595\|224-490: S260-L469 | |
| | | | | | Protein phosphatase 2C signature: M55-G63 | MOTIFS |
| 39 | 7503227CD1 | 273 | S5 S164 S175 S264 | N216 | Ser/Thr protein phosphatase: V68-Q257, L7-R67 | HMMER_PFAM |
| | | | Y115 | | | |
| | | | | | Serine/threonine specific protein phosphatases | BLIMPS_BLOCKS |
| | | | | | proteins BL00125: P48-R84, H56-G101, A120-P166, | |
| | | | | | G180-N234 | |
| | | | | | Serine/threonine specific protein phosphatases | PROFILESCAN |
| | | | | | signature: Q58-S102 | |
| | | | | | Serine/threonine phosphatase family signature | BLIMPS_PRINTS |
| | | | | | PR00114: L76-Y100, E111-I137, L140-E167, D196-N216, | |
| | | | | | T218-N234 | |
| | | | | | PROTEIN PHOSPHATASE SERINE/THREONINE | BLAST_PRODOM |
| | | | | | HYDROLASE IRON MANGANESE SUBUNIT | |
| | | | | | MULTIGENE FAMILY CATALYTIC PD000252: | |
| | | | | | F66-Q257, D6-F66 | |
| | | | | | PHOSPHOPROTEIN PHOSPHATASE DM00133 | BLAST_DOMO |
| | | | | | \|P11084\|4-300: R67-P267, I4-F92 | |
| | | | | | \|S42558\|1-298: R67-P267, I4-V68 | |
| | | | | | \|Q07098\|4-300: R67-K266, D6-R84 | |
| | | | | | \|S52659\|11-307: R67-P263, D6-R84 | |
| | | | | | Serine/threonine specific protein phosphatases | MOTIFS |
| | | | | | signature: I77-E82 | |
| 40 | 7504473CD1 | 222 | S12 S26 S170 T14 | N209 N214 N217 | Ser/Thr protein phosphatase: V43-T86 | HMMER_PFAM |
| | | | T30 T123 T169 | | | |
| | | | | | SUBUNIT PROTEIN PHOSPHATASE | BLAST_PRODOM |
| | | | | | CALCINEURIN SERINE/THREONINE | |
| | | | | | HYDROLASE 2B CATALYTIC IRON | |
| | | | | | MANGANESE PD003520: T86-G215 | |
| | | | | | CALCINEURIN CATALYTIC CHAIN DM01653 | BLAST_DOMO |
| | | | | | \|P48452\|350-510: G87-D212 | |
| | | | | | \|A38193\|346-503: Q74-A190 | |
| | | | | | \|P48455\|346-503: Q74-A190 | |
| | | | | | \|S41743\|407-557: D82-S199 | |
| 41 | 7503200CD1 | 519 | S134 S145 S244 | | Protein kinase domain: T432-F509, E386-Q422, | HMMER_PFAM |
| | | | S295 S383 T45 | | K202-L229 | |
| | | | T307 T490 | | | |
| | | | | | Tyrosine kinase catalytic domain signature | BLIMPS_PRINTS |
| | | | | | PR00109: L217-L230, A402-L420, C435-H457, | |
| | | | | | L473-M495 | |
| | | | | | PROTEIN KINASE DOMAIN | BLAST_DOMO |
| | | | | | DM00004\|P54644\|122-362: D436-L493 | |
| | | | | | DM08046\|P06244\|1-396: E424-V518 | |
| | | | | | DM08046\|P05986\|1-397: E424-V518 | |
| | | | | | DM00004\|A57459\|61-302: T432-G494 | |
| 42 | 7500465CD1 | 77 | S71 T14 T62 | | CASEIN KINASE I, GAMMA I ISOFORM EC | BLAST_PRODOM |
| | | | | | 2.7.1. CKI GAMMA TRANSFERASE | |
| | | | | | SERINE/THREONINE PROTEIN ATP-BINDING | |
| | | | | | MULTIGENE FAMILY PHOSPHORYLATION | |
| | | | | | PD049080: M1-N43 | |
| 43 | 7503256CD1 | 540 | S47 S64 S84 S97 | N71 N493 | Protein kinase domain: Y29-W216 | HMMER_PFAM |
| | | | S267 S309 S349 | | | |
| | | | S385 S417 S495 | | | |
| | | | T211 T224 T235 | | | |
| | | | T433 T442 Y263 | | | |
| | | | | | Protein kinases signatures and profile: Q134-S185 | PROFILESCAN |
| | | | | | Tyrosine kinase catalytic domain signature | BLIMPS_PRINTS |
| | | | | | PR00109: T108-Q121, Y148-L166, G194-L204 | |
| | | | | | PROTEIN KINASE DOMAIN | BLAST_DOMO |
| | | | | | DM00004\|P51957\|8-251: L35-W216 | |
| | | | | | DM00004\|P51954\|6-248: Q33-W216 | |
| | | | | | DM00004\|P41892\|11-249: L35-W216 | |
| | | | | | DM00004\|P51955\|10-261: L35-R274 | |
| | | | | | Serine/Threonine protein kinases active-site signature: | MOTIFS |
| | | | | | I154-L166 | |
| 44 | 7503257CD1 | 609 | S47 S64 S84 S97 | N71 N562 | Protein kinase domain: Y29-L287 | HMMER_PFAM |
| | | | S251 S273 S277 | | | |
| | | | S372 S418 S454 | | | |
| | | | S486 S564 T211 | | | |
| | | | T302 T329 T340 | | | |
| | | | T502 T511 Y368 | | | |
| | | | | | Protein kinases signatures and profile: Q134-C186 | PROFILESCAN |
| | | | | | Tyrosine kinase catalytic domain signature PR00109: | BLIMPS_PRINTS |
| | | | | | T108-Q121, Y148-L166, G194-L204, S213-S235, | |
| | | | | | Y256-A278 | |
| | | | | | PROTEIN KINASE DOMAIN | BLAST_DOMO |
| | | | | | DM00004\|P51957\|8-251: L35-S277 | |
| | | | | | DM00004\|P51954\|6-248: Q33-S277 | |
| | | | | | DM00004\|P22209\|27-333: L170-S277, Q33-H150 | |
| | | | | | DM00004\|P51955\|10-261: L35-S277 | |
| | | | | | Serine/Threonine protein kinases active-site signature: | MOTIFS |
| | | | | | I154-L166 | |
| 45 | 7504472CD1 | 725 | S3 S89 S110 S166 | N27 N164 N270 | Hr1 repeat motif (REM repeat), a Protein kinase C- | HMMER_PFAM |
| | | | S324 S361 S372 | | related kinase homology region: D47-P119 | |
| | | | S455 S644 S685 | | | |
| | | | S708 T73 T74 | | | |
| | | | T305 T346 T677 | | | |
| | | | Y177 | | | |
| | | | | | Protein kinase domain: F398-F657 | HMMER_PFAM |
| | | | | | Protein kinase C terminal domain: R658-C725 | HMMER_PFAM |
| | | | | | Tyrosine kinase catalytic domain signature | BLIMPS_PRINTS |
| | | | | | PR00L09: M478-H491, Y513-L531, V579-D601, | |
| | | | | | L621-A643 | |
| | | | | | PRK2, Kinase C-related ATP-Binding protein-kinase | BLAST_PRODOM |
| | | | | | phosphorylation transferase serine/threonine-protein C | |
| | | | | | like PD083610: P280-Q392 | |
| | | | | | KINASE PROTEIN PKN F46F6.2 C LIKE | BLAST_PRODOM |
| | | | | | TRANSFERASE ATP-BINDING PRK2 | |
| | | | | | PHOSPHORYLATION C RELATED | |
| | | | | | PD014425: Q42-L172, G154-H201, E9-L103 | |
| | | | | | KINASE PROTEIN PKN C RELATED PRK-SD | BLAST_PRODOM |
| | | | | | SERINE/THREONINE SERINE/THREONINE | |
| | | | | | PROTEIN C LIKE TRANSFERASE ATP-BINDING | |
| | | | | | PD010847: L207-T346 | |
| | | | | | PRK2, Kinase C-related ATP-Binding protein-kinase | BLAST_PRODOM |
| | | | | | phosphorylation transferase serine/threonine-protein C | |
| | | | | | like PD142160: M1-V41 | |
| | | | | | PROTEIN KINASE DOMAIN | BLAST_DOMO |
| | | | | | DM00004\|JC2129\|616-858: A402-G642 | |
| | | | | | DM00004\|S48705\|153-395: A402-G642 | |
| | | | | | DM00004\|S53726\|576-817: A402-G642 | |
| | | | | | PROTEIN KINASE C ALPHA | BLAST_DOMO |
| | | | | | DM04692\|P05773\|1-672: R390-I721, C131-S167 | |
| | | | | | Leucine zipper pattern: L79-L100 | MOTIFS |
| | | | | | Protein kinases ATP-binding region signature | MOTIFS |
| | | | | | L404-K427 | |
| | | | | | Serine/Threonine protein kinases active-site signature: | MOTIFS |
| | | | | | I519-L531 | |
| 46 | 7504475CD1 | 498 | S11 S26 S30 S95 | | Protein kinase domain: Y165-I435 | HMMER_PFAM |
| | | | S100 S137 S197 | | | |
| | | | S358 S367 S452 | | | |
| | | | S460 T56 T145 | | | |
| | | | T166 T280 T362 | | | |
| | | | Y234 Y305 | | | |
| | | | | | Protein kinases signatures and profile: Q288-S340 | PROFILESCAN |
| | | | | | Tyrosine kinase catalytic domain signature | BLIMPS_PRINTS |
| | | | | | PR00109: | |
| | | | | | Y302-V320, G349-L359, L371-E393, I415-V437 | |
| | | | | | CA+/CALMODULIN-DEPENDENT PROTEIN | BLAST_PRODOM |
| | | | | | KINASE KINASE BETA CAM KINASE KINASE | |
| | | | | | BETA | |
| | | | | | PD174840: M1-E80 | |
| | | | | | KINASE PROTEIN BETA CA2+/CALMODULIN- | BLAST_PRODOM |
| | | | | | DEPENDENT CA+/CALMODULIN-DEPENDENT | |
| | | | | | CAM KINASE IV ISOFORM PHOSPHORYLASE B | |
| | | | | | PD031900: A69-Q164 | |
| | | | | | PROTEIN KINASE DOMAIN | BLAST_DOMO |
| | | | | | DM00004\|A57156\|130-399: L167-V437 | |
| | | | | | DM00004\|P50526\|136-399: E170-V437 | |
| | | | | | DM00004\|JC1446\|20-261: E231-V437 | |
| | | | | | DM00004\|P38990\|135-438: E170-E357, S334-V437 | |
| | | | | | Protein kinases ATP-binding region signature: | MOTIFS |
| | | | | | I171-K194 | |
| | | | | | Serine/Threonine protein kinases active-site signature: | MOTIFS |
| | | | | | I308-V320 | |
| 47 | 7503104CD1 | 142 | Y37 Y50 | N24 | PHOSPHATASE PROTEIN TYROSINE PTP | BLAST_PRODOM |
| | | | | | CAAX1 NUCLEAR 4A2 MPRL2 PROTEIN | |
| | | | | | TYROSINE CLONE HH72 | |
| | | | | | PD007217: M1-E33 | |
| | | | | | PROTEIN TYROSINE PHOSPHATASE PD166489: | BLAST_PRODOM |
| | | | | | L34-L63 | |
| | | | | | PHOSPHATASE PROTEIN TYROSINE 4A2 | BLAST_PRODOM |
| | | | | | MPRL2 PROTEIN TYROSINE CLONE HH72 | |
| | | | | | PD008124: S115-Q142 | |
| 48 | 7503106CD1 | 206 | S85 S162 S177 | | Serine/Threonine protein phosphatase: E17-K179 | HMMER_PFAM |
| | | | S190 T29 | | | |
| | | | | | Serine/threonine specific protein phosphatases | BLIMPS_BLOCKS |
| | | | | | proteins BL00125: A41-P87, S102-N156 | |
| | | | | | Serine/threonine phosphatase family signature | BLIMPS_PRINTS |
| | | | | | PR00114: M61-D88, D118-K138, Q140-N156, D32-L58 | |
| | | | | | PROTEIN PHOSPHATASE SERINE/THREONINE | BLAST_PRODOM |
| | | | | | HYDROLASE IRON MANGANESE SUBUNIT | |
| | | | | | MULTIGENE FAMILY CATALYTIC | |
| | | | | | PD000252: E17-K180 | |
| | | | | | F58G1.3 PROTEIN SERINE/THREONINE | BLAST_PRODOM |
| | | | | | SPECIFIC PROTEIN PHOSPHATASE | |
| | | | | | PD000297: V101-P176 | |
| | | | | | SERINE/THREONINE PROTEIN PHOSPHATASE | BLAST_PRODOM |
| | | | | | PP1 BETA CATALYTIC SUBUNIT EC 3.1.3.16 | |
| | | | | | PP1B HYDROLASE GLYCOGEN METABOLISM | |
| | | | | | MULTIGENE FAMILY CELL DIVISION | |
| | | | | | PD160488: K180-R206 | |
| | | | | | PHOSPHOPROTEIN PHOSPHATASE | BLAST_DOMO |
| | | | | | DM00133\|P48462\|14-309: E17-N189 | |
| | | | | | DM00133\|S13828\|14-309: E17-N189 | |
| | | | | | DM00133\|P08128\|1-291: E17-N189 | |
| | | | | | DM00133\|P36873\|15-310: E17-T195 | |
| 49 | 7503176CD1 | 274 | S28 S56 S70 S97 | N125 | Signal Peptide: M43-A60 | HMMER |
| | | | S252 T65 T258 | | | |
| | | | | | PAP2 superfamily: K83-K234 | HMMER_PFAM |
| | | | | | Cytosolic domains: | TMHMMER |
| | | | | | T65-N76, R172-P183, K234-S274 | |
| | | | | | Transmembrane domains: | |
| | | | | | L42-Y64, Y77-T99, S149-A171, T184-V201, V211-F233 | |
| | | | | | Non-cytosolic domains: | |
| | | | | | M1-G41, D100-L148, S202-D210 | |
| | | | | | PHOSPHATIDIC ACID PHOSPHATASE | BLAST_PRODOM |
| | | | | | HYDROLASE PROTEIN PHOSPHOHYDROLASE | |
| | | | | | TRANSMEMBRANE PHOSPHATIDATE 2A TYPE 2 | |
| | | | | | PD005298: L6-G138 | |
| | | | | | PHOSPHATIDIC ACID PHOSPHOHYDROLASE | BLAST_PRODOM |
| | | | | | TYPE 2C HYDROLASE | |
| | | | | | PD096504: V226-S274 | |
| | | | | | PROTEIN TRANSMEMBRANE PHOSPHATIDIC | BLAST_PRODOM |
| | | | | | ACID PHOSPHATASE HYDROLASE | |
| | | | | | MEMBRANE TRANSPORT PERMEASE | |
| | | | | | INTEGRAL | |
| | | | | | PD002093: N139-V226 | |
| 50 | 7503202CD1 | 515 | S180 S382 S469 | N511 | Serine/Threonine protein phosphatase: V52-H348 | HMMER_PFAM |
| | | | T35 T39 T86 T170 | | | |
| | | | T217 T261 T388 | | | |
| | | | T432 Y184 | | | |
| | | | | | Serine/threonine specific protein phosphatases | BLIMPS_BLOCKS |
| | | | | | proteins BL00125: | |
| | | | | | S135-S180, A198-P244, S266-Y320, P93-V129 | |
| | | | | | Serine/threonine specific protein phosphatases | PROFILESCAN |
| | | | | | signature: I136-E181 | |
| | | | | | Serine/threonine phosphatase family signature | BLIMPS_PRINTS |
| | | | | | PR00114: P93-T120, Y122-Y149, L155-Y179, E189-I215 | |
| | | | | | L218-S245, N282-K302, S310-N326 | |
| | | | | | PROTEIN PHOSPHATASE SERINE/THREONINE | BLAST_PRODOM |
| | | | | | HYDROLASE IRON MANGANESE SUBUNIT | |
| | | | | | MULTIGENE FAMILY CATALYTIC PD000252: | |
| | | | | | L59-P347 | |
| | | | | | SUBUNIT PROTEIN PHOSPHATASE | BLAST_PRODOM |
| | | | | | CALCINEURIN SERINE/THREONINE | |
| | | | | | HYDROLASE 2B CATALYTIC IRON | |
| | | | | | MANGANESE | |
| | | | | | PD003520: A401-N511, H348-Q393 | |
| | | | | | SIMILAR TO SERINE/THREONINE PROTEIN | BLAST_PRODOM |
| | | | | | PHOSPHATASE PD112269: H58-P150 | |
| | | | | | PHOSPHOPROTEIN PHOSPHATASE | BLAST_DOMO |
| | | | | | DM00133\|P48452\|39-348: G48-V358 | |
| | | | | | DM00133\|P48455\|36-344: P50-V358 | |
| | | | | | DM00133\|A38193\|36-344: P50-V358 | |
| | | | | | DM00133\|P48456\|34-343: G48-V358 | |
| | | | | | Serine/threonine specific protein phosphatases | MOTIFS |
| | | | | | signature: L156-E161 | |
| 51 | 7503249CD1 | 317 | S35 S166 S170 | N249 | Dual specificity phosphatase. catalytic domain: | HMMER_PFAM |
| | | | S177 S220 S251 | | F193-T282 | |
| | | | S264 S287 T137 | | | |
| | | | T179 T210 | | | |
| | | | | | Rhodanese-like domain: S5-E130 | HMMER_PFAM |
| | | | | | Rhodanese proteins BL00380: L24-S35, S35-I45, | BLIMPS_BLOCKS |
| | | | | | A80-W92 | |
| | | | | | PHOSPHATASE DUAL SPECIFICITY PROTEIN | BLAST_PRODOM |
| | | | | | HYDROLASE KINASE MAP PYST1 MITOGEN | |
| | | | | | ACTIVATED MKP3 | |
| | | | | | PD016181: G122-F193 | |
| | | | | | PHOSPHATASE DUAL SPECIFICITY PROTEIN | BLAST_PRODOM |
| | | | | | HYDROLASE KINASE MAP PYST1 DUSP6 ALT | |
| | | | | | MITOGEN ACTIVATED | |
| | | | | | PD021468: G47-Q121 | |
| | | | | | DUAL SPECIFICITY PROTEIN PHOSPHATASE 9 | BLAST_PRODOM |
| | | | | | EC 3.1.3.48 3.1.3.16 MITOGEN ACTIVATED | |
| | | | | | KINASE 4 MAP MKP4 HYDROLASE | |
| | | | | | PD086355: S101-P187 | |
| | | | | | VH1-TYPE DUAL SPECIFICITY PHOSPHATASE | BLAST_DOMO |
| | | | | | DM03823\|I38890\|29-320: | |
| | | | | | L212-E280, R27-C135, E169-L215 | |
| | | | | | DM03823\|A56115\|51-336: | |
| | | | | | L212-E280, R27-G157, L174-D216 | |
| | | | | | DM03823\|P28562\|169-314: L212-E280, P194-D216 | |
| | | | | | DM03823\|Q02256\|1-174: R219-G284 | |
| 52 | 7505890CD1 | 318 | S269 S274 S287 | N140 | Signal Peptide: M1-A32 | HMMER |
| | | | S309 | | | |
| | | | | | PAP2 superfamily: R103-Q264 | HMMER_PFAM |
| | | | | | Cytosolic domains: | TMHMMER |
| | | | | | M1-R103, S208-P213, Q264-T318 | |
| | | | | | Transmembrane domains: | |
| | | | | | F104-T126, A185-G207, S214-V231, V241-F263 | |
| | | | | | Non-cytosolic domains: G127-D184, A232-D240 | |
| | | | | | T06D8.3 PROTEIN PA-PHOSPHATASE RELATED | BLAST_PRODOM |
| | | | | | PHOSPHOESTERASE | |
| | | | | | PD137487: F24-K291 | |

TABLE 4

| Polynucleotide | |
|---|---|
| SEQ ID NO:/ | |
| Incyte ID/Sequence | |
| Length | Sequence Fragments |
| 53/7499969CB1/ | 1-433, 1-501, 1-502, 1-570, 1-599, 1-609, 1-610, 1-615, 1-632, 1-638, 1-648, 1-656, 1-684, 5-530, 14-175, 14-265, |
| 1928 | 15-266, 16-287, 18-261, 19-258, 19-263, 19-288, 19-360, 19-398, 19-451, 19-493, 19-547, 19-574, 20-1676, 23-274, |
| | 23-308, 35-323, 46-292, 47-324, 54-339, 56-328, 56-534, 56-586, 56-697, 56-884, 56-926, 72-353, 84-583, 106-390, |
| | 106-719, 106-734, 116-385, 128-486, 155-380, 155-744, 208-679, 218-716, 323-602, 384-671, 439-650, 439-709, |
| | 495-735, 497-663, 635-901, 635-1090, 635-1120, 635-1137, 635-1139, 635-1170, 635-1197, 635-1210, 635-1231, |
| | 635-1304, 645-1282, 745-1010, 745-1180, 761-1204, 800-1375, 826-1448, 830-1360, 835-1375, 859-1379, 899-1312, |
| | 903-1160, 915-1217, 916-1552, 922-1350, 932-1507, 935-1141, 942-1221, 971-1122, 996-1269, 998-1302, |
| | 1004-1479, 1004-1641, 1015-1308, 1018-1928, 1019-1623, 1021-1604, 1023-1259, 1038-1587, 1043-1594, 1046-1327, |
| | 1049-1615, 1056-1257, 1063-1681, 1064-1482, 1064-1670, 1072-1636, 1088-1590, 1091-1546, 1092-1604, |
| | 1093-1403, 1095-1429, 1102-1585, 1119-1385, 1130-1402, 1133-1435, 1141-1336, 1141-1368, |
| | 1141-1655, 1160-1354, 1165-1630, 1172-1362, 1176-1450, 1181-1396, 1181-1689, 1182-1458, 1192-1524, 1202-1685, |
| | 1233-1434, 1263-1642, 1266-1571, 1281-1359, 1322-1589, 1327-1668, 1392-1667, 1400-1680, 1427-1583, |
| | 1462-1676 |
| 54/7499974CB1/ | 1-647, 4-468, 9-519, 70-647, 110-468, 113-468, 155-468, 157-656, 157-663, 161-663, 166-482, 184-665, 469-1226, |
| 7152 | 469-1239, 469-1264, 469-1284, 469-1297, 492-663, 536-1391, 568-1235, 570-1052, 570-1056, 570-1176, 570-1235, |
| | 571-1056, 571-1059, 571-1061, 571-1107, 571-1116, 571-1117, 571-1129, 571-1147, 571-1151, 571-1154, 571-1223, |
| | 573-1039, 577-1172, 601-666, 601-812, 601-886, 601-891, 601-907, 601-982, 602-979, 634-1276, 683-1173, |
| | 684-937, 684-938, 698-1405, 699-1217, 765-1012, 777-1146, 810-1329, 816-1248, 821-1543, 821-1621, 860-1212, |
| | 870-1145, 873-1104, 874-1466, 875-1405, 909-1145, 915-1499, 921-1303, 936-1228, 949-1150, 954-1146, 975-1566, |
| | 981-1718, 981-1719, 1033-1607, 1131-1516, 1146-1619, 1161-1898, 1166-1891, 1234-1979, 1234-2099, 1256-2230, |
| | 1280-1892, 1288-1862, 1302-2308, 1313-2081, 1322-1516, 1370-2310, 1373-2209, 1381-1708, 1381-1711, |
| | 1406-1893, 1423-2099, 1423-2108, 1438-2102, 1446-1855, 1490-1855, 1563-1894, 1672-2088, 1672-2092, 1672-2095, |
| | 1672-2098, 1680-1999, 1681-1899, 1681-2068, 1681-2108, 1682-2108, 1710-2108, 1715-2053, |
| | 1743-2080, 1752-2067, 1767-2108, 1768-2278, 1810-2383, 1847-2253, 1879-2180, 1880-2164, 1901-2139, 2023-2646, |
| | 2023-2706, 2035-2108, 2132-2270, 2178-2709, 2276-2715, 2379-3000, 2474-3034, 2646-3249, 2658-3305, |
| | 2659-3299, 2665-3235, 2675-3310, 2716-3306, 2744-3331, 2771-3451, 2786-3396, 2896-3528, 2926-3557, 2962-3168, |
| | 2971-3307, 2986-3599, 3004-3579, 3053-3660, 3063-3627, 3072-3572, 3097-3696, 3100-3659, 3107-3724, |
| | 3127-3684, 3177-3648, 3208-3821, 3209-3772, 3213-3686, 3219-3819, 3229-3782, 3230-3753, 3277-3797, 3277-3935, |
| | 3311-3903, 3311-3946, 3312-3754, 3382-4033, 3384-4048, 3393-3958, 3394-3975, 3398-3944, 3419-3903, |
| | 3428-4081, 3456-4062, 3482-3816, 3500-4141, 3502-4113, 3524-3844, 3524-4129, 3530-4182, 3550-4223, 3569-4195, |
| | 3583-4139, 3584-4148, 3597-4258, 3616-4094, 3642-4184, 3667-4307, 3680-4095, 3680-4307, 3687-4282, |
| | 3719-4297, 3726-4300, 3807-4401, 3808-4404, 3822-4404, 3822-4423, 3826-4041, 3836-4404, 3838-4404, 3840-4064, |
| | 3848-4405, 3856-4404, 3857-4404, 3867-4404, 3867-4424, 3869-4432, 3875-4400, 3875-4404, 3877-4406, |
| | 3885-4404, 3897-4366, 3899-4404, 3915-4404, 3936-4404, 3938-4404, 3955-4404, 3972-4404, 3998-4609, |
| | 4037-4286, 4126-4704, 4140-4655, 4185-4799, 4235-4424, 4248-4494, 4248-4512, 4258-4404, 4262-4908, 4285-4781, |
| | 4347-4593, 4450-4738, 4450-4886, 4489-5285, 4489-5301, 4489-5302, 4489-5375, 4489-5405, 4492-5256, |
| | 4529-5320, 4737-5327, 4780-5362, 4847-5638, 4867-5383, 4870-5430, 4871-5500, 4878-5474, 4885-5233, 4894-5530, |
| | 4899-5638, 4902-5638, 4908-5221, 4908-5502, 4918-5638, 4921-5601, 4955-5601, 4998-5477, 4999-5463, |
| | 5043-5448, 5048-5682, 5077-5667, 5089-5652, 5096-5271, 5113-5648, 5144-5638, 5144-5715, 5187-5435, 5195-5866, |
| | 5198-5461, 5204-6015, 5271-5794, 5272-5847, 5293-5547, 5294-5910, 5302-5914, 5308-5974, 5316-5868, |
| | 5379-5665, 5419-5903, 5429-6075, 5435-6089, 5451-6033, 5466-5855, 5471-5948, 5474-5931, 5490-5733, 5494-5745, |
| | 5506-6057, 5512-5989, 5524-5782, 5552-6091, 5555-6043, 5556-6283, 5556-6440, 5570-6212, 5587-5818, |
| | 5600-5882, 5627-6238, 5678-6222, 5714-6252, 5717-6363, 5720-6013, 5724-5989, 5732-5979, 5756-6015, 5756-6207, |
| | 5766-6029, 5768-5972, 5793-6409, 5798-6009, 5813-6083, 5824-6389, 5846-6346, 5859-6132, 5860-6376, |
| | 5860-6566, 5864-6090, 5872-6119, 5872-6427, 5877-6152, 5882-6524, 5909-6187, 5914-6068, 5925-6072, |
| | 5974-6079, 5994-6260, 5994-6265, 6007-6269, 6028-6291, 6032-6645, 6057-6457, 6059-6661, 6079-6652, 6086-6346, |
| | 6091-6524, 6112-6688, 6118-6412, 6119-6462, 6127-6728, 6129-6796, 6138-6632, 6147-6384, 6149-6736, |
| | 6154-6443, 6169-6419, 6169-6462, 6169-6723, 6169-6733, 6169-6801, 6169-6808, 6192-6579, 6192-6702, 6194-6413, |
| | 6194-6452, 6213-6798, 6215-6741, 6215-6760, 6215-6917, 6232-6842, 6234-6828, 6261-6530, 6289-6515, |
| | 6292-6570, 6303-6595, 6303-6823, 6315-6796, 6316-6649, 6319-6593, 6325-6656, 6329-7135, 6357-6646, 6360-6636, |
| | 6376-6637, 6376-6658, 6376-6661, 6376-6667, 6379-6670, 6387-6670, 6401-7043, 6410-6517, 6424-6588, |
| | 6434-6703, 6450-6909, 6457-6713, 6457-6732, 6483-7025, 6486-6884, 6493-7014, 6498-7095, 6532-6917, 6540-6728, |
| | 6540-7009, 6553-6829, 6556-6929, 6558-6792, 6560-7106, 6584-6818, 6584-7047, 6592-6845, 6636-6804, |
| | 6639-6879, 6651-6884, 6694-6958, 6707-6992, 6741-6993, 6765-7125, 6765-7135, 6768-7004, 6771-7013, 6771-7025, |
| | 6771-7051, 6771-7058, 6771-7077, 6771-7133, 6771-7135, 6772-7048, 6772-7068, 6774-7017, 6775-7091, |
| | 6775-7104, 6777-7135, 6786-7087, 6789-6939, 6790-7035, 6794-7135, 6795-7064, 6796-7076, 6797-7061, |
| | 6803-7055, 6805-7053, 6807-7042, 6809-7135, 6810-7106, 6818-7046, 6819-7117, 6820-7096, 6822-7000, 6822-7032, |
| | 6834-7053, 6843-7108, 6844-6993, 6844-7076, 6844-7135, 6849-7088, 6851-7131, 6860-7135, 6888-7132, |
| | 6888-7135, 6891-7106, 6891-7109, 6916-7135, 6916-7152, 6935-7107 |
| 55/7499976CB1/ | 1-566, 172-624, 234-871, 268-1662, 284-624, 337-625, 380-1028, 400-624, 417-521, 417-659, 417-804, 417-851, |
| 1669 | 417-853, 417-874, 417-892, 417-914, 417-929, 417-938, 417-951, 417-1055, 417-1117, 462-1113, 478-1124, 480-696, |
| | 639-762, 651-831, 659-1350, 662-864, 694-1325, 694-1405, 722-1102, 731-965, 741-1385, 748-1334, 752-1342, |
| | 757-1359, 761-1081, 764-1280, 770-1388, 775-1200, 780-1017, 781-1307, 789-1274, 824-1301, 827-1289, |
| | 828-1427, 831-1406, 831-1522, 832-1202, 834-1227, 856-1253, 857-1342, 868-1252, 904-1148, 928-1157, 947-1669, |
| | 973-1483, 988-1645, 1000-1623, 1013-1285, 1015-1662, 1037-1424, 1041-1652, 1042-1211, 1043-1661, 1047-1192, |
| | 1055-1652, 1074-1398, 1082-1325, 1082-1332, 1097-1266, 1097-1640, 1109-1660, 1117-1669, 1150-1373, |
| | 1153-1595, 1153-1658, 1156-1661, 1159-1628, 1162-1669, 1185-1669, 1186-1662, 1190-1661, 1193-1389, 1195-1462, |
| | 1203-1379, 1203-1660, 1204-1501, 1207-1661, 1238-1669, 1306-1501, 1307-1661, 1311-1669, 1334-1616, |
| | 1339-1669, 1343-1647, 1373-1669, 1384-1658, 1417-1658, 1527-1669 |
| 56/7499954CB1/ | 1-517, 1-728, 1-830, 1-910, 3-624, 35-270, 137-3289, 201-531, 201-757, 215-846, 219-608, 286-1159, 296-1159, |
| 3591 | 316-1159, 318-1159, 412-659, 447-1159, 497-1158, 609-1159, 673-931, 733-1000, 736-1018, 741-1339, 796-1158, |
| | 867-1152, 901-1145, 901-1290, 901-1362, 901-1394, 901-1494, 922-1478, 922-1525, 943-1283, 1003-1666, 1036-1295, |
| | 1036-1300, 1036-1496, 1036-1564, 1036-1584, 1036-1643, 1036-1644, 1062-1259, 1062-1337, 1062-1900, |
| | 1071-1339, 1085-1680, 1103-1341, 1103-1447, 1106-1449, 1106-1585, 1123-1549, 1166-1722, 1173-1426, 1183-1405, |
| | 1227-1489, 1227-1899, 1246-1869, 1259-1547, 1279-1454, 1315-1424, 1374-1998, 1388-1852, 1406-2036, |
| | 1407-2013, 1414-1971, 1424-2099, 1441-2056, 1447-2070, 1448-2068, 1453-2146, 1459-1736, 1461-2193, 1481-2230, |
| | 1483-1723, 1492-2046, 1496-1768, 1501-2133, 1507-2140, 1511-1700, 1558-1845, 1582-1981, 1582-2092, |
| | 1598-2279, 1609-1972, 1619-2230, 1619-2242, 1647-2279, 1648-1883, 1649-1888, 1655-2306, 1662-2144, 1676-1970, |
| | 1676-2114, 1683-1939, 1683-2191, 1709-1973, 1712-1979, 1713-2259, 1723-2206, 1723-2368, 1723-2405, |
| | 1749-2385, 1753-2043, 1760-1972, 1760-2260, 1768-2054, 1769-2056, 1774-2456, 1775-2228, 1776-2136, 1791-1990, |
| | 1815-2103, 1827-2446, 1838-2453, 1839-2096, 1841-2441, 1854-2133, 1869-2173, 1887-2103, 1887-2398, |
| | 1887-2427, 1888-2447, 1901-2132, 1901-2455, 1921-2153, 1921-2382, 1947-2221, 1947-2456, 1954-2248, 1962-2151, |
| | 1962-2232, 1978-2145, 2000-2455, 2014-2336, 2014-2403, 2014-2441, 2020-2467, 2049-2345, 2049-2488, |
| | 2055-2455, 2064-2155, 2077-2354, 2087-2327, 2091-2189, 2091-2487, 2102-2448, 2152-2435, 2160-2427, 2165-2450, |
| | 2223-2413, 2237-2839, 2243-2527, 2244-2857, 2248-2321, 2265-2486, 2270-2728, 2310-2598, 2317-2559, |
| | 2317-2888, 2317-2890, 2317-2989, 2321-2944, 2323-2921, 2328-2582, 2328-2588, 2328-2649, 2328-2837, 2328-2941, |
| | 2328-2943, 2333-2480, 2333-2548, 2333-2604, 2333-2703, 2333-2809, 2333-2816, 2333-2867, 2333-2869, |
| | 2333-2874, 2333-2878, 2333-2893, 2333-2918, 2333-2933, 2333-2935, 2336-2582, 2336-3000, 2341-2582, 2345-2455, |
| | 2351-2617, 2352-2568, 2353-2549, 2354-2448, 2354-2475, 2354-2817, 2355-2889, 2358-2705, 2361-2870, |
| | 2380-2657, 2380-2661, 2380-2735, 2385-2648, 2396-2758, 2405-3153, 2415-2893, 2419-2657, 2419-2734, 2419-2735, |
| | 2419-2825, 2419-2863, 2419-2867, 2419-2868, 2419-2937, 2419-2968, 2420-2937, 2420-3040, 2421-2649, |
| | 2425-2678, 2435-2918, 2437-2580, 2443-2612, 2443-2666, 2445-2615, 2445-2647, 2446-3096, 2484-2741, 2484-3171, |
| | 2488-3046, 2493-2922, 2497-2777, 2502-2779, 2503-3138, 2510-2754, 2510-3133, 2525-2742, 2525-2916, |
| | 2528-2854, 2528-3217, 2533-2938, 2536-2846, 2536-2854, 2538-2846, 2545-2771, 2550-2818, 2550-3167, 2551-3261, |
| | 2553-2825, 2553-2938, 2553-3074, 2553-3078, 2554-3217, 2557-3251, 2569-2853, 2569-3215, 2586-3190, |
| | 2587-2855, 2587-3296, 2596-3028, 2599-3216, 2603-3216, 2606-2850, 2606-3143, 2611-3133, 2613-2900, 2613-3200, |
| | 2642-2939, 2645-3272, 2655-3262, 2656-2855, 2656-2921, 2658-3109, 2659-3109, 2660-3238, 2661-3108, |
| | 2662-2867, 2662-2938, 2669-3097, 2672-3097, 2675-3152, 2675-3273, 2676-2954, 2678-3036, 2682-2954, 2682-3196, |
| | 2682-3251, 2683-2938, 2683-2968, 2683-3016, 2683-3019, 2683-3109, 2683-3180, 2683-3226, 2684-3109, |
| | 2684-3172, 2685-3185, 2689-3282, 2691-3108, 2697-3244, 2697-3302, 2697-3303, 2705-3274, 2716-3071, |
| | 2716-3283, 2726-3181, 2727-2990, 2731-3317, 2732-3179, 2734-3109, 2736-3004, 2736-3109, 2736-3178, 2736-3179, |
| | 2738-3247, 2742-3295, 2744-2944, 2755-2961, 2755-2992, 2755-3015, 2755-3035, 2755-3108, 2757-3274, |
| | 2760-3296, 2760-3304, 2760-3352, 2761-3178, 2770-2951, 2770-3289, 2781-3272, 2781-3308, 2786-3071, 2788-3090, |
| | 2795-3272, 2799-3311, 2799-3351, 2805-3312, 2807-3289, 2807-3309, 2809-3289, 2809-3296, 2809-3302, |
| | 2809-3317, 2809-3352, 2814-3248, 2821-3289, 2821-3304, 2822-3325, 2824-2997, 2825-3127, 2825-3301, 2826-3289, |
| | 2832-3311, 2834-3078, 2834-3358, 2835-3288, 2835-3290, 2835-3321, 2836-3147, 2836-3293, 2838-3294, |
| | 2841-3179, 2849-3294, 2852-3289, 2853-3287, 2857-3217, 2857-3311, 2857-3328, 2859-3285, 2861-3289, 2867-3248, |
| | 2867-3290, 2867-3310, 2868-3247, 2868-3248, 2873-3133, 2873-3170, 2875-3289, 2876-3287, 2876-3295, |
| | 2880-3363, 2881-3289, 2883-3316, 2884-3197, 2885-3294, 2887-3115, 2888-3292, 2889-3289, 2893-3289, 2894-3248, |
| | 2894-3291, 2895-3137, 2895-3289, 2896-3143, 2897-3185, 2897-3294, 2898-3117, 2898-3289, 2898-3291, |
| | 2902-3167, 2902-3305, 2912-3359, 2917-3118, 2921-3289, 2939-3248, 2939-3306, 2942-3289, 2943-3330, |
| | 2948-3294, 2951-3217, 2952-3591, 2953-3239, 2962-3289, 2969-3248, 2978-3289, 2981-3296, 2983-3211, 2990-3289, |
| | 2991-3287, 2992-3118, 2996-3289, 2998-3289, 3013-3302, 3021-3248, 3021-3289, 3043-3303, 3050-3287, |
| | 3058-3289, 3077-3161, 3081-3293, 3084-3306, 3087-3290, 3092-3286, 3097-3291, 3100-3337, 3101-3289, 3107-3289, |
| | 3110-3289, 3115-3289, 3116-3289, 3139-3289, 3167-3307, 3174-3289, 3221-3301, 3223-3289 |
| 57/7500827CB1/ | 1-1884, 102-513, 217-902, 229-860, 234-720, 234-923, 251-451, 259-857, 288-569, 303-577, 309-970, 320-598, 320-990, |
| 1896 | 361-1018, 387-946, 390-686, 390-993, 406-676, 415-996, 424-958, 440-810, 456-635, 463-713, 463-717, 473-1119, |
| | 493-730, 508-1107, 519-985, 526-785, 526-796, 577-850, 615-865, 616-1090, 622-836, 623-1207, 629-1170, |
| | 660-926, 665-907, 678-1281, 696-1188, 697-1099, 716-1019, 716-1269, 716-1323, 726-1327, 731-958, 738-1275, |
| | 743-1216, 754-1054, 776-1281, 814-1337, 826-1275, 833-1047, 861-1008, 864-1130, 879-1281, 883-1166, 930-1271, |
| | 940-1501, 942-1138, 943-1204, 969-1257, 984-1235, 1010-1276, 1028-1565, 1031-1281, 1045-1223, 1050-1281, |
| | 1056-1275, 1059-1281, 1067-1324, 1112-1393, 1124-1808, 1141-1298, 1151-1233, 1158-1687, 1166-1362, |
| | 1178-1833, 1179-1822, 1188-1439, 1193-1442, 1206-1892, 1241-1896, 1243-1513, 1249-1873, 1277-1505, 1281-1876, |
| | 1283-1894, 1284-1652, 1284-1846, 1284-1894, 1285-1818, 1285-1894, 1285-1896, 1287-1860, 1298-1876, |
| | 1302-1789, 1307-1854, 1309-1689, 1310-1872, 1317-1811, 1317-1894, 1319-1874, 1326-1701, 1330-1652, |
| | 1330-1887, 1335-1845, 1340-1799, 1345-1894, 1349-1621, 1352-1605, 1358-1848, 1363-1894, 1370-1894, 1376-1732, |
| | 1384-1670, 1393-1858, 1396-1632, 1399-1894, 1400-1889, 1405-1650, 1407-1893, 1409-1533, 1411-1679, |
| | 1412-1883, 1414-1894, 1417-1880, 1421-1893, 1425-1883, 1428-1876, 1430-1883, 1432-1693, 1432-1795, 1433-1655, |
| | 1433-1883, 1436-1876, 1439-1727, 1439-1740, 1439-1883, 1439-1894, 1440-1734, 1441-1880, 1442-1894, |
| | 1444-1894, 1449-1877, 1449-1883, 1453-1695, 1453-1882, 1453-1894, 1460-1883, 1462-1886, 1463-1894, 1464-1883, |
| | 1467-1876, 1467-1887, 1469-1737, 1469-1878, 1471-1620, 1477-1718, 1478-1878, 1478-1894, 1483-1894, |
| | 1484-1896, 1487-1883, 1489-1875, 1497-1894, 1498-1767, 1501-1777, 1501-1894, 1510-1876, 1525-1878, 1528-1894, |
| | 1538-1804, 1543-1883, 1543-1894, 1549-1894, 1550-1883, 1553-1882, 1559-1886, 1563-1896, 1566-1894, |
| | 1568-1883, 1573-1883, 1580-1888, 1581-1875, 1581-1882, 1581-1894, 1585-1883, 1590-1842, 1598-1796, 1598-1816, |
| | 1600-1811, 1601-1894, 1615-1883, 1615-1889, 1616-1848, 1620-1876, 1632-1883, 1663-1883, 1706-1894, |
| | 1707-1877, 1708-1883, 1709-1869, 1714-1880, 1723-1894, 1727-1894, 1731-1883, 1736-1894, 1754-1883, |
| | 1763-1883, 1818-1894 |
| 58/7948585CB1/ | 1-627, 340-966, 341-691, 516-1042, 517-1038, 517-1127, 744-1432, 755-1268, 961-1395, 961-1558, 1031-1678, |
| 2731 | 1143-1678, 1339-1968, 1344-1739, 1411-1896, 1411-1972, 1433-1570, 1478-2087, 1534-1761, 1611-1912, 1630-2034, |
| | 1650-2324, 1679-1927, 1679-2372, 1726-2152, 1766-2224, 1769-2007, 1893-2369, 1930-2638, 1950-2432, |
| | 1954-2250, 1969-2219, 1971-2629, 1998-2569, 2003-2581, 2015-2578, 2033-2621, 2037-2698, 2040-2560, 2050-2283, |
| | 2077-2302, 2094-2698, 2108-2374, 2108-2463, 2201-2456, 2225-2504, 2244-2722, 2245-2457, 2255-2415, |
| | 2255-2517, 2282-2731, 2357-2723, 2358-2591, 2358-2637, 2389-2631, 2389-2703, 2389-2726, 2466-2705 |
| 59/7500002CB1/941 | 1-941, 82-225, 90-223, 90-336, 95-860, 99-402, 99-665, 116-225, 126-329, 136-223, 137-225, 140-401, 224-279, |
| | 224-382, 224-394, 224-409, 224-439, 224-474, 224-751, 224-784, 224-860, 224-861, 229-516, 231-449, 233-503, |
| | 234-941, 243-501, 248-575, 252-547, 267-843, 270-699, 273-553, 284-751, 290-902, 305-571, 307-862, 309-863, |
| | 318-663, 321-575, 321-585, 321-589, 321-603, 321-637, 321-772, 326-620, 328-585, 331-862, 356-456, 358-862, |
| | 363-798, 367-750, 368-567, 373-742, 379-857, 382-851, 389-608, 396-862, 397-677, 397-682, 398-862, 399-862, |
| | 402-862, 403-861, 404-862, 406-850, 406-862, 407-861, 409-862, 413-862, 414-554, 414-862, 415-671, 416-847, |
| | 416-859, 416-862, 419-861, 419-862, 420-857, 421-861, 421-862, 422-862, 423-862, 424-861, 425-862, 426-644, |
| | 427-861, 428-862, 429-859, 429-861, 431-862, 432-848, 434-863, 437-862, 438-861, 439-862, 440-858, 441-861, |
| | 442-750, 442-862, 448-859, 451-694, 451-861, 454-859, 455-861, 457-857, 457-861, 458-861, 459-862, 460-860, |
| | 463-862, 464-613, 465-862, 467-565, 467-861, 471-841, 473-862, 474-863, 475-861, 475-863, 476-858, |
| | 476-862, 478-848, 479-861, 479-862, 480-580, 483-860, 486-694, 487-861, 489-862, 495-857, 501-860, 503-862, |
| | 504-862, 508-860, 509-862, 510-859, 510-861, 512-862, 515-862, 516-613, 516-861, 517-699, 518-761, 518-809, |
| | 518-861, 518-862, 528-863, 535-862, 536-860, 540-862, 547-862, 549-835, 555-862, 557-862, 568-861, 578-861, |
| | 579-862, 584-855, 586-861, 610-862, 613-862, 637-861, 641-839, 641-861, 647-862, 649-861, 651-862, 652-862, |
| | 657-861, 683-862, 689-860, 689-862, 691-862, 696-861, 697-861, 703-858, 716-861, 718-862, 743-861 |
| 60/7500012CB1/ | 1-218, 1-321, 1-3997, 4-372, 16-248, 82-374, 82-980, 84-359, 84-404, 84-663, 85-336, 100-649, 101-659, 101-687, |
| 4152 | 101-691, 126-401, 138-402, 138-730, 138-832, 138-847, 138-913, 138-925, 138-975, 148-640, 193-827, 290-838, |
| | 300-927, 300-929, 300-935, 302-925, 309-827, 315-755, 333-575, 340-597, 420-1212, 422-948, 449-1142, 462-1086, |
| | 462-1087, 524-717, 540-1145, 573-681, 574-1096, 574-1142, 578-825, 589-1142, 655-1112, 666-1142, 677-1133, |
| | 692-1133, 692-1142, 693-963, 693-1140, 718-1142, 758-1142, 776-1101, 783-1142, 830-1378, 935-1149, 936-1010, |
| | 937-1536, 939-1534, 939-1536, 944-1536, 948-1536, 952-1536, 967-1536, 970-1536, 975-1536, 978-1536, |
| | 979-1536, 986-1536, 988-1536, 996-1835, 1000-1536, 1024-1536, 1026-1535, 1027-1536, 1031-1536, 1033-1536, |
| | 1039-1536, 1042-1536, 1057-1536, 1063-1536, 1066-1343, 1080-1536, 1085-1536, 1102-1742, 1140-1196, 1144-1423, |
| | 1144-1436, 1144-1473, 1144-1484, 1144-1572, 1144-1573, 1144-1581, 1144-1628, 1144-1635, 1148-1605, |
| | 1150-1768, 1151-1535, 1151-1797, 1152-1677, 1152-1720, 1152-1802, 1152-1814, 1152-1829, 1153-1464, |
| | 1153-1482, 1153-1536, 1154-1808, 1155-1679, 1156-1576, 1157-1536, 1159-1701, 1162-1626, 1163-1541, 1163-1577, |
| | 1176-1596, 1182-1536, 1188-1625, 1201-1536, 1209-1644, 1209-1874, 1209-1892, 1226-1442, 1231-1424, |
| | 1235-1479, 1239-1363, 1267-1536, 1294-1806, 1323-1806, 1331-1806, 1374-1705, 1385-1971, 1406-1908, 1418-2272, |
| | 1420-1536, 1463-1719, 1463-1948, 1465-1990, 1465-2245, 1472-2029, 1558-1797, 1564-1852, 1636-2069, |
| | 1645-1908, 1650-2257, 1749-2261, 1847-2259, 1850-2094, 1856-2249, 1888-2399, 1909-2481, 1973-2908, 1992-2105, |
| | 2001-2550, 2017-2498, 2040-2493, 2040-2551, 2040-2682, 2051-2325, 2071-2323, 2073-2318, 2076-2572, |
| | 2097-2397, 2097-2592, 2100-2377, 2122-2346, 2158-2730, 2161-2833, 2178-2670, 2188-2738, 2197-2491, 2197-2832, |
| | 2202-2795, 2207-2842, 2223-2553, 2226-2833, 2249-2865, 2253-2833, 2267-2551, 2267-2562, 2295-2537, |
| | 2296-2833, 2302-2589, 2321-2833, 2322-2763, 2325-2518, 2334-2914, 2336-2578, 2383-2881, 2406-2847, 2407-2657, |
| | 2448-2905, 2448-3006, 2448-3057, 2507-2724, 2507-2756, 2509-2754, 2528-2743, 2553-3034, 2566-2732, |
| | 2593-3212, 2602-2869, 2634-2849, 2638-2770, 2644-3061, 2659-2935, 2663-2842, 2698-2948, 2718-2977, |
| | 2723-2976, 2793-3055, 2803-3052, 2806-2902, 2809-3102, 2814-2997, 2832-3422, 2845-3135, 2845-3300, 2845-3313, |
| | 2845-3438, 2845-3494, 2845-3518, 2845-3546, 2903-3486, 3068-3342, 3068-3651, 3072-3316, 3100-3416, |
| | 3115-3301, 3124-3451, 3136-3451, 3140-3664, 3155-3434, 3185-3740, 3195-3469, 3205-4152, 3207-4152, 3213-3405, |
| | 3224-3484, 3236-3514, 3238-3791, 3252-3393, 3252-3521, 3254-3480, 3278-4152, 3286-3558, 3338-4151, |
| | 3338-4152, 3360-4151, 3363-4149, 3376-3645, 3399-3640, 3418-3691, 3421-3670, 3423-3729, 3425-3690, 3427-3717, |
| | 3439-3650, 3464-3710, 3464-4036, 3475-3656, 3494-3757, 3494-3772, 3499-3767, 3510-3670, 3527-4137, |
| | 3530-4152, 3595-4126, 3620-3893, 3629-3919, 3638-3910, 3671-3949, 3685-3903, 3737-3757, 3744-3975, 3744-3980, |
| | 3744-3986, 3751-4150, 3751-4152, 3755-4034, 3761-4022, 3767-4054, 3780-3990, 3786-4025, 3812-3942 |
| 61/1664071CB1/727 | 1-252, 7-254, 7-256, 7-276, 9-261, 9-285, 9-481, 12-685, 14-266, 15-301, 15-305, 17-261, 17-299, 18-272, 19-261, |
| | 19-262, 20-295, 21-323, 58-306, 58-328, 58-338, 60-209, 65-312, 67-322, 68-348, 68-354, 70-245, 71-353, 73-327, |
| | 110-383, 124-400, 170-608, 170-703, 200-662, 213-704, 224-508, 244-704, 285-566, 298-569, 504-727, 515-704 |
| 62/6214577CB1/ | 1-721, 1-4113, 460-817, 737-1520, 763-1164, 763-1190, 849-1132, 862-1040, 938-1139, 1177-1759, 1179-1469, |
| 4122 | 1309-1397, 1355-1774, 1456-1995, 1501-2191, 1692-1945, 1751-2028, 1765-2045, 1802-1877, 1802-2398, 1873-2453, |
| | 2087-2352, 2149-2436, 2216-2496, 2257-2499, 2262-2527, 2435-2661, 2436-2663, 2440-2991, 2503-2690, |
| | 2526-2861, 2616-2980, 2719-3067, 2871-3071, 2871-3091, 2876-3102, 2876-3412, 2952-3194, 2971-3308, 2974-3428, |
| | 2983-3321, 2984-3221, 2989-3273, 2989-3284, 3075-3300, 3149-3676, 3151-3403, 3154-3425, 3183-3461, |
| | 3183-3730, 3184-3825, 3197-3461, 3271-3553, 3285-3534, 3288-3546, 3309-3562, 3336-4017, 3339-3950, 3401-3654, |
| | 3401-3937, 3420-3670, 3426-3797, 3453-3693, 3472-3697, 3472-3986, 3479-3743, 3480-4000, 3487-4103, |
| | 3508-4095, 3537-3812, 3537-3835, 3581-4080, 3587-3845, 3602-3879, 3617-4122, 3655-4107, 3659-4116, 3678-4112, |
| | 3687-4113, 3689-4112, 3694-4122, 3696-4004, 3703-4112, 3703-4113, 3714-4013, 3726-4111, 3742-3904, |
| | 3760-4113, 3794-4013, 3800-4122, 3809-4112, 3811-4112, 3816-3947, 3816-4086, 3816-4096, 3816-4116, |
| | 3816-4122, 3818-4109, 3967-4087 |
| 63/7502149CB1/ | 1-124, 1-158, 1-191, 1-203, 1-230, 1-257, 1-279, 1-330, 1-345, 1-366, 1-471, 1-549, 393-1150, 440-586, 466-1198, |
| 9522 | 479-1200, 523-1200, 532-1198, 537-1198, 546-1005, 546-1198, 549-1198, 562-1198, 566-1198, 570-1198, 591-1198, |
| | 604-1198, 607-1198, 622-1198, 636-1198, 639-1198, 644-1198, 645-1198, 654-1198, 698-1198, 750-1198, |
| | 763-1198, 769-1040, 855-1198, 867-1199, 883-1198, 889-1344, 900-1198, 983-1660, 1051-1198, 1204-1557, 1204-1563, |
| | 1204-1680, 1204-1709, 1204-1750, 1204-1772, 1204-1776, 1204-1785, 1204-1820, 1204-1828, 1204-1862, |
| | 1204-1871, 1204-1895, 1204-1906, 1204-1953, 1204-1955, 1204-1967, 1204-1995, 1204-2003, 1204-2007, 1204-2013, |
| | 1204-2016, 1204-2064, 1204-2116, 1204-2118, 1205-1798, 1213-2016, 1220-2023, 1225-2197, 1227-2027, |
| | 1271-2113, 1287-1823, 1330-2191, 1351-1858, 1369-2196, 1411-2325, 1415-2095, 1415-2227, 1415-2339, 1502-2172, |
| | 1547-2214, 1575-2342, 1591-2263, 1601-2245, 1603-2255, 1624-2490, 1626-2426, 1664-2489, 1672-2073, |
| | 1762-2585, 1771-2600, 1774-2600, 1789-2568, 1792-2479, 1792-2600, 1818-2600, 1820-2600, 1831-2542, |
| | 1838-2547, 1847-2304, 1850-2319, 1857-2600, 1864-2600, 1865-2599, 1867-2600, 1868-2340, 1873-2600, 1878-2351, |
| | 1878-2600, 1888-2549, 1891-2484, 1894-2600, 1902-2600, 1912-2600, 1914-2501, 1925-2595, 1934-2600, |
| | 1942-2600, 1958-2600, 1968-2441, 1976-2542, 2006-2600, 2009-2600, 2010-2600, 2019-2600, 2023-2600, 2024-2600, |
| | 2025-2395, 2029-2600, 2037-2572, 2050-2639, 2065-2481, 2100-2600, 2145-2600, 2146-2600, 2155-2585, |
| | 2172-2600, 2180-2582, 2213-2897, 2239-2600, 2246-2600, 2249-2600, 2267-2600, 2527-2604, 2613-3118, 2634-3151, |
| | 2814-3398, 2890-3094, 2890-3097, 2890-3373, 2891-3094, 2892-3498, 2895-3373, 2898-3094, 2902-3094, |
| | 2902-3214, 2955-3620, 3011-3627, 3052-3648, 3061-3518, 3159-3392, 3159-3604, 3159-3651, 3159-3656, 3159-3666, |
| | 3159-3677, 3159-3690, 3159-3704, 3182-3849, 3357-3844, 3406-3896, 3416-3676, 3444-3627, 3460-3912, |
| | 3478-3857, 3505-3836, 3531-3787, 3561-4054, 3561-4159, 3561-4160, 3582-4112, 3587-4160, 3590-4153, 3618-4206, |
| | 3621-4241, 3686-3836, 3697-4232, 3711-4429, 3715-4361, 3715-4450, 3730-3984, 3752-4318, 3760-4309, |
| | 3836-4387, 3844-4417, 3852-4417, 3996-4484, 3999-4442, 4024-4419, 4040-4502, 4077-4677, 4141-4781, |
| | 4219-4767, 4227-4888, 4255-4607, 4260-4904, 4262-4745, 4269-4880, 4347-4905, 4361-4755, 4363-4916, 4372-4983, |
| | 4387-5055, 4418-4852, 4432-4928, 4456-5035, 4463-4984, 4507-4584, 4603-5143, 4634-5158, 4661-5215, |
| | 4682-5197, 4690-5216, 4697-5378, 4733-5369, 4734-5203, 4762-5075, 4768-5584, 4784-5215, 4798-5197, 4799-5266, |
| | 4822-5301, 4822-5315, 4825-5239, 4825-5314, 4826-5119, 4826-5239, 4850-5386, 4863-5528, 4879-5155, |
| | 4879-5378, 4951-5502, 4960-5531, 4963-5240, 5010-5311, 5013-5174, 5018-5313, 5052-7322, 5055-5522, 5059-5320, |
| | 5059-5721, 5097-5224, 5100-5887, 5111-5621, 5123-5528, 5465-5995, 5490-6011, 5527-6118, 5542-6049, |
| | 5542-6071, 5543-6050, 5543-6073, 5543-6088, 5554-5946, 5554-5964, 5663-6092, 5851-5981, 5891-6592, 5909-6421, |
| | 5911-6199, 5954-6108, 6057-6152, 6057-6565, 6057-6592, 6066-6740, 6087-6472, 6090-6592, 6220-6414, |
| | 6248-6592, 6258-6509, 6274-6716, 6274-6813, 6280-6843, 6300-6740, 6321-6592, 6336-6592, 6346-6922, 6348-6592, |
| | 6357-6957, 6372-6957, 6384-6592, 6390-6592, 6390-6993, 6407-7024, 6419-6951, 6419-6963, 6426-7127, |
| | 6494-7174, 6527-7065, 6546-7165, 6556-7005, 6606-7369, 6630-7127, 6632-7461, 6667-7191, 6992-7579, 7061-7575, |
| | 7061-7598, 7061-7607, 7061-7690, 7061-7726, 7061-7731, 7061-7761, 7061-7825, 7061-7856, 7061-7941, |
| | 7062-7869, 7064-7923, 7109-7681, 7145-7798, 7158-7710, 7187-8032, 7222-7839, 7265-7986, 7324-8226, 7356-8080, |
| | 7375-8080, 7458-7986, 7465-8396, 7474-7971, 7531-8199, 7532-8170, 7534-8187, 7538-8104, 7601-8162, |
| | 7613-8177, 7629-8179, 7642-8260, 7642-8313, 7719-8324, 7730-8497, 7742-8503, 7752-8271, 7765-8223, 7820-8588, |
| | 7850-8442, 7860-8340, 7891-8371, 7982-8581, 7986-8577, 7987-8762, 8000-8716, 8045-8879, 8067-8710, |
| | 8070-8910, 8072-8842, 8113-8860, 8114-8818, 8115-8626, 8136-8644, 8137-8654, 8164-8731, 8174-8692, 8177-8701, |
| | 8205-8952, 8323-8951, 8360-8875, 8360-9012, 8360-9055, 8458-8969, 8467-9037, 8512-9018, 8519-9184, |
| | 8538-9254, 8540-9201, 8554-9384, 8563-9212, 8583-9166, 8587-9171, 8592-9311, 8593-9390, 8596-9225, 8596-9237, |
| | 8596-9508, 8603-9384, 8604-9248, 8604-9257, 8609-9341, 8610-9162, 8615-9313, 8618-9062, 8634-9393, |
| | 8640-9396, 8644-9124, 8647-9203, 8647-9219, 8657-9254, 8662-9352, 8664-9268, 8678-9268, 8679-9407, |
| | 8700-9287, 8700-9311, 8702-9394, 8711-9351, 8721-9254, 8721-9293, 8722-9319, 8724-9355, 8730-9409, 8737-9327, |
| | 8745-9266, 8763-9298, 8767-9232, 8767-9384, 8770-9509, 8776-9396, 8782-9522, 8783-9393, 8786-9366, |
| | 8794-9396, 8796-9396, 8805-9349, 8806-9345, 8809-9396, 8811-9513, 8843-9342, 8871-9501, 8882-9508, 8895-9396, |
| | 8910-9404, 8914-9508, 9004-9521, 9038-9522, 9047-9522, 9055-9519, 9057-9522, 9069-9515, 9069-9522, |
| | 9072-9519, 9072-9522, 9076-9522, 9080-9522, 9083-9522, 9086-9522 |
| 64/7503480CB1/ | 1-232, 1-563, 1-2992, 8-135, 8-607, 15-267, 16-515, 18-119, 18-483, 22-407, 22-419, 22-484, 22-641, 23-137, 26-586, |
| 3987 | 28-629, 49-648, 65-306, 65-384, 65-402, 65-544, 65-676, 68-149, 68-342, 73-684, 89-535, 90-434, 104-640, |
| | 112-633, 149-614, 171-412, 171-789, 171-790, 171-885, 171-908, 172-682, 172-738, 172-743, 175-933, 178-812, |
| | 179-804, 189-733, 189-736, 190-840, 191-674, 191-822, 197-867, 199-766, 200-499, 200-757, 202-640, 204-857, |
| | 211-528, 216-814, 216-897, 217-480, 217-791, 217-892, 219-909, 223-565, 224-489, 224-559, 224-697, 224-945, |
| | 225-826, 228-945, 238-789, 271-673, 284-473, 286-777, 286-949, 298-788, 307-946, 322-762, 350-1023, 352-710, |
| | 361-411, 385-914, 389-879, 413-630, 460-1198, 463-1065, 479-738, 524-906, 528-1185, 530-903, 575-1015, 593-1058, |
| | 599-1044, 612-794, 674-885, 677-1244, 690-1096, 710-1336, 736-978, 747-981, 767-1245, 776-1097, 778-1314, |
| | 827-1407, 836-1043, 836-1085, 880-1385, 880-1462, 886-1407, 899-1493, 901-1166, 907-1603, 909-1204, |
| | 910-1394, 912-1509, 915-1159, 917-1149, 927-1516, 933-1464, 933-1465, 947-1276, 957-1566, 1009-1693, |
| | 1009-1729, 1020-1399, 1046-1466, 1060-1357, 1060-1637, 1066-1279, 1066-1311, 1066-1329, 1072-1319, 1076-1672, |
| | 1157-1399, 1164-1766, 1184-1659, 1197-1473, 1218-1683, 1218-1808, 1233-1720, 1263-1791, 1264-1870, |
| | 1266-1653, 1300-1969, 1305-1844, 1305-1920, 1311-1816, 1313-1816, 1319-1885, 1336-1836, 1347-1828, 1347-1946, |
| | 1367-1648, 1382-1635, 1382-1817, 1388-1829, 1394-1652, 1394-1876, 1395-1836, 1399-1682, 1401-1922, |
| | 1423-1600, 1424-1588, 1453-2016, 1454-2001, 1461-1892, 1465-1925, 1467-1836, 1502-1665, 1542-1831, 1580-1851, |
| | 1591-1976, 1593-2032, 1601-2035, 1606-1888, 1621-2201, 1634-1870, 1668-1975, 1684-2029, 1700-2157, |
| | 1740-1991, 1757-2002, 1812-2122, 1819-2037, 1898-2199, 2034-2093, 2034-2208, 2034-2279, 2034-2301, 2034-2303, |
| | 2034-2721, 2041-2276, 2092-2444, 2093-2237, 2127-2409, 2129-2386, 2129-2590, 2158-2412, 2159-2400, |
| | 2159-2412, 2186-2218, 2213-2762, 2221-2470, 2227-2888, 2235-2586, 2247-2575, 2272-2577, 2276-2548, 2286-2540, |
| | 2291-2475, 2295-2897, 2319-2984, 2342-3125, 2355-3002, 2356-2631, 2363-2657, 2364-3093, 2369-2839, |
| | 2370-2636, 2374-2801, 2376-3125, 2377-2611, 2379-3126, 2383-3125, 2395-3093, 2396-3125, 2401-3069, |
| | 2414-2978, 2414-3075, 2415-3088, 2423-3094, 2446-3127, 2449-3116, 2472-3125, 2490-2773, 2500-2781, 2500-2987, |
| | 2503-3270, 2525-3094, 2533-3141, 2557-3235, 2616-3218, 2632-3272, 2671-2960, 2671-3141, 2678-3122, |
| | 2684-3128, 2686-3129, 2699-3141, 2700-3128, 2704-3132, 2717-3094, 2726-2977, 2744-3127, 2750-3301, 2752-3127, |
| | 2756-3141, 2808-3124, 2812-3030, 2812-3054, 2812-3066, 2812-3076, 2813-3076, 2813-3086, 2813-3103, |
| | 2813-3138, 2821-3096, 2850-3108, 2867-3134, 2881-2996, 2891-3124, 2909-3683, 2928-3123, 2936-3166, 2936-3187, |
| | 2936-3417, 2965-3129, 2992-3241, 2997-3303, 3034-3578, 3044-3985, 3092-3136, 3153-3349, 3161-3505, |
| | 3193-3566, 3213-3453, 3314-3576, 3321-3987, 3322-3938, 3341-3529, 3354-3583, 3354-3852, 3373-3659, 3375-3709 |
| | 3375-3882, 3375-3959, 3376-3601, 3435-3970, 3443-3687, 3490-3763, 3496-3757, 3502-3736, 3528-3786, |
| | 3534-3787, 3534-3844, 3547-3806, 3567-3802, 3603-3889, 3619-3726, 3638-3876, 3660-3942, 3668-3909, 3697-3854 |
| 65/7500017CB1/ | 1-756, 1-847, 1-857, 1-860, 1-872, 1-992, 130-386, 130-420, 130-471, 130-628, 130-638, 158-409, 158-699, 158-722, |
| 1698 | 171-480, 171-651, 171-704, 171-785, 172-780, 173-706, 181-565, 182-433, 182-440, 182-838, 185-418, 189-602, |
| | 191-451, 191-770, 194-791, 215-481, 221-481, 221-876, 235-403, 235-410, 235-815, 258-477, 308-596, 315-758, |
| | 315-892, 317-572, 317-623, 344-673, 349-1033, 352-940, 359-1033, 362-615, 372-630, 383-973, 386-1031, |
| | 399-634, 401-966, 405-1017, 415-674, 417-1022, 419-666, 421-673, 421-680, 423-921, 432-1008, 441-565, 452-751, |
| | 465-698, 465-1125, 469-938, 471-712, 473-1140, 476-1398, 484-747, 485-895, 488-790, 488-823, 489-1108, |
| | 496-1004, 500-772, 508-1119, 510-777, 510-825, 532-783, 535-1216, 536-1153, 537-709, 538-1398, 543-1397, 547-796, |
| | 550-1143, 553-920, 569-1093, 571-1172, 575-1152, 576-806, 598-1397, 598-1398, 602-1108, 607-1132, 612-1143, |
| | 614-878, 619-863, 625-1401, 631-1189, 637-996, 638-1319, 643-1316, 643-1337, 644-1398, 652-1263, 658-1141, |
| | 659-1179, 660-1236, 664-1250, 670-1187, 672-1077, 676-943, 678-921, 678-1112, 681-885, 682-1161, |
| | 685-927, 687-1240, 689-875, 709-944, 709-980, 711-1031, 711-1459, 714-836, 725-1331, 728-1362, 733-1397, 742-1307, |
| | 753-1289, 757-1426, 765-997, 774-1312, 776-1244, 777-934, 777-954, 780-1401, 783-1426, 791-1234, 802-1459, |
| | 804-894, 804-1022, 810-1411, 822-1056, 827-1399, 829-1454, 833-1094, 842-1108, 850-1199, 872-1115, 885-1151, |
| | 890-1111, 895-1109, 895-1459, 897-1120, 897-1127, 900-1140, 907-1347, 907-1376, 908-1125, 918-1151, |
| | 920-1347, 922-1198, 931-1163, 936-1391, 939-1218, 941-1219, 941-1361, 942-1269, 957-1175, 962-1024, 962-1159, |
| | 967-1315, 970-1163, 973-1199, 973-1374, 980-1260, 980-1264, 982-1317, 986-1249, 986-1300, 990-1266, |
| | 1000-1251, 1001-1465, 1004-1390, 1006-1459, 1016-1163, 1017-1281, 1023-1285, 1026-1304, 1027-1302, 1032-1312, |
| | 1049-1397, 1063-1276, 1072-1317, 1082-1367, 1083-1408, 1085-1337, 1085-1359, 1098-1336, 1098-1339, |
| | 1110-1306, 1110-1326, 1110-1376, 1119-1366, 1125-1359, 1131-1273, 1169-1459, 1174-1451, 1177-1428, 1186-1368, |
| | 1189-1388, 1189-1397, 1198-1444, 1206-1660, 1208-1459, 1226-1459, 1233-1459, 1260-1459, 1267-1459, |
| | 1278-1457, 1299-1458, 1319-1454, 1458-1652, 1458-1677, 1459-1657, 1461-1675, 1471-1676, 1481-1672, |
| | 1484-1662, 1495-1657, 1517-1698, 1521-1657, 1548-1658, 1559-1662, 1588-1698 |
| 66/7499955CB1/ | 1-285, 6-140, 7-122, 27-610, 34-288, 34-327, 34-423, 37-311, 43-450, 45-144, 61-127, 61-128, 65-726, 81-311, 143-2309, |
| 2327 | 144-797, 146-372, 147-775, 149-728, 153-394, 155-458, 158-495, 158-642, 158-718, 164-669, 165-367, 165-382, |
| | 172-837, 176-776, 181-700, 182-401, 183-431, 187-371, 191-574, 192-748, 194-489, 194-713, 195-400, 195-442, |
| | 195-471, 200-461, 202-448, 202-645, 205-331, 209-412, 214-901, 215-470, 219-498, 219-526, 236-612, 237-409, |
| | 237-476, 239-477, 255-619, 256-882, 262-514, 263-688, 264-524, 265-525, 267-664, 267-679, 269-777, 271-526, |
| | 271-775, 278-490, 279-586, 279-945, 282-935, 283-680, 286-607, 287-652, 289-954, 290-543, 290-599, 291-562, |
| | 292-565, 292-749, 293-589, 293-752, 296-560, 296-777, 296-784, 296-973, 296-975, 299-699, 301-553, 301-840, |
| | 302-465, 302-525, 302-952, 302-1153, 303-452, 307-973, 308-526, 308-647, 313-553, 313-565, 320-860, 322-567, |
| | 322-576, 322-610, 324-769, 324-785, 325-809, 326-409, 327-796, 329-559, 330-682, 333-627, 345-769, 348-950, |
| | 350-653, 350-732, 351-606, 354-614, 357-781, 357-809, 358-606, 360-633, 360-650, 360-664, 360-713, |
| | 360-784, 360-787, 360-949, 360-954, 360-958, 363-643, 363-781, 363-787, 363-954, 364-640, 365-556, 365-642, |
| | 369-595, 371-772, 374-685, 376-609, 376-641, 379-639, 380-862, 380-1002, 381-633, 381-682, 382-623, 382-663, |
| | 384-643, 385-1050, 387-663, 387-955, 389-644, 396-664, 397-672, 397-1069, 401-541, 405-647, 405-1008, 406-669, |
| | 408-710, 408-963, 409-584, 409-656, 409-695, 410-709, 412-550, 413-680, 418-870, 418-936, 421-646, 421-655, |
| | 421-661, 421-684, 421-694, 421-704, 421-767, 421-791, 422-665, 422-684, 422-697, 424-1088, 427-1088, 429-923, |
| | 430-707, 432-1055, 434-769, 435-938, 435-954, 436-1250, 440-712, 440-1079, 441-694, 442-704, 445-887, |
| | 445-1023, 452-566, 457-679, 457-693, 457-756, 457-889, 458-692, 465-725, 469-839, 471-765, 471-766, 472-730, |
| | 472-733, 472-742, 472-759, 474-781, 474-949, 474-1022, 475-941, 476-780, 479-716, 479-762, 480-798, 482-1082, |
| | 485-1197, 486-1087, 487-681, 488-743, 488-766, 489-731, 489-1100, 490-751, 490-1035, 490-1122, 491-743, 495-757, |
| | 503-947, 503-1103, 506-770, 509-1111, 513-850, 516-762, 516-951, 516-1040, 516-1044, 516-1251, 518-781, |
| | 520-669, 521-832, 522-951, 527-720, 527-830, 528-784, 533-1259, 535-1115, 543-1042, 545-815, 546-780, 547-799, |
| | 548-812, 551-843, 551-1239, 553-652, 554-861, 554-918, 560-1198, 560-1299, 562-855, 563-1241, 564-798, 565-799, |
| | 565-800, 565-806, 565-1227, 567-815, 570-851, 570-982, 573-1304, 575-1062, 576-784, 576-1231, 578-1197, |
| | 578-1220, 578-1297, 579-845, 579-853, 579-1301, 583-792, 583-1231, 587-818, 587-987, 590-850, 591-1191, 592-1077, |
| | 596-894, 600-838, 600-1228, 601-1296, 602-1256, 603-1191, 603-1199, 604-850, 604-868, 605-915, 606-1005, |
| | 607-922, 608-968, 609-1293, 612-883, 613-889, 615-1073, 619-1092, 621-869, 623-860, 624-911, 628-814, |
| | 628-855, 628-886, 630-1293, 634-836, 634-1295, 637-1118, 637-1188, 640-883, 644-924, 645-932, 648-897, 648-936, |
| | 648-940, 649-890, 650-843, 653-1263, 654-906, 655-901, 655-910, 656-1259, 658-889, 659-911, 659-1255, |
| | 662-898, 662-1291, 665-1227, 667-973, 668-926, 671-970, 671-1249, 675-937, 677-900, 677-904, 680-1225, 682-923, |
| | 687-921, 687-940, 687-1821, 691-947, 691-961, 691-983, 691-1164, 694-941, 696-1214, 702-1291, 708-964, |
| | 709-1291, 710-1180, 714-958, 717-969, 723-1180, 725-1247, 725-1256, 727-855, 731-978, 731-986, 737-991, 738-1112, |
| | 740-1303, 743-955, 743-960, 743-1066, 743-1195, 743-1196, 743-1198, 743-1199, 743-1282, 743-1290, |
| | 743-1305, 744-1221, 745-1239, 746-1097, 747-1200, 750-1303, 754-1303, 759-956, 760-1247, 765-1065, 767-995, |
| | 767-1004, 768-995, 773-1029, 773-1071, 777-1016, 777-1220, 777-1262, 782-1012, 782-1023, 782-1036, 782-1038, |
| | 782-1265, 784-996, 785-963, 785-1003, 785-1304, 787-1186, 787-1294, 788-1051, 789-1265, 791-1108, 794-983, |
| | 795-1266, 797-922, 798-1001, 799-1254, 803-1088, 805-995, 811-1012, 812-1029, 812-1079, 814-1092, 815-1243, |
| | 818-1045, 820-1072, 821-1198, 822-929, 825-1295, 826-1070, 827-1136, 828-1080, 828-1094, 830-1042, 832-1227, |
| | 833-1100, 835-1135, 837-1013, 843-1123, 843-1298, 847-1123, 848-1088, 851-1124, 852-1090, 854-1305, 856-1293, |
| | 857-1063, 857-1064, 857-1096, 857-1114, 857-1121, 857-1148, 859-1155, 866-1106, 876-1111, 876-1176, |
| | 878-1303, 879-1303, 881-1097, 881-1099, 882-1104, 884-1083, 889-1015, 890-1144, 892-1305, 893-1302, 900-1051, |
| | 900-1290, 903-1206, 904-1298, 906-1206, 907-1305, 908-1305, 909-1305, 910-1178, 912-1177, 912-1303, |
| | 912-1305, 913-1173, 914-1303, 915-1303, 923-1200, 929-1303, 939-1190, 940-1249, 944-1168, 944-1187, 944-1217, |
| | 946-1192, 946-1303, 950-1097, 950-1229, 950-1268, 950-1272, 951-1232, 953-1235, 961-1219, |
| | 961-1261, 962-1236, 966-1203, 969-1311, 970-1260, 983-1251, 1003-1303, 1006-1098, 1006-1303, 1013-1040, |
| | 1016-1270, 1016-1303, 1021-1230, 1021-1239, 1021-1298, 1022-1175, 1022-1248, 1022-1255, 1024-1279, 1024-1293, |
| | 1026-1297, 1027-1282, 1028-1156, 1032-1303, 1042-1303, 1049-1298, 1050-1289, 1054-1248, 1065-1285, |
| | 1075-1547, 1076-1589, 1077-1401, 1083-1389, 1085-1193, 1085-1576, 1085-1589, 1085-1669, 1086-1637, 1087-1265, |
| | 1088-1305, 1088-1442, 1088-1446, 1089-1303, 1092-1329, 1095-1651, 1096-1251, 1096-1305, 1096-1474, |
| | 1098-1776, 1099-1538, 1109-1706, 1114-1270, 1116-1297, 1118-1255, 1118-1710, 1122-1691, 1130-1637, 1131-1303, |
| | 1158-1670, 1165-1477, 1171-1251, 1176-1373, 1180-1592, 1180-1614, 1181-1437, 1188-1894, 1197-1303, |
| | 1201-1689, 1203-1703, 1210-1303, 1217-1303, 1225-1651, 1226-1479, 1230-1488, 1233-1850, 1295-1561, 1314-1963, |
| | 1331-1825, 1349-2016, 1350-2037, 1364-1637, 1364-1983, 1393-1670, 1407-1623, 1417-1582, 1440-1670, |
| | 1452-1916, 1465-1704, 1486-1772, 1488-2145, 1493-1969, 1495-1938, 1497-2012, 1500-1999, 1503-2035, |
| | 1521-1981, 1534-1843, 1538-1832, 1539-2145, 1540-1670, 1561-1828, 1597-2010, 1599-1862, 1600-1896, 1601-1944, |
| | 1608-1909, 1637-2235, 1641-2043, 1643-1877, 1650-1829, 1651-2292, 1667-2158, 1680-1962, 1699-2010, |
| | 1702-2155, 1711-1935, 1714-2327, 1724-1845, 1724-1966, 1726-2165, 1729-2296, 1733-1834, 1737-1877, 1752-2044, |
| | 1769-2327, 1778-2141, 1779-2043, 1788-2263, 1805-2049, 1806-2043, 1810-2054, 1818-2155, 1818-2165, |
| | 1831-2047, 1838-2327, 1891-2179, 1895-2174, 1896-2170, 1954-2201, 2010-2278, 2037-2310, 2048-2281, 2050-2183, |
| | 2133-2309 |
| 67/7504025CB1/ | 1-737, 122-874, 285-1140, 376-1047, 482-2862, 494-1336, 524-1260, 537-1001, 537-1055, 537-1092, 537-1198, |
| 3134 | 537-1447, 537-3097, 550-1179, 633-1040, 684-1075, 765-1493, 784-1044, 791-1336, 830-1337, 835-1347, 840-1475, |
| | 848-1266, 869-1167, 881-1427, 902-1254, 930-1260, 932-1552, 935-1574, 957-1526, 964-1524, 968-1538, |
| | 983-1201, 991-1580, 997-1580, 997-1665, 1038-1634, 1091-1726, 1118-1571, 1120-1763, 1134-1679, 1135-1688, |
| | 1172-1880, 1178-1454, 1178-1501, 1178-1697, 1178-1700, 1178-1703, 1180-1759, 1202-1833, 1223-1830, 1223-1842, |
| | 1301-1949, 1302-1544, 1315-1794, 1316-1761, 1331-1572, 1351-1761, 1351-1885, 1377-1702, 1394-1893, |
| | 1399-1732, 1400-1714, 1465-2044, 1511-2043, 1514-1825, 1520-1783, 1521-1804, 1535-1726, 1537-1819, 1554-2184, |
| | 1558-1743, 1561-1883, 1565-1782, 1582-1864, 1590-1702, 1662-2355, 1680-1990, 1684-2376, 1694-2233, |
| | 1698-2332, 1718-2151, 1755-2025, 1767-2053, 1796-2392, 1798-2112, 1809-2414, 1823-2092, 1826-2069, 1826-2070, |
| | 1829-2496, 1834-2094, 1839-2052, 1844-1975, 1865-2424, 1880-2122, 1883-2501, 1896-2400, 1924-2125, |
| | 1946-2548, 1986-2109, 1998-2551, 2005-2544, 2015-2636, 2032-2639, 2043-2600, 2046-2480, 2177-2659, 2182-2681, |
| | 2187-2465, 2187-2470, 2199-2712, 2235-2688, 2308-2557, 2348-3000, 2400-2512, 2409-3041, 2454-3024, |
| | 2454-3075, 2456-2757, 2565-3037, 2567-3125, 2568-3050, 2570-2910, 2583-3082, 2600-3134, 2624-3087, 2639-3082, |
| | 2640-3082, 2640-3083, 2640-3103, 2643-3121, 2646-3081, 2649-3082, 2656-3134, 2659-3102, 2659-3103, |
| | 2669-3071, 2670-2925, 2676-3134, 2687-3082, 2688-3082, 2692-3071, 2692-3084, 2692-3131, 2693-2956, 2693-3071, |
| | 2694-3105, 2708-3101, 2722-3072, 2723-3086, 2723-3088, 2726-2871, 2727-3071, 2729-3071, 2736-3009, |
| | 2743-3071, 2746-3071, 2753-3082, 2772-3045, 2791-3071, 2798-3028, 2798-3079, 2801-3129, 2802-3071, 2802-3088, |
| | 2807-3071, 2808-3082, 2812-3088, 2817-3070, 2818-3071, 2829-3082, 2829-3096, 2860-3052, 2862-3129, |
| | 2870-3071, 2870-3080, 2901-3071, 2903-3084, 2910-3071, 2986-3100 |
| 68/7503203CB1/ | 1-5218, 373-686, 373-689, 448-1239, 514-689, 611-1172, 611-1237, 626-820, 634-1250, 715-1294, 723-1182, 766-1126, |
| 5237 | 766-1238, 766-1311, 766-1372, 766-1428, 766-1495, 766-1525, 766-1561, 766-1564, 767-1374, 780-1126, |
| | 781-1313, 796-1488, 827-1296, 829-1317, 920-1358, 950-1749, 955-1749, 963-1749, 968-1749, 969-1492, 982-1633, |
| | 1007-1749, 1017-1749, 1028-1454, 1040-1581, 1056-1465, 1065-1334, 1065-1666, 1065-1688, 1072-1552, |
| | 1098-1649, 1101-1887, 1102-1887, 1113-1886, 1129-1672, 1131-1749, 1165-1318, 1165-1449, 1204-1637, 1206-1459, |
| | 1213-1485, 1246-1834, 1268-1871, 1280-1647, 1284-1406, 1284-1907, 1307-1749, 1312-1872, 1335-1884, |
| | 1363-1749, 1367-1749, 1401-1630, 1406-1851, 1423-1625, 1444-1944, 1449-1559, 1486-1674, 1495-1887, 1506-1821, |
| | 1560-1944, 1624-1944, 1634-1917, 1646-1944, 1657-1944, 1704-1944, 1720-1939, 1727-1944, 1812-2294, |
| | 1834-1931, 1942-2453, 1945-2315, 2005-2453, 2012-2787, 2026-2787, 2029-2787, 2037-2503, 2063-2649, 2091-2782, |
| | 2093-2774, 2099-2719, 2125-2702, 2125-2746, 2126-2850, 2129-2901, 2135-2787, 2146-2757, 2160-2317, |
| | 2168-2546, 2191-2580, 2193-2781, 2201-2650, 2203-2798, 2204-2881, 2206-2787, 2225-2668, 2234-2787, 2236-2454, |
| | 2236-2481, 2236-2682, 2242-2350, 2246-2499, 2261-2784, 2268-2878, 2297-2919, 2312-2514, 2321-2533, |
| | 2322-2765, 2339-2918, 2363-2787, 2368-2918, 2401-2654, 2402-2687, 2402-2700, 2484-2857, 2508-2924, 2523-3070, |
| | 2540-2754, 2549-2727, 2611-2820, 2628-2774, 2645-2893, 2697-2811, 2698-3326, 2779-3379, 2815-3461, |
| | 2850-3490, 2850-3525, 2873-3458, 2883-3413, 2937-3237, 2959-3114, 2972-3286, 3003-3248, 3012-3400, 3040-3599, |
| | 3043-3384, 3052-3737, 3071-3704, 3090-3280, 3093-3288, 3095-3592, 3096-3338, 3096-3611, 3106-3347, |
| | 3106-3348, 3106-3663, 3135-3732, 3165-3475, 3166-3648, 3185-3305, 3185-3375, 3185-3459, 3185-3543, 3198-3457, |
| | 3199-3588, 3201-3707, 3202-3505, 3202-3674, 3202-3677, 3203-3674, 3204-3674, 3204-3677, 3214-3468, |
| | 3263-3888, 3273-3674, 3274-3828, 3277-3575, 3319-3837, 3385-3662, 3386-3636, 3387-3617, 3447-3716, 3455-3577, |
| | 3466-3851, 3466-3899, 3474-3761, 3488-4118, 3527-3696, 3549-3832, 3553-3835, 3557-3820, 3568-4083, |
| | 3569-3857, 3569-4209, 3571-4129, 3586-3817, 3617-3828, 3635-4290, 3643-3729, 3696-3961, 3709-4041, |
| | 3745-4381, 3777-4047, 3777-4289, 3783-3862, 3787-4072, 3790-4453, 3795-4316, 38 19-4380, 3834-4342, 3834-4377, |
| | 3853-4356, 3854-4509, 3857-4121, 3886-4532, 3893-4446, 3903-4318, 3906-4138, 3915-4426, 3915-4523, |
| | 3920-4485, 3953-4568, 3982-4310, 3982-4591, 3992-4474, 3992-4763, 3992-4789, 3998-4127, 4006-4268, 4007-4332, |
| | 4011-4329, 4026-4629, 4035-4288, 4043-4485, 4045-4347, 4053-4376, 4053-4382, 4058-4513, 4060-4375, |
| | 4067-4391, 4067-4535, 4076-4728, 4086-4340, 4087-4642, 4087-4675, 4100-4402, 4101-4540, 4103-4409, 4129-4688, |
| | 4137-4587, 4160-4420, 4163-4747, 4163-4994, 4165-4754, 4173-4440, 4173-4652, 4175-4602, 4176-4451, |
| | 4176-4744, 4178-4558, 4181-4326, 4211-4837, 4220-4716, 4227-4792, 4233-4508, 4234-4514, 4253-5053, 4267-4824, |
| | 4272-4751, 4272-4956, 4276-4712, 4278-4882, 4282-5076, 4283-4737, 4283-4791, 4285-4895, 4290-4561, |
| | 4291-4538, 4293-4749, 4293-4755, 4293-4770, 4295-4588, 4300-5079, 4314-5052, 43 16-4620, 4328-5043, 4329-4559, |
| | 4338-4585, 4358-4666, 4358-4791, 4364-5048, 4368-5056, 4372-4572, 4372-4919, 4379-4569, 4386-4893, |
| | 4388-4589, 4410-4691, 4412-4675, 4416-4823, 4418-5046, 4424-4666, 4426-4945, 4433-4866, 4445-5101, |
| | 4450-5064, 4451-5118, 4453-4771, 4453-4923, 4462-5075, 4463-5021, 4465-4758, 4469-5116, 4472-4791, 4475-4747, |
| | 4487-5025, 4489-5129, 4492-5048, 4494-4760, 4496-4773, 4496-5118, 4496-5128, 4515-5079, 4530-5075, |
| | 4534-4813, 4539-4756, 4539-5013, 4541-5218, 4546-5019, 4548-5118, 4558-4858, 4559-5127, 4559-5130, 4560-4825, |
| | 4564-5120, 4576-4762, 4577-4997, 4580-5125, 4585-5130, 4588-4884, 4601-5116, 4604-4865, 4604-4885, |
| | 4609-4886, 4616-5054, 4618-4890, 4618-5091, 4618-5127, 4619-5087, 4622-4904, 4630-5115, 4642-4893, 4643-5066, |
| | 4646-4848, 4646-5128, 4651-5121, 4655-5128, 4656-4680, 4656-5128, 4660-5127, 4665-5122, 4665-5128, |
| | 4665-5130, 4668-4929, 4674-4846, 4675-5127, 4676-5128, 4678-5127, 4682-5121, 4689-4925, 4703-4953, 4704-5104, |
| | 4705-5128, 4710-5002, 4710-5059, 4710-5081, 4710-5118, 4710-5127, 4712-5127, 4713-4865, 4714-5128, |
| | 4717-5130, 4722-5237, 4723-5130, 4742-5128, 4746-5084, 4746-5127, 4746-5128, 4747-4994, 4747-5035, 4751-5127, |
| | 4758-5121, 4768-4953, 4774-5122, 4781-5049, 4788-5102, 4790-5128, 4795-5127, 4803-5127, 4803-5130, |
| | 4819-5081, 4819-5110, 4822-5122, 4834-5127, 4840-5108, 4841-5129, 4842-5127, 4846-5127, 4863-5106, |
| | 4865-5111, 4865-5126, 4871-5122, 4871-5129, 4878-5127, 4879-5080, 4879-5118, 4879-5128, 4884-5127, 4885-5128, |
| | 4890-5128, 4896-5127, 4896-5128, 4904-5009, 4904-5128, 4905-5130, 4936-5128, 4940-5127, 4948-5127, |
| | 4951-5124, 4955-5128, 4959-5127, 4982-5130, 5009-5127, 5021-5128, 5028-5122, 5028-5123 |
| 69/7503260CB1/ | 1-1440, 1-5309, 53-406, 53-410, 201-487, 201-690, 201-707, 201-801, 201-817, 289-795, 304-870, 429-1052, 430-837, |
| 5973 | 443-935, 500-996, 545-908, 559-1012, 562-788, 592-1001, 602-866, 622-1356, 638-816, 645-1144, 676-844, |
| | 713-1382, 756-1305, 857-1407, 859-1315, 920-1384, 1082-1448, 1089-1449, 1100-1343, 1110-1207, 1126-1293, |
| | 1362-2158, 1383-1896, 1384-1515, 1448-1590, 1449-2144, 1474-1656, 1478-2158, 1492-1668, 1529-1924, 1529-1925, |
| | 1575-1719, 1599-2061, 1695-1910, 1755-1845, 1822-1909, 1836-1910, 2209-2731, 2307-2515, 2461-2738, |
| | 2461-2820, 2508-2704, 2778-3090, 3001-3625, 3034-3625, 3056-3625, 3292-3878, 3293-3878, 3295-3878, 3296-3878, |
| | 3348-3946, 4353-4502, 4353-5130, 4354-5046, 4563-4924, 4623-4953, 4763-5625, 4937-5490, 4984-5191, |
| | 4995-5533, 5086-5339, 5119-5661, 5146-5767, 5161-5373, 5207-5629, 5217-5973 |
| 70/2969494CB1/ | 1-657, 227-896, 240-817, 241-796, 244-947, 246-746, 250-754, 336-854, 616-1150, 654-1287, 701-1332, 831-1268, |
| 3713 | 885-1556, 890-1140, 910-1337, 922-1605, 1013-1693, 1027-1306, 1117-1354, 1117-1568, 1153-1846, 1155-1398, |
| | 1166-1542, 1190-1558, 1211-1903, 1250-1522, 1252-1886, 1278-1953, 1308-1951, 1327-1951, 1330-1804, 1331-1799, |
| | 1350-1949, 1361-1804, 1437-1814, 1460-1903, 1498-2094, 1545-2293, 1559-2202, 1570-2130, 1623-1887, |
| | 1623-2050, 1623-2089, 1623-2127, 1623-2278, 1666-2379, 1678-2348, 1680-2290, 1709-1921, 1713-2392, 1716-2389, |
| | 1739-1999, 1739-2355, 1790-2101, 1847-2093, 1868-2501, 1924-2615, 1939-2125, 1939-2420, 1939-2467, |
| | 1939-2490, 1950-2488, 1976-2427, 2009-2267, 2059-2550, 2113-2655, 2131-2437, 2157-2755, 2170-2474, 2170-2619, |
| | 2171-2448, 2171-2625, 2179-2634, 2199-2447, 2205-2528, 2214-2667, 2232-2256, 2232-2442, 2235-2772, |
| | 2239-2297, 2247-2842, 2255-2516, 2270-2544, 2270-2757, 2273-2784, 2281-2547, 2301-2617, 2324-2864, 2334-3028, |
| | 2363-2987, 2399-3004, 2419-2607, 2427-2969, 2427-3070, 2444-2641, 2446-2619, 2452-3044, 2465-3066, |
| | 2497-3147, 2643-2889, 2643-3071, 2643-3098, 2677-2891, 2685-2923, 2687-2964, 2722-2970, 2759-2905, 2825-3354, |
| | 2894-3207, 2897-3181, 2905-3174, 2936-3208, 2973-3186, 2979-3293, 2995-3671, 3019-3051, 3020-3229, |
| | 3029-3304, 3040-3068, 3040-3287, 3042-3332, 3064-3663, 3067-3327, 3067-3351, 3076-3332, 3078-3298, 3088-3373, |
| | 3096-3323, 3102-3350, 3112-3366, 3121-3266, 3121-3368, 3121-3390, 3121-3398, 3121-3402, 3131-3691, |
| | 3134-3404, 3134-3408, 3142-3672, 3144-3368, 3146-3652, 3147-3374, 3147-3678, 3155-3668, 3157-3366, 3210-3462, |
| | 3217-3505, 3247-3688, 3250-3713, 3259-3646, 3277-3643, 3282-3543, 3282-3574, 3292-3589, 3305-3668, |
| | 3305-3674, 3306-3645, 3308-3674, 3317-3547, 3334-3678, 3354-3595, 3354-3625, 3354-3697, 3370-3673, 3372-3666, |
| | 3391-3674, 3405-3681, 3405-3705, 3435-3689, 3463-3665, 3480-3645, 3530-3678 |
| 71/7503201CB1/ | 1-614, 5-594, 5-603, 18-558, 19-590, 20-576, 21-420, 24-519, 30-270, 30-311, 34-295, 36-454, 38-564, 39-292, 41-268, |
| 1982 | 41-559, 50-538, 50-553, 58-635, 59-566, 61-340, 69-321, 70-355, 84-641, 84-723, 84-813, 84-873, 84-885, 84-898, |
| | 88-616, 88-855, 99-714, 118-412, 122-212, 124-682, 124-688, 127-914, 156-434, 158-947, 180-947, 181-947, |
| | 193-947, 198-947, 199-666, 201-370, 201-580, 201-720, 213-487, 232-846, 242-454, 242-846, 244-1094, 246-842, |
| | 246-846, 253-845, 256-846, 263-845, 276-561, 280-492, 280-580, 280-616, 280-680, 280-902, 280-914, 280-915, |
| | 285-881, 286-365, 286-407, 286-563, 286-577, 286-581, 286-656, 286-750, 286-761, 286-765, 286-807, 286-820, |
| | 286-853, 286-855, 286-867, 286-873, 286-878, 286-890, 286-904, 286-922, 286-935, 315-950, 316-947, 320-564, |
| | 320-591, 343-583, 344-846, 353-947, 356-947, 364-947, 365-947, 371-846, 374-945, 379-942, 379-947, 380-947, |
| | 392-846, 392-847, 407-691, 407-908, 408-947, 427-928, 472-910, 514-838, 522-785, 547-792, 548-819, 581-809, |
| | 590-848, 626-1201, 670-947, 739-947, 786-947, 806-1452, 826-1111, 861-1126, 927-1168, 1068-1381, 1068-1695, |
| | 1082-1684, 1119-1354, 1213-1344, 1213-1454, 1220-1510, 1247-1472, 1332-1696, 1337-1575, 1364-1452, 1372-1671, |
| | 1398-1638, 1421-1675, 1429-1644, 1432-1628, 1497-1743, 1498-1771, 1503-1764, 1507-1788, 1524-1752, |
| | 1534-1777, 1534-1797, 1544-1770, 1552-1759, 1555-1840, 1556-1931, 1574-1806, 1684-1982 |
| 72/7503262CB1/ | 1-275, 1-671, 1-2050, 260-877, 287-865, 330-628, 335-603, 394-828, 404-623, 404-654, 404-954, 410-954, 427-668, |
| 2050 | 494-729, 526-944, 540-944, 584-981, 594-871, 635-1334, 681-948, 706-943, 737-1049, 751-983, 756-1334, |
| | 766-1329, 937-1398, 937-1399, 943-1585, 953-1756, 955-1798, 956-1585, 973-1232, 1002-1233, 1002-1512, 1033-1578, |
| | 1058-1334, 1059-1331, 1085-1604, 1106-1582, 1115-1334, 1119-1334, 1129-2000, 1145-1312, 1152-1741, |
| | 1156-1409, 1160-1665, 1166-1668, 1185-1815, 1189-1891, 1196-2042, 1201-1468, 1232-1806, 1232-2039, 1238-1464, |
| | 1249-1529, 1259-1486, 1259-1491, 1284-2039, 1335-1616, 1335-2046, 1336-1904, 1337-1993, 1337-2042, |
| | 1345-1919, 1346-1615, 1346-1740, 1370-2039, 1381-2016, 1384-2039, 1384-2042, 1385-2009, 1403-2042, 1420-1922, |
| | 1422-2042, 1426-2037, 1429-1663, 1430-1620, 1430-1719, 1432-2039, 1461-2039, 1463-1736, 1482-1788, |
| | 1488-2004, 1490-1996, 1505-1950, 1509-2032, 1509-2050, 1534-2043, 1554-2043, 1554-2045, 1554-2050, 1555-1970, |
| | 1555-1997, 1555-2043, 1579-2042, 1579-2043, 1589-2050, 1592-2040, 1593-2043, 1596-2043, 1597-2040, |
| | 1597-2041, 1599-2042, 1603-2041, 1607-2050, 1611-2042, 1613-2042, 1619-2042, 1622-2042, 1624-2046, 1630-2042, |
| | 1631-2043, 1631-2046, 1635-2039, 1637-2043, 1639-2042, 1641-2042, 1641-2043, 1646-2030, 1659-2042, |
| | 1676-2049, 1681-2042, 1682-2032, 1683-2043, 1692-2050, 1693-2043, 1712-2046, 1772-2025, 1785-1976, 1865-2009, |
| | 1865-2038, 1865-2050, 1866-2043, 1927-2005, 1957-2047 |
| 73/7503409CB1/ | 1-105, 1-113, 2-253, 3-1370, 8-105, 10-264, 12-265, 12-346, 46-281, 113-245, 113-327, 113-356, 113-368, 113-372, |
| 1370 | 113-395, 113-408, 113-410, 113-542, 113-669, 114-355, 114-669, 123-334, 123-403, 131-930, 135-759, 143-711, |
| | 147-440, 148-467, 149-367, 161-386, 161-424, 164-582, 179-710, 179-759, 185-675, 186-440, 188-658, 189-796, |
| | 191-449, 194-718, 198-470, 201-741, 207-410, 208-436, 208-781, 213-819, 214-816, 217-491, 221-456, 224-819, |
| | 235-781, 239-548, 243-754, 245-494, 247-752, 248-566, 252-419, 254-419, 262-700, 265-503, 266-488, 283-641, |
| | 286-633, 291-620, 292-831, 301-886, 303-910, 306-760, 309-603, 322-564, 322-590, 322-591, 325-1077, 328-550, |
| | 328-576, 333-609, 333-613, 338-845, 339-541, 339-911, 345-592, 345-608, 373-604, 373-635, 376-845, 377-849, |
| | 388-619, 395-867, 396-580, 400-662, 430-881, 431-647, 434-1036, 435-723, 437-1083, 439-633, 439-694, 444-723, |
| | 452-1013, 464-715, 469-632, 469-761, 476-960, 476-995, 476-1044, 490-827, 512-760, 519-805, 520-803, 523-1114, |
| | 523-1133, 524-838, 530-747, 551-1010, 561-825, 573-834, 591-806, 593-835, 601-780, 609-873, 610-899, |
| | 613-1365, 619-1356, 625-862, 625-1033, 625-1116, 625-1344, 642-1137, 651-916, 651-970, 652-943, 659-980, 660-911, |
| | 673-898, 676-1348, 683-953, 683-1287, 686-965, 696-939, 714-825, 716-1094, 719-1354, 721-1056, 721-1102, |
| | 725-976, 731-941, 747-1012, 750-1034, 756-1057, 779-1354, 781-1063, 790-1356, 795-1370, 797-1369, 798-1053, |
| | 798-1084, 798-1344, 799-1370, 804-1335, 804-1345, 806-1331, 808-1370, 810-1368, 814-1035, 824-1342, 827-1337, |
| | 828-1305, 830-1037, 834-1070, 834-1074, 834-1369, 836-1051, 836-1052, 839-1368, 841-1321, 847-1343, |
| | 856-1099, 863-1370, 868-1208, 869-954, 869-1078, 872-1187, 872-1370, 881-1370, 887-1149, 891-1354, 894-1201, |
| | 894-1202, 895-1356, 896-1341, 897-1113, 898-1363, 905-1353, 907-1137, 907-1363, 915-1362, 919-1370, 927-1356, |
| | 928-1370, 930-1355, 930-1356, 932-1356, 933-1355, 933-1356, 934-1356, 938-1224, 939-1191, 943-1356, |
| | 944-1355, 947-1356, 955-1356, 957-1191, 968-1191, 968-1356, 971-1191, 975-1169, 977-1161, 978-1271, 990-1191, |
| | 991-1352, 994-1191, 1003-1221, 1019-1274, 1031-1265, 1033-1199, 1033-1254, 1033-1266, 1033-1277, 1045-1191, |
| | 1046-1191, 1051-1238, 1056-1370, 1060-1355, 1063-1355, 1069-1242, 1069-1355, 1077-1360, |
| | 1079-1191, 1096-1226, 1099-1356, 1111-1339, 1114-1355, 1116-1232, 1120-1356, 1121-1355, 1126-1370, 1131-1356, |
| | 1139-1360, 1142-1355, 1154-1370, 1176-1356, 1192-1320, 1192-1359, 1195-1356, 1205-1355, 1205-1356, |
| | 1205-1358, 1232-1356, 1246-1356, 1300-1356, 1305-1356 |
| 74/7503499CB1/ | 1-170, 1-212, 1-213, 1-264, 1-541, 1-631, 4-273, 21-273, 21-297, 21-1841, 27-147, 29-297, 43-271, 45-297, 224-708, |
| 1855 | 295-804, 295-837, 295-858, 295-908, 297-548, 297-724, 304-855, 316-787, 316-795, 316-814, 318-815, 318-855, |
| | 318-1035, 322-793, 324-968, 326-1000, 342-974, 352-1073, 353-710, 354-845, 364-996, 378-665, 389-731, |
| | 398-667, 407-706, 414-750, 416-701, 424-837, 424-894, 430-673, 452-780, 452-1097, 461-1015, 474-1037, 496-1032, |
| | 503-1028, 548-850, 548-1101, 559-1003, 562-800, 562-1023, 572-1074, 579-1013, 580-1131, 608-1233, 619-1037, |
| | 624-798, 645-943, 653-1135, 671-1207, 695-1219, 727-1037, 736-1274, 767-1315, 817-1049, 833-1207, 839-1035, |
| | 853-1381, 865-1073, 901-1183, 913-1130, 930-1581, 984-1101, 987-1250, 995-1192, 1034-1293, 1053-1493, |
| | 1053-1499, 1064-1343, 1074-1619, 1075-1340, 1078-1285, 1089-1438, 1089-1470, 1095-1658, 1101-1216, 1117-1381, |
| | 1132-1789, 1140-1381, 1145-1770, 1153-1403, 1162-1278, 1176-1725, 1197-1418, 1212-1550, 1213-1515, |
| | 1218-1501, 1222-1840, 1225-1843, 1247-1631, 1251-1526, 1254-1855, 1275-1846, 1288-1811, 1335-1855, |
| | 1348-1592, 1357-1638, 1384-1840, 1386-1671, 1387-1750, 1394-1855, 1425-1848, 1427-1691, 1428-1833, 1431-1850, |
| | 1432-1850, 1435-1837, 1436-1850, 1440-1661, 1440-1663, 1474-1853, 1478-1704, 1483-1855, 1549-1840, |
| | 1554-1853, 1555-1850, 1560-1833, 1608-1840, 1717-1850 |
| 75/90031281CB1/ | 1-309, 1-654, 2-586, 3-841, 4-148, 7-822, 9-610, 9-630, 9-646, 10-431, 10-670, 11-848, 12-302, 12-365, 12-708, 12-759, |
| 2018 | 12-777, 12-783, 12-798, 12-811, 12-907, 12-929, 17-309, 17-311, 17-645, 19-685, 23-808, 33-309, 42-309, 42-311, |
| | 47-633, 55-795, 68-509, 104-806, 349-1315, 484-1410, 621-1433, 652-1315, 697-1431, 707-1171, 713-1435, |
| | 743-1521, 1145-2018 |
| 76/90061570CB1/ | 1-805, 375-1133 |
| 1133 | |
| 77/7500027CB1/ | 1-234, 1-1692, 180-356, 180-363, 180-373, 180-414, 180-423, 180-426, 180-427, 180-430, 180-432, 180-441, 180-474, |
| 1692 | 180-499, 180-506, 180-686, 180-751, 180-755, 180-765, 180-778, 180-816, 180-983, 182-585, 182-906, 185-751, |
| | 190-755, 193-433, 193-449, 195-426, 198-481, 203-461, 205-992, 236-720, 320-593, 339-639, 367-1059, 368-956, |
| | 368-958, 496-956, 510-793, 557-820, 559-989, 573-848, 620-966, 695-976, 711-897, 711-954, 715-965, 722-995, |
| | 726-923, 727-956, 754-1062, 762-1059, 814-1067, 816-935, 838-1063, 838-1067, 871-1067, 886-1613, 892-1049, |
| | 893-972, 913-1104, 915-1045, 959-1227, 1064-1244, 1064-1284, 1064-1295, 1064-1324, 1064-1335, 1064-1423, |
| | 1064-1429, 1064-1459, 1064-1494, 1070-1267, 1071-1330, 1075-1370, 1082-1358, 1090-1286, 1094-1365, |
| | 1098-1234, 1105-1584, 1112-1358, 1112-1578, 1112-1655, 1116-1314, 1116-1335, 1116-1366, 1141-1408, 1143-1362, |
| | 1145-1575, 1146-1509, 1156-1410, 1159-1355, 1176-1376, 1179-1409, 1183-1274, 1200-1471, 1205-1465, |
| | 1207-1338, 1208-1338, 1232-1376, 1255-1434, 1255-1624, 1260-1538, 1289-1393, 1294-1455, 1321-1533, |
| | 1326-1542, 1327-1522, 1329-1548, 1332-1572, 1356-1558, 1471-1496, 1471-1497, 1471-1503 |
| 78/7504546CB1/ | 1-347, 1-1341, 76-357, 92-278, 92-335, 96-346, 103-376, 107-304, 108-337, 135-443, 143-440, 144-383, 195-448, |
| 1859 | 197-316, 219-444, 219-448, 252-448, 252-449, 267-1103, 274-353, 294-485, 340-608, 445-625, 445-665, 445-676, |
| | 445-705, 445-716, 445-807, 445-813, 445-843, 445-878, 445-998, 451-648, 451-1029, 452-711, 454-1084, 456-751, |
| | 463-739, 471-667, 475-746, 479-615, 486-967, 493-739, 493-965, 497-695, 497-716, 497-747, 501-1029, 522-792, |
| | 524-743, 526-972, 527-893, 536-1028, 537-794, 540-736, 552-1284, 557-760, 560-793, 564-655, 567-1293, 581-855, |
| | 586-849, 587-1117, 588-719, 589-719, 613-758, 629-1358, 636-818, 636-993, 636-1092, 639-1091, 641-922, |
| | 658-1323, 670-776, 675-839, 682-1320, 686-1004, 687-1063, 688-1301, 690-1004, 692-1203, 696-1114, 702-917, |
| | 707-926, 708-906, 710-934, 713-956, 715-1318, 728-1312, 729-1012, 737-942, 744-1301, 744-1326, 747-1235, 751-1320, |
| | 756-1287, 761-1020, 761-1372, 767-1331, 775-1319, 775-1320, 782-1317, 782-1331, 788-1332, 790-1065, |
| | 793-1330, 797-1076, 806-1083, 819-998, 820-1037, 820-1070, 821-1358, 822-1130, 854-1105, 854-1163, 855-880, |
| | 855-881, 855-887, 856-1036, 858-991, 862-1029, 873-1130, 873-1151, 876-1125, 876-1341, 886-1318, 889-915, |
| | 889-919, 889-921, 893-921, 893-1401, 896-1344, 897-1345, 898-1175, 902-1340, 906-1175, 911-1340, 912-1340, |
| | 913-1124, 913-1390, 917-1341, 918-1326, 918-1362, 918-1377, 919-1379, 923-1345, 937-1344, 944-1340, 954-1340, |
| | 962-1340, 966-1340, 973-1242, 973-1340, 979-1179, 984-1362, 997-1091, 997-1420, 1001-1282, 1002-1085, |
| | 1004-1366, 1011-1340, 1032-1342, 1033-1361, 1038-1294, 1038-1340, 1039-1299, 1039-1354, 1042-1338, 1042-1340, |
| | 1046-1136, 1048-1338, 1058-1340, 1062-1340, 1067-1344, 1083-1306, 1102-1369, 1117-1373, 1120-1362, |
| | 1133-1376, 1139-1340, 1154-1340, 1158-1345, 1160-1345, 1165-1397, 1189-1340, 1189-1345, 1190-1340, 1223-1375, |
| | 1241-1859 |
| 79/7503246CB1/ | 1-256, 1-501, 1-552, 1-573, 1-771, 1-2894, 17-363, 22-244, 266-819, 367-701, 384-1016, 401-893, 429-665, 430-784, |
| 2948 | 437-1107, 458-1011, 496-1090, 500-1176, 531-1190, 543-1186, 550-1197, 575-1173, 681-1308, 681-1495, 694-926, |
| | 696-1134, 717-1004, 741-993, 741-1066, 741-1182, 741-1250, 765-1585, 777-1022, 777-1028, 788-1111, 809-1402, |
| | 815-1282, 838-1400, 855-1147, 861-1423, 867-1099, 867-1147, 899-1086, 932-1540, 978-1671, 985-1620, |
| | 998-1356, 998-1428, 999-1612, 1017-1653, 1018-1630, 1031-1401, 1112-1832, 1131-1393, 1135-1239, 1141-1873, |
| | 1149-1372, 1149-1381, 1149-1387, 1156-1520, 1171-1745, 1192-1750, 1199-1859, 1219-1523, 1248-1523, 1248-1706, |
| | 1274-1731, 1291-1553, 1291-1869, 1301-1859, 1350-1869, 1356-1619, 1385-2102, 1387-1770, 1391-1742, |
| | 1394-1522, 1394-1845, 1461-1981, 1461-1997, 1462-1719, 1463-1845, 1468-2086, 1485-2094, 1501-2075, 1518-1765, |
| | 1518-1851, 1531-1822, 1538-1819, 1543-1720, 1627-2015, 1653-2130, 1678-2258, 1686-2118, 1690-2124, |
| | 1700-2262, 1757-2175, 1932-2471, 2004-2064, 2004-2079, 2006-2611, 2088-2587, 2135-2787, 2154-2264, |
| | 2282-2617, 2298-2720, 2322-2538, 2328-2908, 2339-2604, 2348-2938, 2356-2933, 2376-2619, 2393-2939, 2393-2945, |
| | 2411-2649, 2432-2948, 2439-2894, 2457-2948, 2465-2903, 2484-2672, 2558-2774, 2568-2705, 2610-2890 |
| 80/7505729CB1/ | 1-3141, 387-473, 387-638, 390-715, 391-602, 817-922, 817-2112, 1534-2182, 1631-2238, 1634-2233, 1790-2085, |
| 10662 | 1868-2067, 2169-2439, 2220-2439, 2441-2619, 2441-2658, 2491-2598, 2880-3141, 2882-3352, 2882-3449, 2882-3506, |
| | 2882-3523, 2882-3588, 2882-3590, 2882-3603, 2882-3636, 2882-3710, 2882-3711, 2882-10648, 2944-9624, |
| | 3003-3692, 3004-3189, 3004-3294, 3004-3379, 3004-3424, 3004-3429, 3004-3430, 3004-3475, 3004-3484, 3004-3492, |
| | 3004-3499, 3004-3513, 3004-3527, 3004-3537, 3004-3559, 3004-3576, 3004-3577, 3004-3607, 3004-3621, |
| | 3004-3628, 3004-3653, 3009-3585, 3025-3673, 3025-3725, 3138-3510, 3218-3782, 3240-3701, 3253-3758, 3270-3533, |
| | 3285-3784, 3288-3460, 3335-3979, 3558-4144, 3603-4242, 3604-4055, 3604-4283, 3620-4066, 3649-4168, |
| | 3650-4129, 3660-3784, 3666-4168, 3816-3966, 3903-4412, 4000-4608, 4048-4601, 4241-4765, 4527-5305, 4588-5369, |
| | 4727-5318, 5239-5589, 5275-5952, 5759-6365, 5787-6373, 6027-6617, 6255-6825, 6416-7020, 6863-6895, |
| | 6885-7363, 6885-7492, 7108-7302, 7288-7649, 7288-7656, 7309-7979, 7354-7979, 7428-7529, 7607-7856, |
| | 7625-7880, 7719-7967, 7765-8356, 7821-7851, 7891-8123, 7954-8443, 8103-8333, 8103-8657, 8205-8882, 8644-8992, |
| | 8739-9291, 8746-8992, 8806-8991, 8806-8992, 8834-8992, 8849-9056, 8849-9072, 8849-9411, 8849-9422, |
| | 8849-9462, 8867-9243, 8887-8992, 9092-9323, 9092-9334, 9184-9455, 9272-9587, 9272-9877, 9296-9727, 9311-9879, |
| | 9358-9675, 9396-9693, 9417-9640, 9473-9674, 9533-10142, 9538-10031, 9547-9955, 9547-10007, 9555-9858, |
| | 9599-9912, 9599-10041, 9599-10106, 9601-10171, 9615-9667, 9630-9901, 9636-10112, 9664-10317, 9669-9910, |
| | 9678-10317, 9751-10269, 9761-9963, 9761-9993, 9794-10193, 9798-10282, 9799-10282, 9830-10307, 9832-10337, |
| | 9886-10497, 9894-10354, 9901-10171, 9919-10218, 9930-10388, 9932-10184, 9937-10358, 9943-10497, |
| | 9965-10157, 9978-10151, 9980-10402, 10003-10230, 10021-10290, 10021-10301, 10029-10462, 10029-10490, |
| | 10032-10489, 10044-10253, 10062-10368, 10070-10662, 10079-10627, 10091-10638, 10101-10660, 10102-10393, |
| | 10106-10443, 10114-10603, 10187-10482, 10188-10442, 10188-10652, 10195-10635, 10199-10442, 10214-10493, |
| | 10230-10494, 10240-10460, 10240-10632, 10244-10603, 10258-10551, 10276-10546, 10283-10430, 10284-10662, |
| | 10303-10591, 10326-10646, 10344-10625, 10369-10653, 10374-10623, 10467-10662, 10519-10662 |
| 81/7487334CB1/ | 1-189, 1-484, 1-662, 1-690, 1-698, 25-669, 28-476, 28-588, 54-709, 59-690, 60-690, 74-695, 76-653, 76-676, 76-690, |
| 3311 | 76-695, 78-689, 106-690, 168-541, 170-696, 185-749, 199-680, 208-690, 214-680, 245-701, 264-925, 414-1111, |
| | 456-1207, 589-1046, 691-1122, 691-1124, 691-1129, 691-1164, 691-1173, 697-1459, 699-1414, 841-1066, |
| | 942-1586, 967-1190, 1015-1499, 1078-1719, 1118-1753, 1136-1750, 1142-1711, 1147-1405, 1175-1703, 1175-1751, |
| | 1182-1751, 1194-1660, 1195-1796, 1235-1703, 1314-1375, 1363-1630, 1366-1981, 1443-2037, 1445-2020, |
| | 1449-2133, 1453-2075, 1501-2057, 1515-2162, 1613-2040, 1624-2074, 1647-1742, 1648-1908, 1676-2219, 1688-1983, |
| | 1728-2160, 1740-2193, 1741-2333, 1742-1961, 1756-2026, 1770-2181, 1780-2207, 1780-2209, 1812-2196, |
| | 1838-2254, 1843-2384, 1877-2129, 1897-2541, 1898-2414, 1914-2560, 1940-2350, 1940-2352, 1975-2233, 1991-2656, |
| | 2001-2629, 2004-2602, 2011-2572, 2012-2260, 2033-2522, 2038-2601, 2039-2451, 2060-2342, 2063-2726, |
| | 2071-2340, 2071-2685, 2073-2492, 2076-2492, 2082-2683, 2088-2568, 2089-2553, 2091-2359, 2094-2624, |
| | 2099-2696, 2103-2371, 2123-2362, 2126-2325, 2126-2370, 2126-2381, 2126-2398, 2132-2409, 2139-2380, 2139-2410, |
| | 2139-2602, 2142-2345, 2144-2351, 2149-2550, 2157-2355, 2157-2412, 2163-2391, 2168-2436, 2181-2379, |
| | 2181-2453, 2187-2600, 2201-2500, 2207-2447, 2216-2512, 2222-2615, 2222-2626, 2236-2533, 2238-2548, 2252-2538, |
| | 2254-2508, 2254-2774, 2261-2901, 2286-2888, 2294-2870, 2308-2939, 2316-2601, 2323-2890, 2345-2888, |
| | 2355-2941, 2485-2694, 2498-3164, 2502-3075, 2505-2754, 2520-2774, 2525-2774, 2525-3033, 2530-2773, 2530-3106, |
| | 2531-2885, 2535-2863, 2540-2765, 2541-2774, 2614-2865, 2614-2875, 2614-2921, 2617-3080, 2623-2873, |
| | 2623-3060, 2624-2850, 2626-2918, 2629-2876, 2629-2913, 2629-2945, 2635-2894, 2636-2907, 2639-2900, 2641-2923, |
| | 2644-3288, 2646-2872, 2646-2928, 2646-3121, 2647-2872, 2650-2998, 2654-2923, 2659-2870, 2667-3197, |
| | 2771-2982, 2771-3276, 2771-3286, 2772-3260, 2777-3279, 2778-3302, 2792-2820, 2792-3273, 2809-3283, 2810-3062, |
| | 2813-3046, 2915-3294, 2917-3303, 2918-3302, 2921-3305, 2933-3194, 2936-3194, 2937-3209, 2938-3198, |
| | 2938-3207, 2940-3304, 2942-3296, 2967-3106, 2971-3250, 2991-3295, 2993-3296, 2997-3305, 3017-3311, |
| | 3036-3305, 3042-3290, 3046-3311, 3050-3303, 3054-3311, 3058-3311, 3063-3307, 3074-3249, 3074-3311, 3084-3311, |
| | 3085-3303, 3086-3311, 3087-3293, 3087-3294, 3087-3295, 3087-3296, 3090-3297 3118-3297, 3158-3282 |
| 82/7503109CB1/ | 1-802, 18-3950, 19-820, 20-3965, 64-480, 270-934, 286-591, 411-810, 413-978, 454-1052, 602-1237, 664-1379, 711-1271, |
| 4039 | 750-1318, 827-1340, 831-1398, 880-1609, 954-1101, 956-1402, 978-1422, 1009-1620, 1015-1652, 1019-1570, |
| | 1034-1267, 1076-1097, 1109-1731, 1109-1866, 1119-1694, 1119-1761, 1121-1655, 1123-1664, 1133-1348, |
| | 1133-1613, 1143-1623, 1145-1617, 1152-1720, 1174-1755, 1210-1848, 1262-1912, 1267-1449, 1290-1801, 1321-1451, |
| | 1363-1949, 1376-1970, 1382-1923, 1390-1661, 1485-2112, 1511-2107, 1511-2117, 1515-2180, 1541-1617, |
| | 1541-2167, 1549-2098, 1562-2292, 1567-1934, 1611-2223, 1658-1930, 1658-2232, 1663-2255, 1674-2165, 1674-2355, |
| | 1694-1938, 1700-1943, 1700-1953, 1723-2335, 1735-2399, 1739-2214, 1752-2288, 1770-1849, 1770-2201, |
| | 1770-2380, 1776-2028, 1868-2280, 1910-2378, 1927-2372, 1929-2385, 1936-2528, 1951-2201, 2024-2266, 2026-2264, |
| | 2063-2333, 2092-2385, 2099-2356, 2127-2282, 2381-2655, 2393-2658, 2405-2678, 2668-2883, 2671-2860, |
| | 2812-3044, 2848-3115, 2896-3135, 2974-3216, 2982-3585, 3059-3548, 3080-3877, 3081-3569, 3082-3878, |
| | 3085-3894, 3194-3723, 3197-3540, 3199-3902, 3220-3860, 3222-3951, 3227-3892, 3227-3911, 3244-3929, 3255-3746, |
| | 3255-3852, 3262-3967, 3268-3835, 3272-3865, 3272-3963, 3274-3753, 3287-3562, 3297-3937, 3299-3972, |
| | 3309-3940, 3337-3625, 3341-3923, 3346-3936, 3347-3660, 3350-3921, 3364-3878, 3372-3620, 3375-3941, 3378-3916, |
| | 3386-3937, 3388-3626, 3395-3649, 3396-3950, 3398-3886, 3413-3445, 3415-3965, 3415-3966, 3416-3642, |
| | 3416-3694, 3416-3816, 3416-3944, 3420-3935, 3422-3690, 3422-3939, 3423-3939, 3423-3942, 3425-3940, 3426-3676, |
| | 3428-3736, 3430-3717, 3431-3942, 3433-3742, 3435-3780, 3435-3940, 3437-3944, 3437-3965, 3441-3970, |
| | 3444-3721, 3445-3754, 3450-3708, 3450-3949, 3470-3940, 3477-3726, 3481-3730, 3481-3963, 3482-3967, 3484-3758, |
| | 3487-3965, 3494-3953, 3495-3951, 3505-3951, 3507-3952, 3510-3820, 3516-3935, 3516-3948, 3527-3775, |
| | 3533-3894, 3534-3950, 3535-3712, 3535-3800, 3536-3725, 3549-3951, 3549-3955, 3550-3950, 3552-3736, 3552-3912, |
| | 3553-3950, 3563-3916, 3567-3762, 3568-3949, 3575-3858, 3579-3956, 3580-3864, 3581-3689, 3582-3648, |
| | 3594-3954, 3605-3855, 3608-3823, 3611-3878, 3613-3888, 3618-3731, 3619-3835, 3620-3779, 3625-3866, |
| | 3661-3864, 3664-3950, 3670-3961, 3673-3952, 3674-3951, 3679-3825, 3681-3873, 3714-3914, 3720-3985, 3737-3990, |
| | 3759-3994, 3762-4039, 3768-3999, 3772-3955, 3782-3950, 3805-4010, 3819-3968 |
| 83/7503128CB1/ | 1-675, 193-940, 193-941, 193-2621, 232-512, 253-917, 254-532, 254-799, 268-808, 272-992, 283-761, 299-557, 347-621, |
| 2622 | 379-961, 451-732, 469-659, 469-1063, 491-792, 511-767, 545-952, 588-1027, 597-902, 652-962, 660-764, 677-995, |
| | 681-1051, 714-916, 714-954, 714-1068, 751-988, 824-936, 862-1052, 868-1071, 964-992, 1004-1255, 1006-1281, |
| | 1068-1240, 1068-1286, 1068-1357, 1177-1704, 1202-1441, 1233-1402, 1275-1549, 1275-1556, 1275-1803, |
| | 1276-1586, 1277-1391, 1277-1414, 1277-1427, 1277-1479, 1277-1530, 1277-1541, 1277-1546, 1277-1552, 1277-1578, |
| | 1279-1390, 1279-1542, 1279-1554, 1282-1422, 1283-1553, 1283-1563, 1284-1534, 1288-1542, 1289-1841, |
| | 1292-1648, 1293-1410, 1293-1748, 1304-1558, 1316-1521, 1321-1512, 1321-1727, 1327-1567, 1329-1697, 1340-1588, |
| | 1345-1790, 1346-1614, 1369-1538, 1371-1606, 1376-1608, 1406-1664, 1412-1709, 1414-1656, 1419-1612, |
| | 1423-1969, 1436-1650, 1442-1676, 1443-1728, 1450-1771, 1450-1785, 1453-1661, 1457-1744, 1458-1956, 1462-1734, |
| | 1462-1747, 1463-1688, 1463-1722, 1469-1722, 1471-1598, 1477-1737, 1477-1785, 1479-1954, |
| | 1481-1687, 1492-2025, 1493-1656, 1494-1759, 1495-1771, 1495-1772, 1510-2188, 1551-1835, 1554-1767, 1554-1997, |
| | 1566-1826, 1634-1990, 1639-2090, 1642-2267, 1643-2080, 1645-2212, 1646-2268, 1669-2381, 1674-2192, |
| | 1680-2037, 1682-1919, 1682-2006, 1682-2273, 1684-1888, 1684-1946, 1711-2163, 1712-1925, 1719-1996, 1733-2311, |
| | 1740-2237, 1741-2013, 1741-2037, 1744-1988, 1746-2274, 1748-2339, 1750-2162, 1771-2546, 1774-2026, |
| | 1776-2254, 1776-2377, 1786-2015, 1793-2041, 1793-2044, 1799-2235, 1800-2020, 1805-2092, 1811-2102, 1820-2083, |
| | 1829-2114, 1829-2135, 1836-2425, 1841-2067, 1841-2072, 1841-2075, 1841-2084, 1841-2141, 1841-2431, |
| | 1842-2108, 1845-2016, 1845-2322, 1847-2105, 1847-2109, 1847-2253, 1847-2268, 1847-2321, 1847-2410, 1848-2067, |
| | 1848-2108, 1848-2285, 1853-1885, 1853-2425, 1855-2220, 1857-2085, 1859-2540, 1862-2116, 1864-2052, |
| | 1864-2122, 1864-2198, 1865-2180, 1866-2046, 1868-2358, 1872-2108, 1873-2120, 1873-2193, 1881-2082, 1883-2104, |
| | 1890-2205, 1890-2620, 1896-2150, 1896-2435, 1899-2540, 1905-2083, 1915-2110, 1915-2147, 1918-2538, |
| | 1923-2145, 1925-2549, 1928-2135, 1929-2564, 1930-2287, 1936-2577, 1937-2172, 1937-2182, 1939-2528, 1941-2178, |
| | 1942-2226, 1944-2558, 1945-2211, 1949-2152, 1949-2203, 1949-2204, 1949-2216, 1949-2501, 1950-2218, |
| | 1952-2173, 1959-2283, 1965-2244, 1965-2283, 1965-2481, 1967-2526, 1974-2210, 1975-2186, 1975-2202, 1976-2622, |
| | 1977-2598, 1979-2558, 1986-2622, 1987-2617, 1989-2243, 1990-2220, 1990-2280, 1991-2273, 1991-2613, |
| | 2005-2244, 2006-2263, 2008-2238, 2011-2260, 2011-2597, 2011-2600, 2014-2281, 2016-2236, 2016-2310, 2018-2622, |
| | 2022-2292, 2024-2398, 2026-2614, 2031-2596, 2032-2243, 2046-2479, 2049-2547, 2050-2312, 2050-2317, |
| | 2050-2544, 2050-2582, 2055-2446, 2055-2532, 2057-2348, 2057-2411, 2057-2537, 2057-2547, 2057-2567, 2057-2572, |
| | 2057-2614, 2057-2618, 2057-2622, 2059-2622, 2060-2566, 2060-2596, 2072-2620, 2076-2614, 2082-2329, |
| | 2084-2622, 2087-2612, 2108-2344, 2110-2337, 2111-2355, 2111-2361, 2111-2596, 2111-2604, 2111-2622, 2117-2597, |
| | 2119-2366, 2119-2383, 2119-2622, 2120-2383, 2122-2596, 2125-2393, 2127-2610, 2133-2611, 2137-2578, |
| | 2141-2613, 2141-2614, 2142-2622, 2145-2420, 2146-2552, 2147-2397, 2147-2400, 2147-2406, 2147-2583, |
| | 2147-2597, 2148-2420, 2149-2374, 2149-2421, 2150-2614, 2151-2456, 2151-2558, 2154-2622, 2156-2616, 2157-2296, |
| | 2157-2420, 2157-2612, 2158-2612, 2159-2446, 2159-2620, 2160-2430, 2161-2611, 2162-2430, 2162-2611, |
| | 2164-2342, 2165-2393, 2165-2622, 2166-2622, 2167-2360, 2168-2615, 2168-2616, 2169-2422, 2169-2479, 2169-2553, |
| | 2169-2614, 2169-2622, 2171-2611, 2174-2612, 2175-2453, 2177-2452, 2179-2613, 2180-2609, 2180-2617, |
| | 2181-2611, 2183-2611, 2184-2612, 2185-2444, 2185-2612, 2185-2614, 2187-2610, 2189-2610, 2190-2614, 2192-2609, |
| | 2192-2610, 2194-2617, 2195-2434, 2196-2611, 2197-2622, 2201-2565, 2201-2621, 2201-2622, 2210-2407, |
| | 2210-2427, 2210-2614, 2211-2614, 2213-2562, 2214-2478, 2214-2486, 2214-2613, 2215-2619, 2219-2584, 2219-2612, |
| | 2219-2613, 2221-2618, 2221-2622, 2228-2613, 2232-2483, 2234-2611, 2235-2614, 2237-2477, 2239-2606, |
| | 2247-2610, 2247-2612, 2248-2533, 2248-2612, 2248-2614, 2249-2496, 2249-2612, 2251-2615, 2259-2529, 2260-2611, |
| | 2261-2611, 2263-2613, 2263-2618, 2264-2614, 2265-2611, 2266-2612, 2266-2622, 2269-2519, 2269-2533, |
| | 2271-2603, 2277-2593, 2284-2604, 2285-2611, 2291-2611, 2297-2564, 2302-2603, 2304-2578, 2306-2614, |
| | 2308-2615, 2313-2622, 2314-2611, 2319-2613, 2321-2614, 2324-2552, 2325-2578, 2330-2561, 2333-2596, 2335-2610, |
| | 2335-2612, 2337-2465, 2339-2611, 2344-2612, 2346-2622, 2350-2615, 2353-2611, 2355-2614, 2356-2622, |
| | 2357-2589, 2357-2606, 2357-2622, 2362-2622, 2363-2611, 2363-2622, 2368-2612, 2369-2574, 2369-2614, 2394-2612, |
| | 2395-2622, 2396-2610, 2396-2622, 2406-2603, 2406-2608, 2418-2611, 2420-2614, 2423-2617, 2425-2611, |
| | 2425-2612, 2428-2614, 2429-2612, 2431-2611, 2432-2622, 2434-2622, 2435-2619, 2435-2622, 2437-2622, 2442-2594, |
| | 2463-2611, 2469-2622, 2475-2611, 2476-2622, 2516-2622, 2518-2622 |
| 84/7503191CB1/ | 1-420, 3-231, 8-258, 15-294, 18-193, 19-296, 19-1942, 22-929, 24-212, 24-286, 29-273, 200-472, 234-806, 247-835, |
| 1959 | 283-576, 295-881, 296-836, 298-780, 355-770, 355-901, 393-861, 438-860, 462-861, 561-796, 639-876, 639-1156, |
| | 648-918, 653-892, 686-993, 710-983, 749-1314, 761-1019, 805-1014, 805-1094, 814-1074, 814-1094, 818-1086, |
| | 824-1053, 832-1638, 836-1111, 842-1098, 866-1020, 911-1160, 912-1157, 1015-1159, 1018-1159, 1158-1387, 1158-1396, |
| | 1158-1562, 1158-1642, 1158-1762, 1158-1788, 1162-1802, 1164-1762, 1175-1752, 1176-1534, 1187-1517, |
| | 1193-1477, 1216-1412, 1225-1772, 1249-1742, 1262-1527, 1269-1535, 1275-1539, 1293-1513, 1331-1804, 1341-1799, |
| | 1341-1847, 1363-1762, 1385-1796, 1390-1844, 1392-1860, 1393-1800, 1410-1791, 1413-1680, 1413-1802, |
| | 1414-1677, 1414-1937, 1414-1952, 1415-1799, 1421-1703, 1445-1847, 1453-1932, 1462-1872, 1488-1796, 1492-1735, |
| | 1497-1800, 1499-1782, 1508-1941, 1511-1941, 1518-1773, 1520-1949, 1522-1868, 1526-1942, 1537-1773, |
| | 1537-1938, 1539-1762, 1548-1804, 1553-1775, 1554-1941, 1560-1933, 1625-1762, 1638-1762, |
| | 1662-1903, 1663-1800, 1671-1928, 1858-1959, 1892-1942 |
| 85/7503196CB1/ | 1-183, 1-186, 1-389, 1-390, 1-498, 1-499, 1-603, 1-609, 1-643, 1-2918, 13-440, 13-466, 13-475, 22-359, 27-460, 28-507, |
| 3177 | 35-429, 41-377, 54-138, 54-541, 72-300, 140-186, 142-186, 205-224, 205-456, 205-657, 212-512, 241-641, 444-1071, |
| | 485-1016, 510-1206, 536-1230, 536-1267, 549-826, 593-886, 658-891, 711-1295, 800-1048, 818-1216, 862-1518, |
| | 1011-1047, 1066-1287, 1107-1732, 1110-1380, 1187-1708, 1226-1498, 1402-1883, 1449-1883, 1768-2016, |
| | 1812-2434, 1812-2443, 1843-2521, 1937-2343, 1958-2635, 1999-2684, 2036-2566, 2036-2567, 2038-2271, 2088-2414, |
| | 2088-2696, 2097-2631, 2101-2281, 2127-2592, 2127-2715, 2148-2270, 2180-2532, 2217-2491, 2258-2870, |
| | 2279-2901, 2307-2570, 2307-2575, 2344-2593, 2401-2627, 2405-2683, 2411-2701, 2430-2654, 2441-2679, 2441-2701, |
| | 2542-2669, 2557-2841, 2571-2727, 2571-3052, 2571-3177, 2588-2891, 2588-2910, 2669-2936, 2674-2939 |
| 86/7503254CB1/ | 1-166, 1-167, 1-294, 1-407, 1-440, 1-463, 1-2389, 2-446, 105-682, 112-204, 120-376, 124-411, 136-429, 136-805, |
| 2389 | 190-580, 202-300, 222-444, 248-869, 251-496, 261-809, 263-514, 296-767, 296-807, 297-594, 302-479, 322-548, |
| | 326-842, 351-482, 385-612, 461-809, 467-767, 468-763, 468-799, 470-700, 484-1173, 486-785, 505-759, 827-1367, |
| | 830-1216, 860-1110, 867-1587, 869-1131, 869-1143, 886-1364, 955-1208, 955-1473, 1058-1328, 1207-1464, 1254-1881, |
| | 1304-2017, 1322-1961, 1335-1817, 1343-1859, 1360-1804, 1376-1857, 1383-1887, 1385-1637, 1428-1707, |
| | 1461-2036, 1484-1702, 1488-1726, 1488-1986, 1489-1960, 1491-1787, 1500-1655, 1500-2055, 1502-2088, 1517-1747, |
| | 1548-2018, 1548-2049, 1563-2185, 1577-2076, 1605-1702, 1605-2048, 1607-2128, 1615-2184, 1626-2386, |
| | 1638-1769, 1659-2148, 1661-1942, 1661-2245, 1694-2331, 1701-2266, 1716-2360, 1718-2312, 1754-2386, 1780-2389, |
| | 1799-1969, 1816-2325, 1817-2046, 1819-2383, 1850-2379, 1853-2364, 1932-2351, 1935-2367, 1962-2230, |
| | 1964-2389, 2001-2167, 2033-2369, 2065-2349, 2099-2370, 2137-2372, 2148-2350, 2169-2372, 2202-2368 |
| 87/7503531CB1/969 | 1-287, 1-473, 1-969, 3-292, 50-309, 490-783, 521-753 |
| 88/7490021CB1/ | 1-756, 50-703, 229-932, 338-937, 338-1077, 338-1144, 339-1004, 339-1052, 339-1127, 339-1194, 341-1155, 378-1102, |
| 2024 | 405-431, 459-485, 951-1150, 1072-1888, 1784-2024 |
| 89/7503180CB1/ | 1-225, 17-257, 18-284, 22-284, 25-265, 28-294, 29-321, 29-333, 30-210, 31-308, 34-341, 36-236, 36-274, 36-281, |
| 1070 | 36-287, 36-306, 36-318, 36-325, 36-329, 37-141, 38-196, 38-247, 38-291, 47-323, 49-336, 50-286, 50-295, 50-330, |
| | 50-341, 51-272, 51-320, 53-283, 53-321, 62-300, 73-346, 74-271, 74-302, 74-328, 77-338, 77-699, 77-702, 101-257, |
| | 104-341, 390-656, 441-660, 441-765, 533-1070, 616-830, 627-759 |
| 90/7503206CB1/ | 1-280, 1-426, 1-2138, 3-432, 5-798, 11-429, 22-848, 24-588, 24-2154, 29-859, 32-707, 35-310, 43-647, 44-265, 44-891, |
| 2167 | 46-339, 47-315, 47-353, 51-441, 51-535, 51-559, 51-606, 51-717, 51-745, 51-779, 51-790, 51-793, 51-805, 51-847, |
| | 51-848, 51-877, 51-909, 51-918, 51-937, 51-945, 52-845, 57-369, 57-846, 64-307, 64-359, 64-368, 64-413, 64-466, |
| | 64-514, 64-524, 64-553, 64-567, 64-609, 64-702, 64-716, 64-804, 64-976, 65-871, 66-335, 66-569, 77-646, 77-898, |
| | 84-432, 84-646, 87-558, 88-383, 90-558, 102-307, 102-663, 141-577, 160-552, 164-664, 176-955, 187-467, 190-426, |
| | 212-474, 228-906, 230-688, 234-558, 236-501, 237-688, 263-549, 313-532, 319-743, 325-998, 326-845, 333-583, |
| | 335-579, 341-638, 344-635, 375-596, 449-904, 461-515, 468-904, 470-724, 473-931, 486-1215, 493-1009, 499-1034, |
| | 501-1153, 533-906, 546-782, 546-813, 546-863, 546-906, 546-915, 549-915, 554-811, 564-1200, 611-730, |
| | 611-1075, 629-1060, 728-1267, 784-1094, 785-1077, 801-1061, 802-1332, 807-1059, 819-1425, 820-1037, 824-1172, |
| | 833-1106, 900-1106, 907-1039, 1087-1610, 1101-1360, 1112-1524, 1112-1602, 1117-1459, 1118-1393, |
| | 1119-1267, 1138-1407, 1145-1396, 1146-1584, 1218-1510, 1224-1484, 1228-1356, 1268-1536, 1286-1543, 1287-1537, |
| | 1295-1537, 1295-1563, 1296-1558, 1323-1595, 1324-1531, 1332-1565, 1356-1599, 1385-1610, 1406-1722, |
| | 1406-1971, 1407-2062, 1408-2044, 1585-2124, 1612-2134, 1612-2140, 1614-2166, 1615-2121, 1625-1896, 1625-2147, |
| | 1626-2134, 1627-1885, 1635-2151, 1639-2167, 1645-2155, 1650-2134, 1656-2134, 1661-1907, 1669-2140, |
| | 1673-2134, 1676-2134, 1678-2136, 1680-2140, 1681-2140, 1683-2134, 1683-2147, 1686-2135, 1688-2143, 1692-2139, |
| | 1692-2144, 1707-2143, 1709-2140, 1714-2166, 1717-2136, 1719-1915, 1720-2127, 1723-2140, 1725-2134, |
| | 1725-2140, 1727-1901, 1728-2132, 1728-2167, 1729-2143, 1730-2126, 1736-2144, 1740-2141, 1740-2167, 1743-2139, |
| | 1745-2133, 1745-2134, 1746-2120, 1746-2134, 1746-2140, 1746-2142, 1749-2136, 1749-2144, 1773-2144, |
| | 1783-2144, 1786-2144, 1801-2147, 1812-2102, 1812-2109, 1812-2110, 1812-2118, 1853-2140, 1904-2135, 1936-2140, |
| | 1949-2142, 1998-2142, 1999-2136, 2000-2145, 2002-2135, 2035-2140, 2064-2134 |
| 91/7503227CB1/ | 1-148, 1-202, 1-212, 1-229, 1-234, 1-253, 1-259, 1-276, 1-288, 1-291, 1-1297, 9-196, 10-134, 10-267, 14-282, 15-281, |
| 1297 | 29-205, 309-832, 342-832, 342-845, 342-882, 342-891, 342-893, 342-967, 342-1048, 343-932, 345-633, 346-876, |
| | 349-625, 349-641, 351-629, 352-598, 354-881, 355-627, 356-982, 357-535, 358-832, 363-622, 369-695, 374-709, |
| | 379-762, 381-917, 381-1062, 388-663, 390-495, 396-665, 396-858, 406-1088, 408-614, 416-658, 416-903, 426-696, |
| | 428-829, 428-892, 432-914, 438-936, 441-687, 441-726, 443-699, 444-975, 446-1139, 447-906, 448-1017, 455-598, |
| | 456-720, 459-702, 459-704, 459-714, 460-721, 476-648, 478-966, 481-714, 481-936, 493-754, 496-682, 500-739, |
| | 503-951, 504-951, 507-791, 513-769, 513-1249, 514-693, 514-773, 514-803, 516-821, 518-766, 518-957, 522-982, |
| | 522-1283, 524-694, 525-891, 527-1283, 528-807, 530-1184, 531-761, 532-847, 533-791, 533-1203, 535-798, |
| | 535-799, 536-827, 536-948, 540-791, 551-787, 554-947, 556-622, 561-761, 563-1154, 567-826, 571-802, 571-814, |
| | 571-832, 575-891, 576-973, 576-982, 577-871, 577-982, 577-985, 578-854, 581-1048, 585-891, 587-936, 588-1107, |
| | 590-780, 590-1130, 591-827, 591-832, 591-879, 592-1203, 593-1225, 595-865, 601-1193, 602-1250, 603-851, 603-1255, |
| | 610-1148, 613-769, 616-915, 617-704, 617-914, 619-1041, 621-885, 629-1251, 630-681, 630-875, 631-908, |
| | 632-922, 639-1220, 652-910, 666-925, 668-1249, 671-1213, 672-1062, 677-897, 679-1280, 680-883, 684-1273, 687-897, |
| | 690-1081, 690-1187, 691-952, 696-961, 696-967, 698-812, 698-868, 699-956, 705-1297, 706-912, 706-935, |
| | 706-1272, 706-1297, 708-1191, 728-979, 729-947, 729-982, 735-1058, 742-975, 743-1297, 744-1177, 748-1243, |
| | 749-982, 750-1022, 751-982, 757-1030, 758-1038, 758-1205, 760-1174, 761-1262, 765-949, 765-1181, 765-1260, |
| | 767-1056, 773-1163, 773-1277, 774-1054, 776-1018, 777-1170, 781-1297, 788-1267, 795-1061, 796-1049, 798-1295, |
| | 800-1242, 802-1275, 805-1119, 806-1092, 806-1242, 810-1094, 811-1067, 814-1134, 816-1284, 817-1068, |
| | 822-1284, 828-1297, 829-1250, 829-1297, 832-1297, 833-1283, 834-1297, 836-1282, 837-1251, 837-1264, 838-1288, |
| | 843-1133, 843-1267, 845-1104, 845-1106, 848-1263, 851-1297, 852-1150, 856-1282, 856-1297, 858-1275, |
| | 860-1254, 860-1284, 864-1283, 866-1128, 866-1215, 869-1126, 869-1135, 872-1284, 873-1149, 874-1141, |
| | 874-1283, 875-1121, 876-1275, 882-1283, 882-1297, 883-1284, 883-1297, 885-1283, 891-1213, 891-1283, 894-1275, |
| | 899-1188, 901-1166, 902-1167, 902-1297, 903-1284, 905-1269, 911-1296, 912-1175, 913-1284, 923-1283, |
| | 928-1284, 929-1297, 942-1210, 942-1282, 943-1283, 945-1290, 946-1282, 949-1284, 974-1279, 974-1282, 974-1284, |
| | 978-1290, 979-1284, 980-1283, 980-1284, 981-1273, 983-1272, 986-1178, 986-1228, 990-1263, 1002-1274, |
| | 1010-1275, 1016-1136, 1021-1242, 1029-1284, 1031-1284, 1038-1290, 1047-1235, 1047-1267, 1049-1180, 1049-1251, |
| | 1049-1284, 1055-1282, 1055-1284, 1056-1270, 1057-1297, 1058-1263, 1059-1297, 1068-1272, 1078-1262, |
| | 1084-1297, 1093-1297, 1094-1191, 1134-1297, 1135-1238, 1135-1292, 1135-1297, 1140-1205, 1167-1284, 1172-1284, |
| | 1190-1267, 1199-1297 |
| 92/7504473CB1/ | 1-1286, 97-1290, 320-727, 346-748, 374-590, 420-549, 420-550, 454-743, 552-965, 560-748, 580-704, 583-753, 583-765, |
| 1330 | 592-855, 599-754, 664-797, 715-814, 719-1133, 725-928, 725-1000, 727-874, 728-863, 728-954, 729-894, 729-1074, |
| | 729-1210, 730-863, 730-943, 731-1161, 732-863, 732-1030, 733-863, 734-863, 737-897, 738-863, 743-1266, |
| | 744-1266, 746-897, 748-1076, 749-1019, 750-1268, 751-1017, 751-1099, 751-1225, 754-1035, 755-1099, 755-1137, |
| | 755-1176, 755-1212, 757-1098, 757-1154, 758-1169, 758-1289, 760-1051, 760-1293, 761-1327, 762-1269, 766-954, |
| | 767-1265, 772-1286, 778-1076, 783-1267, 786-1039, 786-1174, 790-1167, 792-1245, 800-1056, 800-1321, 803-986, |
| | 809-967, 811-1247, 831-1099, 834-1263, 842-1256, 848-1211, 855-1092, 855-1327, 864-1267, 865-1019, 868-1125, |
| | 870-1144, 871-1148, 880-1116, 883-1103, 892-1153, 899-1242, 910-1155, 912-1168, 913-1161, 924-1178, 937-1205, |
| | 939-1093, 939-1175, 939-1194, 939-1330, 974-1191, 980-1258, 982-1266, 988-1187, 990-1255, 992-1257, |
| | 993-1258, 1009-1255, 1014-1286, 1020-1285, 1021-1272, 1021-1287, 1032-1288, 1032-1308, |
| | 1038-1330, 1039-1266, 1055-1288, 1071-1288, 1083-1318, 1105-1238, 1141-1281, 1155-1280, 1157-1271 |
| 93/7503200CB1/ | 1-278, 1-448, 1-611, 1-2641, 8-596, 14-281, 22-551, 22-681, 35-669, 35-677, 36-266, 36-621, 579-893, 604-893, |
| 2654 | 652-1347, 652-1349, 690-1349, 706-1349, 714-1349, 866-1349, 939-1403, 939-1445, 948-1601, 1357-1621, 1731-2499, |
| | 1732-2397, 1732-2452, 1732-2497, 1732-2654, 1850-2395, 1976-2279, 2138-2420, 2138-2428, 2138-2641, |
| | 2205-2524, 2215-2469, 2379-2654, 2393-2627 |
| 94/7500465CB1/ | 1-281, 1-1648, 8-160, 15-151, 15-186, 15-277, 15-281, 15-282, 15-283, 23-121, 24-281, 34-281, 36-530, 36-664, 36-712, |
| 1661 | 36-792, 36-831, 36-842, 36-860, 36-877, 42-281, 55-281, 57-171, 57-281, 62-281, 65-281, 77-278, 77-281, 83-281, |
| | 89-377, 106-189, 106-333, 106-544, 106-545, 151-281, 155-281, 550-1210, 567-1150, 581-1169, 583-794, 583-866, |
| | 595-1515, 610-1357, 627-1314, 636-1582, 641-1582, 646-794, 648-1335, 648-1582, 658-1582, 660-1308, 663-1582, |
| | 664-1236, 693-1336, 723-1271, 745-1392, 746-1336, 753-1345, 760-1541, 770-1448, 771-1463, 772-1582, |
| | 774-1582, 775-1380, 775-1582, 779-1361, 782-1419, 785-1582, 794-1582, 795-1582, 822-1465, 828-1435, 834-1504, |
| | 858-1582, 859-1093, 866-1582, 870-1582, 885-1474, 885-1565, 891-1511, 903-1582, 905-1382, 905-1582, |
| | 931-1473, 933-1582, 936-1083, 946-1548, 960-1461, 968-1646, 981-1642, 1002-1613, 1036-1661, 1039-1582, 1086-1582, |
| | 1127-1582, 1133-1554, 1133-1596, 1146-1655, 1190-1582, 1257-1582, 1258-1502, 1273-1567 |
| 95/7503256CB1/ | 1-96, 1-374, 1-416, 1-432, 1-554, 1-568, 1-573, 1-581, 1-598, 1-647, 1-648, 1-651, 1-666, 1-2210, 3-586, 17-474, 17-647, |
| 2483 | 22-478, 22-480, 45-753, 67-901, 68-577, 86-475, 86-478, 86-480, 86-663, 86-704, 90-613, 154-813, 272-474, |
| | 272-478, 682-1039, 857-1344, 898-1036, 898-1139, 965-1653, 1006-1248, 1029-1272, 1041-1114, 1042-1588, 1053-1144, |
| | 1065-1334, 1091-1684, 1117-1777, 1141-1684, 1153-1745, 1192-1733, 1204-1861, 1213-1859, 1220-1886, |
| | 1226-1866, 1230-1759, 1280-1788, 1307-2011, 1330-1894, 1332-1938, 1343-2056, 1348-1901, 1372-1787, 1399-2066, |
| | 1412-2057, 1414-1968, 1415-1823, 1415-2148, 1427-1936, 1428-1897, 1441-1885, 1450-1968, 1465-1714, |
| | 1501-2182, 1506-2066, 1507-2153, 1509-1739, 1509-2182, 1520-1776, 1534-1893, 1541-2060, 1546-2146, 1598-1816, |
| | 1619-2112, 1622-2005, 1637-2109, 1645-2131, 1646-2192, 1656-1963, 1668-1861, 1701-2018, 1701-2214, |
| | 1709-1911, 1709-1932, 1748-1968, 1748-2072, 1777-2117, 1873-2164, 1900-2333, 1900-2412, 2035-2483 |
| 96/7503257CB1/ | 1-85, 1-96, 1-374, 1-416, 1-432, 1-554, 1-568, 1-573, 1-581, 1-598, 1-647, 1-648, 1-651, 1-666, 1-2417, 1-2688, 3-586, |
| 2688 | 5-85, 17-474, 17-647, 22-478, 22-480, 45-753, 48-372, 52-420, 67-897, 68-577, 86-475, 86-478, 86-480, 86-663, |
| | 86-704, 90-613, 138-817, 154-813, 155-735, 155-766, 248-906, 249-944, 272-474, 272-478, 294-852, 300-839, |
| | 357-1028, 374-1028, 381-1028, 405-1061, 411-1028, 443-1129, 458-1028, 470-1102, 484-793, 484-1037, 484-1064, |
| | 484-1173, 532-1241, 533-1205, 551-1243, 572-1276, 585-1219, 585-1226, 587-737, 589-1248, 607-1212, 611-1314, |
| | 625-1212, 632-1189, 632-1344, 633-1212, 666-1373, 697-1363, 708-1282, 720-1363, 721-1119, 734-1421, 739-1278, |
| | 739-1412, 753-1339, 795-1363, 812-1243, 881-1541, 887-1094, 915-1283, 976-1226, 999-1701, 1101-1753, |
| | 1127-1748, 1129-1753, 1136-1753, 1190-1753, 1213-1351, 1356-2093, 1369-1542, 1427-1940, 1427-2066, 1427-2068, |
| | 1433-2073, 1437-1966, 1487-1995, 1514-2218, 1539-2145, 1550-2263, 1555-2108, 1579-1994, 1606-2273, |
| | 1619-2264, 1621-2175, 1622-2030, 1622-2317, 1634-2143, 1635-2104, 1648-2092, 1657-2175, |
| | 1672-1921, 1708-2389, 1713-2273, 1714-2360, 1716-1946, 1716-2389, 1727-1983, 1741-2100, 1748-2267, 1753-2353, |
| | 1805-2023, 1826-2319, 1829-2212, 1844-2316, 1852-2338, 1853-2399, 1875-2068, 1908-2225, 1908-2421, |
| | 1916-2118, 1916-2139, 1955-2175, 1955-2279, 1984-2324, 2080-2371, 2107-2538, 2107-2617, 2242-2688 |
| 97/7504472CB1/ | 1-224, 1-286, 1-432, 1-486, 1-2579, 9-109, 9-225, 9-725, 9-786, 9-862, 10-109, 11-109, 12-109, 12-278, 12-391, 12-783, |
| 2852 | 13-109, 15-779, 15-837, 17-109, 22-270, 29-512, 30-338, 150-532, 532-730, 760-969, 846-1060, 852-1486, 924-1126, |
| | 948-1505, 958-1491, 983-1260, 984-1262, 1067-1318, 1126-1416, 1167-1417, 1178-1410, 1243-1492, 1243-1804, |
| | 1255-1516, 1255-1793, 1255-1912, 1343-1624, 1353-1863, 1355-1573, 1362-2000, 1414-1904, 1479-2024, |
| | 1578-2072, 1586-2406, 1588-2485, 1590-2491, 1594-2067, 1605-2491, 1652-2488, 1653-2491, 1656-2484, 1663-2485, |
| | 1678-2488, 1682-1942, 1689-1907, 1689-2169, 1695-1945, 1696-2488, 1710-1908, 1710-1931, 1711-2429, |
| | 1720-2488, 1723-2483, 1757-2491, 1763-1992, 1767-2423, 1786-2493, 1814-2488, 1814-2491, 1833-2066, 1835-2491, |
| | 1845-2563, 1862-2452, 1871-2491, 1897-2483, 1897-2485, 1897-2491, 1899-2485, 1899-2489, 1899-2491, |
| | 1902-2488, 1911-2491, 1911-2503, 1921-2152, 1928-2498, 1935-2515, 1943-2350, 1951-2515, 1961-2441, 1975-2515, |
| | 2002-2291, 2005-2363, 2029-2577, 2038-2515, 2047-2269, 2072-2498, 2107-2491, 2146-2498, |
| | 2164-2529, 2165-2496, 2194-2498, 2201-2489, 2226-2498, 2259-2558, 2265-2513, 2287-2542, 2301-2543, 2322-2495, |
| | 2326-2489, 2326-2563, 2327-2501, 2329-2852, 2364-2497 |
| 98/7504475CB1/ | 1-52, 1-502, 1-2411, 3-52, 9-53, 22-52, 177-441, 177-729, 178-360, 178-462, 179-656, 183-1013, 186-875, 202-1013, |
| 2411 | 210-462, 217-477, 227-497, 230-922, 282-1036, 300-1013, 302-1013, 337-639, 337-769, 337-971, 380-885, |
| | 380-893, 388-583, 408-555, 426-861, 491-1111, 516-1172, 528-812, 528-825, 532-1096, 556-1072, 560-1163, 570-1013, |
| | 600-1173, 641-1188, 657-1131, 672-945, 677-1325, 682-929, 683-1260, 689-958, 712-1054, 716-862, 717-1181, |
| | 744-1083, 747-1559, 748-1140, 758-1025, 768-1370, 779-997, 779-1221, 809-1429, 814-1448, 815-1257, 824-1112, |
| | 824-1124, 824-1375, 824-1550, 824-1553, 824-1561, 824-1562, 858-1467, 925-1496, 945-1494, 961-1202, |
| | 976-1455, 977-1215, 1008-1593, 1008-1595, 1035-1255, 1037-1269, 1037-1391, 1037-1540, 1042-1328, 1042-1564, |
| | 1062-1514, 1078-1388, 1100-1376, 1126-1364, 1137-1372, 1137-1422, 1143-1447, 1150-1780, 1158-1433, |
| | 1166-1564, 1178-1431, 1193-1363, 1212-1552, 1217-1445, 1240-1564, 1246-1403, 1246-1533, 1262-1490, 1266-1517, |
| | 1291-1518, 1291-1564, 1295-1558, 1296-1691, 1308-1549, 1309-1564, 1322-1495, 1324-1970, |
| | 1420-1672, 1420-1701, 1424-1951, 1447-1931, 1469-2011, 1505-2011, 1557-1997, 1563-1953, 1564-1791, 1564-1803, |
| | 1564-1997, 1567-2011, 1570-1956, 1579-1916, 1587-1998, 1592-1711, 1592-1780, 1592-2002, 1592-2003, |
| | 1602-1996, 1602-1999, 1605-1994, 1611-1909, 1637-2002, 1647-1963, 1655-1771, 1660-2131, 1666-1986, 1666-1997, |
| | 1666-1999, 1666-2010, 1668-2131, 1685-1997, 1716-1997, 1788-1962, 1807-1995, 1818-1997, 1819-2110, |
| | 1849-1981, 1857-2061, 1857-2409, 1861-1997, 1876-2013, 1876-2105, 1876-2107, 1892-1995, 1958-2373, 2015-2296, |
| | 2017-2290, 2029-2272, 2029-2316, 2029-2409, 2036-2275, 2037-2358, 2039-2313, 2050-2299, 2080-2345, |
| | 2092-2380, 2156-2366 |
| 99/7503104CB1/ | 1-286, 1-458, 1-511, 1-587, 1-595, 1-621, 1-640, 66-335, 66-902, 67-542, 83-598, 107-625, 111-371, 119-369, 121-520, |
| 1636 | 126-390, 127-434, 128-573, 131-419, 131-593, 132-390, 133-548, 136-243, 136-340, 136-359, 136-381, 136-390, |
| | 136-424, 136-466, 136-483, 136-500, 137-1594, 138-722, 146-398, 146-407, 148-301, 148-444, 149-397, 151-389, |
| | 151-554, 153-405, 153-430, 153-450, 154-414, 154-431, 176-430, 196-477, 199-762, 200-465, 204-484, 205-724, |
| | 211-329, 211-492, 213-543, 218-519, 219-396, 226-415, 226-486, 234-520, 248-475, 253-743, 255-567, 257-424, |
| | 262-385, 262-475, 262-527, 262-544, 263-471, 263-511, 263-517, 271-397, 281-810, 281-812, 287-389, 295-545, |
| | 297-394, 303-547, 305-877, 307-796, 315-585, 326-643, 328-604, 336-581, 337-614, 341-589, 344-581, 351-662, |
| | 352-595, 352-600, 359-604, 360-622, 397-655, 399-749, 403-599, 403-1086, 404-991, 412-680, 412-681, 412-682, |
| | 416-690, 420-581, 421-544, 426-693, 431-646, 446-715, 446-1077, 448-693, 450-642, 463-737, 463-907, 463-930, |
| | 463-934, 464-717, 467-726, 487-735, 487-771, 489-759, 490-660, 500-873, 501-824, 505-757, 511-774, |
| | 511-1124, 518-992, 521-729, 528-756, 532-797, 532-931, 534-815, 534-820, 534-1124, 537-764, 537-826, 547-769, |
| | 547-792, 549-792, 550-1076, 564-761, 564-821, 564-863, 565-770, 573-1092, 583-820, 583-947, 583-983, 583-987, |
| | 583-1028, 583-1074, 583-1093, 584-838, 589-930, 593-833, 600-863, 600-1065, 600-1076, 600-1095, 600-1100, |
| | 600-1124, 602-922, 602-927, 603-811, 603-848, 603-1107, 617-892, 624-820, 624-822, 624-873, 624-1113, 625-900, |
| | 626-852, 626-1115, 627-1045, 631-930, 635-798, 637-874, 637-877, 637-889, 646-928, 665-900, 665-944, 666-865, |
| | 666-890, 666-930, 666-950, 668-938, 670-926, 672-942, 684-986, 686-893, 687-931, 687-965, 690-1124, 698-932, |
| | 698-989, 699-972, 707-1038, 710-943, 710-964, 710-1121, 715-1052, 717-902, 723-997, 724-923, 724-975, |
| | 724-1028, 725-1123, 725-1124, 732-1020, 734-1001, 734-1008, 735-1025, 737-1047, 739-974, 746-1025, 747-1120, |
| | 747-1121, 761-1222, 762-1057, 767-1006, 768-1054, 769-992, 769-1059, 771-1004, 771-1038, 771-1057, 771-1110, |
| | 777-902, 786-1079, 789-940, 789-1062, 789-1068, 789-1117, 790-981, 799-1049, 802-1059, 807-1124, 810-1058, |
| | 811-994, 812-1072, 816-1091, 818-1074, 820-1089, 824-1033, 824-1109, 826-954, 826-1087, 827-1071, |
| | 831-1099, 837-1080, 837-1087, 848-1120, 877-1014, 919-1114, 946-1427, 994-1431, 1016-1124, 1030-1444, 1047-1328, |
| | 1054-1277, 1054-1427, 1054-1440, 1057-1416, 1133-1379, 1135-1237, 1135-1368, 1141-1432, 1154-1633, |
| | 1156-1370, 1169-1358, 1170-1268, 1170-1464, 1174-1422, 1174-1631, 1175-1437, 1179-1440, 1215-1448, 1222-1486, |
| | 1227-1434, 1229-1526, 1229-1531, 1242-1446, 1244-1527, 1244-1538, 1245-1504, 1251-1496, 1257-1471, |
| | 1292-1444, 1305-1510, 1316-1612, 1319-1595, 1346-1557, 1352-1626, 1353-1423, 1365-1636, 1369-1603, 1412-1583 |
| 100/7503106CB1/ | 1-248, 12-261, 61-310, 62-1507, 75-302, 194-458, 194-598, 194-674, 194-718, 194-815, 247-342, 320-732, 324-516, |
| 1681 | 324-873, 329-961, 331-573, 334-872, 343-967, 352-924, 357-603, 371-922, 386-944, 399-1013, 406-710, 409-1041, |
| | 417-654, 417-828, 424-978, 426-714, 427-702, 438-938, 443-597, 450-705, 468-730, 473-1069, 475-571, 488-1064, |
| | 510-1227, 521-968, 554-1414, 555-805, 578-1110, 583-1226, 587-814, 598-1148, 606-1149, 610-1054, 620-877, |
| | 639-883, 646-1138, 654-953, 654-1253, 663-825, 663-932, 663-936, 672-1334, 673-888, 690-952, 700-987, |
| | 700-1163, 716-1150, 722-1224, 744-957, 744-1092, 747-957, 751-1421, 753-1625, 754-1354, 759-1153, 785-1311, |
| | 788-1370, 794-1299, 795-895, 801-1302, 805-1446, 811-1348, 813-1102, 833-1161, 839-1216, 850-1100, 863-1138, |
| | 869-1012, 876-1318, 884-1452, 886-1168, 889-1262, 891-1094, 896-1204, 897-1174, 898-1235, 902-1254, 962-1157, |
| | 962-1212, 969-1208, 978-1223, 1004-1412, 1021-1265, 1027-1271, 1027-1309, 1027-1681, 1063-1551, 1097-1266, |
| | 1097-1371, 1111-1360, 1111-1486, 1113-1347, 1118-1412, 1122-1338, 1154-1312, 1154-1448, |
| | 1164-1328, 1170-1436, 1173-1395, 1180-1461, 1181-1436, 1189-1424, 1193-1447, 1199-1470, 1204-1485, 1208-1491, |
| | 1209-1398, 1209-1457, 1218-1478, 1226-1368, 1231-1532, 1234-1488, 1256-1507, 1267-1610, 1279-1458, |
| | 1280-1471, 1290-1507, 1290-1556, 1305-1543, 1311-1518 |
| 101/7503176CB1/ | 1-258, 1-1226, 2-237, 55-241, 55-317, 59-659, 66-357, 84-346, 104-292, 115-376, 115-378, 115-399, 156-422, 164-456, |
| 1301 | 189-494, 198-414, 198-479, 207-476, 212-484, 213-413, 215-413, 219-466, 240-644, 243-549, 246-800, 255-483, |
| | 266-723, 280-747, 280-782, 280-799, 287-548, 302-539, 304-604, 317-591, 326-586, 345-886, 348-634, 353-871, |
| | 358-659, 376-859, 379-900, 387-557, 394-595, 394-607, 400-647, 409-623, 409-1219, 414-662, 419-1032, 439-883, |
| | 439-937, 446-549, 450-705, 454-647, 454-828, 464-1045, 466-668, 466-968, 474-734, 481-894, 483-799, 488-761, |
| | 493-856, 497-742, 523-1032, 544-1145, 546-1198, 547-801, 551-1234, 564-1216, 568-771, 574-658, 576-863, |
| | 578-838, 580-841, 581-1196, 582-1100, 582-1126, 589-1197, 597-984, 603-1198, 604-1206, 611-1166, 623-1222, |
| | 633-883, 635-1133, 650-1229, 653-1196, 655-888, 655-890, 696-970, 700-901, 700-1222, 705-1166, 712-1211, 730-1005, |
| | 731-1000, 735-1211, 738-1244, 749-1211, 751-1211, 751-1217, 766-1170, 781-1211, 782-1064, 782-1227, |
| | 784-1229, 785-1211, 787-1195, 789-1213, 791-1211, 792-1212, 796-1212, 798-1213, 800-984, 805-1211, |
| | 807-1225, 815-1209, 817-1212, 827-1215, 831-1211, 870-1211, 882-1214, 891-1210, 913-1213, 914-1210, 916-1211, |
| | 919-1209, 919-1229, 924-1212, 927-1208, 928-1211, 937-1209, 946-1207, 960-1209, 974-1225, 997-1211, |
| | 1008-1209, 1008-1257, 1011-1216, 1047-1212, 1058-1211, 1060-1215, 1063-1225, 1064-1225, 1067-1213, 1071-1301, |
| | 1076-1226, 1080-1210, 1084-1211, 1088-1209, 1095-1207, 1095-1221, 1095-1264, 1125-1236 |
| 102/7503202CB1/ | 1-1839, 249-706, 1056-1314, 1071-1333, 1127-1383, 1137-1398, 1153-1389, 1153-1405, 1168-1824, 1188-1608, |
| 1848 | 1196-1486, 1201-1458, 1214-1486, 1234-1485, 1239-1486, 1333-1526, 1333-1847, 1448-1675, 1468-1749, 1468-1754, |
| | 1483-1755, 1524-1589, 1528-1724, 1549-1848, 1638-1848 |
| 103/7503249CB1/ | 1-132, 1-133, 5-581, 5-1502, 5-1547, 30-133, 139-417, 139-689, 139-710, 139-818, 397-490, 462-600, 668-909, 668-922, |
| 1547 | 668-941, 668-959, 668-1245, 668-1263, 676-905, 676-1376, 678-1164, 685-1164, 699-976, 728-1408, 736-1186, |
| | 739-1292, 749-1334, 769-1369, 774-1056, 810-959, 810-1060, 817-1399, 817-1426, 818-1286, 836-1192, 887-1380, |
| | 919-1082, 989-1501, 1008-1493, 1073-1332, 1241-1502 |
| 104/7505890CB1/ | 1-264, 1-516, 1-2614, 5-209, 10-285, 40-297, 59-348, 210-650, 229-784, 394-962, 395-1094, 402-1063, 404-1035, |
| 2614 | 455-1061, 476-1090, 495-1088, 563-1176, 666-1188, 687-1362, 771-1042, 825-1373, 872-1038, 874-1110, 944-1183, |
| | 944-1226, 944-1300, 964-1522, 965-1522, 987-1608, 1100-1364, 1100-1593, 1100-1596, 1105-1335, 1105-1370, |
| | 1181-1820, 1245-1503, 1246-1459, 1246-1596, 1250-1821, 1285-1853, 1362-2088, 1364-1944, 1378-1802, |
| | 1382-1997, 1404-1690, 1415-1663, 1418-2132, 1429-1670, 1437-1760, 1461-1741, 1462-1488, 1462-1777, 1463-1880, |
| | 1469-1606, 1480-1605, 1481-2115, 1482-2013, 1496-2041, 1523-1759, 1541-2216, 1551-2254, 1566-1843, |
| | 1566-1849, 1572-1814, 1573-1824, 1577-2044, 1585-1805, 1585-1806, 1585-1813, 1585-2094, 1585-2221, 1586-2162, |
| | 1594-1900, 1600-2150, 1601-2088, 1629-1865, 1644-2583, 1660-2197, 1666-1938, 1667-2355, 1672-1974, |
| | 1672-1981, 1672-1984, 1674-2193, 1678-1892, 1678-1930, 1679-2105, 1683-1979, 1688-1961, 1688-2293, 1694-1908, |
| | 1698-2354, 1704-2189, 1733-2353, 1753-2034, 1754-2012, 1763-2012, 1769-2334, 1776-2448, 1780-2063, |
| | 1782-2072, 1782-2311, 1793-2109, 1794-1919, 1794-2395, 1794-2472, 1813-2068, 1828-2282, 1828-2353, 1828-2464, |
| | 1838-2086, 1838-2369, 1839-2478, 1840-2605, 1842-2111, 1842-2390, 1851-2126, 1854-2425, 1861-2130, |
| | 1862-2587, 1863-2308, 1867-2079, 1869-2552, 1878-2458, 1886-2556, 1887-2055, 1889-2559, 1892-2123, 1894-2611, |
| | 1896-2061, 1896-2166, 1896-2219, 1905-2526, 1907-2174, 1917-2357, 1919-2583, 1923-2589, 1925-2232, |
| | 1927-2219, 1938-2576, 1939-2592, 1945-2188, 1958-2505, 1963-2219, 1971-2222, 1978-2578, 1988-2603, 2000-2134, |
| | 2001-2559, 2002-2612, 2009-2217, 2009-2253, 2010-2268, 2014-2529, 2017-2311, 2017-2482, 2020-2514, |
| | 2022-2269, 2025-2592, 2027-2304, 2029-2317, 2031-2274, 2031-2535, 2035-2273, 2037-2206, 2050-2592, 2057-2593, |
| | 2074-2329, 2081-2612, 2086-2372, 2087-2342, 2087-2387, 2087-2594, 2087-2604, 2088-2305, 2097-2386, |
| | 2107-2394, 2112-2584, 2116-2604, 2121-2580, 2122-2392, 2123-2369, 2123-2370, 2123-2432, 2127-2614, 2130-2588, |
| | 2131-2614, 2132-2368, 2132-2394, 2132-2591, 2136-2422, 2136-2436, 2137-2380, 2137-2609, 2137-2614, |
| | 2142-2599, 2142-2614, 2149-2595, 2154-2406, 2163-2599, 2164-2614, 2165-2389, 2171-2570, 2174-2587, |
| | 2178-2614, 2185-2595, 2185-2599, 2194-2599, 2195-2595, 2200-2477, 2210-2614, 2214-2532, 2214-2535, 2224-2473, |
| | 2226-2595, 2299-2418, 2491-2526, 2519-2614, 2532-2594 |

TABLE 5

| | | |
|---|---|---|
| Polynucleotide SEQ | | |
| ID NO: | Incyte Project ID: | Representative Library |

| | | |
|---|---|---|
| 53 | 7499969CB1 | NOSEDIC02 |
| 54 | 7499974CB1 | BRAUNOR01 |
| 55 | 7499976CB1 | TESTTUT02 |
| 56 | 7499954CB1 | BRAHNON05 |
| 57 | 7500827CB1 | LNODNON02 |
| 58 | 7948585CB1 | BRAIFEC01 |
| 59 | 7500002CB1 | LIVRNON08 |
| 60 | 7500012CB1 | BMARTXE01 |
| 61 | 1664071CB1 | DRGTNON04 |
| 62 | 6214577CB1 | PGANNON02 |
| 63 | 7502149CB1 | BRAXTDR15 |
| 64 | 7503480CB1 | PROSTMC01 |
| 65 | 7500017CB1 | BLADTUT04 |
| 66 | 7499955CB1 | TESTTUT02 |
| 67 | 7504025CB1 | HNT2TXN01 |
| 68 | 7503203CB1 | HNT2AGT01 |
| 69 | 7503260CB1 | SINTNOR01 |
| 70 | 2969494CB1 | CONRTUE01 |
| 71 | 7503201CB1 | BRACNOK02 |
| 72 | 7503262CB1 | BRAINOY02 |
| 73 | 7503409CB1 | HEAONOE01 |
| 74 | 7503499CB1 | CARGNOT01 |
| 75 | 90031281CB1 | BRSMTXF01 |
| 77 | 7500027CB1 | LNODNOT02 |
| 78 | 7504546CB1 | CARDNOT01 |
| 79 | 7503246CB1 | COLRTUE01 |
| 80 | 7505729CB1 | SKIRNOR01 |
| 81 | 7487334CB1 | SINTNOR01 |
| 82 | 7503109CB1 | COLNNOT16 |
| 83 | 7503128CB1 | BRSTNOT07 |
| 84 | 7503191CB1 | THP1PLB02 |
| 85 | 7503196CB1 | BRSTTUT13 |
| 86 | 7503254CB1 | BEPINON01 |
| 87 | 7503531CB1 | SMCCNOS01 |
| 89 | 7503180CB1 | LUNGNON03 |
| 90 | 7503206CB1 | GPCRDPV01 |
| 91 | 7503227CB1 | THYRDIE01 |
| 92 | 7504473CB1 | PROSNOT16 |
| 93 | 7503200CB1 | BRAINOT14 |
| 94 | 7500465CB1 | BRAVUNT02 |
| 95 | 7503256CB1 | LATRTUT02 |
| 96 | 7503257CB1 | LATRTUT02 |
| 97 | 7504472CB1 | NEUTFMT01 |
| 98 | 7504475CB1 | THP1NOT03 |
| 99 | 7503104CB1 | LIVRDIR01 |
| 100 | 7503106CB1 | UTRMTMT01 |
| 101 | 7503176CB1 | EPIPNON05 |
| 102 | 7503202CB1 | BRAINOT22 |
| 103 | 7503249CB1 | BONEUNT01 |
| 104 | 7505890CB1 | NGANNOT01 |

TABLE 6

| Library | Vector | Library Description |
|---|---|---|
| BEPINON01 | PSPORT | Normalized library was constructed from 5.12 million independent |
| | | clones from a bronchial epithelium library. RNA was |
| | | made from a bronchial epithelium primary cell line derived from a |
| | | 54-year-old Caucasian male. The normalization and hybridization conditions |
| | | were adapted from Soares et al., PNAS (1994) 91: 9228, using a longer |
| | | (24-hour) reannealing hybridization period. |
| BLADTUT04 | pINCY | Library was constructed using RNA isolated from bladder tumor tissue |
| | | removed from a 60-year-old Caucasian male during a radical cystectomy, |
| | | prostatectomy, and vasectomy. Pathology indicated grade 3 transitional |
| | | cell carcinoma in the left bladder wall. Carcinoma in-situ was |
| | | identified in the dome and trigone. Patient history included tobacco |
| | | use. Family history included type I diabetes, malignant neoplasm of the |
| | | stomach, atherosclerotic coronary artery disease, and acute myocardial infarction. |
| BMARTXE01 | pINCY | This 5′ biased random primed library was constructed using RNA isolated |
| | | from treated SH-SY5Y cells derived from a metastatic bone marrow |
| | | neuroblastoma, removed from a 4-year-old Caucasian female (Schering AG). |
| | | The medium was MEM/HAM'S F12 with 10% fetal calf serum. After reaching |
| | | about 80% confluency cells were treated with 6-Hydroxydopamine |
| | | (6-OHDA) at 100 microM for 8 hours. |
| BONEUNT01 | pINCY | Library was constructed using RNA isolated from Saos-2, a primary |
| | | osteogenic sarcoma cell line (ATCC HTB-85) derived |
| | | from an 11-year-old Caucasian female. |
| BRACNOK02 | PSPORT1 | This amplified and normalized library was constructed using RNA isolated from |
| | | posterior cingulate tissue removed from an 85-year-old Caucasian female |
| | | who died from myocardial infarction and retroperitoneal hemorrhage. |
| | | Pathology indicated atherosclerosis, moderate to severe, involving the |
| | | circle of Willis, middle cerebral, basilar and vertebral arteries; infarction, |
| | | remote, left dentate nucleus; and amyloid plaque deposition consistent |
| | | with age. There was mild to moderate leptomeningeal fibrosis, especially |
| | | over the convexity of the frontal lobe. There was mild generalized atrophy |
| | | involving all lobes. The white matter was mildly thinned. Cortical thickness |
| | | in the temporal lobes, both maximal and minimal, was slightly reduced. The |
| | | substantia nigra pars compacta appeared mildly depigmented. Patient history |
| | | included COPD, hypertension, and recurrent deep venous thrombosis. 6.4 million |
| | | independent clones from this amplified library were normalized in one round using |
| | | conditions adapted from Soares et al., PNAS (1994) 91: 9228-9232 and Bonaldo et al., |
| | | Genome Research 6 (1996): 791. |
| BRAHNON05 | pINCY | This normalized hippocampus tissue library was constructed from 1.6 |
| | | million independent clones from a hippocampus tissue library. Starting RNA |
| | | was made from posterior hippocampus removed from a 35-year-old Caucasian male who died |
| | | from cardiac failure. Pathology indicated moderate leptomeningeal fibrosis |
| | | and multiple microinfarctions of the cerebral neocortex. The cerebral |
| | | hemisphere revealed moderate fibrosis of the leptomeninges with focal |
| | | calcifications. There was evidence of shrunken and slightly eosinophilic pyramidal |
| | | neurons throughout the cerebral hemispheres. There were small microscopic areas |
| | | of cavitation with gliosis, scattered through the cerebral cortex. Patient |
| | | history included cardiomyopathy, CHF, cardiomegaly, an enlarged spleen and liver. |
| | | Patient medications included simethicone, Lasix, Digoxin, Colace, Zantac, captopril, |
| | | and Vasotec. The library was normalized in two rounds using conditions adapted |
| | | from Soares et al., PNAS (1994) 91: 9228 and Bonaldo et al., Genome Research 6 (1996): |
| | | 791, except that a significantly longer (48 hours/round) reannealing hybridization was used. |
| BRAIFEC01 | pINCY | This large size-fractionated library was constructed using RNA isolated |
| | | from brain tissue removed from a Caucasian male fetus who was stillborn |
| | | with a hypoplastic left heart at 23 weeks' gestation. |
| BRAINOT14 | pINCY | Library was constructed using RNA isolated from brain tissue removed |
| | | from the left frontal lobe of a 40-year-old Caucasian female during excision of a cerebral |
| | | meningeal lesion. Pathology for the associated tumor tissue indicated grade |
| | | 4 gemistocytic astrocytoma. |
| BRAINOT22 | pINCY | Library was constructed using RNA isolated from right temporal lobe tissue |
| | | removed from a 45-year-old Black male during a brain lobectomy. Pathology for |
| | | the associated tumor tissue indicated dysembryoplastic neuroepithelial tumor of the right |
| | | temporal lobe. The right temporal region dura was consistent with calcifying pseudotumor |
| | | of the neuraxis. Family history included obesity, benign hypertension, cirrhosis of |
| | | the liver, obesity, hyperlipidemia, cerebrovascular disease, and type II diabetes. |
| BRAINOY02 | pINCY | This large size-fractionated and normalized library was constructed using |
| | | pooled cDNA generated using mRNA isolated from midbrain, inferior temporal cortex, |
| | | medulla, and posterior parietal cortex tissues removed from a 35-year-old |
| | | Caucasian male who died from cardiac failure. Pathology indicated moderate leptomeningeal |
| | | fibrosis and multiple microinfarctions of the cerebral neocortex. Microscopically, |
| | | the cerebral hemisphere revealed moderate fibrosis of the leptomeninges with focal |
| | | calcifications. There was evidence of shrunken and slightly eosinophilic pyramidal neurons |
| | | throughout the cerebral hemispheres. Scattered throughout the cerebral cortex, there |
| | | were multiple small microscopic areas of cavitation with surrounding gliosis. Patient |
| | | history included dilated cardiomyopathy, congestive heart failure, cardiomegaly and an |
| | | enlarged spleen and liver, 0.28 million independent clones from this size-selected library |
| | | were normalized in two rounds using conditions adapted from Soares et al., PNAS (1994) |
| | | 91: 9228-9232 and Bonaldo et al., Genome Research 6 (1996): 791, except that a |
| | | significantly longer (48 hours/round) reannealing hybridization was used. |
| BRAUNOR01 | pINCY | This random primed library was constructed using RNA isolated from striatum, |
| | | globus pallidus and posterior putamen tissue removed from an 81-year-old Caucasian |
| | | female who died from a hemorrhage and ruptured thoracic aorta due to atherosclerosis. |
| | | Pathology indicated moderate atherosclerosis involving the internal carotids, bilaterally; |
| | | microscopic infarcts of the frontal cortex and hippocampus; and scattered diffuse amyloid |
| | | plaques and neurofibrillary tangles, consistent with age. Grossly, the leptomeninges |
| | | showed only mild thickening and hyalinization along the superior sagittal sinus. The |
| | | remainder of the leptomeninges was thin and contained some congested blood vessels. |
| | | Mild atrophy was found mostly in the frontal poles and lobes, and temporal lobes, |
| | | bilaterally. Microscopically, there were pairs of Alzheimer type II astrocytes within |
| | | the deep layers of the neocortex. There was increased satellitosis around neurons |
| | | in the deep gray matter in the middle frontal cortex. The amygdala contained |
| | | rare diffuse plaques and neurofibrillary tangles. The posterior hippocampus contained a |
| | | microscopic area of cystic cavitation with hemosiderin-laden |
| | | macrophages surrounded by reactive |
| BRAVUNT02 | PSPORT1 | Library was constructed using pooled RNA isolated from separate |
| | | populations of unstimulated astrocytes. |
| BRAXTDR15 | PCDNA2.1 | This random primed library was constructed using RNA isolated from |
| | | superior parietal neocortex tissue removed from a 55-year-old Caucasian female who |
| | | died from cholangiocarcinoma. Pathology indicated mild meningeal fibrosis predominately |
| | | over the convexities, scattered axonal spheroids in the white matter of the cingulate |
| | | cortex and the thalamus, and a few scattered neurofibrillary tangles in the |
| | | entorhinal cortex and the periaqueductal gray region. Pathology for the associated |
| | | tumor tissue indicated well-differentiated cholangiocarcinoma of the liver with |
| | | residual or relapsed tumor. Patient history included cholangiocarcinoma, post- |
| | | operative Budd-Chiari syndrome, biliary ascites, hydrothorax, dehydration, |
| | | malnutrition, oliguria and acute renal failure. Previous surgeries included |
| | | cholecystectomy and resection of 85% of the liver. |
| BRSMTXF01 | pRARE | This 5′ cap isolated full-length library was constructed using RNA isolated |
| | | from an Hs 578T cell line derived from a breast tumor, removed from a |
| | | 74-year-old Caucasian female. The cells were treated with 50 ng/mL of EGF |
| | | for 8 hours. Pathology indicated ductal carcinoma. |
| BRSTNOT07 | pINCY | Library was constructed using RNA isolated from diseased breast tissue removed |
| | | from a 43-year-old Caucasian female during a unilateral extended simple mastectomy. |
| | | Pathology indicated mildly proliferative fibrocystic changes with epithelial |
| | | hyperplasia, papillomatosis, and duct ectasia. Pathology for the associated tumor |
| | | tissue indicated invasive grade 4, nuclear grade 3 mammary adenocarcinoma with |
| | | extensive comedo necrosis. Family history included epilepsy, |
| | | cardiovascular disease, and type II diabetes. |
| BRSTTUT13 | pINCY | Library was constructed using RNA isolated from breast tumor tissue |
| | | removed from the right breast of a 46-year-old Caucasian female during a |
| | | unilateral extended simple mastectomy with breast reconstruction. Pathology |
| | | indicated an invasive grade 3 adenocarcinoma, ductal type with apocrine features |
| | | and greater than 50% intraductal component. Patient history included breast cancer. |
| CARDNOT01 | PBLUESCRIPT | Library was constructed using RNA isolated from the cardiac muscle of a |
| | | 65-year-old Caucasian male, who died from a gunshot wound |
| CARGNOT01 | pINCY | Library was constructed using RNA isolated from pooled cartilage obtained from |
| | | four donors: a 57-year-old Caucasian male who died of a heart attack; a 34-year-old |
| | | Caucasian male who died from cardiac failure; a 32-year-old Caucasian male |
| | | who died from a gunshot wound; and a 17-year-old female who died from an aortic aneurysm. |
| COLNNOT16 | pINCY | Library was constructed using RNA isolated from sigmoid colon tissue removed |
| | | from a 62-year-old Caucasian male during a sigmoidectomy and permanent colostomy. |
| COLRTUE01 | PSPORT1 | This 5′ biased random primed library was constructed using RNA isolated |
| | | from rectum tumor tissue removed from a 50-year-old Caucasian male |
| | | during closed biopsy of rectum and resection of rectum. Pathology indicated |
| | | grade 3 colonic adenocarcinoma which invades through the muscularis propria to |
| | | involve pericolonic fat. Tubular adenoma with low grade dysplasia was also |
| | | identified. The patient presented with malignant rectal neoplasm, blood in stool, |
| | | and constipation. Patient history included benign neoplasm of the large bowel, |
| | | hyperlipidemia. benign hypertension, alcohol abuse, and tobacco abuse. Previous |
| | | surgeries included above knee amputation and vasectomy. Patient medications included |
| | | allopurinol, Zantac, Darvocet, Centrum vitamins, and an unspecified stool softener. |
| | | Family history included congestive heart failure in the mother; and benign neoplasm |
| | | of the large bowel and polypectomy in the sibling(s). |
| CONRTUE01 | PCDNA2.1 | This 5′ biased random primed library was constructed using RNA |
| | | isolated from para-aortic soft tissue tumor tissue removed from a 74-year-old |
| | | Caucasian female during exploratory laparotomy and soft tissue excision. |
| | | Pathology indicated low-grade, leiomyosarcoma forming a well circumscribed mass |
| | | situated approximately 3.5 cm from the retroperitoneum. Paraffin section |
| | | immunostains for desmin actin and vimentin are positive in neoplastic cells. |
| | | The patient presented with soft tissue cancer. Patient history included benign |
| | | hypertension, hyperlipidemia and normal delivery. Previous surgeries |
| | | included closed liver biopsy and total abdominal hysterectomy. Patient medications |
| | | included atenolol and aspirin. Family history included congestive heart failure |
| | | in the mother; congestive heart failure in the father; and congestive heart failure, |
| | | multiple myloma, and type II diabetes in the sibling(s). |
| DRGTNON04 | pINCY | The normalized dorsal root ganglion tissue library was constructed |
| | | from 5.64 million independent clones from the a dorsal root ganglion library. |
| | | Starting RNA was made from thoracic dorsal root ganglion tissue from a 32-year-old |
| | | Caucasian male, who died from acute pulmonary edema, acute bronchopneumonia, |
| | | pleural and pericardial effusion, and lymphoma. The patient presented with pyrexia, |
| | | fatigue, and GI bleeding. Patient history included probable cytomegalovirus infection, |
| | | liver congestion and steatosis, splenomegaly, hemorrhagic cystitis, thyroid hemorrhage, |
| | | respiratory failure, pneumonia, natural killer cell lymphoma of the pharynx, |
| | | Bell' spalsy, and tobacco and alcohol abuse. The library was normalized in one |
| | | round using conditions adapted from Soares et al., PNAS (1994) 91: 9228 and Bonaldo |
| | | et al., Genome Research 6 (1996): 791, except that a significantly longer |
| | | (48-hours/round) reannealing hybridization was used. The library was then |
| | | linearized and recircularized to select for insert containing clones as follows: |
| | | plasmid DNA was prepped from approximately 1 million clones from the |
| | | normalized dorsal root ganglion tissue library following soft agar transformation. |
| EPIPNON05 | pINCY | This normalized prostate epithelial cell tissue library was constructed from |
| | | 2.36 million independent clones from a prostate epithelial cell tissue library. |
| | | Starting RNA was made from untreated prostatic epithelial cell issue removed from a |
| | | 17-year-old Hispanic male. The library was normalized in two rounds using conditions |
| | | adapted from Soares et al., PNAS (1994) 91: 9228 and Bonaldo et al., Genome Research |
| | | (1996) 6: 791, except that a significantly longer (48-hours/round) |
| | | reannealing hybridization was used. |
| GPCRDPV01 | PCR2-TOPOTA | Library was constructed using pooled cDNA from different donors. cDNA was |
| | | generated using mRNA isolated from the following: aorta, cerebellum, lymph nodes, muscle, |
| | | tonsil (lymphoid hyperplasia), bladder tumor (invasive grade 3 transitional cell |
| | | carcinoma.), diseased breast (proliferative fibrocystic changes without atypia |
| | | characterized by epithelial ductal hyperplasia, testicle tumor (embryonal carcinoma), |
| | | spleen, ovary, parathyroid, ileum, breast skin, sigmoid colon, penis tumor |
| | | (fungating invasive grade 4 squamous cell carcinoma), fetal lung, breast, fetal |
| | | small intestine, fetal liver, fetal pancreas, fetal lung, fetal skin, fetal penis, |
| | | fetal bone, fetal ribs, frontal brain tumor (grade 4 gemistocytic astrocytoma), |
| | | ovary (stromal hyperthecosis), bladder, bladder tumor (invasive grade 3 transitional |
| | | cell carcinoma), stomach, lymph node tumor (metastatic basaloid squamous cell carcinoma), |
| | | tonsil (reactive lymphoid hyperplasia), periosteum from the tibia, |
| | | fetal brain, fetal spleen, uterus tumor, endometrial (grade 3 adenosquamous |
| | | carcinoma), seminal vesicle, liver, aorta, adrenal gland, lymph node (metastatic |
| | | grade 3 squamous cell carcinoma), glossal muscle, esophagus, esophagus tumor (inv |
| HEAONOE01 | PCDNA2.1 | This 5′ biased random primed library was constructed using RNA isolated |
| | | from the aorta of a 39-year-old Caucasian male, who died from a gunshot wound. |
| | | Serology was positive for cytomegalovirus (CMV). Patient history included tobacco abuse |
| | | (one pack of cigarettes per day for 25 years), and occasionally |
| | | cocaine, marijuana, and alcohol use. |
| HNT2AGT01 | PBLUESCRIPT | Library was constructed at Stratagene (STR937233), using RNA |
| | | isolated from the hNT2 cell line derived from a human teratocarcinoma that |
| | | exhibited properties characteristic of a committed neuronal precursor. Cells |
| | | were treated with retinoic acid for 5 weeks and with mitotic inhibitors for two |
| | | weeks and allowed to mature for an additional 4 weeks in conditioned medium. |
| HNT2TXN01 | pRARE | This normalized NT2 cell line library was constructed from |
| | | independent clones from a treated NT2 cell line library. |
| | | Starting RNA was made from an NT2 cell line derived from a human |
| | | teratocarcinoma, which exhibited properties characteristic of a committed |
| | | neuronal precursor at an early stage of development. Cells were treated for 4 |
| | | hours with 10 ng/mL each of Interleukin-3, Interleukin-4, Interleukin-5, Interleukin-7, |
| | | GM-CSF, and TGF beta; 50 ng/mL of Interleukin 10, 100 ng/mL of G-CSF, 20 ng/mL of |
| | | LIF, and 100 nM of Leptin pooled together. The library was normalized in one round |
| | | using conditions adapted from Soares et al., PNAS (1994) 91: 9228-9232 and Bonaldo |
| | | et al., Genome Research 6 (1996): 791, except that a significantly longer (48 hours/ |
| | | round) reannealing hybridization was used. |
| LATRTUT02 | pINCY | Library was constructed using RNA isolated from a myxoma removed |
| | | from the left atrium of a 43-year-old Caucasian male during annuloplasty. |
| | | Pathology indicated atrial myxoma. Patient history included pulmonary |
| | | insufficiency, acute myocardial infarction, atherosclerotic coronary |
| | | artery disease, hyperlipidemia, and tobacco use. Family history included benign |
| | | hypertension, acute myocardial infarction, atherosclerotic coronary |
| | | artery disease, and type II diabetes. |
| LIVRDIR01 | pINCY | The library was constructed using RNA isolated from diseased liver tissue |
| | | removed from a 63-year-old Caucasian female during a liver transplant. Patient |
| | | history included primary biliary cirrhosis diagnosed in 1989. Serology was |
| | | positive for anti-mitochondrial antibody. |
| LIVRNON08 | pINCY | This normalized library was constructed from 5.7 million independent |
| | | clones from a pooled liver tissue library. Starting RNA was made from pooled |
| | | liver tissue removed from a 4-year-old Hispanic male who died from anoxia and |
| | | a 16 week female fetus who died after 16-weeks gestation from anencephaly. |
| | | Serologies were positive for cytolomegalovirus in the 4-year-old. Patient |
| | | history included asthma in the 4-year-old. Family history included taking daily |
| | | prenatal vitamins and mitral valve prolapse in the mother of the fetus. The |
| | | library was normalized in 2 rounds using conditions adapted from Soares et |
| | | al., PNAS (1994) 91: 9228 and Bonaldo et al., Genome Research 6 (1996): 791, except |
| | | that a significantly longer (48 hours/round) reannealing hybridization was used. |
| LNODNON02 | pINCY | This normalized lymph node tissue library was constructed from .56 million |
| | | independent clones from a lymph node tissue library. Starting RNA was made |
| | | from lymph node tissue removed from a 16-month-old Caucasian male who died from |
| | | head trauma. Serologies were negative. Patient history included bronchitis. |
| | | Patient medications included Dopamine, Dobutamine, Vancomycin, Vasopressin, |
| | | Proventil, and Atarax. The library was normalized in two rounds using conditions |
| | | adapted from Soares et al., PNAS (1994) 91: 9228-9932 and Bonaldo et al., Genome |
| | | Research 6 (1996): 791, except that a significantly longer |
| | | (48 hours/round) reannealing hybridization was used. |
| LNODNOT02 | PSPORT1 | Library was constructed using RNA isolated from the lymph node tissue |
| | | of a 42-year-old Caucasian female, who died of cardiac arrest. |
| LUNGNON03 | PSPORT1 | This normalized library was constructed from 2.56 million |
| | | independent clones from a lung tissue library. RNA was made from lung tissue |
| | | removed from the left lobe of a 58-year-old Caucasian male during a |
| | | segmental lung resection. Pathology for the associated tumor tissue |
| | | indicated a metastatic grade 3 (of 4) osteosarcoma. Patient history |
| | | included soft tissue cancer, secondary cancer of the lung, prostate |
| | | cancer, and an acute duodenal ulcer with hemorrhage. Patient also received |
| | | radiation therapy to the retroperitoneum. Family history included prostate cancer, |
| | | breast cancer, and acute leukemia. The normalization and hybridization |
| | | conditions were adapted from Soares et al., PNAS (1994) 91: 9228; Swaroop |
| | | et al., NAR (1991) 19: 1954; and Bonaldo et al., Genome Research (1996) 6: 791. |
| NEUTFMT01 | PBLUESCRIPT | Library was constructed using total RNA isolated from peripheral |
| | | blood granulocytes collected by density gradient centrifugation through |
| | | Ficoll-Hypaque. The cells were isolated from buffy coat units obtained |
| | | from unrelated male and female donors. Cells were cultured in 10 nM fMLP for |
| | | 30 minutes, lysed in GuSCN, and spun through CsCl to obtain RNA for library |
| | | construction. Because this library was made from total RNA, it has an |
| | | unusually high proportion of unique singleton sequences, |
| | | which may not all come from polyA RNA species. |
| NGANNOT01 | PSPORT1 | Library was constructed using RNA isolated from tumorous |
| | | neuroganglion tissue removed from a 9-year-old Caucasian male during a soft tissue |
| | | excision of the chest wall. Pathology indicated a ganglioneuroma. |
| | | Family history included asthma. |
| NOSEDIC02 | PSPORT1 | This large size fractionated library was constructed |
| | | using RNA isolated from nasal polyp tissue. |
| PGANNON02 | PSPORT1 | This normalized paraganglion library was constructed with |
| | | 5.48 million independent clones from a paraganglionic tissue |
| | | library. Starting RNA was made from paraganglionic tissue removed from a |
| | | 46-year-old Caucasian male during exploratory laparotomy. Pathology indicated |
| | | a benign paraganglioma and was associated with a grade 2 renal cell carcinoma. The |
| | | normalization and hybridization conditions were adapted from Soares et al. (PNAS |
| | | (1994) 91: 9228-9232) using a significantly longer |
| | | (48-hour) reannealing hybridization period. |
| PROSNOT16 | pINCY | Library was constructed using RNA isolated from diseased prostate |
| | | tissue removed from a 68-year-old Caucasian male during a radical prostatectomy. |
| | | Pathology indicated adenofibromatous hyperplasia. Pathology for the associated tumor |
| | | tissue indicated an adenocarcinoma (Gleason grade 3 + 4). The patient |
| | | presented with elevated prostate specific antigen (PSA). During this |
| | | hospitalization, the patient was diagnosed with myasthenia gravis. |
| | | Patient history included osteoarthritis, and type II diabetes. Family history |
| | | included benign hypertension, acute myocardial infarction, |
| | | hyperlipidemia, and arteriosclerotic coronary artery disease. |
| PROSTMC01 | pINCY | Library was constructed using polyA RNA isolated from diseased prostate |
| | | tissue removed from a 55-year-old Caucasian male during a radical |
| | | prostatectomy, regional lymph node excision, and prostate needle biopsy. |
| | | Pathology indicated adenofibromatous hyperplasia. Pathology for the matched |
| | | tumor tissue indicated adenocarcinoma, Gleason grade 5 + 4, |
| | | forming a predominant mass involving the left side peripherally with |
| | | extension into the right posterior superior region. The |
| | | tumor invaded and perforated the capsule to involve periprostatic tissue |
| | | in the left posterior superior region. The left inferior and superior |
| | | posterior surgical margins were positive. The right and left seminal vesicles, |
| | | bladder neck tissue (after re-excision), and multiple pelvic lymph nodes |
| | | were negative for tumor. One (of 9) left pelvic lymph nodes was metastatically |
| | | involved. The patient presented with elevated prostate specific antigen (PSA). |
| | | Patient history included calculus of the kidney. Previous surgeries |
| | | included an adenotonsillectomy. Patient medications included Khats claw, an |
| | | herbal preparation. Family history included breast cancer in the mother; |
| | | lung cancer in the father; and breast cancer in the si |
| SINTNOR01 | PCDNA2.1 | This random primed library was constructed using RNA isolated from small |
| | | intestine tissue removed from a 31-year-old Caucasian female during Roux-en-Y |
| | | gastric bypass. Patient history included clinical obesity. |
| SKIRNOR01 | PCDNA2.1 | This random primed library was constructed using RNA isolated from |
| | | skin tissue removed from the breast of a 17-year-old Caucasian female |
| | | during bilateral reduction mammoplasty. Patient history included breast hypertrophy. |
| | | Family history included benign hypertension. |
| SMCCNOS01 | pINCY | This subtracted coronary artery smooth muscle cell library was |
| | | constructed using 7.56 × 10e6 clones from a coronary |
| | | artery smooth muscle cell library and was subjected to two rounds |
| | | of subtraction hybridization for 48 hours with 6.12 × 10e6 |
| | | clones from a second coronary artery smooth muscle cell library. The starting |
| | | library for subtraction was constructed using RNA isolated from coronary artery |
| | | smooth muscle cells removed from a 3-year-old Caucasian male. The cells were |
| | | treated with TNF alpha & IL-1 beta 10 ng/ml each for 20 hours. The |
| | | hybridization probe for subtraction was derived from a similarly constructed |
| | | library from RNA isolated from untreated coronary artery smooth |
| | | muscle cells from the same donor. Subtractive hybridization conditions were |
| | | based on the methodologies of Swaroop et al.,(NAR (1991) 19: 1954) and |
| | | Bonaldo, et al. (Genome Research (1996) 6: 791-806). |
| TESTTUT02 | pINCY | Library was constructed using RNA isolated from testicular tumor |
| | | removed from a 31-year-old Caucasian male during unilateral orchiectomy. |
| | | Pathology indicated embryonal carcinoma. |
| THP1NOT03 | pINCY | Library was constructed using RNA isolated from untreated THP-1 cells. |
| | | THP-1 is a human promonocyte line derived from the peripheral blood of a 1-year-old |
| | | Caucasian male with acute monocytic leukemia (ref: Int. J. Cancer (1980) 26: 171). |
| THP1PLB02 | PBLUESCRIPT | Library was constructed using RNA isolated from THP-1 cells cultured |
| | | for 48 hours with 100 ng/ml phorbol ester (PMA), followed by a 4-hour culture |
| | | in media containing 1 ug/ml LPS. THP-1 is a human promonocyte line derived from the |
| | | peripheral blood of a 1-year-old male with acute monocytic leukemia. |
| THYRDIE01 | PCDNA2.1 | This 5′ biased random primed library was constructed using RNA |
| | | isolated from diseased thyroid tissue removed from a 22-year-old Caucasian |
| | | female during closed thyroid biopsy, partial thyroidectomy, and regional |
| | | lymph node excision. Pathology indicated adenomatous hyperplasia. The patient |
| | | presented with malignant neoplasm of the thyroid. Patient history included |
| | | normal delivery, alcohol abuse, and tobacco abuse. Previous surgeries included |
| | | myringotomy. Patient medications included an unspecified type of |
| | | birth control pills. Family history included hyperlipidemia and depressive |
| | | disorder in the mother; and benign hypertension, congestive heart failure, and |
| | | chronic leukemia in the grandparent(s). |
| UTRMTMT01 | pINCY | Library was constructed using RNA isolated from myometrial |
| | | tissue removed from a 45-year-old Caucasian female during vaginal |
| | | hysterectomy and bilateral salpingo-oophorectomy. Pathology indicated the |
| | | myometrium was negative for tumor. Pathology for the matched tumor tissue |
| | | indicated multiple (23) subserosal, intramural, and submucosal leiomyomata. The |
| | | endometrium was in proliferative phase. The patient presented with stress |
| | | incontinence. Patient history included extrinsic asthma without status asthmaticus |
| | | and normal delivery. Previous surgeries included adenotonsillectomy. Patient |
| | | medications included Motrin, iron sulfate, Premarin, prednisone, Tylenol #3, |
| | | and Colace. Family history included cerebrovascular disease in the mother; |
| | | depression in the sibling(s); and atherosclerotic coronary artery disease and |
| | | depression in the grandparent(s). |

TABLE 7

| Program | Description | Reference | Parameter Threshold |
|---|---|---|---|
| ABI | A program that removes vector sequences and masks | Applied Biosystems, | |
| FACTURA | ambiguous bases in nucleic acid sequences. | Foster City, CA. | |
| ABI/ | A Fast Data Finder useful in | Applied Biosystems, | Mismatch < 50% |
| PARACEL | comparing and annotating amino | Foster City, CA; | |
| FDF | acid or nucleic acid sequences. | Paracel Inc., Pasadena, CA. | |
| ABI | A program that assembles nucleic acid sequences. | Applied Biosystems, | |
| AutoAssembler | | Foster City, CA. | |
| BLAST | A Basic Local Alignment Search Tool useful in | Altschul, S. F. et al. (1990) | ESTs: Probability |
| | sequence similarity search for amino acid and nucleic | J. Mol. Biol. 215: 403-410; | value = 1.0E−8 |
| | acid sequences. BLAST includes five functions: | Altschul, S. F. et al. (1997) | or less; |
| | blastp, blastn, blastx, tblastn, and tblastx. | Nucleic Acids Res. 25: 3389-3402. | Full Length sequences: |
| | | | Probability value = |
| | | | 1.0E−10 or less |
| FASTA | A Pearson and Lipman algorithm that searches for | Pearson, W. R. and | ESTs: fasta E |
| | similarity between a query sequence and a group of | D. J. Lipman (1988) Proc. Natl. | value = 1.06E−6; |
| | sequences of the same type. FASTA comprises as | Acad Sci. USA 85: 2444-2448; | Assembled ESTs: fasta |
| | least five functions: fasta, tfasta, fastx, tfastx, and | Pearson, W. R. (1990) Methods Enzymol. 183: 63-98; | Identity = 95% or |
| | ssearch. | and Smith, T. F. and M. S. Waterman (1981) | greater and |
| | | Adv. Appl. Math. 2: 482-489. | Matchlength = |
| | | | 200 bases or greater; |
| | | | fastx E value = |
| | | | 1.0E−8 or less; |
| | | | Full Length sequences: |
| | | | fastx score = |
| | | | 100 or greater |
| BLIMPS | A BLocks IMProved Searcher that matches a | Henikoff, S. and J. G. Henikoff (1991) | Probability value = |
| | sequence against those in BLOCKS, PRINTS, | Nucleic Acids Res. 19: 6565-6572; Henikoff, | 1.0E−3 or less |
| | DOMO, PRODOM, and PFAM databases to search | J. G. and S. Henikoff (1996) Methods | |
| | for gene families, sequence homology, and structural | Enzymol. 266: 88-105; and Attwood, T. K. et | |
| | fingerprint regions. | al. (1997) J. Chem. Inf. Comput. Sci. 37: 417-424. | |
| HMMER | An algorithm for searching a query sequence against | Krogh, A. et al. (1994) J. Mol. Biol. | PFAM, INCY, |
| | hidden Markov model (HMM)-based databases of | 235: 1501-1531; Sonnhammer, E. L. L. et al. | SMART or |
| | protein family consensus sequences, such as PFAM, | (1988) Nucleic Acids Res. 26: 320-322; | TIGRFAM hits: |
| | INCY, SMART and TIGRFAM. | Durbin, R. et al. (1998) Our World View, in | Probability |
| | | a Nutshell, Cambridge Univ. Press, pp. 1-350. | value = |
| | | | 1.0E−3 or less; |
| | | | Signal peptide hits: |
| | | | Score = 0 or greater |
| ProfileScan | An algorithm that searches for structural and | Gribskov, M. et al. (1988) CABIOS 4: 61-66; | Normalized quality |
| | sequence motifs in protein sequences that match | Gribskov, M. et al. (1989) Methods | score ≧ GCG |
| | sequence patterns defined in Prosite. | Enzymol. 183: 146-159; Bairoch, A. et al. | specified “HIGH” |
| | | (1997) Nucleic Acids Res. 25: 217-221. | value for that |
| | | | particular |
| | | | Prosite motif. |
| | | | Generally, score = |
| | | | 1.4-2.1. |
| Phred | A base-calling algorithm that examines automated | Ewing, B. et al. (1998) Genome Res. 8: 175-185; | |
| | sequencer traces with high sensitivity and probability. | Ewing, B. and P. Green (1998) Genome | |
| | | Res. 8: 186-194. | |
| Phrap | A Phils Revised Assembly Program including | Smith, T. F. and M. S. Waterman (1981) Adv. | Score = 120 or greater; |
| | SWAT and CrossMatch, programs based on efficient | Appl. Math. 2: 482-489; Smith, T. F. and | Match length = |
| | implementation of the Smith-Waterman algorithm, | M. S. Waterman (1981) J. Mol. Biol. 147: 195-197; | 56 or greater |
| | useful in searching sequence homology and | and Green, P., University of | |
| | assembling DNA sequences. | Washington, Seattle, WA. | |
| Consed | A graphical tool for viewing and editing Phrap | Gordon, D. et al. (1998) Genome Res. 8: 195-202. | |
| | assemblies. | | |
| SPScan | A weight matrix analysis program that scans protein | Nielson, H. et al. (1997) Protein Engineering | Score = 3.5 or greater |
| | sequences for the presence of secretory signal | 10: 1-6; Claverie, J. M. and S. Audic (1997) | |
| | peptides. | CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos | |
| | determine orientation. | (1996) Protein Sci. 5: 363-371. | |
| TMHMMER | A program that uses a hidden Markov model (HMM) | Sonnhammer, E.L. et al. (1998) Proc. Sixth | |
| | to delineate transmembrane segments on protein | Intl. Conf. On Intelligent Systems for Mol. | |
| | sequences and determine orientation. | Biol., Glasgow et al., eds., The Am. Assoc. | |
| | | for Artificial Intelligence (AAAI) Press, | |
| | | Menlo Park, CA, and MIT Press, Cambridge, | |
| | | MA, pp. 175-182. | |
| Motifs | A program that searches amino acid sequences for | Bairoch, A. et al. (1997) Nucleic Acids Res. | |
| | patterns that matched those defined in Prosite. | 25: 217-221; Wisconsin Package Program | |
| | | Manual, version 9, page M51-59, Genetics | |
| | | Computer Group, Madison, WI. | |

1. An isolated polypeptide selected from the group consisting of:
a) a polypeptide comprising an amino acid sequence selected from the group consisting of SEQ ID NO:1-51,
b) a polypeptide comprising a naturally occurring amino acid sequence at least 90% identical to an amino acid sequence selected from the group consisting of SEQ ID NO:1, SEQ ID NO:3-4, SEQ ID NO:7-9, SEQ ID NO:14-16, SEQ ID NO:21, SEQ ID NO:23, SEQ ID NO:26, SEQ ID NO:28-31, SEQ ID NO:33-34, and SEQ ID NO:39
c) a polypeptide consisting essentially of a naturally occurring amino acid sequence at least 99% identical to the amino acid sequence of SEQ ID NO:27,
d) a polypeptide comprising a naturally occurring amino acid sequence at least 98% identical to an amino acid sequence selected from the group consisting of SEQ ID NO:2, SEQ ID NO:22, SEQ ID NO:36, and SEQ ID NO:48,
e) a polypeptide comprising a naturally occurring amino acid sequence at least 97% identical to an amino acid sequence selected from the group consisting of SEQ ID NO:13 and SEQ ID NO:24,
f) a polypeptide comprising a naturally occurring amino acid sequence at least 96% identical to the amino acid sequence of SEQ ID NO:10,
g) a polypeptide comprising a naturally occurring amino acid sequence at least 95% identical to the amino acid sequence of SEQ ID NO:40,
h) a polypeptide comprising a naturally occurring amino acid sequence at least 94% identical to the amino acid sequence of SEQ ID NO:45,
i) a polypeptide comprising a naturally occurring amino acid sequence at least 92% identical to the amino acid sequence of SEQ ID NO:47,
j) a polypeptide comprising a naturally occurring amino acid sequence at least 91% identical to the amino acid sequence of SEQ ID NO:17,
k) a polypeptide consisting essentially of a naturally occurring amino acid sequence at least 90% identical to an amino acid sequence selected from the group consisting of SEQ ID NO:18-20, SEQ ID NO:32, SEQ ID NO:37-38, SEQ ID NO:41-44, SEQ ID NO:49-51
l) a biologically active fragment of a polypeptide having an amino acid sequence selected from the group consisting of SEQ ID NO:1-51, and
m) an immunogenic fragment of a polypeptide having an amino acid sequence selected from the group consisting of SEQ ID NO:1-51.

2. An isolated polypeptide of claim 1 comprising an amino acid sequence selected from the group consisting of SEQ ID NO:1-51.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:52-102.

6. A recombinant polynucleotide comprising a promoter sequence operably linked to a polynucleotide of claim 3.

7. A cell transformed with a recombinant polynucleotide of claim 6.

8. (canceled)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-51.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:52-102,
b) a polynucleotide comprising a naturally occurring polynucleotide sequence at least 90% identical to a polynucleotide sequence selected from the group consisting of SEQ ID NO:52-67, SEQ ID NO:69-75, SEQ ID NO:77-85, SEQ ID NO:88-97, and SEQ ID NO:99-101,
c) a polynucleotide comprising a naturally occurring polynucleotide sequence at least 98% identical to a polynucleotide sequence selected from the group consisting of SEQ ID NO:87 and SEQ ID NO:102,
d) a polynucleotide comprising a naturally occurring polynucleotide sequence at least 97% identical to the polynucleotide sequence of SEQ ID NO:68,
e) a polynucleotide comprising a naturally occurring polynucleotide sequence at least 93% identical to the polynucleotide sequence of SEQ ID NO:76,
f) a polynucleotide comprising a naturally occurring polynucleotide sequence at least 92% identical to a polynucleotide sequence selected from the group consisting of SEQ ID NO:86 and SEQ ID NO:98,
g) a polynucleotide complementary to a polynucleotide of a),
h) a polynucleotide complementary to a polynucleotide of b),
i) a polynucleotide complementary to a polynucleotide of c),
j) a polynucleotide complementary to a polynucleotide of d),
k) a polynucleotide complementary to a polynucleotide of e),
l) a polynucleotide complementary to a polynucleotide of f), and
m) an RNA equivalent of a)-l).

13. (canceled)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (canceled)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-51.

19. (canceled)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. (canceled)

22. (canceled)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. (canceled)

25. (canceled)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (canceled)

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-159. (canceled)
